(19) United States
(12) Patent Application Publication
(10) Pub. No.: US 2011/0054013 A1
(43) Pub. Date: Mar. 3, 2011

(54) TETRAHYDROPYRANOCHROMENE GAMMA SECRETASE INHIBITORS

(76) Inventor: John W. Clader, Cranford, NJ (US)

(21) Appl. No.: 12/869,010

(22) Filed: Aug. 26, 2010

Related U.S. Application Data
(60) Provisional application No. 61/236,939, filed on Aug. 26, 2009.

(51) Int. Cl. A61K 31/382; C07D 493/04; A61P 25/28

(52) U.S. Cl. 514/455; 549/387

(57) ABSTRACT

Disclosed are novel gamma secretase inhibitors of the formula:

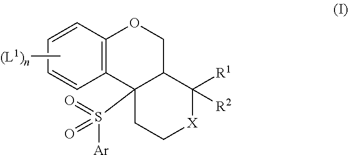

wherein at least one (e.g., 1-4, 1-3, 1-2, 2 or 1) H is replaced by deuterium. Also disclosed are methods of inhibiting gamma-secretase, methods of treating neurodegenerative diseases, and methods of treating Alzheimer's Disease.

### BACKGROUND

WO 00/50391, published Aug. 13, 2000, discloses compounds having a sulfonamide moiety that are useful for the treatment and prevention of Alzheimer's Disease and other diseases relating to the deposition of amyloid protein.

McCombie et al., Tetrahedron Letters, Vol. 34, No. 50, pp. 8033-8036 (1993) describe methods of preparing chromans and thiochromans. However, the chromans and thiochromans described therein are quite different from the compounds of the present invention.

In view of the present interest in the treatment or prevention of neurodegenerative diseases, such as Alzheimer's Disease, a welcome contribution to the art would be compounds for use in such treatment or prevention. This invention provides such a contribution.

### SUMMARY OF THE INVENTION

This invention provides compounds that are inhibitors (e.g., antagonists) of gamma-secretase (also termed “γ-secretase”) and have the Formula (I)

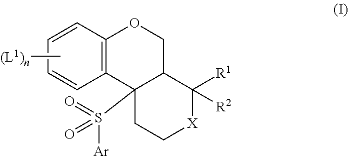

or a pharmaceutically acceptable salt, solvate, or ester thereof, wherein at least one (e.g., 1-4, 1-3, 1-2, 2 or 1) H in formula (I) is replaced by deuterium (D), and X, L1, R¹, R² and Ar are independently selected and are as defined below.

Thus, this invention is directed to compounds of formula (I) having at least one (e.g., 1-4, 1-3, 1-2, 2 or 1) deuterium atom.

In general the deuterium atoms in the compounds of formula (I) are those that have been added using deuterated reagents in the preparations of the compounds of formula (I).

This invention also provides compounds of formula (I) that have the formula (I.A1):

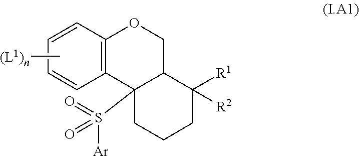

or a pharmaceutically acceptable salt, solvate, or ester thereof, wherein at least one (e.g., 1-4, 1-3, 1-2, 2 or 1) H in formula (I.A1) is replaced by deuterium (D), and L¹, R¹, R² and Ar are as defined below.

This invention also provides compounds of formula (I) that have the formula (I.A2):

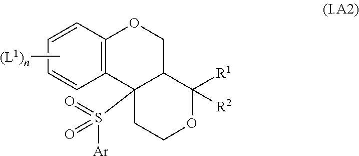

or a pharmaceutically acceptable salt, solvate, or ester thereof, wherein at least one (e.g., 1-4, 1-3, 1-2, 2 or 1) H in formula (I.A2) is replaced by deuterium (D), L¹, R¹, R² and Ar are as defined below.

This invention also provides the compounds of formula (I) in pure and isolated form.

This invention also provides compounds 12, 13, 22A to 22N, 22B-rac, 26A, 26B, 27, 28, 31, 33(−), 36, 37, 39, 44A, 44B, 55 to 57, 60, 70A, 71A(−), 75A-rac, 75B(−), 76A(−), 76A(+), 76B(−), 78A(−), 79A(−), 80A(+), 81A-rac, 81A(−), 81A(+), 81B(−), 82A, 83A, 84A, 85A, 85B(−), 86A, 87B, 95B-rac, 95B(−), 95B(+), 106(−), 107(−), 108(−), and 111(−) wherein at least one (e.g., 1-4, 1-3, 1-2, 2 or 1) H is replaced by deuterium.

This invention also provides compounds D-12, D-13, D1-22A, D2-22A, D3-22A and D-55.

This invention also provides a pharmaceutical composition comprising an effective amount of one or more compounds of Formula (I) and at least one pharmaceutically acceptable carrier.

This invention also provides (1) a method for inhibiting gamma-secretase, (2) a method of treating one or more neurodegenerative diseases, (3) a method of inhibiting the deposition of amyloid protein (e.g., amyloid beta protein) in, on or around neurological tissue (e.g., the brain), and (4) a method of treating Alzheimer's disease comprising administering an effective (i.e., therapeutically effective) amount of one or more compounds of formula (I) to a patient in need of treatment.

This invention also provides combination therapies for (1) inhibiting gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of one or more (e.g. one) compounds of formula (I) and the administration of one or more (e.g., one) other pharmaceutical active ingredients (e.g., drugs). The compounds of formula (I) and the other drugs can be administered separately (i.e., each is in its own separate dosage form), or the compounds of formula (I) can be combined with the other drugs in the same dosage form.

This invention also provides a kit comprising, in separate containers, in a single package, pharmaceutical compositions for use in combination, wherein one container comprises an effective amount of a compound of formula (I) (e.g., a compound of formula (I.A1), or a compound of formula (I.A2)) in a pharmaceutically acceptable carrier, and another container (i.e., a second container) comprises an effective amount of another pharmaceutically active ingredient (as described above), the combined quantities of the compound of formula (I) and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) inhibit gamma-secretase.

### DETAILED DESCRIPTION OF THE INVENTION

This invention provides novel compounds, useful as gamma secretase inhibitors, of the formula:

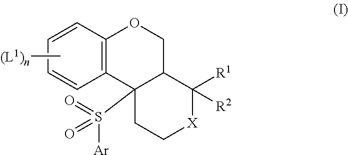

or a pharmaceutically acceptable salt, solvate, or ester thereof, wherein at least one (e.g., 1-4, 1-3, 1-2, 2 or 1) H in formula (I) is replaced by deuterium (D), wherein:

X is selected from the group consisting of O and CH₂;

R¹ is selected from the group consisting of: (1) -alkylene-S(O)₂—(C₁-C₆)alkyl, (2) -alkylene-S(O)₂—(C₁-C₆)haloalkyl; (3) -alkylene-S(O)₂—R⁶, (4) -alkylene-S(O)₂—R⁸, (5) -alkylene-S(O)₂-substituted(C₁-C₆)alkyl, (6) -alkylene-(tetrahydrothiophene 1,1-dioxide), (7) -alkenyl-S(O)₂—(C₁-C₆)alkyl, and (8) -cycloalkyl-S(O)₂—(C₁-C₆)alkyl;

wherein said -alkylene-S(O)₂-substituted(C₁-C₆)alkyl R¹ group is substituted with one or more substituents independently selected from the group consisting of: —OH, halo, —CN, —CF₃, —O—(C₁-C₆)alkyl (e.g., —OCH₃), and —O-(halo(C₁-C₆)alkyl) (e.g., —OCF₃), and preferably —OH, and more preferably one —OH, and wherein an example of said -alkylene-S(O)₂-substituted(C₁-C₆)alkyl R¹ group is -alkylene-S(O)₂—(C₁-C₆)hydroxyalkyl;

R² is selected from the group consisting of: H and alkyl (e.g., C₁-C₆alkyl or (C₁-C₂)alkyl), and in one example R² is H, and in another example R² is methyl;

R⁶ is selected from the group consisting of: (1) unsubstituted (C₆-C₁₄)aryl, (2) (C₆-C₁₄)aryl substituted with one or more L^{1A} groups, (3) unsubstituted (C₅-C₁₄)heteroaryl, (4) (C₅-C₁₄)heteroaryl substituted with one or more L^{1A} groups, (5) unsubstituted (C₅-C₁₄)heteroarylalkyl-, and (5) (C₅-C₁₄)heteroarylalkyl-substituted with one or more L^{1A} groups;

R⁸ is selected from the group consisting of unsubstituted cycloalkyl and cycloalkyl substituted with one or more L³ groups (wherein examples of said cycloalkyl groups (unsubstituted or substituted) include C₃-C₁₀ cycloalkyl rings);

each L³ is independently selected from the group consisting of: (1) —CN, (2) ═O, (3) —CH₂OH, (4) amino (i.e., —NH₂), (5) halo (e.g., Cl, F, and Br), (6) —CH₂NH₂, (7) —CH₂NHalkyl (such as, for example, —CH₂NH(C₁-C₆)alkyl), (8) —C(O)OH, (9) -alkylene-C(O)NH(C₁ to C₆)alkyl, (10) -alkylene-C(O)N((C₁ to C₆)alkyl)₂ wherein each alkyl is independently selected, (11) -alkylene-C(O)NH(C₁ to C₆)haloalkyl, and (12) -alkylene-C(O)N((C₁ to C₆)haloalkyl)₂ wherein each alkyl is independently selected);

Ar is selected from the group consisting of: (1) unsubstituted aryl (e.g., unsubstituted phenyl), (2) aryl (e.g., phenyl) substituted with one or more L^{1A} groups, (3) unsubstituted heteroaryl (e.g., pyridyl), and (4) substituted heteroaryl (e.g., substituted pyridyl) substituted with one or more L^{1A} groups;

each L1 is independently selected from the group consisting of: halogen, alkyl (e.g., C₁-C₆ alkyl), —CN, —CF₃, —O—(C₁-C₆)alkyl (e.g., —OCH₃), —O-(halo(C₁-C₆)alkyl) (e.g., —OCF₃ and —OCH₂CF₃), —C(O)—O—(C₁-C₆)alkyl (e.g., —C(O)OCH₃), -alkylene-OH (e.g., —CH₂OH), halo(C₁-C₆)alkyl (e.g., —CF₃), hydroxyalkoxy- (e.g., HOCH₂CH₂O—), alkoxyalkoxy- (e.g., CH₃OCH₂CH₂O—), and —S(O)₂(C₁-C₆)alkyl (e.g., —S(O)₂CH₂CH₃);

each L^{1A} is independently selected from the group consisting of: halogen, alkyl (e.g., C₁-C₆ alkyl), —CN, —CF₃, —O—(C₁-C₆)alkyl (e.g., —OCH₃), —O-(halo(C₁-C₆)alkyl) (e.g., —OCF₃ and —OCH₂CF₃), —C(O)—O—(C₁-C₆)alkyl (e.g., —C(O)OCH₃), -alkylene-OH (e.g., —CH₂OH), halo(C₁-C₆)alkyl (e.g., —CF₃), hydroxyalkoxy- (e.g., HOCH₂CH₂O—), alkoxyalkoxy- (e.g., CH₃OCH₂CH₂O—), and —S(O)₂(C₁-C₆)alkyl (e.g., —S(O)₂CH₂CH₃); and

n is 0, 1, 2 or 3.

In the compounds of this invention, the —S(O)₂— moiety of the R¹ substituents can be bound to any carbon of the alkylene chain. In general the —S(O)₂— moiety of the R¹ substituents is bound to the terminal carbon of the alkylene chain.

An example of a tetrahydrothiophene 1,1-dioxide moiety is:

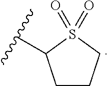

Thus, and example of the -alkylene-(tetrahydrothiophene 1,1-dioxide) R1 moiety is:

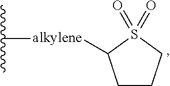

such as, for example,

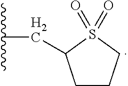

An example of the -alkenyl-S(O)₂—(C₁-C₆)alkyl R¹ moiety is —(C₂ to C₆)alkenyl-S(O)₂—(C₁-C₆)alkyl, such as, for example, —(C₂ to C₃)alkenyl-S(O)₂—(C₁-C₆)alkyl, such as, for example, —CH═CH—S(O)₂—(C₁-C₆)alkyl.

An example of the -cycloalkyl-S(O)₂—(C₁-C₆)alkyl R¹ moiety is —(C₃ to C₆)cycloalkyl-S(O)₂—(C₁-C₆)alkyl, such as for example, —(C₃ to C₅)cycloalkyl-S(O)₂—(C₁-C₆)alkyl, such as, for example, -cyclopropyl-S(O)₂—(C₁-C₆)alkyl.

An example of the heteroarylalkyl moiety of the R⁶ unsubstituted (C₅-C₁₄)heteroarylalkyl or the substituted (C₅-C₁₄)heteroarylalkyl substituent is furanyl-alkyl-, such as, for example, furanyl-CH₂—, such as, for example,

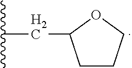

Compounds of formula (I) include compounds of formula (Ii):

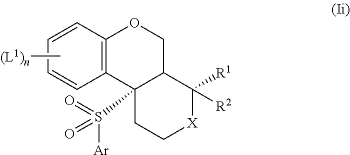

wherein at least one (e.g., 1-4, 1-3, 1-2, 2 or 1) H in formula (I) is replaced by deuterium (D).

Compounds of formula (I) include compounds of formula (Iii):

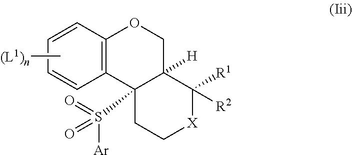

wherein at least one (e.g., 1-4, 1-3, 1-2, 2 or 1) H in formula (I) is replaced by deuterium (D).

Compounds of formula (I) include compounds of formula (Iiii):

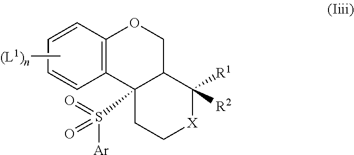

wherein at least one (e.g., 1-4, 1-3, 1-2, 2 or 1) H in formula (I) is replaced by deuterium (D).

Compounds of formula (I) include compounds of formula (Iiv):

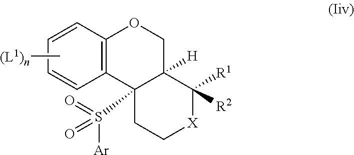

wherein at least one (e.g., 1-4, 1-3, 1-2, 2 or 1) H in formula (I) is replaced by deuterium (D).

Compounds of formula (I) include compounds of formula (Iv):

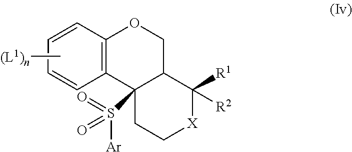

wherein at least one (e.g., 1-4, 1-3, 1-2, 2 or 1) H in formula (I) is replaced by deuterium (D).

Compounds of formula (I) include compounds of formula (Ivi):

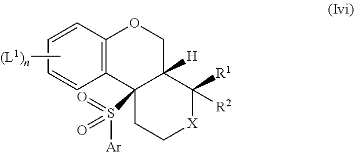

wherein at least one (e.g., 1-4, 1-3, 1-2, 2 or 1) H in formula (I) is replaced by deuterium (D).

Compounds of formula (I) include compounds of formula (Ivii):

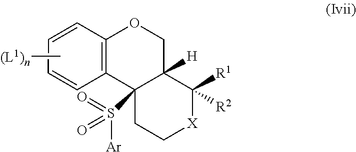

wherein at least one (e.g., 1-4, 1-3, 1-2, 2 or 1) H in formula (I) is replaced by deuterium (D).

The phrase “any one of the compounds” used below for the compounds of formulas (Ii) to (Ivii), (I.A1a) to (I.A1h), and (I.A2a) to (I.A2h), unless stated otherwise, means that such description applies to each compound mentioned in the description just as if each compound mentioned was separately described. Thus, for example, the description below that the “compounds of formula (I) also include any one of the compounds of formulas (Ii) to (Ivii) wherein n is 2” is intended to describe an embodiment directed to a compound of formula (Ii) wherein n is 2. It is also intended to describe an embodiment directed to a compound of formula (III) wherein n is 2. It is also intended to describe an embodiment directed to a compound of formula Nip wherein n is 2. It is also intended to describe an embodiment directed to a compound of formula (Iiv) wherein n is 2. It is also intended to describe an embodiment directed to a compound of formula (Iv) wherein n is 2. It is also intended to describe an embodiment directed to a compound of formula (Ivi) wherein n is 2. It is also intended to describe an embodiment directed to a compound of formula (Ivii) wherein n is 2.

Compounds of formula (I) also include any one of the compounds of formulas (Ii) to (Ivii) above wherein n is 2.

Compounds of formula (I) also include any one of the compounds of formulas (I.A1) or (I.A2) above wherein n is 2, and the L¹ groups are bound to the phenyl moiety as shown in (IIA):

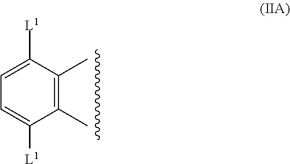

(wherein the squiggly line [symbol] represents the rest of the formula).

Compounds of formula (I) also include any one of the compounds of formulas (Ii) to (Ivii) above wherein n is 2, and the L¹ groups are bound to the phenyl moiety as as shown in (IIA):

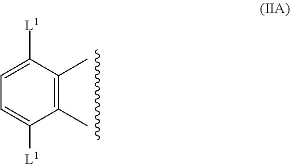

(wherein the squiggly line [symbol] represents the rest of the formula).

Compounds of formula (I) also include any one of the compounds of formulas (Ii) to (Ivii) above wherein n is 2, and the L¹ groups are bound to the phenyl moiety as shown in (IIA) and each L¹ is the same or different halo.

Compounds of formula (I) also include any one of the compounds of formulas (Ii) to (Ivii) above wherein n is 2, and the L¹ groups are bound to the phenyl moiety as shown in (IIA) and each L¹ is F.

Compounds of formula (I) also include any one of the compounds of formulas (Ii) to (Ivi) above wherein n is 2, and the L¹ groups are bound to the phenyl moiety as shown in (IIA) and Ar is selected from the group consisting of p-Cl-phenyl-, p-CN-phenyl-, p-CF₃-phenyl, p-CH₃CH₂SO₂phenyl, p-Br-phenyl, m,p-di-F-phenyl, m,p-di-CN-phenyl, p-CH₃O-phenyl, p-CF₃CH₂Ophenyl, pyridyl, and pyridyl substituted with 1 or 2 substituents independently selected from the group consisting of: halogen, alkyl (e.g., C₁-C₆ alkyl), —CN, —CF₃, —O—(C₁-C₆)alkyl (e.g., —OCH₃), —O-(halo(C₁-C₆)alkyl) (e.g., —OCF₃ and —OCH₂CF₃), —C(O)—O—(C₁-C₆)alkyl (e.g., —C(O)OCH₃), -alkylene-OH (e.g., —CH₂OH), halo(C₁-C₆)alkyl (e.g., —CF₃), hydroxyalkoxy- (e.g., HOCH₂CH₂O—), and alkoxyalkoxy- (e.g., CH₃OCH₂CH₂O—).

Compounds of formula (I) also include any one of the compounds of formulas (Ii) to (Ivii) above wherein n is 2, and the L¹ groups are bound to the phenyl moiety as shown in (IIA) and Ar is selected from the group consisting of p-Cl-phenyl-, p-CN-phenyl-, p-CF₃-phenyl, p-CH₃CH₂SO₂phenyl, p-Br-phenyl, m,p-di-F-phenyl, m,p-di-CN-phenyl, p-CH₃O-phenyl, p-CF₃CH₂Ophenyl, pyridyl, and pyridyl substituted with 1 or 2 substituents independently selected from the group consisting of: halogen, alkyl (e.g., C₁-C₆ alkyl), —CN, —CF₃, —O—(C₁-C₆)alkyl (e.g., —OCH₃), —O-(halo(C₁-C₆)alkyl) (e.g., —OCF₃ and —OCH₂CF₃), —C(O)—O—(C₁-C₆)alkyl (e.g., —C(O)OCH₃), -alkylene-OH (e.g., —CH₂OH), halo(C₁-C₆)alkyl (e.g., —CF₃), hydroxyalkoxy- (e.g., HOCH₂CH₂O—), and alkoxyalkoxy- (e.g., CH₃OCH₂CH₂O—).

Compounds of formula (I) also include any one of the compounds of formulas (Ii) to (Ivii) above wherein n is 2, and the L¹ groups are bound to the phenyl moiety as shown in (IIA) and Ar is selected from the group consisting of p-Cl-phenyl-, p-CN-phenyl-, p-CF₃-phenyl, pyridyl, and pyridyl substituted with 1 or 2 substituents independently selected from the group consisting of: halogen, alkyl (e.g., C₁-C₆ alkyl), —CN, —CF₃, —O—(C₁-C₆)alkyl (e.g., —OCH₃), —O-(halo(C₁-C₆)alkyl) (e.g., —OCF₃), —C(O)—O—(C₁-C₆)alkyl (e.g., —C(O)OCH₃), -alkylene-OH (e.g., —CH₂OH), halo(C₁-C₆)alkyl (e.g., —CF₃), hydroxyalkoxy- (e.g., HOCH₂CH₂O—), and alkoxyalkoxy- (e.g., CH₃OCH₂CH₂O—).

Compounds of formula (I) also include any one of the compounds of formulas (Ii) to (Ivii) above wherein n is 2, and the L¹ groups are bound to the phenyl moiety as shown in (IIA) and Ar is selected from the group consisting of p-Cl-phenyl-, p-CN-phenyl-, p-CF₃-phenyl, pyridyl, and pyridyl substituted with 1 substituent selected from the group consisting of: —Cl, —CF₃ and —CN.

Compounds of formula (I) also include any one of the compounds of formulas (Ii) to (Ivii) above wherein n is 2, and the L¹ groups are bound to the phenyl moiety as shown in (IIA) and Ar is selected from the group consisting of p-Cl-phenyl-, p-CN-phenyl-, and p-CF₃-phenyl.

Compounds of formula (I) also include any one of the compounds of formulas (Ii) to (Ivii) above wherein n is 2, and the L¹ groups are bound to the phenyl moiety as shown in (IIA) and Ar is selected from the group consisting of: pyridyl, and pyridyl substituted with 1 substituent selected from the group consisting of: —Cl, —CF₃ and —CN.

Compounds of formula (I) also include any one of the compounds of formulas (Ii) to (Ivii) above wherein n is 2, and the L¹ groups are bound to the phenyl moiety as shown in (IIA), each L¹ is the same or different halo, and Ar is selected from the group consisting of p-Cl-phenyl-, p-CN-phenyl-, p-CF₃-phenyl, p-CH₃CH₂SO₂phenyl, p-Br-phenyl, m,p-di-F-phenyl, m,p-di-CN-phenyl, p-CH₃O-phenyl, p-CF₃CH₂Ophenyl, pyridyl, and pyridyl substituted with 1 or 2 substituents independently selected from the group consisting of: halogen, alkyl (e.g., C₁-C₆ alkyl), —CN, —CF₃, —O—(C₁-C₆)alkyl (e.g., —OCH₃), —O-(halo(C₁-C₆)alkyl) (e.g., —OCF₃ and —OCH₂CF₃), —C(O)—O—(C₁-C₆)alkyl (e.g., —C(O)OCH₃), -alkylene-OH (e.g., —CH₂OH), halo(C₁-C₆)alkyl (e.g., —CF₃), hydroxyalkoxy- (e.g., HOCH₂CH₂O—), and alkoxyalkoxy- (e.g., CH₃OCH₂CH₂O—).

Compounds of formula (I) also include any one of the compounds of formulas (Ii) to (Ivii) above wherein n is 2, and the L¹ groups are bound to the phenyl moiety as shown in (IIA), each L¹ is the same or different halo, and Ar is selected from the group consisting of p-Cl-phenyl-, p-CN-phenyl-, p-CF₃-phenyl, pyridyl, and pyridyl substituted with 1 or 2 substituents independently selected from the group consisting of: halogen, alkyl (e.g., C₁-C₆ alkyl), —CN, —CF₃, —O—(C₁-C₆)alkyl (e.g., —OCH₃), —O-(halo(C₁-C₆)alkyl) (e.g., —OCF₃), —C(O)—O—(C₁-C₆)alkyl (e.g., —C(O)OCH₃), -alkylene-OH (e.g., —CH₂OH), halo(C₁-C₆)alkyl (e.g., —CF₃), hydroxyalkoxy- (e.g., HOCH₂CH₂O—), and alkoxyalkoxy- (e.g., CH₃OCH₂CH₂O—).

Compounds of formula (I) also include any one of the compounds of formulas (Ii) to (Ivii) above wherein n is 2, and the L¹ groups are bound to the phenyl moiety as shown in (IIA), each L¹ is the same or different halo, and Ar is selected from the group consisting of p-Cl-phenyl-, p-CN-phenyl-, p-CF₃-phenyl, pyridyl, and pyridyl substituted with 1 substituent selected from the group consisting of: —Cl, —CF₃ and —CN.

Compounds of formula (I) also include any one of the compounds of formulas (Ii) to (Ivii) above wherein n is 2, and the L¹ groups are bound to the phenyl moiety as shown in (IIA), each L¹ is the same or different halo, and Ar is selected from the group consisting of p-Cl-phenyl-, p-CN-phenyl-, and p-CF₃-phenyl.

Compounds of formula (I) also include any one of the compounds of formulas (Ii) to (Ivii) above wherein n is 2, and the L¹ groups are bound to the phenyl moiety as shown in (IIA), each L¹ is the same or different halo, and Ar is selected from the group consisting of: pyridyl, and pyridyl substituted with 1 substituent selected from the group consisting of: —Cl, —CF₃ and —CN.

Compounds of formula (I) also include any one of the compounds of formulas (Ii) to (Ivii) above wherein n is 2, and the L¹ groups are bound to the phenyl moiety as shown in (IIA), each L¹ is F, and Ar is selected from the group consisting of p-Cl-phenyl-, p-CN-phenyl-, p-CF₃-phenyl, p-CH₃CH₂SO₂phenyl, p-Br-phenyl, m,p-di-F-phenyl, m,p-di-CN-phenyl, p-CH₃O-phenyl, p-CF₃CH₂Ophenyl, pyridyl, and pyridyl substituted with 1 or 2 substituents independently selected from the group consisting of: halogen, alkyl (e.g., C₁-C₆ alkyl), —CN, —CF₃, —O—(C₁-C₆)alkyl (e.g., —OCH₃), —O-(halo(C₁-C₆)alkyl) (e.g., —OCF₃ and —OCH₂CF₃), —C(O)—O—(C₁-C₆)alkyl (e.g., —C(O)OCH₃), -alkylene-OH(—CH₂OH), halo(C₁-C₆)alkyl (e.g., —CF₃), hydroxyalkoxy- (e.g., HOCH₂CH₂O—), and alkoxyalkoxy- (e.g., CH₃OCH₂CH₂O—).

Compounds of formula (I) also include any one of the compounds of formulas (Ii) to (Ivii) above wherein n is 2, and the L¹ groups are bound to the phenyl moiety as shown in (IIA), each L¹ is F, and Ar is selected from the group consisting of p-Cl-phenyl-, p-CN-phenyl-, p-CF₃-phenyl, pyridyl, and pyridyl substituted with 1 or 2 substituents independently selected from the group consisting of: halogen, alkyl (e.g., C₁-C₆ alkyl), —CN, —CF₃, —O—(C₁-C₆)alkyl (e.g., —OCH₃), —O-(halo(C₁-C₆)alkyl) (e.g., —OCF₃), —C(O)—O—(C₁-C₆)alkyl (e.g., —C(O)OCH₃), -alkylene-OH (e.g., —CH₂OH), halo(Cₜ—C₆)alkyl (e.g., —CF₃), hydroxyalkoxy- (e.g., HOCH₂CH₂O—), and alkoxyalkoxy- (e.g., CH₃OCH₂CH₂O—).

Compounds of formula (I) also include any one of the compounds of formulas (Ii) to (Ivii) above wherein n is 2, and the L¹ groups are bound to the phenyl moiety as shown in (IIA), each L¹ is F, and Ar is selected from the group consisting of p-Cl-phenyl-, p-CN-phenyl-, p-CF₃-phenyl, pyridyl, and pyridyl substituted with 1 substituent selected from the group consisting of: —Cl, —CF₃ and —CN.

Compounds of formula (I) also include any one of the compounds of formulas (Ii) to (Ivii) above wherein n is 2, and the L¹ groups are bound to the phenyl moiety as shown in (IIA), each L¹ is F, and Ar is selected from the group consisting of p-Cl-phenyl-, p-CN-phenyl-, and p-CF₃-phenyl.

Compounds of formula (I) also include any one of the compounds of formulas (Ii) to (Ivii) above wherein n is 2, and the L¹ groups are bound to the phenyl moiety as shown in (IIA), each L¹ is F, and Ar is selected from the group consisting of: pyridyl, and pyridyl substituted with substituent selected from the group consisting of: —Cl, —CF₃ and —CN.

Compounds of formula (I) include compounds of formula (I.A1a):

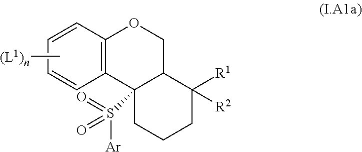

wherein all substituents are as defined for formula (I), and wherein at least one (e.g., 1-4, 1-3, 1-2, 2 or 1) H in formula (I.A1a) is replaced by deuterium (D).

Compounds of formula (I) include compounds of formula (I.A1b):

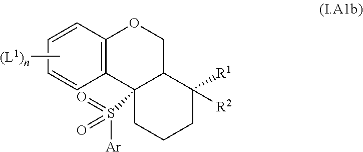

wherein all substituents are as defined for formula (I), and wherein at least one (e.g., 1-4, 1-3, 1-2, 2 or 1) H in formula (I.A1b) is replaced by deuterium (D).

Compounds of formula (I) include compounds of formula (I.A1c):

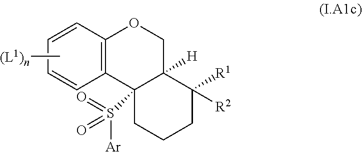

wherein all substituents are as defined for formula (I), and wherein at least one (e.g., 1-4, 1-3, 1-2, 2 or 1) H in formula (I.A1c) is replaced by deuterium (D).

Compounds of formula (I) incluse compounds of formula (I.A1d):

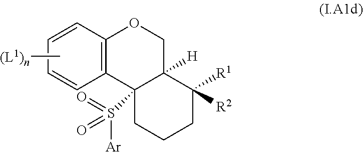

wherein all substituents are as defined for formula (I), and wherein at least one (e.g., 1-4, 1-3, 1-2, 2 or 1) H in formula (I.A1d) is replaced by deuterium (D).

Compounds of formula (I) include compounds of formula (I.A1e):

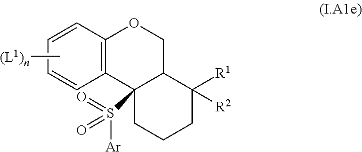

wherein all substituents are as defined for formula (I), and wherein at least one (e.g., 1-4, 1-3, 1-2, 2 or 1) H in formula (I.A1f) is replaced by deuterium (D).

Compounds of formula (I) include compounds of formula (I.A1f):

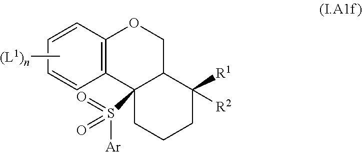

wherein all substituents are as defined for formula (I), and wherein at least one (e.g., 1-4, 1-3, 1-2, 2 or 1) H in formula (I.A1f) is replaced by deuterium (D).

Compounds of formula (I) include compounds of formula (I.A1g):

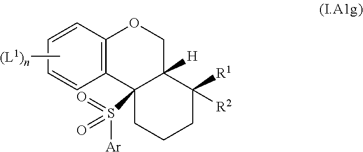

wherein all substituents are as defined for formula (I), and wherein at least one (e.g., 1-4, 1-3, 1-2, 2 or 1) H in formula (I.A1g) is replaced by deuterium (D).

Compounds of formula (I) include compounds of formula (I.A1h):

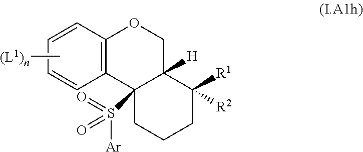

wherein all substituents are as defined for formula (I), and wherein at least one (e.g., 1-4, 1-3, 1-2, 2 or 1) H in formula (I.A1h) is replaced by deuterium (D).

Compounds of formula (I) include compounds of formula (I.A1i):

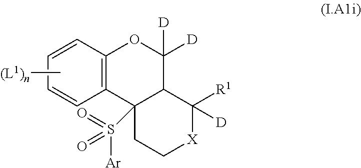

wherein all substituents are as defined for formula (I).

Compounds of formula (I) include compounds of formula (I.A1j):

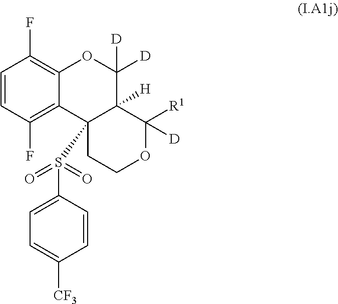

wherein all substituents are as defined for formula (I).

Compounds of formula (I) include compounds of formula (I.A1k):

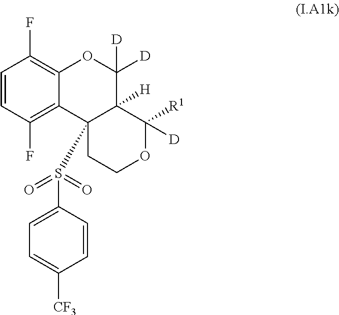

wherein all substituents are as defined for formula (I).

Compounds of formula (I) include compounds of formula (I.A1l):

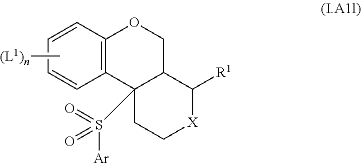

wherein all substituents are as defined for formula (I), and wherein 1 to 3H in the R¹ substituent are replaced with deuterium. In one example X is O.

Compounds of formula (I) include compounds of formula (I.A1m):

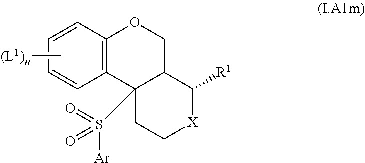

wherein all substituents are as defined for formula (I), and wherein 1 to 3H in the R¹ substituent are replaced with deuterium. In one example X is O.

Compounds of formula (I) include compounds of formula (I.A1n):

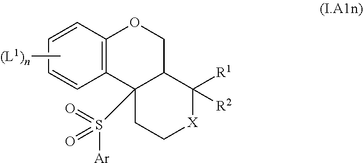

wherein all substituents are as defined for formula (I), and wherein R² is alkyl and 1 to 3H in said R² alkyl substituent are replaced with deuterium. In one example X is O. In one example, R² is —CD₃. In one example X is O and R² is —CD₃.

Compounds of formula (I) include compounds of formula (I.A1o):

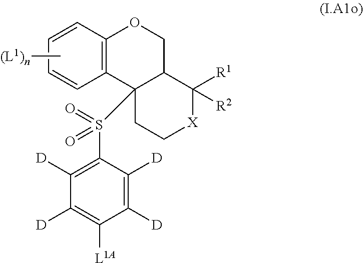

wherein all substituents are as defined for formula (I). In one example X is O. In one example, L^{1A} is Cl. In one example X is O and L^{1A} is Cl.

Compounds of formula (I) include compounds of formula (I.A1p):

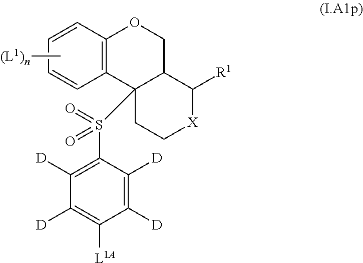

wherein all substituents are as defined for formula (I). In one example X is O. In one example, L^{1A} is Cl. In one example X is O and L^{1A} is Cl.

Compounds of formula (I) include compounds of formula (I.A1q):

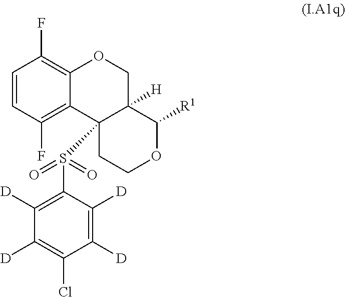

wherein all substituents are as defined for formula (I). In one example X is O. In one example, L^{1A} is Cl. In one example X is O and L^{1A} is Cl.

Compounds of formula (I) include compounds of formula (I.A1r):

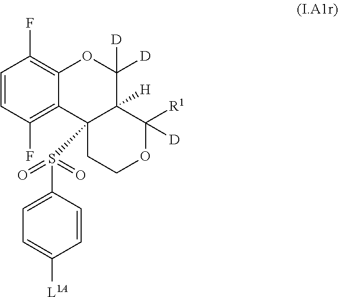

wherein all substituents are as defined for formula (I).

Compounds of formula (I) include compounds of formula (I.A1s):

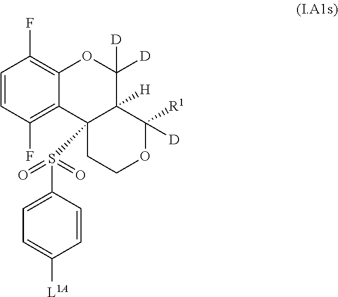

wherein all substituents are as defined for formula (I).

Compounds of formula (I) also include any one of the compounds of formulas I.A1i, I.A1l, I.A1m, I.A1n, I.A1o, and I.A1p above wherein n is 2, and the L¹ groups are bound to the phenyl moiety as shown in (IIA) and each L¹ is the same or different halo.

Compounds of formula (I) also include any one of the compounds of formulas I.A1i, I.A1l, I.A1m, I.A1n, I.A1o, and I.A1p above wherein n is 2, and the L¹ groups are bound to the phenyl moiety as shown in (IIA) and each L¹ is F.

Compounds of formula (I) also include any one of the compounds of formulas I.A1i, I.A1l, I.A1m, I.A1n, I.A1o, and I.A1p above wherein n is 2, and the L¹ groups are bound to the phenyl moiety as shown in (IIA) and Ar is selected from the group consisting of p-Cl-phenyl-, p-CN-phenyl-, p-CF₃-phenyl, p-CH₃CH₂SO₂phenyl, p-Br-phenyl, m,p-di-F-phenyl, m,p-di-CN-phenyl, p-CH₃O-phenyl, p-CF₃CH₂Ophenyl, pyridyl, and pyridyl substituted with 1 or 2 substituents independently selected from the group consisting of: halogen, alkyl (e.g., C₁-C₆ alkyl), —CN, —CF₃, —O—(C₁-C₆)alkyl (e.g., —OCH₃), —O-(halo(C₁-C₆)alkyl) (e.g., —OCF₃ and —OCH₂CF₃), —C(O)—O—(C₁-C₆)alkyl (e.g., —C(O)OCH₃), -alkylene-OH (e.g., —CH₂OH), halo(C₁-C₆)alkyl (e.g., —CF₃), hydroxyalkoxy- (e.g., HOCH₂CH₂O—), and alkoxyalkoxy- (e.g., CH₃OCH₂CH₂O—).

Compounds of formula (I) also include any one of the compounds of formulas I.A1i, I.A1l, I.A1m, I.A1n, I.A1o, and I.A1p above wherein n is 2, and the L¹ groups are bound to the phenyl moiety as shown in (IIA) and Ar is selected from the group consisting of p-Cl-phenyl-, p-CN-phenyl-, p-CF₃-phenyl, pyridyl, and pyridyl substituted with 1 or 2 substituents independently selected from the group consisting of: halogen, alkyl (e.g., C₁-C₆ alkyl), —CN, —CF₃, —O—(C₁-C₆)alkyl (e.g., —OCH₃), —O-(halo(C₁-C₆)alkyl) (e.g., —OCF₃), —C(O)—O—(C₁-C₆)alkyl (e.g., —C(O)OCH₃), -alkylene-OH (e.g., —CH₂OH), halo(C₁-C₆)alkyl (e.g., —CF₃), hydroxyalkoxy- (e.g., HOCH₂CH₂O—), and alkoxyalkoxy- (e.g., CH₃OCH₂CH₂O—).

Compounds of formula (I) also include any one of the compounds of formulas I.A1i, I.A1l, I.A1m, I.A1n, I.A1o, and I.A1p above wherein n is 2, and the L¹ groups are bound to the phenyl moiety as shown in (IIA) and Ar is selected from the group consisting of p-Cl-phenyl-, p-CN-phenyl-, p-CF₃-phenyl, pyridyl, and pyridyl substituted with 1 substituent selected from the group consisting of: —Cl, —CF₃ and —CN.

Compounds of formula (I) also include any one of the compounds of formulas I.A1i, I.A1l, I.A1m, I.A1n, I.A1o, and I.A1p above wherein n is 2, and the L¹ groups are bound to the phenyl moiety as shown in (IIA) and Ar is selected from the group consisting of p-Cl-phenyl-, p-CN-phenyl-, and p-CF₃-phenyl.

Compounds of formula (I) also include any one of the compounds of formulas I.A1i, I.A1l, I.A1m, I.A1n, I.A1o, and I.A1p above wherein n is 2, and the L¹ groups are bound to the phenyl moiety as shown in (IIA) and Ar is selected from the group consisting of: pyridyl, and pyridyl substituted with 1 substituent selected from the group consisting of: —Cl, —CF₃ and —CN.

Compounds of formula (I) also include any one of the compounds of formulas I.A1i, I.A1l, I.A1m, I.A1n, I.A1o, and I.A1p above wherein n is 2, and the L¹ groups are bound to the phenyl moiety as shown in (IIA), each L¹ is the same or different halo, and Ar is selected from the group consisting of p-Cl-phenyl-, p-CN-phenyl-, p-CF₃-phenyl, p-CH₃CH₂SO₂phenyl, p-Br-phenyl, m,p-di-F-phenyl, m,p-di-CN-phenyl, p-CH₃O-phenyl, p-CF₃CH₂Ophenyl, pyridyl, and pyridyl substituted with 1 or 2 substituents independently selected from the group consisting of: halogen, alkyl (e.g., C₁-C₆ alkyl), —CN, —CF₃, —O—(C₁-C₆)alkyl (e.g., —OCH₃), —O-(halo(C₁-C₆)alkyl) (e.g., —OCF₃ and —OCH₂CF₃), —C(O)—O—(C₁-C₆)alkyl (e.g., —C(O)OCH₃), -alkylene-OH (e.g., —CH₂OH), halo(C₁-C₈)alkyl (e.g., —CF₃), hydroxyalkoxy- (e.g., HOCH₂CH₂O—), and alkoxyalkoxy- (e.g., CH₃OCH₂CH₂O—).

Compounds of formula (I) also include any one of the compounds of formulas I.A1i, I.A1l, I.A1m, I.A1n, I.A1o, and I.A1p above wherein n is 2, and the L¹ groups are bound to the phenyl moiety as shown in (IIA), each L¹ is the same or different halo, and Ar is selected from the group consisting of p-Cl-phenyl-, p-CN-phenyl-, p-CF₃-phenyl, pyridyl, and pyridyl substituted with 1 or 2 substituents independently selected from the group consisting of: halogen, alkyl (e.g., C₁-C₆ alkyl), —CN, —CF₃, —O—(C₁-C₆)alkyl (e.g., —OCH₃), —O-(halo(C₁-C₆)alkyl) (e.g., —OCF₃), —C(O)—O—(C₁-C₆)alkyl (e.g., —C(O)OCH₃), -alkylene-OH (e.g., —CH₂OH), halo(C₁-C₆)alkyl (e.g., —CF₃), hydroxyalkoxy- (e.g., HOCH₂CH₂O—), and alkoxyalkoxy- (e.g., CH₃OCH₂CH₂O—).

Compounds of formula (I) also include any one of the compounds of formulas I.A1i, I.A1l, I.A1m, I.A1n, I.A1o, and I.A1p above wherein n is 2, and the L¹ groups are bound to the phenyl moiety as shown in (IIA), each L¹ is the same or different halo, and Ar is selected from the group consisting of p-Cl-phenyl-, p-CN-phenyl-, p-CF₃-phenyl, pyridyl, and pyridyl substituted with 1 substituent selected from the group consisting of: —Cl, —CF₃ and —CN.

Compounds of formula (I) also include any one of the compounds of formulas I.A1i, I.A1l, I.A1m, I.A1n, I.A1o, and I.A1p above wherein n is 2, and the L¹ groups are bound to the phenyl moiety as shown in (IIA), each L¹ is the same or different halo, and Ar is selected from the group consisting of p-Cl-phenyl-, p-CN-phenyl-, and p-CF₃-phenyl.

Compounds of formula (I) also include any one of the compounds of formulas I.A1i, I.A1l, I.A1m, I.A1n, I.A1o, and I.A1p above wherein n is 2, and the L¹ groups are bound to the phenyl moiety as shown in (IIA), each L¹ is the same or different halo, and Ar is selected from the group consisting of: pyridyl, and pyridyl substituted with 1 substituent selected from the group consisting of: —Cl, —CF₃ and —CN.

Compounds of formula (I) also include any one of the compounds of formulas I.A1i, I.A1l, I.A1m, I.A1n, I.A1o, and I.A1p above wherein n is 2, and the L¹ groups are bound to the phenyl moiety as shown in (IIA), each L¹ is F, and Ar is selected from the group consisting of p-Cl-phenyl-, p-CN-phenyl-, p-CF₃-phenyl, p-CH₃CH₂SO₂phenyl, p-Br-phenyl, m,p-di-F-phenyl, m,p-di-CN-phenyl, p-CH₃O-phenyl, p-CF₃CH₂Ophenyl, pyridyl, and pyridyl substituted with 1 or 2 substituents independently selected from the group consisting of: halogen, alkyl (e.g., C₁-C₆ alkyl), —CN, —CF₃, —O—(C₁-C₆)alkyl (e.g., —OCH₃), —O-(halo(C₁-C₆)alkyl) (e.g., —OCF₃ and —OCH₂CF₃), —C(O)—O—(C₁-C₆)alkyl (e.g., —C(O)OCH₃), -alkylene-OH (e.g., —CH₂OH), halo(C₁-C₆)alkyl (e.g., —CF₃), hydroxyalkoxy- (e.g., HOCH₂CH₂O—), and alkoxyalkoxy- (e.g., CH₃OCH₂CH₂O—).

Compounds of formula (I) also include any one of the compounds of formulas I.A1i, I.A1l, I.A1m, I.A1n, I.A1o, and I.A1p above wherein n is 2, and the L¹ groups are bound to the phenyl moiety as shown in (IIA), each L¹ is F, and Ar is selected from the group consisting of p-Cl-phenyl-, p-CN-phenyl-, p-CF₃-phenyl, pyridyl, and pyridyl substituted with 1 or 2 substituents independently selected from the group consisting of: halogen, alkyl (e.g., C₁-C₆ alkyl), —CN, —CF₃, —O—(C₁-C₆)alkyl (e.g., —OCH₃), —O-(halo(C₁-C₆)alkyl) (e.g., —OCF₃), —C(O)—O—(C₁-C₆)alkyl (e.g., —C(O)OCH₃), -alkylene-OH (e.g., —CH₂OH), halo(C₁-C₆)alkyl (e.g., —CF₃), hydroxyalkoxy- (e.g., HOCH₂CH₂O—), and alkoxyalkoxy- (e.g., CH₃OCH₂CH₂O—).

Compounds of formula (I) also include any one of the compounds of formulas I.A1i, I.A1l, I.A1m, I.A1n, I.A1o, and I.A1p above wherein n is 2, and the L¹ groups are bound to the phenyl moiety as shown in (IIA), each L¹ is F, and Ar is selected from the group consisting of p-Cl-phenyl-, p-CN-phenyl-, p-CF₃-phenyl, pyridyl, and pyridyl substituted with 1 substituent selected from the group consisting of: —Cl, —CF₃ and —CN.

Compounds of formula (I) also include any one of the compounds of formulas I.A1i, I.A1l, I.A1m, I.A1n, I.A1o, and I.A1p above wherein n is 2, and the L¹ groups are bound to the phenyl moiety as shown in (IIA), each L¹ is F, and Ar is selected from the group consisting of p-Cl-phenyl-, p-CN-phenyl-, and p-CF₃-phenyl.

Compounds of formula (I) also include any one of the compounds of formulas I.A1i, I.A1l, I.A1m, I.A1n, I.A1o, and I.A1p above wherein n is 2, and the L¹ groups are bound to the phenyl moiety as shown in (IIA), each L¹ is F, and Ar is selected from the group consisting of: pyridyl, and pyridyl substituted with 1 substituent selected from the group consisting of: —Cl, —CF₃ and —CN.

Compounds of formula (I) also include any one of the compounds of formulas I.A1i, I.A1l, I.A1m, I.A1n, I.A1o, and I.A1p above wherein n is 2, and the L¹ groups are bound to the phenyl moiety as shown in (IIA), each L¹ is F, and Ar is p-Cl-phenyl-.

Compounds of formula (I) also include any one of the compounds of formulas I.A1i, I.A1l, I.A1m, I.A1n, I.A1o, and I.A1p above wherein n is 2, and the L¹ groups are bound to the phenyl moiety as shown in (IIA), each L¹ is F, and Ar is p-CN-phenyl-.

Compounds of formula (I) also include any one of the compounds of formulas I.A1i, I.A1l, I.A1m, I.A1n, I.A1o, and I.A1p above wherein n is 2, and the L¹ groups are bound to the phenyl moiety as shown in (IIA), each L¹ is F, and Ar is p-CF₃-phenyl.

Compounds of formula (I) also include any one of the compounds of formulas I.A1i, I.A1l, I.A1m, I.A1n, I.A1o, and I.A1p above wherein n is 2, and the L¹ groups are bound to the phenyl moiety as shown in (IIA), each L¹ is F, and Ar is p-CH₃CH₂SO₂phenyl.

Compounds of formula (I) also include any one of the compounds of formulas I.A1i, I.A1l, I.A1m, I.A1n, I.A1o, and I.A1p above wherein n is 2, and the L¹ groups are bound to the phenyl moiety as shown in (IIA), each L¹ is F, and Ar is p-Br-phenyl.

Compounds of formula (I) also include any one of the compounds of formulas I.A1i, I.A1l, I.A1m, I.A1n, I.A1o, and I.A1p above wherein n is 2, and the L¹ groups are bound to the phenyl moiety as shown in (IIA), each L¹ is F, and Ar is m,p-di-F-phenyl.

Compounds of formula (I) also include any one of the compounds of formulas I.A1i, I.A1l, I.A1m, I.A1n, I.A1o, and I.A1p above wherein n is 2, and the L¹ groups are bound to the phenyl moiety as shown in (IIA), each L¹ is F, and Ar is m,p-di-CN-phenyl.

Compounds of formula (I) also include any one of the compounds of formulas I.A1i, I.A1l, I.A1m, I.A1n, I.A1o, and I.A1p above wherein n is 2, and the L¹ groups are bound to the phenyl moiety as shown in (IIA), each L¹ is F, and Ar is p-CH₃O-phenyl.

Compounds of formula (I) also include any one of the compounds of formulas I.A1i, I.A1l, I.A1m, I.A1n, I.A1o, and I.A1p above wherein n is 2, and the L¹ groups are bound to the phenyl moiety as shown in (IIA), each L¹ is F, and Ar is p-CF₃CH₂Ophenyl.

Compounds of formula (I) also include any one of the compounds of formulas (I.A1a) to (I.A1h) above wherein n is 2, and the L¹ groups are bound to the phenyl moiety as shown in (IIA) and each L¹ is the same or different halo.

Compounds of formula (I) also include any one of the compounds of formulas (I.A1a) to (I.A1h) above wherein n is 2, and the L¹ groups are bound to the phenyl moiety as shown in (IIA) and each L¹ is F.

Compounds of formula (I) also include any one of the compounds of formulas (I.A1a) to (I.A1h) above wherein n is 2, and the L¹ groups are bound to the phenyl moiety as shown in (IIA) and Ar is selected from the group consisting of p-Cl-phenyl-, p-CN-phenyl-, p-CF₃-phenyl, p-CH₃CH₂SO₂phenyl, p-Br-phenyl, m,p-di-F-phenyl, m,p-di-CN-phenyl, p-CH₃O-phenyl, p-CF₃CH₂Ophenyl, pyridyl, and pyridyl substituted with 1 or 2 substituents independently selected from the group consisting of: halogen, alkyl (e.g., C₁-C₆ alkyl), —CN, —CF₃, —O—(C₁-C₆)alkyl (e.g., —OCH₃), —O-(halo(C₁-C₆)alkyl) (e.g., —OCF₃ and —OCH₂CF₃), —C(O)—O—(C₁-C₆)alkyl (e.g., —C(O)OCH₃), -alkylene-OH (e.g., —CH₂OH), halo(C₁-C₆)alkyl (e.g., —CF₃), hydroxyalkoxy- (e.g., HOCH₂CH₂O—), and alkoxyalkoxy- (e.g., CH₃OCH₂CH₂O—).

Compounds of formula (I) also include any one of the compounds of formulas (I.A1a) to (I.A1h) above wherein n is 2, and the L¹ groups are bound to the phenyl moiety as shown in (IIA) and Ar is selected from the group consisting of p-Cl-phenyl-, p-CN-phenyl-, p-CF₃-phenyl, pyridyl, and pyridyl substituted with 1 or 2 substituents independently selected from the group consisting of: halogen, alkyl (e.g., C₁-C₆ alkyl), —CN, —CF₃, —O—(C₁-C₆)alkyl (e.g., —OCH₃), —O-(halo(C₁-C₆)alkyl) (e.g., —OCF₃), —C(O)—O—(C₁-C₆)alkyl (e.g., —C(O)OCH₃), -alkylene-OH (e.g., —CH₂OH), halo(C₁-C₆)alkyl (e.g., —CF₃), hydroxyalkoxy- (e.g., HOCH₂CH₂O—), and alkoxyalkoxy- (e.g., CH₃OCH₂CH₂O—).

Compounds of formula (I) also include any one of the compounds of formulas (I.A1a) to (I.A1h) above wherein n is 2, and the L¹ groups are bound to the phenyl moiety as shown in (IIA) and Ar is selected from the group consisting of p-Cl-phenyl-, p-CN-phenyl-, p-CF₃-phenyl, pyridyl, and pyridyl substituted with 1 substituent selected from the group consisting of: —Cl, —CF₃ and —CN.

Compounds of formula (I) also include any one of the compounds of formulas (I.A1a) to (I.A1h) above wherein n is 2, and the L¹ groups are bound to the phenyl moiety as shown in (IIA) and Ar is selected from the group consisting of p-Cl-phenyl-, p-CN-phenyl-, and p-CF₃-phenyl.

Compounds of formula (I) also include any one of the compounds of formulas (I.A1a) to (I.A1h) above wherein n is 2, and the L¹ groups are bound to the phenyl moiety as shown in (IIA) and Ar is selected from the group consisting of: pyridyl, and pyridyl substituted with 1 substituent selected from the group consisting of: —Cl, —CF₃ and —CN.

Compounds of formula (I) also include any one of the compounds of formulas (I.A1a) to (I.A1h) above wherein n is 2, and the L¹ groups are bound to the phenyl moiety as shown in (IIA), each L¹ is the same or different halo, and Ar is selected from the group consisting of p-Cl-phenyl-, p-CN-phenyl-, p-CF₃-phenyl, p-CH₃CH₂SO₂phenyl, p-Br-phenyl, m,p-di-F-phenyl, m,p-di-CN-phenyl, p-CH₃O-phenyl, p-CF₃CH₂Ophenyl, pyridyl, and pyridyl substituted with 1 or 2 substituents independently selected from the group consisting of: halogen, alkyl (e.g., C₁-C₆ alkyl), —CN, —CF₃, —O—(C₁-C₆)alkyl (e.g., —OCH₃), —O-(halo(C₁-C₆)alkyl) (e.g., —OCF₃ and —OCH₂CF₃), —C(O)—O—(C₁-C₆)alkyl (e.g., —C(O)OCH₃), -alkylene-OH (e.g., —CH₂OH), halo(C₁-C₆)alkyl (e.g., —CF₃), hydroxyalkoxy- (e.g., HOCH₂CH₂O—), and alkoxyalkoxy- (e.g., CH₃OCH₂CH₂O—).

Compounds of formula (I) also include any one of the compounds of formulas (I.A1a) to (I.A1h) above wherein n is 2, and the L¹ groups are bound to the phenyl moiety as shown in (IIA), each L¹ is the same or different halo, and Ar is selected from the group consisting of p-Cl-phenyl-, p-CN-phenyl-, p-CF₃-phenyl, pyridyl, and pyridyl substituted with 1 or 2 substituents independently selected from the group consisting of: halogen, alkyl (e.g., C₁-C₆ alkyl), —CN, —CF₃, —O—(C₁-C₆)alkyl (e.g., —OCH₃), —O-(halo(C₁-C₆)alkyl) (e.g., —OCF₃), —C(O)—O—(C₁-C₆)alkyl (e.g., —C(O)OCH₃), -alkylene-OH (e.g., —CH₂OH), halo(C₁-C₆)alkyl (e.g., —CF₃), hydroxyalkoxy- (e.g., HOCH₂CH₂O—), and alkoxyalkoxy- (e.g., CH₃OCH₂CH₂O—).

Compounds of formula (I) also include any one of the compounds of formulas (I.A1a) to (I.A1h) above wherein n is 2, and the L¹ groups are bound to the phenyl moiety as shown in (IIA), each L¹ is the same or different halo, and Ar is selected from the group consisting of p-Cl-phenyl-, p-CN-phenyl-, p-CF₃-phenyl, pyridyl, and pyridyl substituted with 1 substituent selected from the group consisting of: —Cl, —CF₃ and —CN.

Compounds of formula (I) also include any one of the compounds of formulas (I.A1a) to (I.A1h) above wherein n is 2, and the L¹ groups are bound to the phenyl moiety as shown in (IIA), each L¹ is the same or different halo, and Ar is selected from the group consisting of p-Cl-phenyl-, p-CN-phenyl-, and p-CF₃-phenyl.

Compounds of formula (I) also include any one of the compounds of formulas (I.A1a) to (I.A1h) above wherein n is 2, and the L¹ groups are bound to the phenyl moiety as shown in (IIA), each L¹ is the same or different halo, and Ar is selected from the group consisting of: pyridyl, and pyridyl substituted with 1 substituent selected from the group consisting of: —Cl, —CF₃ and —CN.

Compounds of formula (I) also include any one of the compounds of formulas (I.A1a) to (I.A1h) above wherein n is 2, and the L¹ groups are bound to the phenyl moiety as shown in (IIA), each L¹ is F, and Ar is selected from the group consisting of p-Cl-phenyl-, p-CN-phenyl-, p-CF₃-phenyl, p-CH₃CH₂SO₂phenyl, p-Br-phenyl, m,p-di-F-phenyl, m,p-di-CN-phenyl, p-CH₃O-phenyl, p-CF₃CH₂Ophenyl, pyridyl, and pyridyl substituted with 1 or 2 substituents independently selected from the group consisting of: halogen, alkyl (e.g., C₁-C₆ alkyl), —CN, —CF₃, —O—(C₁-C₆)alkyl (e.g., —OCH₃), —O-(halo(C₁-C₆)alkyl) (e.g., —OCF₃ and —OCH₂CF₃), —C(O)—O—(C₁-C₆)alkyl (e.g., —C(O)OCH₃), -alkylene-OH (e.g., —CH₂OH), halo(C₁-C₆)alkyl (e.g., —CF₃), hydroxyalkoxy- (e.g., HOCH₂CH₂O—), and alkoxyalkoxy- (e.g., CH₃OCH₂CH₂O—).

Compounds of formula (I) also include any one of the compounds of formulas (I.A1a) to (I.A1h) above wherein n is 2, and the L¹ groups are bound to the phenyl moiety as shown in (IIA), each L¹ is F, and Ar is selected from the group consisting of p-Cl-phenyl-, p-CN-phenyl-, p-CF₃-phenyl, pyridyl, and pyridyl substituted with 1 or 2 substituents independently selected from the group consisting of: halogen, alkyl (e.g., C₁-C₆ alkyl), —CN, —CF₃, —O—(C₁-C₆)alkyl (e.g., —OCH₃), —O-(halo(C₁-C₆)alkyl) (e.g., —OCF₃), —C(O)—O—(C₁-C₆)alkyl (e.g., —C(O)OCH₃), -alkylene-OH (e.g., —CH₂OH), halo(C₁-C₆)alkyl (e.g., —CF₃), hydroxyalkoxy- (e.g., HOCH₂CH₂O—), and alkoxyalkoxy- (e.g., CH₃OCH₂CH₂O—).

Compounds of formula (I) also include any one of the compounds of formulas (I.A1a) to (I.A1h) above wherein n is 2, and the L¹ groups are bound to the phenyl moiety as shown in (IIA), each L¹ is F, and Ar is selected from the group consisting of p-Cl-phenyl-, p-CN-phenyl-, p-CF₃-phenyl, pyridyl, and pyridyl substituted with 1 substituent selected from the group consisting of: —Cl, —CF₃ and —CN.

Compounds of formula (I) also include any one of the compounds of formulas (I.A1a) to (I.A1h) above wherein n is 2, and the L¹ groups are bound to the phenyl moiety as shown in (IIA), each L¹ is F, and Ar is selected from the group consisting of p-Cl-phenyl-, p-CN-phenyl-, and p-CF₃-phenyl.

Compounds of formula (I) also include any one of the compounds of formulas (I.A1a) to (I.A1h) above wherein n is 2, and the L¹ groups are bound to the phenyl moiety as shown in (IIA), each L¹ is F, and Ar is selected from the group consisting of: pyridyl, and pyridyl substituted with 1 substituent selected from the group consisting —CF₃ and —CN.

Compounds of formula (I) also include any one of the compounds of formulas (I.A1a) to (I.A1h) above wherein n is 2, and the L¹ groups are bound to the phenyl moiety as shown in (IIA), each L¹ is F, and Ar is p-Cl-phenyl-.

Compounds of formula (I) also include any one of the compounds of formulas (I.A1a) to (I.A1h) above wherein n is 2, and the L¹ groups are bound to the phenyl moiety as shown in (IIA), each L¹ is F, and Ar is p-CN-phenyl-.

Compounds of formula (I) also include any one of the compounds of formulas (I.A1a) to (I.A1h) above wherein n is 2, and the L¹ groups are bound to the phenyl moiety as shown in (IIA), each L¹ is F, and Ar is p-CF₃-phenyl.

Compounds of formula (I) also include any one of the compounds of formulas (I.A1a) to (I.A1h) above wherein n is 2, and the L¹ groups are bound to the phenyl moiety as shown in (IIA), each L¹ is F, and Ar is p-CH₃CH₂SO₂phenyl.

Compounds of formula (I) also include any one of the compounds of formulas (I.A1a) to (I.A1h) above wherein n is 2, and the L¹ groups are bound to the phenyl moiety as shown in (IIA), each L¹ is F, and Ar is p-Br-phenyl.

Compounds of formula (I) also include any one of the compounds of formulas (I.A1a) to (I.A1h) above wherein n is 2, and the L¹ groups are bound to the phenyl moiety as shown in (IIA), each L¹ is F, and Ar is m,p-di-F-phenyl.

Compounds of formula (I) also include any one of the compounds of formulas (I.A1a) to (I.A1h) above wherein n is 2, and the L¹ groups are bound to the phenyl moiety as shown in (IIA), each L¹ is F, and Ar is m,p-di-CN-phenyl.

Compounds of formula (I) also include any one of the compounds of formulas (I.A1a) to (I.A1h) above wherein n is 2, and the L¹ groups are bound to the phenyl moiety as shown in (IIA), each L¹ is F, and Ar is p-CH₃O-phenyl.

Compounds of formula (I) also include any one of the compounds of formulas (I.A1a) to (I.A1h) above wherein n is 2, and the L¹ groups are bound to the phenyl moiety as shown in (IIA), each L¹ is F, and Ar is p-CF₃CH₂Ophenyl.

Compounds of formula (I) also include any one of the compounds of formulas (I.A1a) to (I.A1h) described above wherein R² is H.

Compounds of formula (I) also include any one of the compounds of formulas (I.A1a) to (I.A1h) described above wherein R² is alkyl.

Compounds of formula (I) also include any one of the compounds of formulas (I.A1a) to (I.A1h) described above wherein R² is methyl.

Compounds of formula (I) also include any one of the compounds of formulas (I.A1a) to (I.A1h) described above wherein R² is —CD₃.

Compounds of formula (I) include compounds of formula (I.A2a):

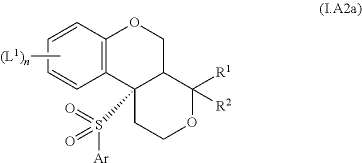

wherein all substituents are as defined for formula (I), and wherein at least one (e.g., 1-4, 1-3, 1-2, 2 or 1) H in formula (I.A2a) is replaced by deuterium (D).

Compounds of formula (I) include compounds of formula (I.A2b):

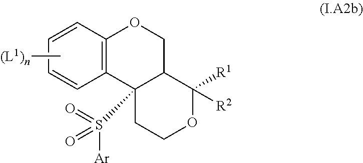

wherein all substituents are as defined for formula (I), and wherein at least one (e.g., 1-4, 1-3, 1-2, 2 or 1) H in formula (I.A2b) is replaced by deuterium (D).

Compounds of formula (I) include compounds of formula (I.A2c):

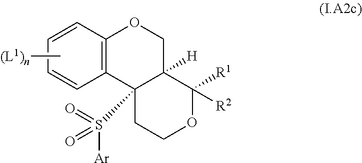

wherein all substituents are as defined for formula (I), and wherein at least one (e.g., 1-4, 1-3, 1-2, 2 or 1) H in formula (I.A2c) is replaced by deuterium (D).

Compounds of formula (I) incluse compounds of formula (I.A2d):

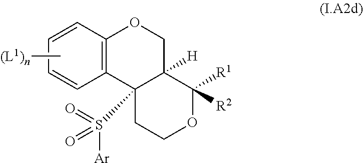

wherein all substituents are as defined for formula (I), and wherein at least one (e.g., 1-4, 1-3, 1-2, 2 or 1) H in formula (I.A2d) is replaced by deuterium (D).

Compounds of formula (I) include compounds of formula (I.A2e):

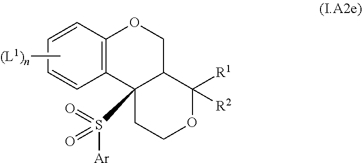

wherein all substituents are as defined for formula (I), and wherein at least one (e.g., 1-4, 1-3, 1-2, 2 or 1) H in formula (I.A2e) is replaced by deuterium (D).

Compounds of formula (I) include compounds of formula (I.A2f):

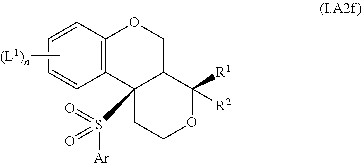

wherein all substituents are as defined for formula (I), and wherein at least one (e.g., 1-4, 1-3, 1-2, 2 or 1) H in formula (I.A2f) is replaced by deuterium (D).

Compounds of formula (I) include compounds of formula (I.A2g):

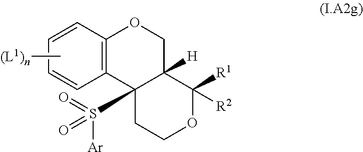

wherein all substituents are as defined for formula (I), and wherein at least one (e.g., 1-4, 1-3, 1-2, 2 or 1) H in formula (I.A2g) is replaced by deuterium (D).

Compounds of formula (I) include compounds of formula (I.A2h):

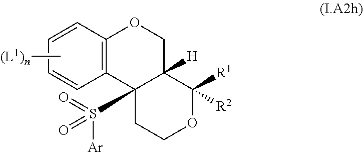

wherein all substituents are as defined for formula (I), and wherein at least one (e.g., 1-4, 1-3, 1-2, 2 or 1) H in formula (I.A2h) is replaced by deuterium (D).

Compounds of formula (I) also include any one of the compounds of formulas (I.A2a) to (I.A2h) above wherein n is 2, and the L¹ groups are bound to the phenyl moiety as shown in (IIA) and each L¹ is the same or different halo.

Compounds of formula (I) also include any one of the compounds of formulas (I.A2a) to (I.A2h) above wherein n is 2, and the L¹ groups are bound to the phenyl moiety as shown in (IIA) and each L¹ is F.

Compounds of formula (I) also include any one of the compounds of formulas (I.A2a) to (I.A2h) above wherein n is 2, and the L¹ groups are bound to the phenyl moiety as shown in (IIA) and Ar is selected from the group consisting of p-Cl-phenyl-, p-CN-phenyl-, p-CF₃-phenyl, p-CH₃CH₂SO₂phenyl, p-Br-phenyl, m,p-di-F-phenyl, m,p-di-CN-phenyl, p-CH₃O-phenyl, p-CF₃CH₂Ophenyl, pyridyl, and pyridyl substituted with 1 or 2 substituents independently selected from the group consisting of: halogen, alkyl (e.g., C₁-C₆ alkyl), —CN, —CF₃, —O—(C₁-C₆)alkyl (e.g., —OCH₃), —O-(halo(C₁-C₆)alkyl) (e.g., —OCF₃ and —OCH₂CF₃), —C(O)—O—(C₁-C₆)alkyl (e.g., —C(O)OCH₃), -alkylene-OH (e.g., —CH₂OH), halo(C₁-C₆)alkyl (e.g., —CF₃), hydroxyalkoxy- (e.g., HOCH₂CH₂O—), and alkoxyalkoxy- (e.g., CH₃OCH₂CH₂O—).

Compounds of formula (I) also include any one of the compounds of formulas (I.A2a) to (I.A2h) above wherein n is 2, and the L¹ groups are bound to the phenyl moiety as shown in (IIA) and Ar is selected from the group consisting of p-Cl-phenyl-, p-CN-phenyl-, p-CF₃-phenyl, pyridyl, and pyridyl substituted with 1 or 2 substituents independently selected from the group consisting of: halogen, alkyl (e.g., C₁-C₆ alkyl), —CN, —CF₃, —O—(C₁-C₆)alkyl (e.g., —OCH₃), —O-(halo(C₁-C₆)alkyl) (e.g., —OCF₃), —C(O)—O—(C₁-C₆)alkyl (e.g., —C(O)OCH₃), -alkylene-OH (e.g., —CH₂OH), halo(C₁-C₆)alkyl (e.g., —CF₃), hydroxyalkoxy- (e.g., HOCH₂CH₂O—), and alkoxyalkoxy- (e.g., CH₃OCH₂CH₂O—).

Compounds of formula (I) also include any one of the compounds of formulas (I.A2a) to (I.A2h) above wherein n is 2, and the L¹ groups are bound to the phenyl moiety as shown in (IIA) and Ar is selected from the group consisting of p-Cl-phenyl-, p-CN-phenyl-, p-CF₃-phenyl, pyridyl, and pyridyl substituted with 1 substituent selected from the group consisting of: —Cl, —CF₃ and —CN.

Compounds of formula (I) also include any one of the compounds of formulas (I.A2a) to (I.A2h) above wherein n is 2, and the L¹ groups are bound to the phenyl moiety as shown in (IIA) and Ar is selected from the group consisting of p-Cl-phenyl-, p-CN-phenyl-, and p-CF₃-phenyl.

Compounds of formula (I) also include any one of the compounds of formulas (I.A2a) to (I.A2h) above wherein n is 2, and the L¹ groups are bound to the phenyl moiety as shown in (IIA) and Ar is selected from the group consisting of: pyridyl, and pyridyl substituted with 1 substituent selected from the group consisting of: —Cl, —CF₃ and —CN.

Compounds of formula (I) also include any one of the compounds of formulas (I.A2a) to (I.A2h) above wherein n is 2, and the L¹ groups are bound to the phenyl moiety as shown in (HA), each L¹ is the same or different halo, and Ar is selected from the group consisting of p-Cl-phenyl-, p-CN-phenyl-, p-CF₃-phenyl, p-CH₃CH₂SO₂phenyl, p-Br-phenyl, m,p-di-F-phenyl, m,p-di-CN-phenyl, p-CH₃O-phenyl, p-CF₃CH₂Ophenyl, pyridyl, and pyridyl substituted with 1 or 2 substituents independently selected from the group consisting of: halogen, alkyl (e.g., C₁-C₆ alkyl), —CN, —CF₃, —O—(C₁-C₆)alkyl (e.g., —OCH₃), —O-(halo(C₁-C₆)alkyl) (e.g., —OCF₃ and —OCH₂CF₃), —C(O)—O—(C₁-C₆)alkyl (e.g., —C(O)OCH₃), -alkylene-OH (e.g., —CH₂OH), halo(C₁-C₆)alkyl (e.g., —CF₃), hydroxyalkoxy- (e.g., HOCH₂CH₂O—), and alkoxyalkoxy- (e.g., CH₃OCH₂CH₂O—).

Compounds of formula (I) also include any one of the compounds of formulas (I.A2a) to (I.A2h) above wherein n is 2, and the L¹ groups are bound to the phenyl moiety as shown in (IIA), each L¹ is the same or different halo, and Ar is selected from the group consisting of p-Cl-phenyl-, p-CN-phenyl-, p-CF₃-phenyl, pyridyl, and pyridyl substituted with 1 or 2 substituents independently selected from the group consisting of: halogen, alkyl (e.g., C₁-C₆ alkyl), —CN, —CF₃, —O—(C₁-C₆)alkyl (e.g., —OCH₃), —O-(halo(C₁-C₆)alkyl) (e.g., —OCF₃), —C(O)—O—(C₁-C₆)alkyl (e.g., —C(O)OCH₃), -alkylene-OH (e.g., —CH₂OH), halo(C₁-C₆)alkyl (e.g., —CF₃), hydroxyalkoxy- (e.g., HOCH₂CH₂O—), and alkoxyalkoxy- (e.g., CH₃OCH₂CH₂O—).

Compounds of formula (I) also include any one of the compounds of formulas (I.A2a) to (I.A2h) above wherein n is 2, and the L¹ groups are bound to the phenyl moiety as shown in (IIA), each L¹ is the same or different halo, and Ar is selected from the group consisting of p-Cl-phenyl-, p-CN-phenyl-, p-CF₃-phenyl, pyridyl, and pyridyl substituted with 1 substituent selected from the group consisting of: —Cl, —CF₃ and —CN.

Compounds of formula (I) also include any one of the compounds of formulas (I.A2a) to (I.A2h) above wherein n is 2, and the L¹ groups are bound to the phenyl moiety as shown in (IIA), each L¹ is the same or different halo, and Ar is selected from the group consisting of p-Cl-phenyl-, p-CN-phenyl-, and p-CF₃-phenyl.

Compounds of formula (I) also include any one of the compounds of formulas (I.A2a) to (I.A2h) above wherein n is 2, and the L¹ groups are bound to the phenyl moiety as shown in (HA), each L¹ is the same or different halo, and Ar is selected from the group consisting of: pyridyl, and pyridyl substituted with 1 substituent selected from the group consisting of: —Cl, —CF₃ and —CN.

Compounds of formula (I) also include any one of the compounds of formulas (I.A2a) to (I.A2h) above wherein n is 2, and the L¹ groups are bound to the phenyl moiety as shown in (IIA), each L¹ is F, and Ar is selected from the group consisting of p-Cl-phenyl-, p-CN-phenyl-, p-CF₃-phenyl, p-CH₃CH₂SO₂phenyl, p-Br-phenyl, m,p-di-F-phenyl, m,p-di-CN-phenyl, p-CH₃O-phenyl, p-CF₃CH₂Ophenyl, pyridyl, and pyridyl substituted with 1 or 2 substituents independently selected from the group consisting of: halogen, alkyl (e.g., C₁-C₆ alkyl), —CN, —CF₃, —O—(C₁-C₆)alkyl (e.g., —OCH₃), —O-(halo(C₁-C₆)alkyl) (e.g., —OCF₃ and —OCH₂CF₃), —C(O)—O—(C₁-C₆)alkyl (e.g., —C(O)OCH₃), -alkylene-OH (e.g., —CH₂OH), halo(C₁-C₆)alkyl (e.g., —CF₃), hydroxyalkoxy- (e.g., HOCH₂CH₂O—), and alkoxyalkoxy- (e.g., CH₃OCH₂CH₂O—).

Compounds of formula (I) also include any one of the compounds of formulas (I.A2a) to (I.A2h) above wherein n is 2, and the L¹ groups are bound to the phenyl moiety as shown in (HA), each L¹ is F, and Ar is selected from the group consisting of p-Cl-phenyl-, p-CN-phenyl-, p-CF₃-phenyl, pyridyl, and pyridyl substituted with 1 or 2 substituents independently selected from the group consisting of: halogen, alkyl (e.g., C₁-C₆ alkyl), —CN, —CF₃, —O—(C₁-C₆)alkyl (e.g., —OCH₃), —O-(halo(C₃-C₆)alkyl) (e.g., —OCF₃), —C(O)—O—(C₁-C₆)alkyl (e.g., —C(O)OCH₃), -alkylene-OH (e.g., —CH₂OH), halo(C₁-C₆)alkyl (e.g., —CF₃), hydroxyalkoxy- (e.g., HOCH₂CH₂O—), and alkoxyalkoxy- (e.g., CH₃OCH₂CH₂O—).

Compounds of formula (I) also include any one of the compounds of formulas (I.A2a) to (I.A2h) above wherein n is 2, and the L¹ groups are bound to the phenyl moiety as shown in (IIA), each L¹ is F, and Ar is selected from the group consisting of p-Cl-phenyl-, p-CN-phenyl-, p-CF₃-phenyl, pyridyl, and pyridyl substituted with 1 substituent selected from the group consisting of: —Cl, —CF₃ and —CN.

Compounds of formula (I) also include any one of the compounds of formulas (I.A2a) to (I.A2h) above wherein n is 2, and the L¹ groups are bound to the phenyl moiety as shown in (IIA), each L¹ is F, and Ar is selected from the group consisting of p-Cl-phenyl-, p-CN-phenyl-, and p-CF₃-phenyl.

Compounds of formula (I) also include any one of the compounds of formulas (I.A2a) to (I.A2h) above wherein n is 2, and the L¹ groups are bound to the phenyl moiety as shown in (IIA), each L¹ is F, and Ar is selected from the group consisting of: pyridyl, and pyridyl substituted with 1 substituent selected from the group consisting of: —Cl, —CF₃ and —CN.

Compounds of formula (I) also include any one of the compounds of formulas (I.A2a) to (I.A2h) above wherein n is 2, and the L¹ groups are bound to the phenyl moiety as shown in (IIA), each L¹ is F, and Ar is p-Cl-phenyl-.

Compounds of formula (I) also include any one of the compounds of formulas (I.A2a) to (I.A2h) above wherein n is 2, and the L¹ groups are bound to the phenyl moiety as shown in (IIA), each L¹ is F, and Ar is p-CN-phenyl-.

Compounds of formula (I) also include any one of the compounds of formulas (I.A2a) to (I.A2h) above wherein n is 2, and the L¹ groups are bound to the phenyl moiety as shown in (IIA), each L¹ is F, and Ar is p-CF₃-phenyl.

Compounds of formula (I) also include any one of the compounds of formulas (I.A2a) to (I.A2h) above wherein n is 2, and the L¹ groups are bound to the phenyl moiety as shown in (IIA), each L¹ is F, and Ar is p-CH₃CH₂SO₂phenyl.

Compounds of formula (I) also include any one of the compounds of formulas (I.A2a) to (I.A2h) above wherein n is 2, and the L¹ groups are bound to the phenyl moiety as shown in (IIA), each L¹ is F, and Ar is p-Br-phenyl.

Compounds of formula (I) also include any one of the compounds of formulas (I.A2a) to (I.A2h) above wherein n is 2, and the L¹ groups are bound to the phenyl moiety as shown in (IIA), each L¹ is F, and Ar is m,p-di-F-phenyl.

Compounds of formula (I) also include any one of the compounds of formulas (I.A2a) to (I.A2h) above wherein n is 2, and the L¹ groups are bound to the phenyl moiety as shown in (IIA), each L¹ is F, and Ar is m,p-di-CN-phenyl.

Compounds of formula (I) also include any one of the compounds of formulas (I.A2a) to (I.A2h) above wherein n is 2, and the L¹ groups are bound to the phenyl moiety as shown in (IIA), each L¹ is F, and Ar is p-CH₃O-phenyl.

Compounds of formula (I) also include any one of the compounds of formulas (I.A2a) to (I.A2h) above wherein n is 2, and the L¹ groups are bound to the phenyl moiety as shown in (IIA), each L¹ is F, and Ar is p-CF₃CH₂Ophenyl.

Compounds of formula (I) also include any one of the compounds of formulas (I.A2a) to (I.A2h) described above wherein R² is H.

Compounds of formula (I) also include any one of the compounds of formulas (I.A2a) to (I.A2h) described above wherein R² is alkyl.

Compounds of formula (I) also include any one of the compounds of formulas (I.A2a) to (I.A2h) described above wherein R² is methyl.

Compounds of formula (I) also include any one of the compounds of formulas (I.A2a) to (I.A2h) described above wherein R² is —CD₃.

Compounds of formula (I) also include any one of the compounds of formulas (Ii) to (Ivii), (I.A1), (I.A1a) to (I.A1h), (I.A2), and (I.A2a) to (I.A2h) described above wherein R¹ is -alkylene-(tetrahydrothiophene 1,1-dioxide).

Compounds of formula (I) also include any one of the compounds of formulas (Ii) to (Ivii), (I.A1), (I.A1a) to (I.A1h), (I.A2), and (I.A2a) to (I.A2h) described above R¹ is -alkenyl-S(O)₂—(C₁-C₆)alkyl.

Compounds of formula (I) also include any one of the compounds of formulas (Ii) to (Ivii), (I.A1), (I.A1a) to (I.A1h), (I.A2), and (I.A2a) to (I.A2h) described above wherein R¹ is -cycloalkyl-S(O)₂—(C₁-C₆)alkyl.

Compounds of formula (I) also include any one of the compounds of formulas (Ii) to (Ivii), (I.A1), (I.A1a) to (I.A1h), (I.A2), and (I.A2a) to (I.A2h) described above wherein R¹ is an -alkylene-S(O)₂—(C₁-C₆)alkyl group.

Compounds of formula (I) also include any one of the compounds of formulas (Ii) to (Ivii), (I.A1), (I.A1a) to (I.A1h), (I.A2), and (I.A2a) to (I.A2h) described above wherein R¹ is a —(C₁ to C₂) alkylene-S(O)₂—(C₁-C₆)alkyl group.

Compounds of formula (I) also include any one of the compounds of formulas (Ii) to (Ivii), (I.A1), (I.A1a) to (I.A1h), (I.A2), and (I.A2a) to (I.A2h) described above wherein R¹ is a —(C₁ to C₂) alkylene-S(O)₂—(C₁-C₃)alkyl group.

Compounds of formula (I) also include any one of the compounds of formulas (Ii) to (Ivii), (I.A1), (I.A1a) to (I.A1h), (I.A2), and (I.A2a) to (I.A2h) described above wherein R₁ is a —(C₂) alkylene-S(O)₂—(C₁-C₆)alkyl group.

Compounds of formula (I) also include any one of the compounds of formulas (I.A1), (I.A2), (I.A1a), (I.A1e), (I.A2a) or (I.A2e) wherein R¹ is selected from the group consisting of:

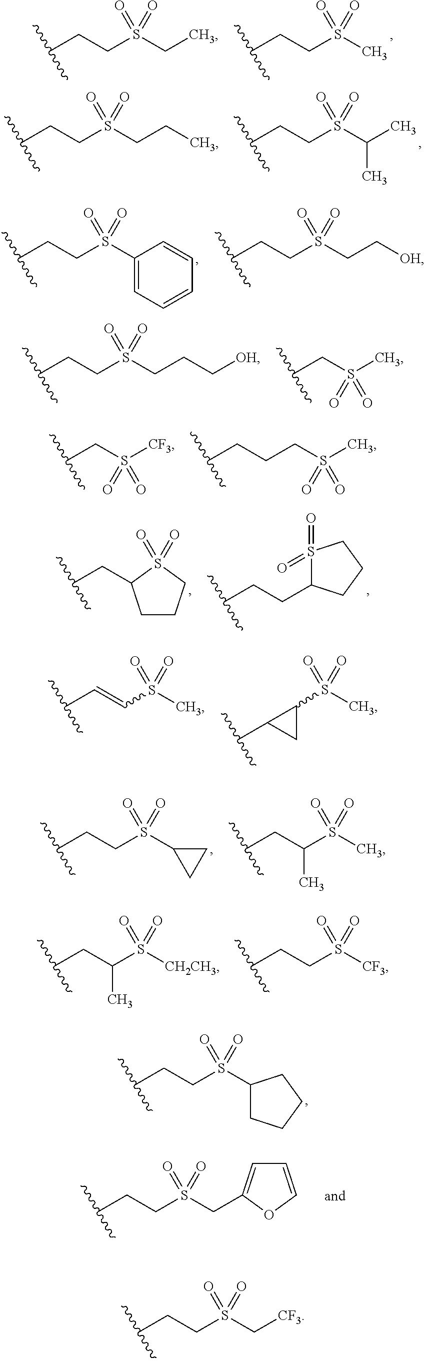

Compounds of formula (I) also include any one of the compounds of formulas (Ii), (Iii), (Iiii), (Iiv), (I.A1b), (I.A1c), (I.A1d), (I.A2b), (I.A2c), or (I.A1d) wherein R¹ is selected from the group consisting of:

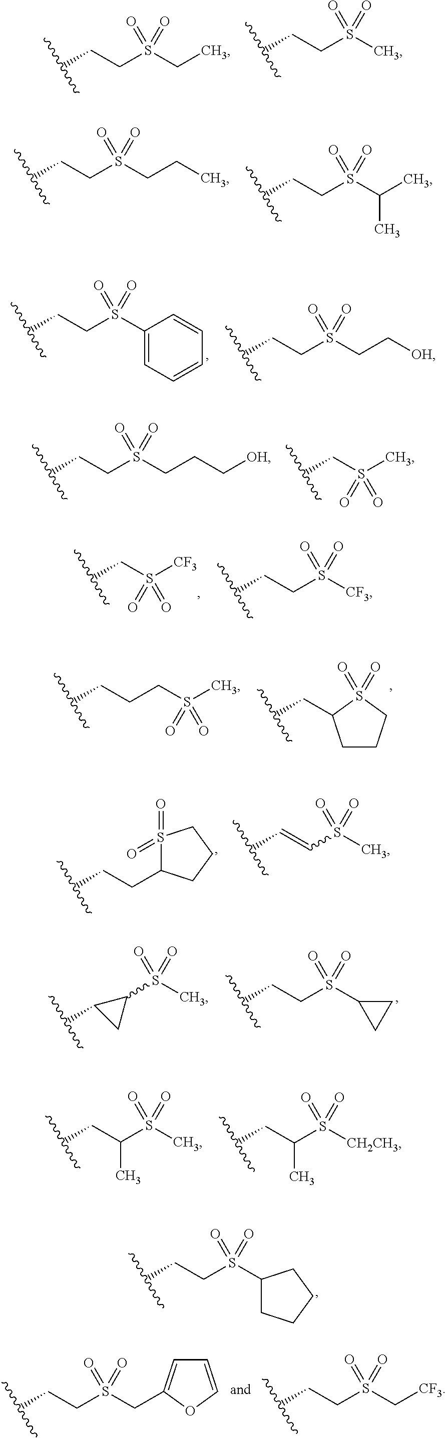

Compounds of formula (I) also include any one of the compounds of formulas (Ii), (Iii), (Iiii), (Iiv), (I.A1b), (I.A1c), (I.A1d), (I.A2b), (I.A2c), or (I.A1d), and R¹ is

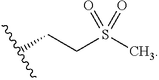

Compounds of formula (I) also include any one of the compounds of formulas (Ii), (Iii), (Iiii), (Iiv), (I.A1b), (I.A1c), (I.A1d), (I.A2b), (I.A2c), or (I.A1d), and R¹ is

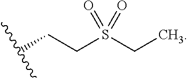

Compounds of formula (I) also include any one of the compounds of formulas (Ii), (Iii), (Iiii), (Iiv), (I.A1b), (I.A1c), (I.A1d), (I.A2b), (I.A2c), or (I.A1d), and R¹ is

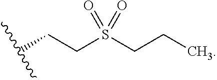

Compounds of formula (I) also include any one of the compounds of formulas (Ii), (Iii), (Iiii), (Iiv), (I.A1b), (I.A1c), (I.A1d), (I.A2b), (I.A2c), or (I.A1d), and R1 is

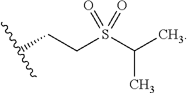

Compounds of formula (I) also include any one of the compounds of formulas (Ii), (Iii), (Iiii), (Iiv), (I.A1b), (I.A1c), (I.A1d), (I.A2b), (I.A2c), or (I.A1d), and R1 is

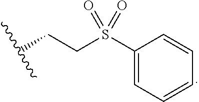

Compounds of formula (I) also include any one of the compounds of formulas (Ii), (Iii), (Iiii), (Iiv), (I.A1b), (I.A1c), (I.A1d), (I.A2b), (I.A2c), or (I.A1d), and R1 is

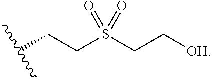

Compounds of formula (I) also include any one of the compounds of formulas (Ii), (Iii), (Iiii), (Iiv), (I.A1b), (I.A1c), (I.A1d), (I.A2b), (I.A2c), or (I.A1d), and R1 is

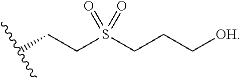

Compounds of formula (I) also include any one of the compounds of formulas (Ii), (Iii), (Iiii), (Iiv), (I.A1b), (I.A1c), (I.A1d), (I.A2b), (I.A2c), or (I.A1d), and R1 is

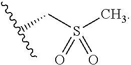

Compounds of formula (I) also include any one of the compounds of formulas (Ii), (Iii), (Iiii), (Iiv), (I.A1b), (I.A1c), (I.A1d), (I.A2b), (I.A2c), or (I.A1d), and R1 is

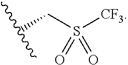

Compounds of formula (I) also include any one of the compounds of formulas (Ii), (Iii), (Iiii), (Iiv), (I.A1b), (I.A1c), (I.A1d), (I.A2b), (I.A2c), or (I.A1d), and R1 is

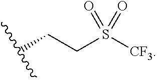

Compounds of formula (I) also include any one of the compounds of formulas (Ii), (Iii), (Iiii), (Iiv), (I.A1b), (I.A1c), (I.A1d), (I.A2b), (I.A2c), or (I.A1d), and R1 is

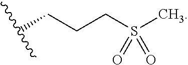

Compounds of formula (I) also include any one of the compounds of formulas (Ii), (Iii), (Iiii), (Iiv), (I.A1b), (I.A1c), (I.A1d), (I.A2b), (I.A2c), or (I.A1d), and R1 is

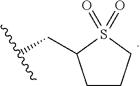

Compounds of formula (I) also include any one of the compounds of formulas (Ii), (Iii), (Iiii), (Iiv), (I.A1b), (I.A1c), (I.A1d), (I.A2b), (I.A2c), or (I.A1d), and R1 is

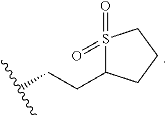

Compounds of formula (I) also include any one of the compounds of formulas (Ii), (Iii), (Iiii), (Iiv), (I.A1b), (I.A1), (I.A1d), (I.A2b), (I.A2c), or (I.A1d), and R1 is

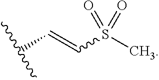

Compounds of formula (I) also include any one of the compounds of formulas (Ii), (Iii), (Iiii), (Iiv), (I.A1b), (I.A1c), (I.A1d), (I.A2b), (I.A2c), or (I.A1d), and R1 is

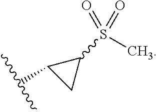

Compounds of formula (I) also include any one of the compounds of formulas (Ii), (Iii), (Iiii), (Iiv), (I.A1b), (I.A1c), (I.A1d), (I.A2b), (I.A2c), or (I.A1d), and R1 is

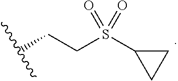

Compounds of formula (I) also include any one of the compounds of formulas (Ii), (Iii), (Iiii), (Iiv), (I.A1b), (I.A1c), (I.A1d), (I.A2b), (I.A2c), or (I.A1d), and R1 is

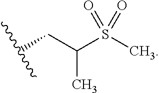

Compounds of formula (I) also include any one of the compounds of formulas (Ii), (Iii), (Iiii), (Iiv), (I.A1b), (I.A1c), (I.A1d), (I.A2b), (I.A2c), or (I.A1d), and R1 is

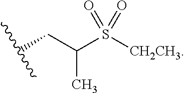

Compounds of formula (I) also include any one of the compounds of formulas (Ii), (Iii), (Iiii), (Iiv), (I.A1b), (I.A1c), (I.A1d), (I.A2b), (I.A2c), or (I.A1c), and R1 is

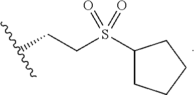

Compounds of formula (I) also include any one of the compounds of formulas (Ii), (Iii), (Iiii), (Iiv), (I.A1b), (I.A1c), (I.A1d), (I.A2b), (I.A2c), or (I.A1d), and R1 is

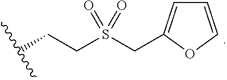

Compounds of formula (I) also include any one of the compounds of formulas (Ii), (Iii), (Iiii), (Iiv), (I.A1b), (I.A1c), (I.A1d), (I.A2b), (I.A2c), or (I.A1d), and R1 is

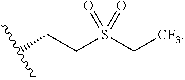

Compounds of formula (I) also include any one of the compounds of formulas (Iv), (Ivi), (Ivii), (I.A1f), (I.A1g), (I.A1h), (I.A2f), (I.A2g), or (I.A2h) wherein R1 is selected from the group consisting of:

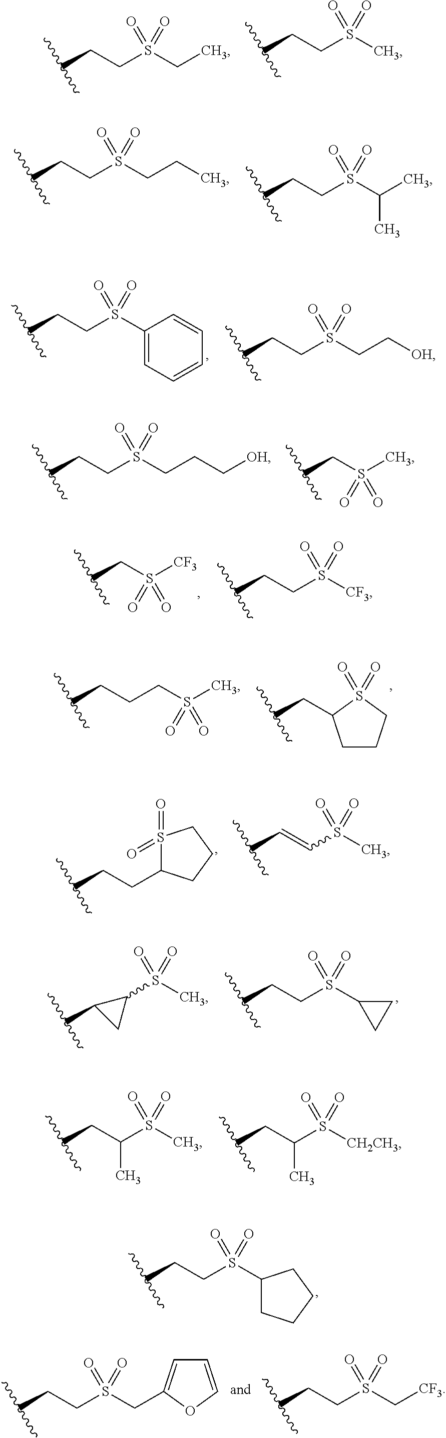

Compounds of formula (I) also include any one of the compounds of formulas (Iv), (Ivi), (Ivii), (I.A1f), (I.A1g), (I.A1h), (I.A2f), (I.A2g), or (I.A2h) wherein R1 is

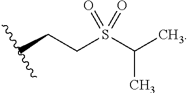

Compounds of formula (I) also include any one of the compounds of formulas (Iv), (Ivi), (Ivii), (I.A1f), (I.A1g), (I.A1h), (I.A2f), (I.A2g), or (I.A2h) wherein R1 is

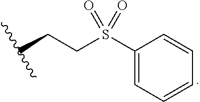

Compounds of formula (I) also include any one of the compounds of formulas (Iv), (Ivi), (Ivii), (I.A1f), (I.A1g), (I.A1h), (I.A2f), (I.A2g), or (I.A2h) wherein R1 is

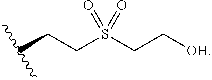

Compounds of formula (I) also include any one of the compounds of formulas (Iv), (Ivi), (Ivii), (I.A1f), (I.A1g), (I.A1h), (I.A2f), (I.A2g), or (I.A2h) wherein R1 is

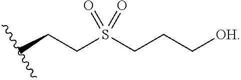

Compounds of formula (I) also include any one of the compounds of formulas (Iv), (Ivi), (Ivii), (I.A1f), (I.A1g), (I.A1h), (I.A2f), (I.A2g), or (I.A2h) wherein R1 is

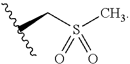

Compounds of formula (I) also include any one of the compounds of formulas (Iv), (Ivi), (Ivii), (I.A1f), (I.A1g), (I.A1h), (I.A2f), (I.A2g), or (I.A2h) wherein R1 is

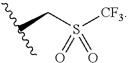

Compounds of formula (I) also include any one of the compounds of formulas (Iv), (Ivi), (Ivii), (I.A1f), (I.A1g), (I.A1h), (I.A2f), (I.A2g), or (I.A2h) wherein R1 is

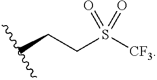

Compounds of formula (I) also include any one of the compounds of formulas (Iv), (Ivi), (Ivii), (I.A1f), (I.A1g), (I.A1h), (I.A2f), (I.A2g), or (I.A2h) wherein R1 is

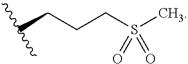

Compounds of formula (I) also include any one of the compounds of formulas (Iv), (Ivi), (Ivii), (I.A1f), (I.A1g), (I.A1h), (I.A2f), (I.A2g), or (I.A2h) wherein R1 is

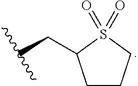

Compounds of formula (I) also include any one of the compounds of formulas (Iv), (Ivi), (Ivii), (I.A1f), (I.A1g), (I.A1h), (I.A2f), (I.A2g), or (I.A2h) wherein R1 is

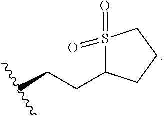

Compounds of formula (I) also include any one of the compounds of formulas (Iv), (Ivi), (Ivii), (I.A1f), (I.A1g), (I.A1h), (I.A2f), (I.A2g), or (I.A2h) wherein R1 is

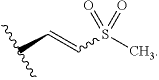

Compounds of formula (I) also include any one of the compounds of formulas (Iv), (Ivi), (Ivii), (I.A1f), (I.A1g), (I.A1h), (I.A2f), (I.A2g), or (I.A2h) wherein R1 is

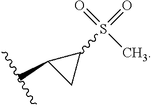

Compounds of formula (I) also include any one of the compounds of formulas (Iv), (Ivi), (Ivii), (I.A1f), (I.A1g), (I.A1h), (I.A2f), (I.A2g), or (I.A2h) wherein R1 is

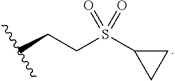

Compounds of formula (I) also include any one of the compounds of formulas (Iv), (Ivi), (Ivii), (I.A1f), (I.A1g), (I.A1h), (I.A2f), (I.A2g), or (I.A2h) wherein R1 is

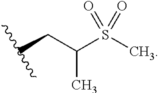

Compounds of formula (I) also include any one of the compounds of formulas (Iv), (Ivi), (Ivii), (I.A1f), (I.A1g), (I.A1h), (I.A2f), (I.A2g), or (I.A2h) wherein R1 is

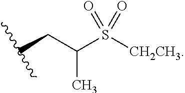

Compounds of formula (I) also include any one of the compounds of formulas (Iv), (Ivi), (Ivii), (I.A1f), (I.A1g), (I.A1h), (I.A2f), (I.A2g), or (I.A2h) wherein R1 is

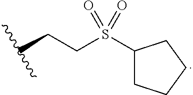

Compounds of formula (I) also include any one of the compounds of formulas (Iv), (Ivi), (Ivii), (I.A1f), (I.A1g), (I.A1h), (I.A2f), (I.A2g), or (I.A2h) wherein R1 is

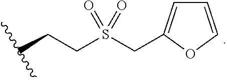

Compounds of formula (I) also include any one of the compounds of formulas (Iv), (Ivi), (Ivii), (I.A1f), (I.A1g), (I.A1h), (I.A2f), (I.A2g), or (I.A2h) wherein R1 is

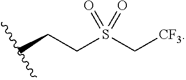

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

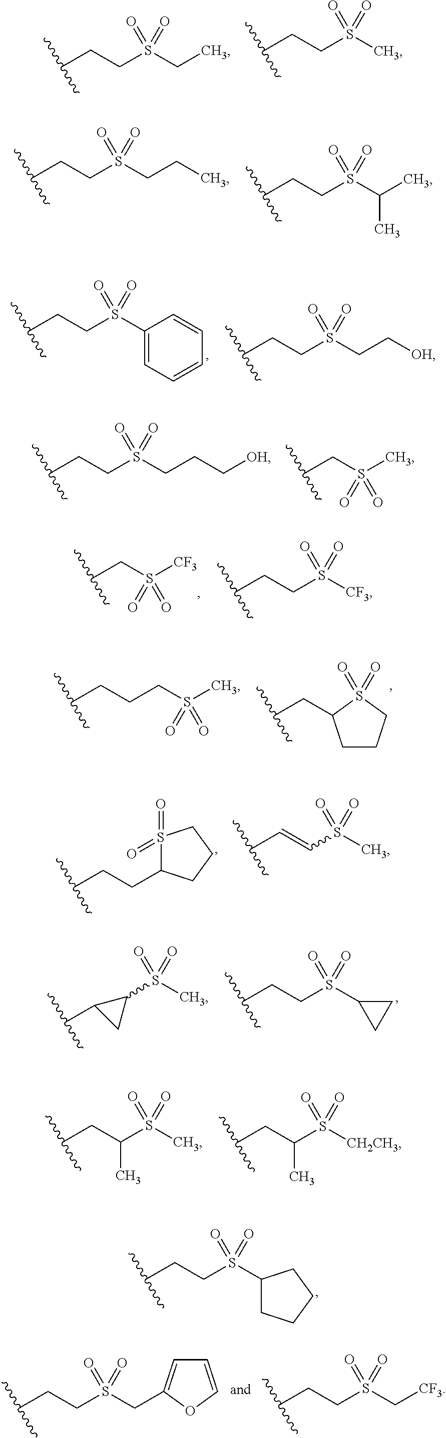

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

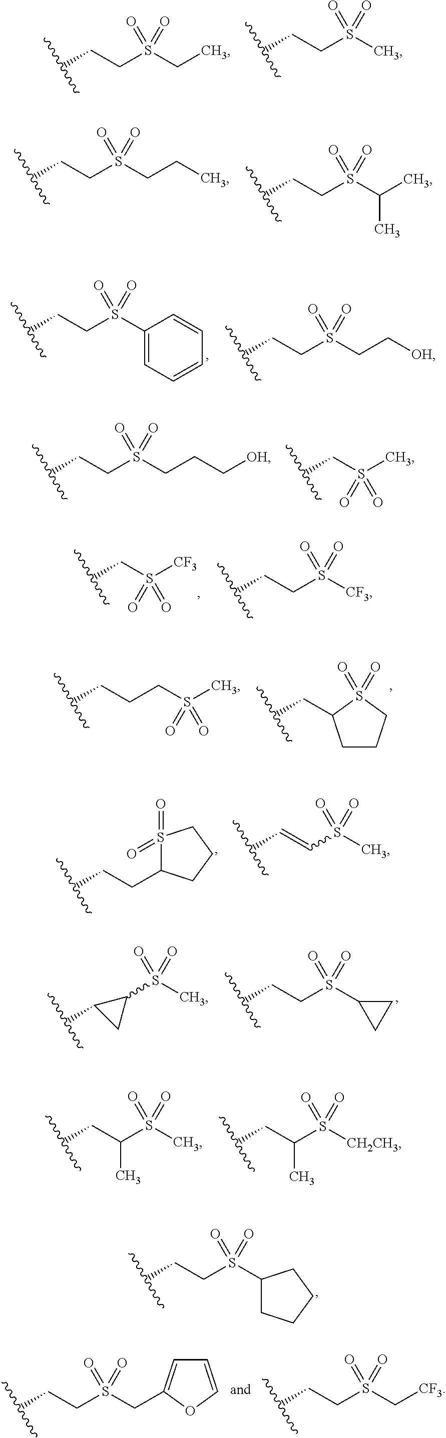

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

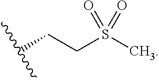

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

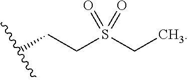

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

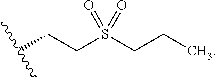

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

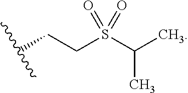

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

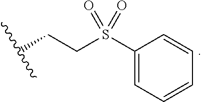

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

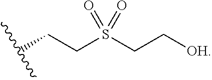

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

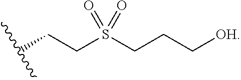

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

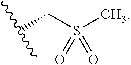

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

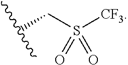

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

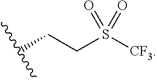

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

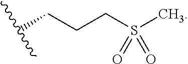

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

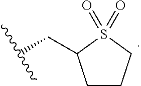

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

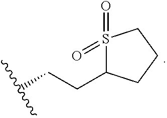

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

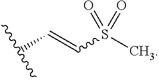

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

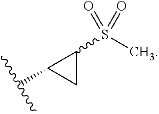

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

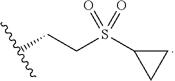

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

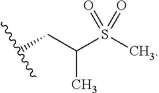

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

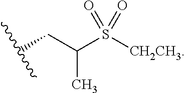

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

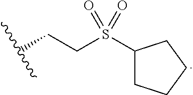

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

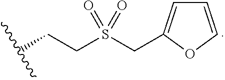

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

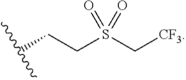

Compounds of formula (I) also include any one of the compounds of formulas I.A1i, I.A1j, I.A1k, I.A1n, I.A1o, I.A1p, I.A1q, I.A1r, or I.A1s, wherein R¹ is selected from the group consisting of:

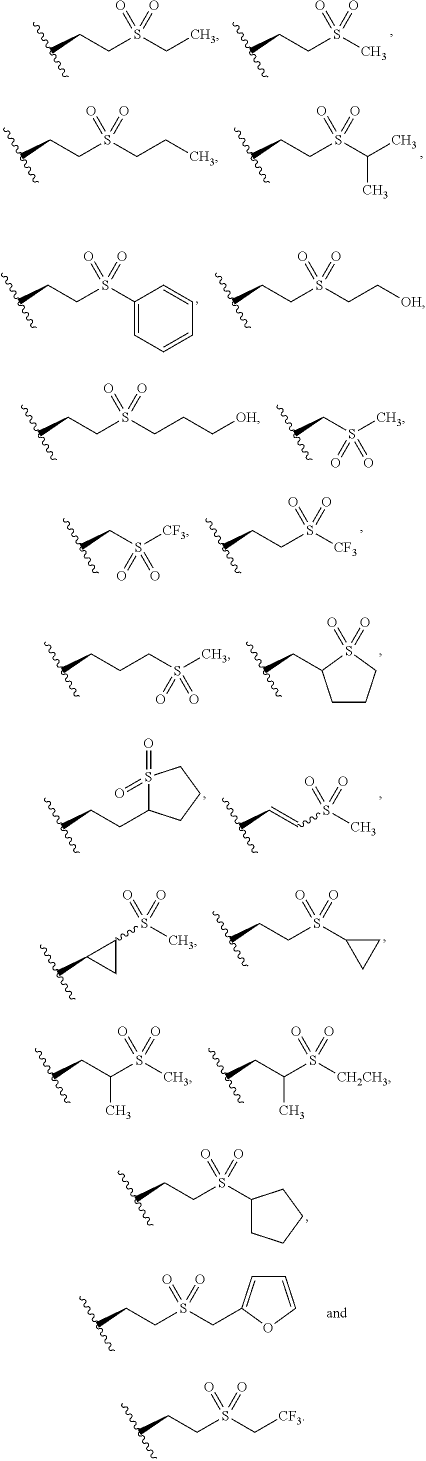

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

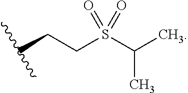

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

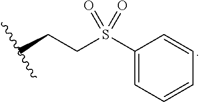

Compounds of formula (I) also include any one of the compounds of formulas I.A1i, I.A1j, I.A1k, I.A1n, I.A1o, I.A1p, I.A1q, I.A1r, or I.A1s, wherein R¹ is selected from the group consisting of:

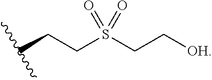

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

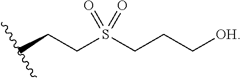

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

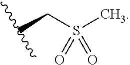

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

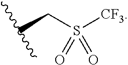

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

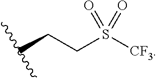

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the wow consisting of:

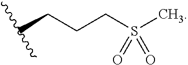

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

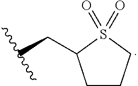

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

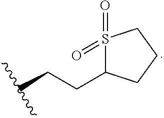

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

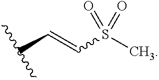

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

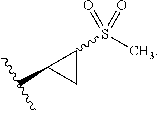

Compounds of formula (I) also include any one of the compounds of formulas I.A1i, I.A1j, I.A1k, I.A1n, I.A1o, I.A1p, I.A1q, I.A1r, I.A1s, wherein R1 is selected from the group consisting of:

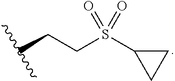

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

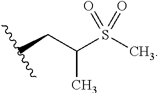

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

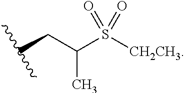

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

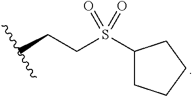

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

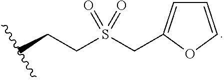

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

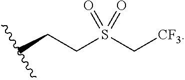

Compounds of formula (I) also include any one of the compounds of formulas I.A1i, I.A1j, I.A1k, I.A1l, I.A1m, I.A1n, I.A1o, I.A1p, I.A1q, I.A1r, or I.A1s, wherein R1 is selected from the group consisting of:

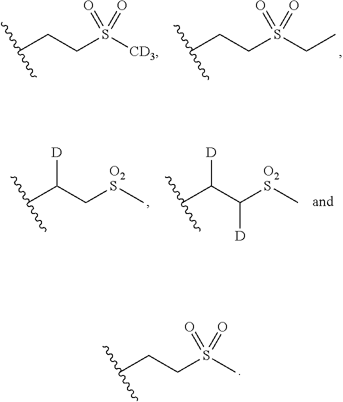

Compounds of formula (I) also include any one of the compounds of formulas I.A1i, I.A1j, I.A1k, I.A1l, I.A1m, I.A1n, I.A1o, I.A1p, I.A1q, I.A1r, or I.A1s, wherein R1 is selected from the group consisting of:

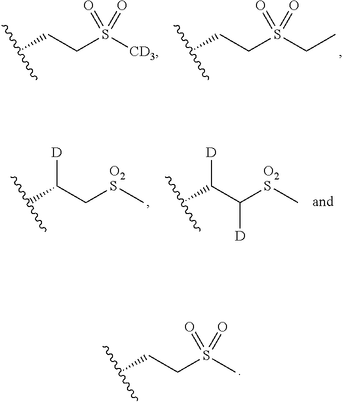

Compounds of formula (I) also include any one of the compounds of formulas I.A1i, I.A1j, I.A1k, I.A1l, I.A1m, I.A1n, I.A1o, I.A1p, I.A1q, I.A1r, or I.A1s, wherein R1 is selected from the group consisting of:

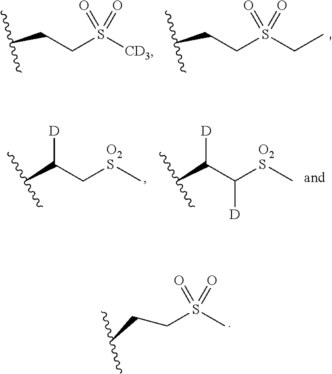

Compounds of formula (I) also include any one of the compounds of formulas, I.A1l or I.A1m, wherein R1 is selected from the a group consisting of:

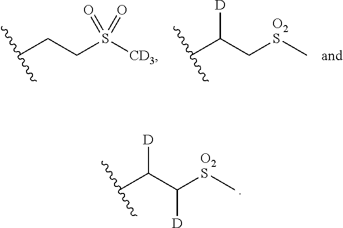

Compounds of formula (I) also include any one of the compounds of formulas, I.A1l or I.A1m, wherein R1 is selected from the group consisting of:

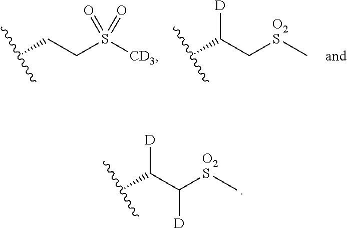

Compounds of formula (I) also include any one of the compounds of formulas, I.A1l or I.A1m, wherein R1 is selected from the group consisting of:

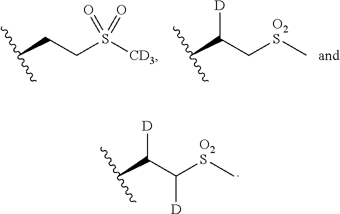

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

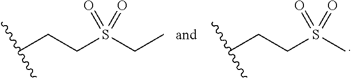

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

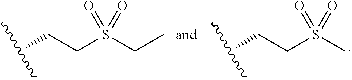

Compounds of formula (I) also include any one of the compounds of formulas I.A1i, I.A1j, I.A1k, I.A1n, I.A1o, I.A1p, I.A1q, I.A1r, or I.A1s, wherein R1 is selected from the group consisting of:

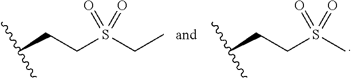

Other embodiments of this invention are directed to any one of the above embodiments directed to any of the definitions of R¹ above wherein at least one (e.g., 1-4, 1-3, 1-2, 2 or 1) H in formula (I.A2) is replaced by deuterium (D)

Examples of compounds wherein at least one (e.g., 1-4, 1-3, 1-2, 2 or 1) H can be replaced by deuterium (D) include, but are not limited to the compounds 12, 13, 22A to 22N, 22B-rac, 26A, 26B, 27, 28, 31, 33(−), 36, 37, 39, 44A, 44B, 55 to 57, 60, 70A, 71A(−), 75A-rac, 75B(−), 76A(−), 76A(+), 76B(−), 78A(−), 79A(−), 80A(+), 81A-rac, 81A(−), 81A(+), 81B(−), 82A, 83A, 84A, 85A, 85B(−), 86A, 87B, 95B-rac, 95B(−), 95B(+), 106(−), 107(−), 108(−), and 111(−), identified in the table below.

| Compd | |
|---|---|
| No. | Structure |
| 12 | 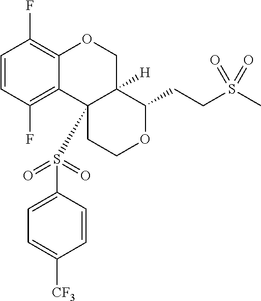 |
| 13 | 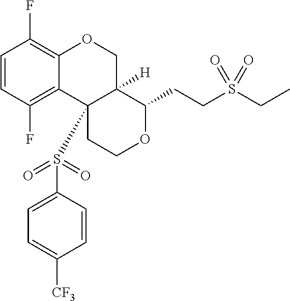 |
| 22A | 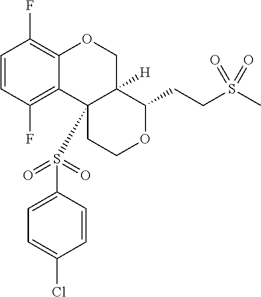 |
| 22B | 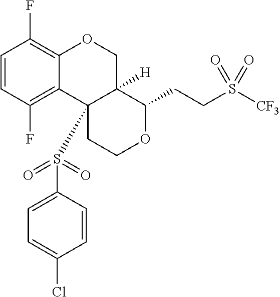 |
| 22C | 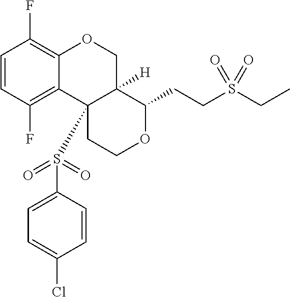 |
| 22D | 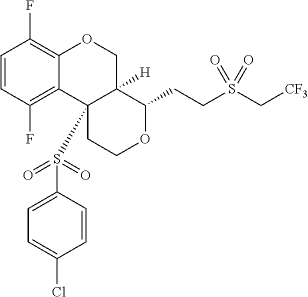 |
| 22E | 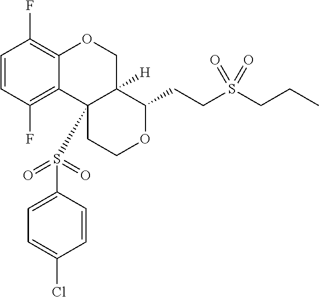 |
| 22F | 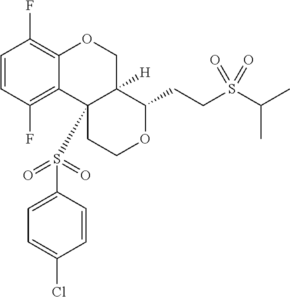 |
| 22G | 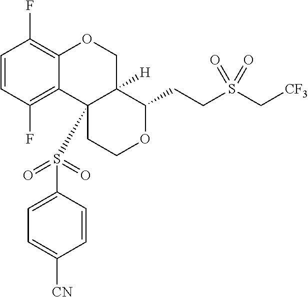 |
| 22H | 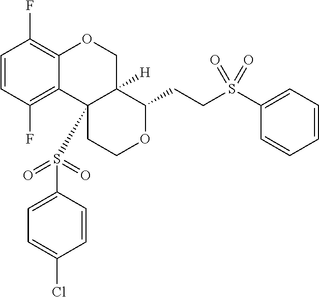 |
| 22I | 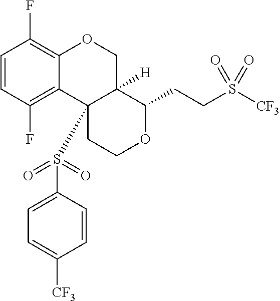 |
| 22J | 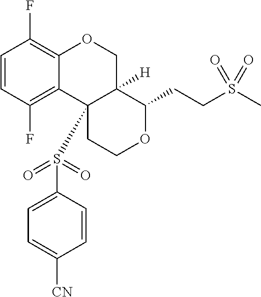 |
| 22K | 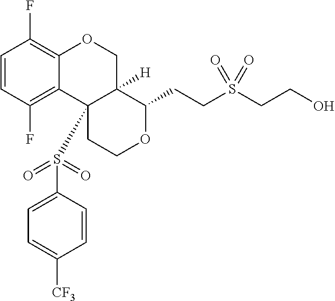 |
| 22L | 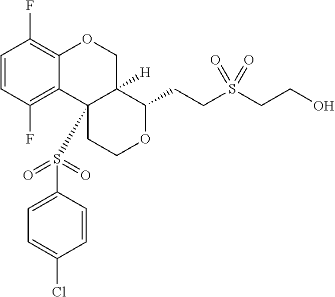 |
| 22M | 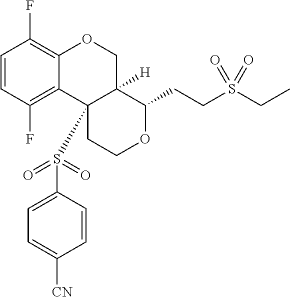 |
| 22N | 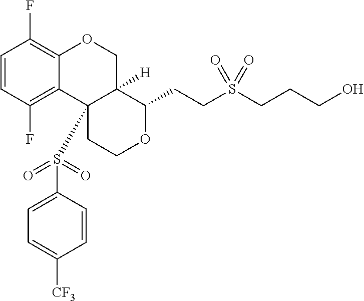 |
| 26A | 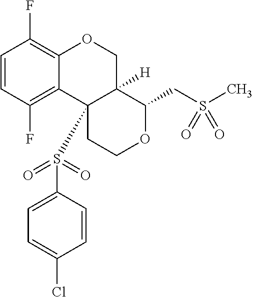 |
| 26B | 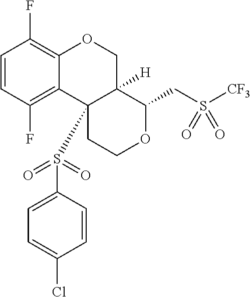 |
| 27 | 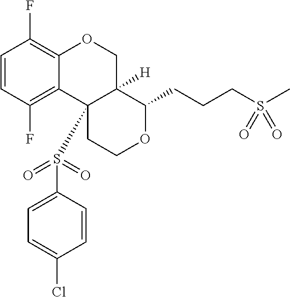 |
| 28 | 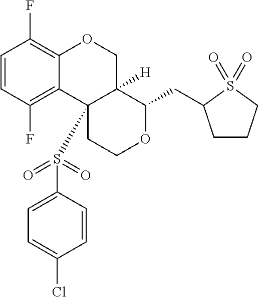 |
| 31 | 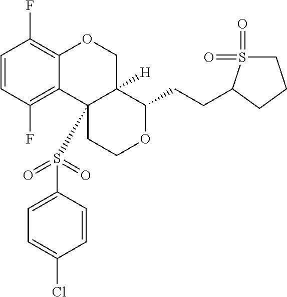 |
| 33 (−) | 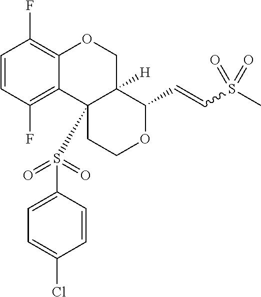 |
| 36 | 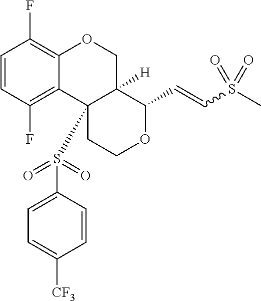 |
| 37 | 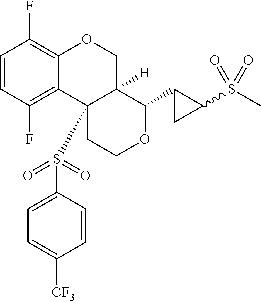 |
| 39 | 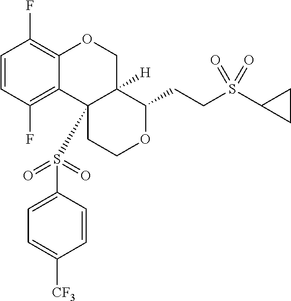 |
| 44A | 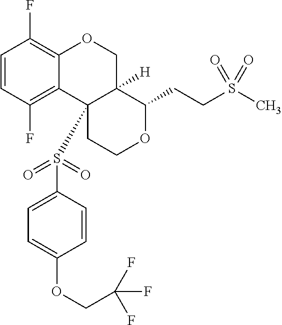 |
| 44B | 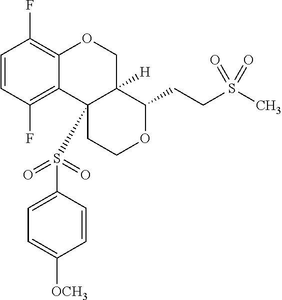 |
| 55 | 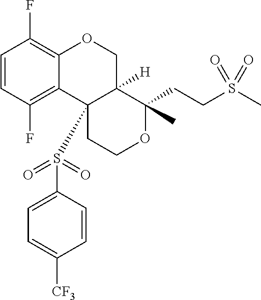 |
| 56 | 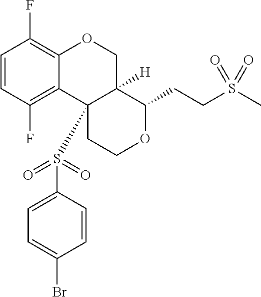 |
| 57 | 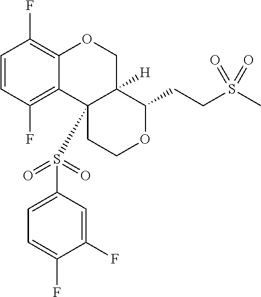 |
| 60 | 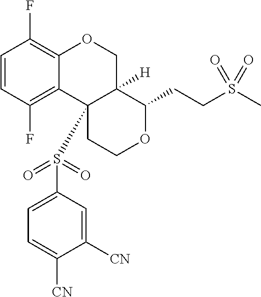 |
| 70A | 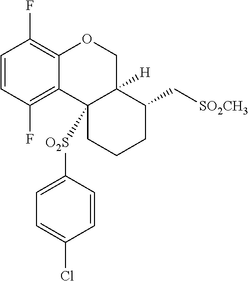 |
| 71A (−) | 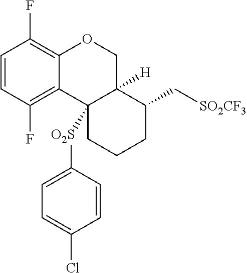 |
| 75A-rac | 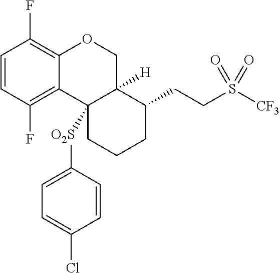 |
| 75B (−) | 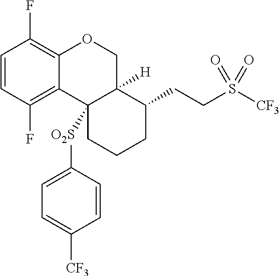 |
| 76A (−) | 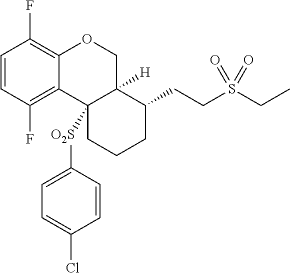 |
| 76 (+) | 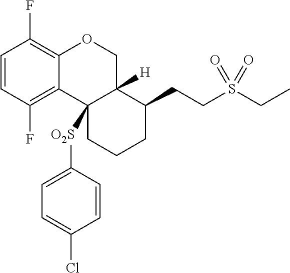 |
| 76B (−) | 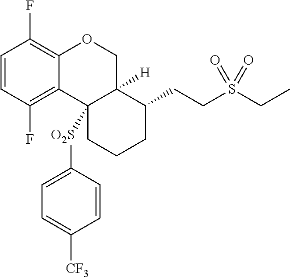 |
| 78A (−) | 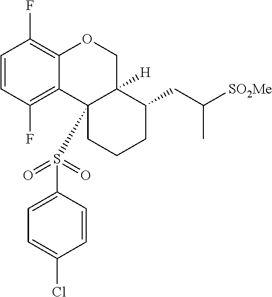 |
| 79A (−) | 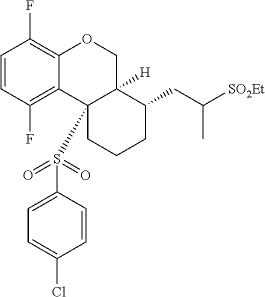 |
| 80A (−) | 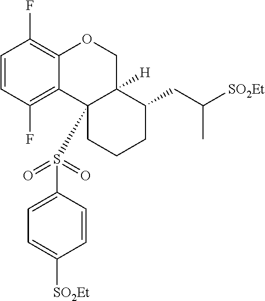 |
| 81A-rac | 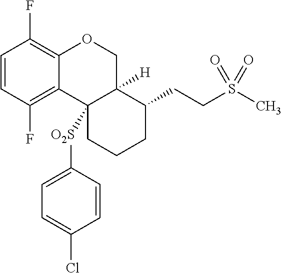 |
| 81A (−) | 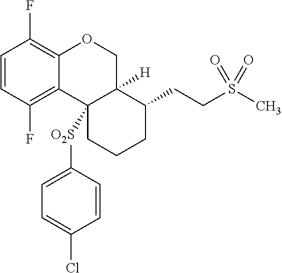 |
| 81A (+) | 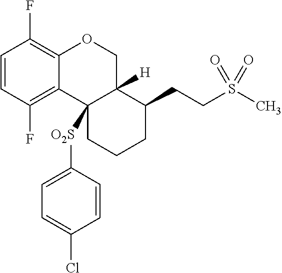 |
| 81B (−) | 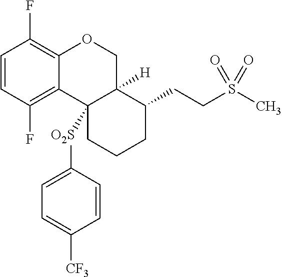 |
| 82A | 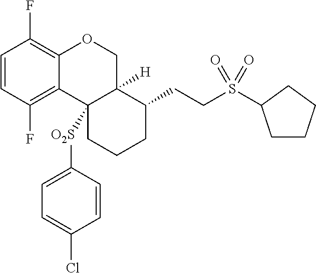 |
| 83A | 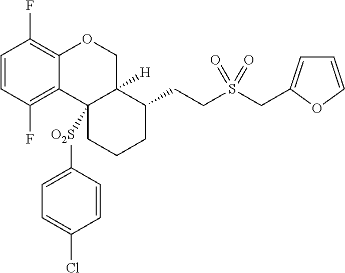 |
| 84A | 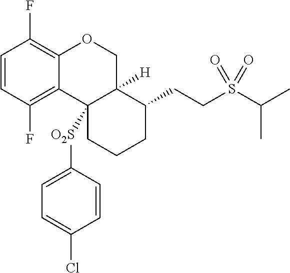 |
| 85A | 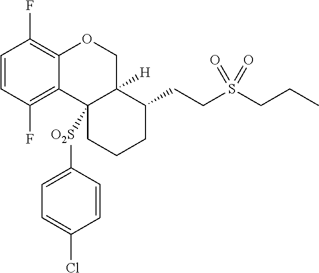 |
| 85B (−) | 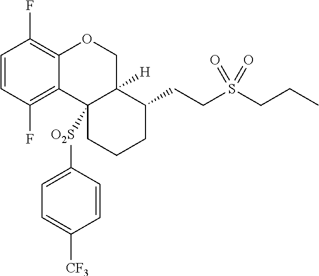 |
| 86A | 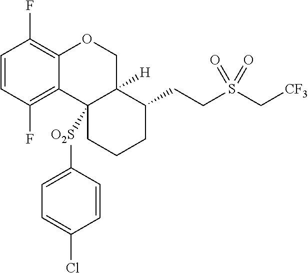 |
| 87B | 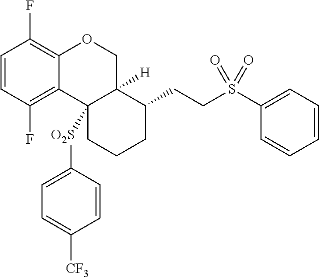 |
| 95B-rac | 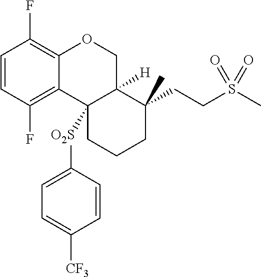 |
| 95B (−) | 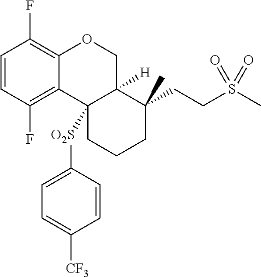 |
| 95B (+) | 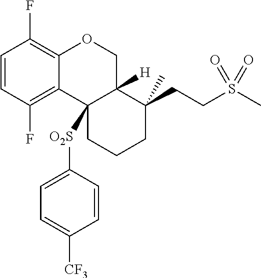 |
| 106 (−) | 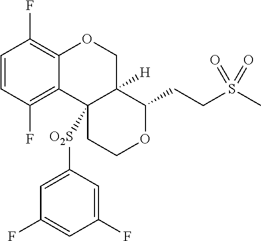 |
| 107 (−) | 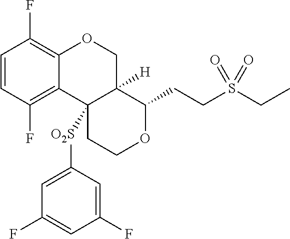 |
| 108 (−) | 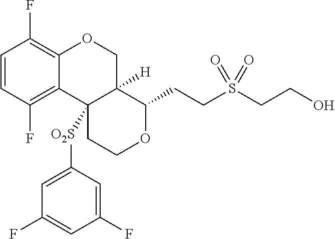 |
| 111 (−) | 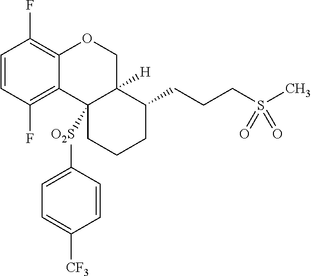 |

Representative compounds of this invention include but are not limited to:

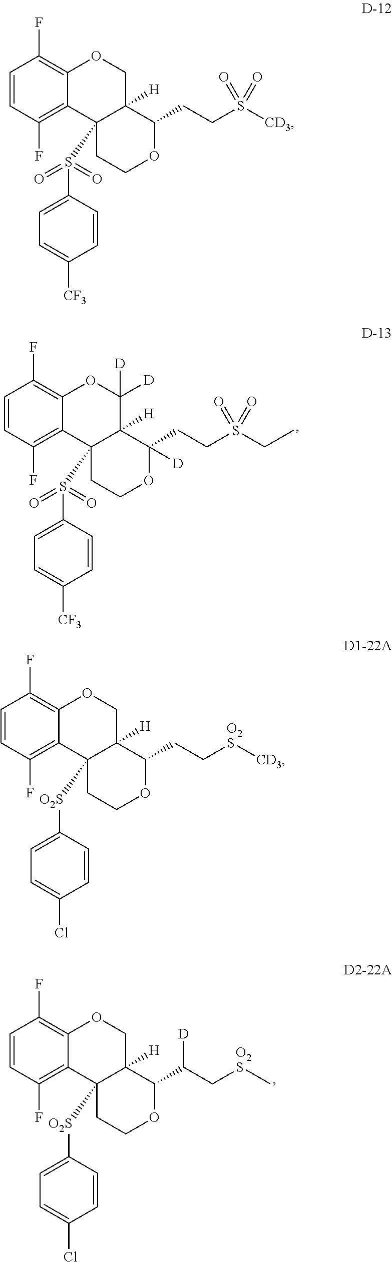

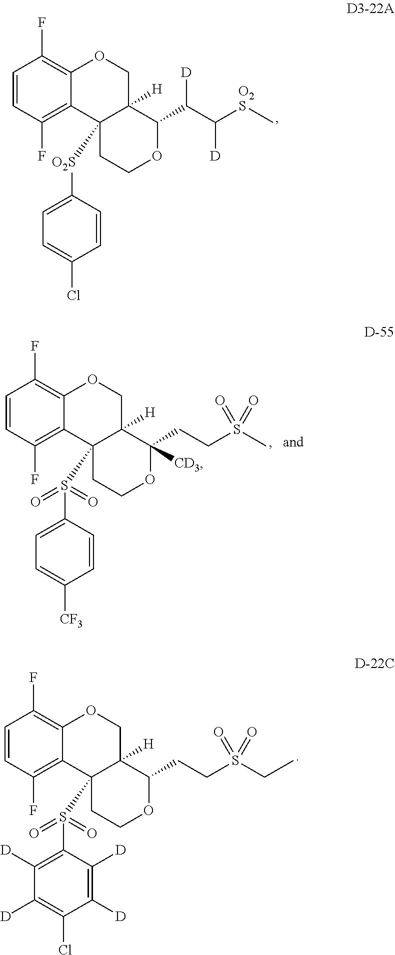

One embodiment of the present invention is directed to compounds of Formula (I) in pure form.

Another embodiment of the present invention is directed to compounds of Formula (I) in isolated form.

Another embodiment of the present invention is directed to compounds of formula (I.A1) in pure and isolated form.

Another embodiment of the present invention is directed to compounds of formula (I.A1) in pure form.

Another embodiment of the present invention is directed to compounds of formula (I.A1) in isolated form.

Another embodiment of the present invention is directed to compounds of formula (I.A2) in pure and isolated form.

Another embodiment of the present invention is directed to compounds of formula (I.A2) in pure form.

Another embodiment of the present invention is directed to compounds of formula (I.A2) in isolated form.

Another embodiment of the present invention is directed to compounds of Formula (I), or pharmaceutically acceptable salts, solvates or esters thereof.

Another embodiment of this invention is directed to compounds of formula (I).

Another embodiment of this invention is directed to pharmaceutically acceptable salts of the compounds of formula (I).

Another embodiment of this invention is directed to a pharmaceutically acceptable salt of the compounds of formula (I).

Another embodiment of this invention is directed to a pharmaceutically acceptable salt of the compounds of formula (I.A1).

Another embodiment of this invention is directed to a pharmaceutically acceptable salt of the compounds of formula (I.A2).

Another embodiment of this invention is directed to a pharmaceutically acceptable salt of a compound of formula (I.A1i).

Another embodiment of this invention is directed to a pharmaceutically acceptable salt of a compound of formula (I.A1j).

Another embodiment of this invention is directed to a pharmaceutically acceptable salt of a compound of formula (I.A1k).

Another embodiment of this invention is directed to a pharmaceutically acceptable salt of a compound of formula (I.A1l).

Another embodiment of this invention is directed to a pharmaceutically acceptable salt of a compound of formula (I.A1m).

Another embodiment of this invention is directed to a pharmaceutically acceptable salt of a compound of formula (I.A1n).

Another embodiment of this invention is directed to a pharmaceutically acceptable salt of a compound of formula (I.A1o).

Another embodiment of this invention is directed to a pharmaceutically acceptable salt of a compound of formula (I.A1p).

Another embodiment of this invention is directed to a pharmaceutically acceptable salt of a compound of formula (I.A1q).

Another embodiment of this invention is directed to a pharmaceutically acceptable salt of a compound of formula (I.A1r).

Another embodiment of this invention is directed to a pharmaceutically acceptable salt of a compound of formula (I.A1s).

Another embodiment of this invention is directed to solvates of the compounds of formula (I). Another embodiment of this invention is directed to a solvate of a compound of formula (I).

Another embodiment of this invention is directed to solvates of the compounds of formula (I.A1). Another embodiment of this invention is directed to a solvate of a compound of formula (I.A1).

Another embodiment of this invention is directed to solvates of the compounds of formula (I.A2). Another embodiment of this invention is directed to a solvate of a compound of formula (I.A2).

Another embodiment of this invention is directed to a solvate of a compound of formula (I.A1i).

Another embodiment of this invention is directed to a solvate of a compound of formula (I.A1j).

Another embodiment of this invention is directed to a solvate of a compound of formula (I.A1k).

Another embodiment of this invention is directed to a solvate of a compound of formula (I.A1l).

Another embodiment of this invention is directed to a solvate of a compound of formula (I.A1m).

Another embodiment of this invention is directed to a solvate of a compound of formula (I.A1n).

Another embodiment of this invention is directed to a solvate of a compound of formula (I.A1o).

Another embodiment of this invention is directed to a solvate of a compound of formula (I.A1p).

Another embodiment of this invention is directed to a solvate of a compound of formula (I.A1q).

Another embodiment of this invention is directed to a solvate of a compound of formula (I.A1r).

Another embodiment of this invention is directed to a solvate of a compound of formula (I.A1s).

Another embodiment of this invention is directed to a solvate of a compound selected from the group consisting of compounds 12, 13, 22A to 22N, 22B-rac, 26A, 26B, 27, 28, 31, 33(−), 36, 37, 39, 44A, 44B, 55 to 57, 60, 70A, 71A(−), 75A-rac, 75B(−), 76A(−), 76A(+), 76B(−), 78A(−), 79A(−), 80A(+), 81A-rac, 81A(−), 81A(+), 81B(−), 82A, 83A, 84A, 85A, 85B(−), 86A, 87B, 95B-rac, 95B(−), 95B(+), 106(−), 107(−), 108(−), and 111(−) wherein at least one (e.g., 1-4, 1-3, 1-2, 2 or 1) H is replaced by deuterium in said compound.

Another embodiment of this invention is directed to a solvate of a compound selected from the group consisting of compounds D-12, D-13, D1-22A, D2-22A, D3-22A and D-55.

Another embodiment of this invention is directed to pharmaceutically acceptable esters of the compounds of formula (I). Another embodiment of this invention is directed to a pharmaceutically acceptable ester of a compound of formula (I).

Another embodiment of this invention is directed to a pharmaceutically acceptable ester of a compound of formula (I.A1).

Another embodiment of this invention is directed to a pharmaceutically acceptable ester of a compound of formula (I.A2).

Another embodiment of this invention is directed to a pharmaceutically acceptable ester of a compound of formula I.A1i, I.A1j, I.A1k, I.A1l, I.A1m, I.A1n, I.A1o, I.A1p, I.A1q, I.A1r, or I.A1s.

Another embodiment of the present invention is directed to any one of the compounds of formulas (Ii) to (Ivii), (I.A1a) to (I.A1h), (I.A2a) to (I.A2h) or (I.A1i) to (I.A1s), or pharmaceutically acceptable salts, solvates or esters thereof.

Another embodiment of this invention is directed to any one of the compounds of formulas (Ii) to (Ivii), (I.A1a) to (I.A1h), (I.A2a) to (I.A2h) or (I.A1i) to (I.A1s).

Another embodiment of this invention is directed to pharmaceutically acceptable salts of any one of the compounds of formulas (Ii) to (Ivii), (I.A1a) to (I.A1h), (I.A2a) to (I.A2h) or (I.A1i) to (I.A1s).

Another embodiment of this invention is directed to solvates of any one of the compounds of formulas (Ii) to (Ivii), (I.A1a) to (I.A1h), (I.A2a) to (I.A2h) or (I.A1i) to (I.A1s).

Another embodiment of this invention is directed to pharmaceutically acceptable esters of any one of the compounds of formulas (Ii) to (Ivii), (I.A1a) to (I.A1h), (I.A2a) to (I.A2h) or (I.A1i) to (I.A1s).

Another embodiment of the present invention is directed to any one of the compounds of formulas (Ii) to (Ivii), or pharmaceutically acceptable salts, solvates or esters thereof.

Another embodiment of this invention is directed to any one of the compounds of formulas (Ii) to (Ivii).

Another embodiment of this invention is directed to pharmaceutically acceptable salts of any one of the compounds of formulas (Ii) to (Ivii).

Another embodiment of this invention is directed to solvates of any one of the compounds of formulas (Ii) to (Ivii).

Another embodiment of this invention is directed to pharmaceutically acceptable esters of any one of the compounds of formulas (Ii) to (Ivii). Another embodiment of the present invention is directed to any one of the compounds of formulas (I.A1a) to (I.A1h), or pharmaceutically acceptable salts, solvates or esters thereof.

Another embodiment of this invention is directed to any one of the compounds of formulas (I.A1a) to (I.A1h).

Another embodiment of this invention is directed to pharmaceutically acceptable salts of any one of the compounds of formulas (Ii) to (Ivii).

Another embodiment of this invention is directed to solvates of any one of the compounds of formulas (I.A1a) to (I.A1h).

Another embodiment of this invention is directed to pharmaceutically acceptable esters of any one of the compounds of formulas (I.A1a) to (I.A1h).

Another embodiment of the present invention is directed to any one of the compounds of formulas (I.A2a) to (I.A2h), or pharmaceutically acceptable salts, solvates or esters thereof.

Another embodiment of this invention is directed to any one of the compounds of formulas (I.A2a) to (I.A2h).

Another embodiment of this invention is directed to pharmaceutically acceptable salts of any one of the compounds of formulas (I.A2a) to (I.A2h).

Another embodiment of this invention is directed to solvates of any one of the compounds of formulas (I.A2a) to (I.A2h).

Another embodiment of this invention is directed to pharmaceutically acceptable esters of any one of the compounds of formulas (I.A2a) to (I.A2h).

Another embodiment of the present invention is directed to any one of the compounds of formulas (I.A1i) to (I.A1s), or pharmaceutically acceptable salts, solvates or esters thereof.

Another embodiment of this invention is directed to any one of the compounds of formulas (I.A1i) to (I.A1s).

Another embodiment of this invention is directed to pharmaceutically acceptable salts of any one of the compounds of formulas (I.A1i) to (I.A1s).

Another embodiment of this invention is directed to solvates of any one of the compounds of formulas (I.A1i) to (I.A1s).

Another embodiment of this invention is directed to pharmaceutically acceptable esters of any one of the compounds of formulas (I.A1i) to (I.A1s).

Another embodiment of this invention is directed to a method for inhibiting gamma-secretase comprising administering an effective (i.e., therapeutically effective) amount of one or more compounds of formula (I) to a patient in need of treatment.

Another embodiment of this invention is directed to a method for inhibiting gamma-secretase comprising administering an effective (i.e., therapeutically effective) amount of one or more compounds of formula (I.A1) to a patient in need of treatment.

Another embodiment of this invention is directed to a method for inhibiting gamma-secretase comprising administering an effective (i.e., therapeutically effective) amount of one or more compounds of formula (I.A2) to a patient in need of treatment.

Another embodiment of this invention is directed to a method for inhibiting gamma-secretase comprising administering an effective (i.e., therapeutically effective) amount of a compound of formula (I) to a patient in need of treatment.

Another embodiment of this invention is directed to a method for inhibiting gamma-secretase comprising administering an effective (i.e., therapeutically effective) amount of a compound of formula (I.A1) to a patient in need of treatment.

Another embodiment of this invention is directed to a method for inhibiting gamma-secretase comprising administering an effective (i.e., therapeutically effective) amount of a compound of formula (I.A2) to a patient in need of treatment.

Another embodiment of this invention is directed to a method for inhibiting gamma-secretase comprising administering to a patient in need of treatment an effective (i.e., therapeutically effective) amount of one or more (e.g., one) compounds selected from the group consisting of compounds 12, 13, 22A to 22N, 22B-rac, 26A, 26B, 27, 28, 31, 33(−), 36, 37, 39, 44A, 44B, 55 to 57, 60, 70A, 71A(−), 75A-rac, 75B(−), 76A(−), 76A(+), 76B(−), 78A(−), 79A(−), 80A(+), 81A-rac, 81A(−), 81A(+), 81B(−), 82A, 83A, 84A, 85A, 85B(−), 86A, 87B, 95B-rac, 95B(−), 95B(+), 106(−), 107(−), 108(−), and 111(−) wherein at least one (e.g., 1-4, 1-3, 1-2, 2 or 1) H is replaced by deuterium in said compounds.

Another embodiment of this invention is directed to a method for inhibiting gamma-secretase comprising administering to a patient in need of treatment an effective (i.e., therapeutically effective) amount of one or more (e.g., one) compounds selected from the group consisting of compounds D-12, D-13, D1-22A, D2-22A, D3-22A and D-55.

Another embodiment of this invention is directed to a method of treating one or more neurodegenerative diseases comprising administering an effective (i.e., therapeutically effective) amount of one or more compounds of formula (I) to a patient in need of treatment.

Another embodiment of this invention is directed to a method of treating one or more neurodegenerative diseases comprising administering an effective (i.e., therapeutically effective) amount of one or more compounds of formula (I.A1) to a patient in need of treatment.

Another embodiment of this invention is directed to a method of treating one or more neurodegenerative diseases comprising administering an effective (i.e., therapeutically effective) amount of one or more compounds of formula (I.A2) to a patient in need of treatment.

Another embodiment of this invention is directed to a method of treating one or more neurodegenerative diseases comprising administering an effective (i.e., therapeutically effective) amount of a compound of formula (I) to a patient in need of treatment.

Another embodiment of this invention is directed to a method of treating one or more neurodegenerative diseases comprising administering an effective (i.e., therapeutically effective) amount of a compound of formula (I.A1) to a patient in need of treatment.

Another embodiment of this invention is directed to a method of treating one or more neurodegenerative diseases comprising administering an effective (i.e., therapeutically effective) amount of a compound of formula (I.A2) to a patient in need of treatment.

Another embodiment of this invention is directed to a method for treating one or more neurodegenerative diseases comprising administering to a patient in need of treatment an effective (i.e., therapeutically effective) amount of one or more (e.g., one) compounds selected from the group consisting of compounds 12, 13, 22A to 22N, 22B-rac, 26A, 26B, 27, 28, 31, 33(−), 36, 37, 39, 44A, 44B, 55 to 57, 60, 70A, 71A(−), 75A-rac, 75B(−), 76A(−), 76A(+), 76B(−), 78A(−), 79A(−), 80A(+), 81A-rac, 81A(−), 81A(+), 81B(−), 82A, 83A, 84A, 85A, 85B(−), 86A, 87B, 95B-rac, 95B(−), 95B(+), 106(−), 1070, 108(−), and 111(−) wherein at least one (e.g., 1-4, 1-3, 1-2, 2 or 1) H is replaced by deuterium in said compounds.

Another embodiment of this invention is directed to a method for treating one or more neurodegenerative diseases comprising administering to a patient in need of treatment an effective (i.e., therapeutically effective) amount of one or more (e.g., one) compounds selected from the group consisting of compounds D-12, D-13, D1-22A, D2-22A, D3-22A and D-55.

Another embodiment of this invention is directed to a method of inhibiting the deposition of amyloid protein (e.g., amyloid beta protein) in, on or around neurological tissue (e.g., the brain) comprising administering an effective (i.e., therapeutically effective) amount of one or more compounds of formula (I) to a patient in need of treatment.

Another embodiment of this invention is directed to a method of inhibiting the deposition of amyloid protein (e.g., amyloid beta protein) in, on or around neurological tissue (e.g., the brain) comprising administering an effective (i.e., therapeutically effective) amount of one or more compounds of formula (I.A1) to a patient in need of treatment.

Another embodiment of this invention is directed to a method of inhibiting the deposition of amyloid protein (e.g., amyloid beta protein) in, on or around neurological tissue (e.g., the brain) comprising administering an effective (i.e., therapeutically effective) amount of one or more compounds of formula (I.A2) to a patient in need of treatment.

Another embodiment of this invention is directed to a method of inhibiting the deposition of amyloid protein (e.g., amyloid beta protein) in, on or around neurological tissue (e.g., the brain) comprising administering an effective (i.e., therapeutically effective) amount of a compound of formula (I) to a patient in need of treatment.

Another embodiment of this invention is directed to a method of inhibiting the deposition of amyloid protein (e.g., amyloid beta protein) in, on or around neurological tissue (e.g., the brain) comprising administering an effective (i.e., therapeutically effective) amount of a compound of formula (I.A1) to a patient in need of treatment.

Another embodiment of this invention is directed to a method of inhibiting the deposition of amyloid protein (e.g., amyloid beta protein) in, on or around neurological tissue (e.g., the brain) comprising administering an effective (i.e., therapeutically effective) amount of a compound of formula (I.A2) to a patient in need of treatment.

Another embodiment of this invention is directed to a method of inhibiting the deposition of amyloid protein (e.g., amyloid beta protein) in, on or around neurological tissue (e.g., the brain) comprising administering to a patient in need of treatment an effective (i.e., therapeutically effective) amount of one or more (e.g., one) compounds selected from the group consisting of compounds 12, 13, 22A to 22N, 22B-rac, 26A, 26B, 27, 28, 31, 33(−), 36, 37, 39, 44A, 44B, 55 to 57, 60, 70A, 71A(−), 75A-rac, 75B(−), 76A(−), 76A(+), 76B(−), 78A(−), 79A(−), 80A(+), 81A-rac, 81A(−), 81A(+), 81B(−), 82A, 83A, 84A, 85A, 85B(−), 86A, 87B, 95B-rac, 95B(−), 95B(+), 106(−), 107(−), 108(−), and 111(−) wherein at least one (e.g., 1-4, 1-3, 1-2, 2 or 1) H is replaced by deuterium in said compounds.

Another embodiment of this invention is directed to a method of inhibiting the deposition of amyloid protein (e.g., amyloid beta protein) in, on or around neurological tissue (e.g., the brain) comprising administering to a patient in need of treatment an effective (i.e., therapeutically effective) amount of one or more (e.g., one) compounds selected from the group consisting of compounds D-12, D-13, D1-22A, D2-22A, D3-22A and D-55.

Another embodiment of this invention is directed to a method of treating Alzheimer's disease comprising administering an effective (i.e., therapeutically effective) amount of one or more compounds of formula (I) to a patient in need of treatment.

Another embodiment of this invention is directed to a method of treating Alzheimer's disease comprising administering an effective (i.e., therapeutically effective) amount of one or more compounds of formula (I.A1) to a patient in need of treatment.

Another embodiment of this invention is directed to a method of treating Alzheimer's disease comprising administering an effective (i.e., therapeutically effective) amount of one or more compounds of formula (I.A2) to a patient in need of treatment.

Another embodiment of this invention is directed to a method of treating Alzheimer's disease comprising administering an effective (i.e., therapeutically effective) amount of a compound of formula (I) to a patient in need of treatment.

Another embodiment of this invention is directed to a method of treating Alzheimer's disease comprising administering an effective (i.e., therapeutically effective) amount of a compound of formula (I.A1) to a patient in need of treatment.

Another embodiment of this invention is directed to a method of treating Alzheimer's disease comprising administering an effective (i.e., therapeutically effective) amount of a compound of formula (I.A2) to a patient in need of treatment.

Another embodiment of this invention is directed to a method of treating Alzheimer's disease comprising administering to a patient in need of treatment an effective (i.e., therapeutically effective) amount of one or more (e.g., one) compounds selected from the group consisting of compounds 12, 13, 22A to 22N, 22B-rac, 26A, 26B, 27, 28, 31, 33(−), 36, 37, 39, 44A, 44B, 55 to 57, 60, 70A, 71A(−), 75A-rac, 75B(−), 76A(−), 76A(+), 76B(−), 78A(−), 79A(−), 80A(+), 81A-rac, 81A(−), 81A(+), 81B(−), 82A, 83A, 84A, 85A, 85B(−), 86A, 87B, 95B-rac, 95B(−), 95B(+), 106(−), 107(−), 108(−), and 111(−) wherein at least one (e.g., 1-4, 1-3, 1-2, 2 or 1) H is replaced by deuterium in said compounds.

Another embodiment of this invention is directed to a method of treating Alzheimer's disease comprising administering to a patient in need of treatment an effective (i.e., therapeutically effective) amount of one or more (e.g., one) compounds selected from the group consisting of compounds D-12, D-13, D1-22A, D2-22A, D3-22A and D-55.

Another embodiment of this invention is directed to combination therapies for (1) inhibiting gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of one or more (e.g. one) compounds of formula (I) (e.g., compounds of formula (I.A1), or compounds of formula (I.A2)) and the administration of one or more (e.g., one) other pharmaceutical active ingredients (e.g., drugs). The compounds of formula (I) and the other drugs can be administered separately (i.e., each is in its own separate dosage form), or the compounds of formula (I) can be combined with the other drugs in the same dosage form.

Another embodiment of this invention is directed to any one of the methods of treatment, or methods of inhibiting, described herein, wherein the compound of formula (I) (e.g., the compound of formula (I.A1), or the compound of formula (I.A2)) is used in combination with an effective amount of one or more other pharmaceutically active ingredients selected from the group consisting of: BACE inhibitors (beta secretase inhibitors), muscarinic antagonists (e.g., m₁ or m₂ antagonists), cholinesterase inhibitors (e.g., acetyl- and/or butyrylchlolinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors and cholesterol absorption inhibitors (e.g., ezetimibe).

Another embodiment of this invention is directed to a method of treating Alzheimer's disease comprising administering an effective (i.e., therapeutically effective) amount of one or more compounds of formula (I), in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e, donepezil hydrochloride, available as the Aricept® brand of donepezil hydrochloride), to a patient in need of treatment.

Another embodiment of this invention is directed to a method of treating Alzheimer's disease comprising administering an effective (i.e., therapeutically effective) amount of one or more compounds of formula (I.A1), in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e, donepezil hydrochloride, available as the Aricept® brand of donepezil hydrochloride), to a patient in need of treatment.

Another embodiment of this invention is directed to a method of treating Alzheimer's disease comprising administering an effective (i.e., therapeutically effective) amount of one or more compounds of formula (I.A2), in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e, donepezil hydrochloride, available as the Aricept® brand of donepezil hydrochloride), to a patient in need of treatment.

Another embodiment of this invention is directed to a method of treating Alzheimer's disease comprising administering an effective (i.e., therapeutically effective) amount of a compound of formula (I), in combination with an effective (i.e., therapeutically effective) amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e, donepezil hydrochloride, available as the Aricept® brand of donepezil hydrochloride), to a patient in need of treatment.

Another embodiment of this invention is directed to a method of treating Alzheimer's disease comprising administering an effective (i.e., therapeutically effective) amount of a compound of formula (I.A1), in combination with an effective (i.e., therapeutically effective) amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e, donepezil hydrochloride, available as the Aricept® brand of donepezil hydrochloride), to a patient in need of treatment.

Another embodiment of this invention is directed to a method of treating Alzheimer's disease comprising administering an effective (i.e., therapeutically effective) amount of a compound of formula (I.A2), in combination with an effective (i.e., therapeutically effective) amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e, donepezil hydrochloride, available as the Aricept® brand of donepezil hydrochloride), to a patient in need of treatment.

The phrase “any one of the above methods” as used below means that the description below applies to each method described above just as if each method above was separately described with the scope described below.

Another embodiment of this invention is directed to any one of the above methods wherein a pharmaceutically acceptable salt of a compound of formula (I) is used instead of the compound of formula (I).

Another embodiment of this invention is directed to any one of the above methods wherein a pharmaceutically acceptable salt of a compound of formula (I.A1) is used instead of the compound of formula (I.A1).

Another embodiment of this invention is directed to any one of the above methods wherein a pharmaceutically acceptable salt of a compound of formula (I.A2) is used instead of the compound of formula (I.A2).

Another embodiment of this invention is directed to any one of the above methods wherein a pharmaceutically acceptable ester of a compound of formula (I) is used instead of the compound of formula (I).

Another embodiment of this invention is directed to any one of the above methods wherein a pharmaceutically acceptable ester of a compound of formula (I.A1) is used instead of the compound of formula (I.A1).

Another embodiment of this invention is directed to any one of the above methods wherein a pharmaceutically acceptable ester of a compound of formula (I.A2) is used instead of the compound of formula (I.A2).

Another embodiment of this invention is directed to any one of the above methods wherein a solvate of a compound of formula (I) is used instead of the compound of formula (I).

Another embodiment of this invention is directed to any one of the above methods wherein a solvate of a compound of formula (I.A1) is used instead of the compound of formula (I.A1).

Another embodiment of this invention is directed to any one of the above methods wherein a solvate of a compound of formula (I.A2) is used instead of the compound of formula (I.A2).

Another embodiment of this invention is directed to any one of the methods described above wherein the compound of formula (I) (e.g., I.A1 or I.A2) is selected from the group consisting of the formulas (Ii) to (Ivii), (I.A1a) to (I.A1h), (I.A2a) to (I.A2h), and (I.A1i) to (I.A1s).

Another embodiment of this invention is directed to any one of the methods of treatment described above wherein the compound of formula (I) is any one of the compounds of formulas (Ii) to (Ivii).

Another embodiment of this invention is directed to any one of the methods of treatment described above wherein a pharmaceutically acceptable salt of any one of the compounds of (Ii) to (Ivii) is used.

Another embodiment of this invention is directed to any one of the methods of treatment described above wherein a solvate of any one of the compounds of formulas (Ii) to (Ivii) is used.

Another embodiment of this invention is directed to any one of the methods of treatment described above wherein a pharmaceutically acceptable ester of any one of the compounds of formulas (Ii) to (Ivii) is used.

Another embodiment of this invention is directed to any one of the methods of treatment described above wherein any one of the compounds of formulas (I.A1a) to (I.A1h) is used.

Another embodiment of this invention is directed to any one of the methods of treatment described above wherein a pharmaceutically acceptable salt of any one of the compounds of formulas (I.A1a) to (I.A1h) is used.

Another embodiment of this invention is directed to any one of the methods of treatment described above wherein a solvate of any one of the compounds of formulas (I.A1a) to (I.A1h).

Another embodiment of this invention is directed to any one of the methods of treatment described above wherein a pharmaceutically acceptable ester of any one of the compounds of formulas (I.A1a) to (I.A1h) is used.

Another embodiment of this invention is directed to any one of the methods of treatment described above wherein any one of the compounds of formulas (I.A2a) to (I.A2h) is used.

Another embodiment of this invention is directed to any one of the methods of treatment described above a pharmaceutically acceptable salt of any one of the compounds of formulas (I.A2a) to (I.A2h) is used.

Another embodiment of this invention is directed to any one of the methods of treatment described above wherein a solvate of any one of the compounds of formulas (I.A2a) to (I.A2h) is used.

Another embodiment of this invention is directed to any one of the methods of treatment described above wherein a pharmaceutically acceptable ester of any one of the compounds of formulas (I.A2a) to (I.A2h) is used.

Another embodiment of this invention is directed to any one of the methods of treatment described above wherein the compound of formula (I) is any one of the compounds of formulas (I.A1i) to (I.A1s).

Another embodiment of this invention is directed to any one of the methods of treatment described above wherein a pharmaceutically acceptable salt of any one of the compounds of (I.A1i) to (I.A1s) is used.

Another embodiment of this invention is directed to any one of the methods of treatment described above wherein a solvate of any one of the compounds of formulas (I.A1i) to (I.A1s) is used.

Another embodiment of this invention is directed to any one of the methods of treatment described above wherein a pharmaceutically acceptable ester of any one of the compounds of formulas (I.A1i) to (I.A1s) is used.

Another embodiment of this invention is directed to any one of the methods of treatment described above wherein the compound used is selected from the group consisting of compounds: 12, 13, 22A to 22N, 22B-rac, 26A, 26B, 27, 28, 31, 33(−), 36, 37, 39, 44A, 44B, 55 to 57, 60, 70A, 71A(−), 75A-rac, 75B(−), 76A(−), 76A(+), 76B(−), 78A(−), 79A(−), 80A(+), 81A-rac, 81A(−), 81A(+), 81B(−), 82A, 83A, 84A, 85A, 85B(−), 86A, 87B, 95B-rac, 95B(−), 95B(+), 106(−), 107(−), 108(−), and 111(−) wherein at least one (e.g., 1-4, 1-3, 1-2, 2 or 1) H is replaced by deuterium in said compound.

Another embodiment of this invention is directed to any one of the methods of treatment described above wherein the compound used is selected from the group consisting of: the solvates of compounds 12, 13, 22A to 22N, 22B-rac, 26A, 26B, 27, 28, 31, 33(−), 36, 37, 39, 44A, 44B, 55 to 57, 60, 70A, 71A(−), 75A-rac, 75B(−), 76A(−), 76A(+), 76B(−), 78A(−), 79A(−), 80A(+), 81A-rac, 81A(−), 81A(+), 81B(−), 82A, 83A, 84A, 85A, 85B(−), 86A, 87B, 95B-rac, 95B(−), 95B(+), 106(−), 107(−), 108(−), and 111(−) wherein at least one (e.g., 1-4, 1-3, 1-2, 2 or 1) H is replaced by deuterium in said compound.

Another embodiment of this invention is directed to any one of the methods of treatment described above wherein the compound used is selected from the group consisting of compounds: D-12, D-13, D1-22A, D2-22A, D3-22A and D-55.

Another embodiment of this invention is directed to any one of the methods of treatment described above wherein the compound used is selected from the group consisting of: the solvates of compounds D-12, D-13, D1-22A, D2-22A, D3-22A and D-55.

Another embodiment of this invention is directed to any one of the methods of treatment described above wherein the compound of formula (I) is compound D-12.

Another embodiment of this invention is directed to any one of the methods of treatment described above wherein a solvate of a compound of formula (I) is used, and the solvate is a solvate of compound D-12.

Another embodiment of this invention is directed to any one of the methods of treatment described above wherein the compound of formula (I) is compound D-13.

Another embodiment of this invention is directed to any one of the methods of treatment described above wherein a solvate of a compound of formula (I) is used, and the solvate is a solvate of compound D-13.

Another embodiment of this invention is directed to any one of the methods of treatment described above wherein the compound of formula (I) is compound D1-22A.

Another embodiment of this invention is directed to any one of the methods of treatment described above wherein a solvate of a compound of formula (I) is used, and the solvate is a solvate of compound D1-22A.

Another embodiment of this invention is directed to any one of the methods of treatment described above wherein the compound of formula (I) is compound D2-22A.

Another embodiment of this invention is directed to any one of the methods of treatment described above wherein a solvate of a compound of formula (I) is used, and the solvate is a solvate of compound D2-22A.

Another embodiment of this invention is directed to any one of the methods of treatment described above wherein the compound of formula (I) is compound D3-22A.

Another embodiment of this invention is directed to any one of the methods of treatment described above wherein a solvate of a compound of formula (I) is used, and the solvate is a solvate of compound D3-22A.

Another embodiment of this invention is directed to any one of the methods of treatment described above wherein the compound of formula (I) is compound D-55.

Another embodiment of this invention is directed to any one of the methods of treatment described above wherein a solvate of a compound of formula (I) is used, and the solvate is a solvate of compound D-55.

Another embodiment of this invention is directed to a pharmaceutical composition comprising an effective amount of one or more compounds of Formula (I) and at least one pharmaceutically acceptable carrier.

Another embodiment of this invention is directed to a pharmaceutical composition comprising an effective amount of one or more compounds of Formula (I.A1) and at least one pharmaceutically acceptable carrier.

Another embodiment of this invention is directed to a pharmaceutical composition comprising an effective amount of one or more compounds of Formula (I.A2) and at least one pharmaceutically acceptable carrier.

Another embodiment of this invention is directed to a pharmaceutical composition comprising an effective amount of a compound of Formula (I) and at least one pharmaceutically acceptable carrier.

Another embodiment of this invention is directed to a pharmaceutical composition comprising an effective amount of a compound of Formula (I.A1) and at least one pharmaceutically acceptable carrier.

Another embodiment of this invention is directed to a pharmaceutical composition comprising an effective amount of a compound of Formula (I.A2) and at least one pharmaceutically acceptable carrier.

Another embodiment of this invention is directed to a pharmaceutical composition comprising an effective amount of one or more pharmaceutically acceptable salts of the compounds of Formula (I), and at least one pharmaceutically acceptable carrier.

Another embodiment of this invention is directed to a pharmaceutical composition comprising an effective amount of one or more pharmaceutically acceptable salts of the compounds of Formula (I.A1), and at least one pharmaceutically acceptable carrier.

Another embodiment of this invention is directed to a pharmaceutical composition comprising an effective amount of one or more pharmaceutically acceptable salts of the compounds of Formula (I.A2), and at least one pharmaceutically acceptable carrier.

Another embodiment of this invention is directed to a pharmaceutical composition comprising an effective amount of a pharmaceutically acceptable salt of a compound of Formula (I), and at least one pharmaceutically acceptable carrier.

Another embodiment of this invention is directed to a pharmaceutical composition comprising an effective amount of a pharmaceutically acceptable salt of a compound of Formula (I.A1), and at least one pharmaceutically acceptable carrier.

Another embodiment of this invention is directed to a pharmaceutical composition comprising an effective amount of a pharmaceutically acceptable salt of a compound of Formula (I.A2), and at least one pharmaceutically acceptable carrier.

Another embodiment of this invention is directed to a pharmaceutical composition comprising an effective amount of one or more solvates of the compounds of Formula (I), and at least one pharmaceutically acceptable carrier.

Another embodiment of this invention is directed to a pharmaceutical composition comprising an effective amount of one or more solvates of the compounds of Formula (I.A1), and at least one pharmaceutically acceptable carrier.

Another embodiment of this invention is directed to a pharmaceutical composition comprising an effective amount of one or more solvates of the compounds of Formula (I.A2), and at least one pharmaceutically acceptable carrier.

Another embodiment of this invention is directed to a pharmaceutical composition comprising an effective amount of a solvate of a compound of Formula (I), and at least one pharmaceutically acceptable carrier.

Another embodiment of this invention is directed to a pharmaceutical composition comprising an effective amount of a solvate of a compound of Formula (I.A1), and at least one pharmaceutically acceptable carrier.

Another embodiment of this invention is directed to a pharmaceutical composition comprising an effective amount of a solvate of a compound of Formula (I.A2), and at least one pharmaceutically acceptable carrier.

Another embodiment of this invention is directed to a pharmaceutical composition comprising an effective amount of one or more pharmaceutically acceptable esters of the compounds of Formula (I), and at least one pharmaceutically acceptable carrier.

Another embodiment of this invention is directed to a pharmaceutical composition comprising an effective amount of one or more pharmaceutically acceptable esters of the compounds of Formula (I.A1), and at least one pharmaceutically acceptable carrier.

Another embodiment of this invention is directed to a pharmaceutical composition comprising an effective amount of one or more pharmaceutically acceptable esters of the compounds of Formula (I.A2), and at least one pharmaceutically acceptable carrier.

Another embodiment of this invention is directed to a pharmaceutical composition comprising an effective amount of a pharmaceutically acceptable ester of a compound of Formula (I), and at least one pharmaceutically acceptable carrier.

Another embodiment of this invention is directed to a pharmaceutical composition comprising an effective amount of a pharmaceutically acceptable ester of a compound of Formula (I.A1), and at least one pharmaceutically acceptable carrier.

Another embodiment of this invention is directed to a pharmaceutical composition comprising an effective amount of a pharmaceutically acceptable ester of a compound of Formula (I.A2), and at least one pharmaceutically acceptable carrier.

Another embodiment of this invention is directed to a pharmaceutical composition comprising an effective amount of one or more (e.g., one) compounds of formula (I) (e.g., compounds of formula (I.A1), or compounds of formula (I.A2)), and an effective amount of one or more (e.g., one) other pharmaceutically active ingredients (e.g., drugs), and a pharmaceutically acceptable carrier. Examples of the other pharmaceutically active ingredients include, but are not limited to drugs selected form the group consisting of: (a) drugs useful for the treatment of Alzheimer's disease, (b) drugs useful for inhibiting the deposition of amyloid protein (e.g., amyloid beta protein) in, on or around neurological tissue (e.g., the brain), (c) drugs useful for treating neurodegenerative diseases, and (d) drugs useful for inhibiting gamma-secretase.

Another embodiment of this invention is directed to a pharmaceutical composition comprising an effective amount of one or more (e.g., one) compounds of formula (I) (e.g., compounds of formula (I.A1), or compounds of formula (I.A2)), and effective amount of one or more BACE inhibitors, and a pharmaceutically acceptable carrier.

Another embodiment of this invention is directed to a pharmaceutical composition comprising an effective amount of one or more (e.g., one) compounds of formula (I) (e.g., compounds of formula (I.A1), or compounds of formula (I.A2)), and effective amount of one or more cholinesterase inhibitors (e.g., acetyl- and/or butyrylchlolinesterase inhibitors), and a pharmaceutically acceptable carrier.

Another embodiment of this invention is directed to a pharmaceutical composition comprising an effective amount of one or more (e.g., one) compounds of formula (I) (e.g., compounds of formulas (I.A1), or compounds of formula (I.A2)), and effective amount of one or more muscarinic antagonists (e.g., m₁ or m₂ antagonists), and a pharmaceutically acceptable carrier.

Another embodiment of this invention is directed to any one of the pharmaceutical compositions described above wherein a compound of any one of formulas (Ii) to (Ivii), (I.A1a) to (I.A1h), (I.A2a) to (I.A2h), or (I.A1i) to (I.A1s) is used.

Another embodiment of this invention is directed to any one of the pharmaceutical compositions described above wherein a pharmaceutically acceptable salt of any one of the compounds of formulas (Ii) to (Ivii), (I.A1a) to (I.A1h), (I.A2a) to (I.A2h), or (I.A1i) to (I.A1s) is used.

Another embodiment of this invention is directed to any one of the pharmaceutical compositions described above wherein a solvate of any one of the compounds of formulas (Ii) to (Ivii), (I.A1a) to (I.A1h), (I.A2a) to (I.A2h), or (I.A1i) to (I.A1s) is used.

Another embodiment of this invention is directed to any one of the pharmaceutical compositions described above wherein a pharmaceutically acceptable ester of the compounds of formulas (Ii) to (Ivii), (I.A1a) to (I.A1h), (I.A2a) to (I.A2h), or (I.A1i) to (I.A1s) is used.

Another embodiment of this invention is directed to any one of the pharmaceutical compositions described above wherein a compound of any one of formulas (Ii) to (Ivii) is used.

Another embodiment of this invention is directed to any one of the pharmaceutical compositions described above wherein a pharmaceutically acceptable salt of any one of the compounds of formulas (Ii) to (Ivii) is used.

Another embodiment of this invention is directed to any one of the pharmaceutical compositions described above wherein a solvate of any one of the compounds of formulas (Ii) to (Ivii) is used.

Another embodiment of this invention is directed to any one of the pharmaceutical compositions described above wherein a pharmaceutically acceptable ester of the compounds of formulas (Ii) to (Ivii) is used.

Another embodiment of this invention is directed to any one of the pharmaceutical compositions described above wherein a compound of any one of formulas (I.A1a) to (I.A1h) is used.

Another embodiment of this invention is directed to any one of the pharmaceutical compositions described above wherein a pharmaceutically acceptable salt of any one of the compounds of formulas (I.A1a) to (I.A1h) is used.

Another embodiment of this invention is directed to any one of the pharmaceutical compositions described above wherein a solvate of any one of the compounds of formulas (I.A1a) to (I.A1h) is used.

Another embodiment of this invention is directed to any one of the pharmaceutical compositions described above wherein a pharmaceutically acceptable ester of the compounds of formulas (I.A1a) to (I.A1h) is used.

Another embodiment of this invention is directed to any one of the pharmaceutical compositions described above wherein a compound of any one of formulas (I.A2a) to (I.A2h) is used.

Another embodiment of this invention is directed to any one of the pharmaceutical compositions described above wherein a pharmaceutically acceptable salt of any one of the compounds of formulas (I.A2a) to (I.A2h) is used.

Another embodiment of this invention is directed to any one of the pharmaceutical compositions described above wherein a solvate of any one of the compounds of formulas (I.A2a) to (I.A2h) is used.

Another embodiment of this invention is directed to any one of the pharmaceutical compositions described above wherein a pharmaceutically acceptable ester of the compounds of formulas (I.A2a) to (I.A2h) is used.

Another embodiment of this invention is directed to any one of the pharmaceutical compositions described above wherein a compound of any one of formulas I.A1i, I.A1j, I.A1k, I.A1l, I.A1m, I.A1n, I.A1o, I.A1p, I.A1q, I.A1r or I.A1s is used.

Another embodiment of this invention is directed to any one of the pharmaceutical compositions described above wherein a pharmaceutically acceptable salt of any one of the compounds of formulas I.A1i, I.A1j, I.A1k, I.A1l, I.A1m, I.A1n, I.A1o, I.A1p, I.A1q, I.A1r or I.A1s is used.

Another embodiment of this invention is directed to any one of the pharmaceutical compositions described above wherein a solvate of any one of the compounds of formulas I.A1i, I.A1j, I.A1k, I.A1l, I.A1m, I.A1n, I.A1o, I.A1p, I.A1q, I.A1r or I.A1s is used.

Another embodiment of this invention is directed to any one of the pharmaceutical compositions described above wherein a pharmaceutically acceptable ester of the compounds of formulas I.A1i, I.A1j, I.A1k, I.A1l, I.A1m, I.A1n, I.A1o, I.A1p, I.A1q, I.A1r or I.A1s is used.

Another embodiment of this invention is directed to a pharmaceutical composition comprising a pharmaceutically acceptable carrier and an effective amount of one or more compounds selected from the group consisting of compounds 12, 13, 22A to 22N, 22B-rac, 26A, 26B, 27, 28, 31, 33(−), 36, 37, 39, 44A, 44B, 55 to 57, 60, 70A, 71A(−), 75A-rac, 75B(−), 76A(−), 76A(+), 76B(−), 78A(−), 79A(−), 80A(+), 81A-rac, 81A(−), 81A(+), 81B(−), 82A, 83A, 84A, 85A, 85B(−), 86A, 87B, 95B-rac, 95B(−), 95B(+), 106(−), 107(−), 108(−), and 111(−) wherein at least one (e.g., 1-4, 1-3, 1-2, 2 or 1) H is replaced by deuterium in said compounds.

Another embodiment of this invention is directed to a pharmaceutical composition comprising a pharmaceutically acceptable carrier and an effective amount of a compound selected from the group consisting of compounds 12, 13, 22A to 22N, 22B-rac, 26A, 26B, 27, 28, 31, 33(−), 36, 37, 39, 44A, 44B, 55 to 57, 60, 70A, 71A(−), 75A-rac, 75B(−), 76A(−), 76A(+), 76B(−), 78A(−), 79A(−), 80A(+), 81A-rac, 81A(−), 81A(+), 81B(−), 82A, 83A, 84A, 85A, 85B(−), 86A, 87B, 95B-rac, 95B(−), 95B(+), 106(−), 107(−), 108(−), and 111(−) wherein at least one (e.g., 1-4, 1-3, 1-2, 2 or 1) H is replaced by deuterium in said compounds.

Another embodiment of this invention is directed to a pharmaceutical composition comprising a pharmaceutically acceptable carrier and an effective amount of one or more compounds selected from the group consisting of compounds D-12, D-13, D1-22A, D2-22A, D3-22A and D-55.

Another embodiment of this invention is directed to a pharmaceutical composition comprising a pharmaceutically acceptable carrier and an effective amount of a compound selected from the group consisting of compounds D-12, D-13, D1-22A, D2-22A, D3-22A and D-55.

Another embodiment of this invention is directed to a pharmaceutical composition comprising an effective amount of one or more solvates selected from the group consisting of: the solvates of compounds 12, 13, 22A to 22N, 22B-RAC, 26A, 26B, 27, 28, 31, 33(−), 36, 37, 39, 44A, 44B, 55 to 57, 60, 70A, 71A(−), 75A-rac, 75B(−), 76A(−), 76A(+), 76B(−), 78A(−), 79A(−), 80A(+), 81A-rac, 81A(−), 81A(+), 81B(−), 82A, 83A, 84A, 85A, 85B(−), 86A, 87B, 95B-rac, 95B(−), 95B(+), 106(−), 107(−), 108(−), and 111(−), wherein at least one (e.g., 1-4, 1-3, 1-2, 2 or 1) H is replaced by deuterium in said compounds, and at least one pharmaceutically acceptable carrier.

Another embodiment of this invention is directed to a pharmaceutical composition comprising an effective amount of a solvate selected from the group consisting of: the solvates of compounds 12, 13, 22A to 22N, 22B-RAC, 26A, 26B, 27, 28, 31, 33(−), 36, 37, 39, 44A, 44B, 55 to 57, 60, 70A, 71A(−), 75A-rac, 75B(−), 76A(−), 76A(+), 76B(−), 78A(−), 79A(−), 80A(+), 81A-rac, 81A(−), 81A(+), 81B(−), 82A, 83A, 84A, 85A, 85B(−), 86A, 87B, 95B-rac, 95B(−), 95B(+), 106(−), 107(−), 108(−), and 111(−), wherein at least one (e.g., 1-4, 1-3, 1-2, 2 or 1) H is replaced by deuterium in said compounds, and at least one pharmaceutically acceptable carrier.

Another embodiment of this invention is directed to a pharmaceutical composition comprising an effective amount of one or more solvates selected from the group consisting of: the solvates of compounds D-12, D-13, D1-22A, D2-22A, D3-22A and D-55, and at least one pharmaceutically acceptable carrier.

Another embodiment of this invention is directed to a pharmaceutical composition comprising an effective amount of a solvate selected from the group consisting of: the solvates of compounds D-12, D-13, D1-22A, D2-22A, D3-22A and D-55, and at least one pharmaceutically acceptable carrier.

Another embodiment of this invention is directed to a pharmaceutical composition comprising an effective amount of one or more (e.g., one) compounds selected from the group consisting of compounds 12, 13, 22A to 22N, 22B-rac, 26A, 26B, 27, 28, 31, 33(−), 36, 37, 39, 44A, 44B, 55 to 57, 60, 70A, 71A(−), 75A-rac, 75B(−), 76A(−), 76A(+), 76B(−), 78A(−), 79A(−), 80A(+), 81A-rac, 81A(−), 81A(+), 81B(−), 82A, 83A, 84A, 85A, 85B(−), 86A, 87B, 95B-rac, 95B(−), 95B(+), 106(−), 107(−), 108(−), and 111(−) wherein at least one (e.g., 1-4, 1-3, 1-2, 2 or 1) H is replaced by deuterium in said compounds, and effective amount of one or more (e.g., one) other therapeutically effective pharmaceutical active ingredients (e.g., drugs), and a pharmaceutically acceptable carrier. Examples of the other drugs include, but are not limited to drugs selected form the group consisting of: (a) drugs useful for the treatment of Alzheimer's disease, (b) drugs useful for inhibiting the deposition of amyloid protein (e.g., amyloid beta protein) in, on or around neurological tissue (e.g., the brain), (c) drugs useful for treating neurodegenerative diseases, and (d) drugs useful for inhibiting gamma-secretase.

Another embodiment of this invention is directed to a pharmaceutical composition comprising an effective amount of one or more (e.g., one) compounds selected from the group consisting of compounds D-12, D-13, D1-22A, D2-22A, D3-22A and D-55, and effective amount of one or more (e.g., one) other therapeutically effective pharmaceutical active ingredients (e.g., drugs), and a pharmaceutically acceptable carrier. Examples of the other drugs include, but are not limited to drugs selected form the group consisting of: (a) drugs useful for the treatment of Alzheimer's disease, (b) drugs useful for inhibiting the deposition of amyloid protein (e.g., amyloid beta protein) in, on or around neurological tissue (e.g., the brain), (c) drugs useful for treating neurodegenerative diseases, and (d) drugs useful for inhibiting gamma-secretase.

Another embodiment of this invention is directed to a pharmaceutical composition comprising an effective amount of one or more (e.g., one) compounds selected from the group consisting of compounds 12, 13, 22A to 22N, 22B-rac, 26A, 26B, 27, 28, 31, 33(−), 36, 37, 39, 44A, 44B, 55 to 57, 60, 70A, 71A(−), 75A-rac, 75B(−), 76A(−), 76A(+), 76B(−), 78A(−), 79A(−), 80A(+), 81A-rac, 81A(−), 81A(+), 81B(−), 82A, 83A, 84A, 85A, 85B(−), 86A, 87B, 95B-rac, 95B(−), 95B(+), 106(−), 107(−), 108(−), and 111(−) wherein at least one (e.g., 1-4, 1-3, 1-2, 2 or 1) H is replaced by deuterium in said compounds, and effective amount of one or more BACE inhibitors, and a pharmaceutically acceptable carrier.

Another embodiment of this invention is directed to a pharmaceutical composition comprising an effective amount of one or more (e.g., one) compounds selected from the group consisting of compounds D-12, D-13, D1-22A, D2-22A, D3-22A and D-55, and effective amount of one or more BACE inhibitors, and a pharmaceutically acceptable carrier.

Another embodiment of this invention is directed to a pharmaceutical composition comprising an effective amount of one or more (e.g., one) compounds selected from the group consisting of compounds 12, 13, 22A to 22N, 22B-rac, 26A, 266, 27, 28, 31, 33(−), 36, 37, 39, 44A, 44B, 55 to 57, 60, 70A, 71A(−), 75A-rac, 75B(−), 76A(−), 76A(+), 76B(−), 78A(−), 79A(−), 80A(+), 81A-rac, 81A(−), 81A(+), 81B(−), 82A, 83A, 84A, 85A, 85B(−), 86A, 87B, 95B-rac, 95B(−), 95B(+), 106(−), 107(−), 108(−), and 111(−) wherein at least one (e.g., 1-4, 1-3, 1-2, 2 or 1) H is replaced by deuterium in said compounds, and effective amount of one or more cholinesterase inhibitors (e.g., acetyl- and/or butyrylchlolinesterase inhibitors), and a pharmaceutically acceptable carrier.

Another embodiment of this invention is directed to a pharmaceutical composition comprising an effective amount of one or more (e.g., one) compounds selected from the group consisting of compounds D-12, D-13, D1-22A, D2-22A, D3-22A and D-55unds, and effective amount of one or more cholinesterase inhibitors (e.g., acetyl- and/or butyrylchlolinesterase inhibitors), and a pharmaceutically acceptable carrier.

Another embodiment of this invention is directed to a pharmaceutical composition comprising an effective amount of one or more (e.g., one) compounds selected from the group consisting of compounds 12, 13, 22A to 22N, 22B-rac, 26A, 26B, 27, 28, 31, 33(−), 36, 37, 39, 44A, 44B, 55 to 57, 60, 70A, 71A(−), 75A-rac, 75B(−), 76A(−), 76A(+), 76B(−), 78A(−), 79A(−), 80A(+), 81A-rac, 81A(−), 81A(+), 81B(−), 82A, 83A, 84A, 85A, 85B(−), 86A, 87B, 95B-rac, 95B(−), 95B(+), 106(−), 107(−), 108(−), and 111(−) wherein at least one (e.g., 1-4, 1-3, 1-2, 2 or 1) H is replaced by deuterium in said compounds, and effective amount of one or more muscarinic antagonists (e.g., m₁ or m₂ antagonists), and a pharmaceutically acceptable carrier.

Another embodiment of this invention is directed to a pharmaceutical composition comprising an effective amount of one or more (e.g., one) compounds selected from the group consisting of compounds D-12, D-13, D1-22A, D2-22A, D3-22A and D-55, and effective amount of one or more muscarinic antagonists (e.g., m₁ or m₂ antagonists), and a pharmaceutically acceptable carrier.

Another embodiment of this invention is directed to a kit comprising, in separate containers, in a single package, pharmaceutical compositions for use in combination, wherein one container comprises an effective amount of a compound of formula (I) (e.g., a compound of formula (I.A1), or a compound of formula (I.A2)) in a pharmaceutically acceptable carrier, and another container (i.e., a second container) comprises an effective amount of another pharmaceutically active ingredient (as described above), the combined quantities of the compound of formula (I) and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) inhibit gamma-secretase.

Another embodiment of this invention is directed to a kit comprising, in separate containers, in a single package, pharmaceutical compositions for use in combination, wherein one container comprises an effective amount of a compound selected from the group consisting of compounds I.A1i, I.A1j, I.A1k, I.A1l, I.A1m, I.A1n, I.A1o, I.A1p, I.A1q, I.A1r and I.A1s, in a pharmaceutically acceptable carrier, and another container (i.e., a second container) comprises an effective amount of another pharmaceutically active ingredient (as described above), the combined quantities of the compound of formula (I) and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) inhibit gamma-secretase.

Another embodiment of this invention is directed to a kit comprising, in separate containers, in a single package, pharmaceutical compositions for use in combination, wherein one container comprises an effective amount of a compound selected from the group consisting of compounds D-12, D-13, D1-22A, D2-22A, D3-22A and D-55, in a pharmaceutically acceptable carrier, and another container (i.e., a second container) comprises an effective amount of another pharmaceutically active ingredient (as described above), the combined quantities of the compound of formula (I) and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (e) treat neurodegenerative diseases, or (d) inhibit gamma-secretase.

Another embodiment of this invention is directed to compounds 12, 13, 22A to 22N, 22B-rac, 26A, 26B, 27, 28, 31, 33(−), 36, 37, 39, 44A, 44B, 55 to 57, 60, 70A, 71A(−), 75A-rac, 75B(−), 76A(−), 76A(+), 76B(−), 78A(−), 79A(−), 80A(+), 81A-rac, 81A(−), 81A(+), 81B(−), 82A, 83A, 84A, 85A, 85B(−), 86A, 87B, 95B-rac, 95B(−), 95B(+), 106(−), 107(−), 108(−), and 111(−) in pure form, wherein at least one (e.g., 1-4, 1-3, 1-2, 2 or 1) H is replaced by deuterium.

Another embodiment of this invention is directed to compounds 12, 13, 22A to 22N, 22B-rac, 26A, 26B, 27, 28, 31, 33(−), 36, 37, 39, 44A, 44B, 55 to 57, 60, 70A, 71A(−), 75A-rac, 75B(−), 76A(−), 76A(+), 76B(−), 78A(−), 79A(−), 80A(+), 81A-rac, 81A(−), 81A(+), 81B(−), 82A, 83A, 84A, 85A, 85B(−), 86A, 87B, 95B-rac, 95B(−), 95B(+), 106(−) 107(−), 1080, and 111(−) in isolated form, wherein at least one (e.g., 1-4, 1-3, 1-2, 2 or 1) H is replaced by deuterium in said compounds.

Another embodiment of this invention is directed to compounds D-12, D-13, D1-22A, D2-22A, D3-22A and D-55.

Another embodiment of this invention is directed to compound D-12.

Another embodiment of this invention is directed to compound D-12 in pure form.

Another embodiment of this invention is directed to compound D-12 in isolated form.

Another embodiment of this invention is directed to a solvate of compound D-12.

Another embodiment of this invention is directed to compound D-13.

Another embodiment of this invention is directed to compound D-13 in pure form.

Another embodiment of this invention is directed to compound D-13 in isolated form.

Another embodiment of this invention is directed to a solvate of compound D-13.

Another embodiment of this invention is directed to compound D1-22A.

Another embodiment of this invention is directed to compound D1-22A in pure form.

Another embodiment of this invention is directed to compound D1-22A in isolated form.

Another embodiment of this invention is directed to a solvate of compound D1-22A.

Another embodiment of this invention is directed to compound D2-22A.

Another embodiment of this invention is directed to compound D2-22A in pure form.

Another embodiment of this invention is directed to compound D2-22A in isolated form.

Another embodiment of this invention is directed to a solvate of compound D2-22A.

Another embodiment of this invention is directed to compound D3-22A.

Another embodiment of this invention is directed to compound D3-22A in pure form.

Another embodiment of this invention is directed to compound D3-22A in isolated form.

Another embodiment of this invention is directed to a solvate of compound D3-22A.

Another embodiment of this invention is directed to compound D-55.

Another embodiment of this invention is directed to compound D-55 in pure form.

Another embodiment of this invention is directed to compound D-55 in isolated form.

Another embodiment of this invention is directed to a solvate of compound D-55.

Another embodiment of this invention is directed to a pharmaceutical composition comprising an effective amount of compound D-12, and at least one pharmaceutically acceptable carrier.

Another embodiment of this invention is directed to a pharmaceutical composition comprising an effective amount of compound D-13, and at least one pharmaceutically acceptable carrier.

Another embodiment of this invention is directed to a pharmaceutical composition comprising an effective amount of compound D1-22A, and at least one pharmaceutically acceptable carrier.

Another embodiment of this invention is directed to a pharmaceutical composition comprising an effective amount of compound D2-22A, and at least one pharmaceutically acceptable carrier.

Another embodiment of this invention is directed to a pharmaceutical composition comprising an effective amount of compound D3-22A, and at least one pharmaceutically acceptable carrier.

Another embodiment of this invention is directed to a pharmaceutical composition comprising an effective amount of compound D-55, and at least one pharmaceutically acceptable carrier.

Another embodiment of this invention is directed to a pharmaceutical composition comprising an effective amount of a solvate of compound D-12, and at least one pharmaceutically acceptable carrier.

Another embodiment of this invention is directed to a pharmaceutical composition comprising an effective amount of a solvate of compound D-13, and at least one pharmaceutically acceptable carrier.

Another embodiment of this invention is directed to a pharmaceutical composition comprising an effective amount of a solvate of compound D1-22A, and at least one pharmaceutically acceptable carrier.

Another embodiment of this invention is directed to a pharmaceutical composition comprising an effective amount of a solvate of compound D2-22A, and at least one pharmaceutically acceptable carrier.

Another embodiment of this invention is directed to a pharmaceutical composition comprising an effective amount of a solvate of compound D3-22A, and at least one pharmaceutically acceptable carrier.

Another embodiment of this invention is directed to a pharmaceutical composition comprising an effective amount of a solvate of compound D-55, and at least one pharmaceutically acceptable carrier.

The other pharmaceutically active ingredients (e.g., drugs), used in the pharmaceutical compositions with the compounds of formula (I), as well as being used in the methods of treatment with the compounds of formula (I) (i.e., the combination therapies described herein) include, but are not limited to BACE inhibitors (beta secretase inhibitors), muscarinic antagonists (e.g., m₁ or m₂ antagonists), cholinesterase inhibitors (e.g., acetyl- and/or butyrylchlolinesterase inhibitors); gamma secretase inhibitors; gamma secretase modulators, HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase, PDE-10 inhibitors, cholesterol absorption inhibitors (e.g., ezetimibe), and mTOR inhibitors.

Thus, another embodiment of this invention is directed to pharmaceutical compositions comprising an effective amount of one or more (e.g., one) compounds of formula (I) (e.g., compounds of formula (I.A1), or compounds of formula (I.A2)), and an effective amount of one or more (e.g., one) other pharmaceutically active ingredients (e.g., drugs), and a pharmaceutically acceptable carrier, wherein said other pharmaceutically active ingredients are selected from the group consisting of: BACE inhibitors (beta secretase inhibitors), muscarinic antagonists (e.g., m₁ or m₂ antagonists), cholinesterase inhibitors (e.g., acetyl- and/or butyrylchlolinesterase inhibitors); gamma secretase inhibitors; gamma secretase modulators, HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity, PDE-10 inhibitors, cholesterol absorption inhibitors (e.g., ezetimibe), and mTOR inhibitors.

Another embodiment of this invention is directed to pharmaceutical compositions comprising an effective amount of one or more (e.g., one) compounds selected from the group consisting of I.A1i, I.A1j, I.A1k, I.A1l, I.A1m, I.A1n, I.A1o, I.A1p, I.A1q, I.A1r and I.A1s, and an effective amount of one or more (e.g., one) other pharmaceutically active ingredients (e.g., drugs), and a pharmaceutically acceptable carrier, wherein said other pharmaceutically active ingredients are selected from the group consisting of: BACE inhibitors (beta secretase inhibitors), muscarinic antagonists (e.g., m₁ or m₂ antagonists), cholinesterase inhibitors (e.g., acetyl- and/or butyrylchlolinesterase inhibitors); gamma secretase inhibitors; gamma secretase modulators, HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity, PDE-10 inhibitors, cholesterol absorption inhibitors (e.g., ezetimibe), and mTOR inhibitors.

Another embodiment of this invention is directed to pharmaceutical compositions comprising an effective amount of one or more (e.g., one) compounds selected from the group consisting of D-12, D-13, D1-22A, D2-22A, D3-22A and D-55, and an effective amount of one or more (e.g., one) other pharmaceutically active ingredients (e.g., drugs), and a pharmaceutically acceptable carrier, wherein said other pharmaceutically active ingredients are selected from the group consisting of: BACE inhibitors (beta secretase inhibitors), muscarinic antagonists (e.g., m₁ or m₂ antagonists), cholinesterase inhibitors (e.g., acetyl- and/or butyrylchlolinesterase inhibitors); gamma secretase inhibitors; gamma secretase modulators, HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity, PDE-10 inhibitors, cholesterol absorption inhibitors (e.g., ezetimibe), and mTOR inhibitors.

Examples of cholinesterase inhibitors are tacrine, donepezil, rivastigmine, galantamine, pyridostigmine and neostigmine, with tacrine, donepezil, rivastigmine and galantamine being preferred.

Examples of m₁ antagonists are known in the art. Examples of m₂ antagonists are also known in the art; in particular, m₂ antagonists are disclosed in U.S. Pat. Nos. 5,883,096; 6,037,352; 5,889,006; 6,043,255; 5,952,349; 5,935,958; 6,066,636; 5,977,138; 6,294,554; 6,043,255; and 6,458,812; and in WO 03/031412, all of which are incorporated herein by reference.

Examples of BACE inhibitors include those described in: US2005/0119227 published Jun. 2, 2005 (see also WO2005/016876 published Feb. 24, 2005), US2005/0043290 published Feb. 24, 2005 (see also WO2005/014540 published Feb. 17, 2005), WO2005/058311 published Jun. 30, 2005 (see also US2007/0072852 published Mar. 29, 2007), US2006/0111370 published May 25, 2006 (see also WO2006/065277 published Jun. 22, 2006), U.S. application Ser. No. 11/710,582 filed Feb. 23, 2007, US2006/0040994 published Feb. 23, 2006 (see also WO2006/014762 published Feb. 9, 2006), WO2006/014944 published Feb. 9, 2006 (see also US2006/0040948 published Feb. 23, 2006), WO2006/138266 published Dec. 28, 2006 (see also US2007/0010667 published Jan. 11, 2007), WO2006/138265 published Dec. 28, 2006, WO2006/138230 published Dec. 28, 2006, WO2006/138195 published Dec. 28, 2006 (see also US2006/0281729 published Dec. 14, 2006), WO2006/138264 published Dec. 28, 2006 (see also US2007/0060575 published Mar. 15, 2007), WO2006/138192 published Dec. 28, 2006 (see also US2006/0281730 published Dec. 14, 2006), WO2006/138217 published Dec. 28, 2006 (see also US2006/0287294 published Dec. 21, 2006), US2007/0099898 published May 3, 200 (see also WO2007/050721 published May 3, 2007), WO2007/053506 published May 10, 2007 (see also US2007/099875 published May 3, 2007), U.S. application Ser. No. 11/759,336 filed Jun. 7, 2007, U.S. Application Ser. No. 60/874,362 filed Dec. 12, 2006, and U.S. Application Ser. No. 60/874,419 filed Dec. 12, 2006, the disclosures of each being incorporated herein by reference thereto.

The compounds of this invention can be combined with MTOR inhibitors.

Thus, any of the methods of this invention can optionally include the administration of an effective amount of one or more (e.g., 1, 2 or 3, or 1 or 2, or 1) MTOR inhibitors. The MTOR inhibitors can be administered currently or sequentially with the compounds of the invention and with the optional chemotherapeutic agents.

Examples of mTOR inhibitors include but are not limited to: those disclosed in: US 2007/0112005 (which describes fused bicyclic mTOR inhibitors useful in treatment of cancer), WO 2007/087395 (which describes unsaturated mTOR inhibitors useful in treatment of cancer), WO 2006/090169 (which describes 2,4-diamineo-pyrido-pyrmidine derivatives and their use as mTOR inhibitors), WO 2007/066099 (which describes pyrimidine derivatives useful as mTOR kinase inhibitors for anticancer), US 2005/0222171 (which describes pyrazolo[1,5 a]pyrimidin-7-yl amine derivatives to treat protein kinase dependent diseases), WO 2005/070431 (which describes pyrazolo[1,5 a]pyrimidin-7-yl amine derivatives to treat protein kinase dependent diseases), WO 2007/0570431(which describes pyrazolo[1,5 a]pyrimidin-7-yl amine derivatives to treat protein kinase dependent diseases), WO 2007/009773 (which describes pyrazolo[1,5 a]pyrimidin-7-yl amine derivatives to treat protein kinase dependent diseases), and US 2002/0041880 (which describes pyrazolo[1,5 a]pyrimidin-7-yl derivatives to inhibit kinase insert domain-containing receptor to block angiogenesis).

mTOR ihibitors also include compounds of the formula:

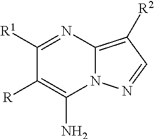

or a pharmaceutically acceptable salt, solvate, ester, or prodrug thereof, wherein: R is independently selected from the group consisting of halo, hydroxyl, amino, —CN, H, —(C₁-C₆)alkyl, alkoxy, —C(═O)alkyl, heteroaryl and aryl, wherein each of said heteroaryl and aryl can be unsubstituted or substituted with one or more alkyl and halo; R¹ is independently selected from the group consisting of heterocycloalkyl, heterocycloalkylalkyl, spiroheterocycloalkyl, heterocyclenyl, —NR³R⁴, cycloalkyl, heteroaryl, aryl, alkyl, alkynyl, heterocyclenylalkyl, cycloalkylalkyl, heteroarylalkyl, heteroarylalkynyl, spiroheterocycloalkylalkyl, —N-heteroaryl, -alkyl-NH-heterocyclyl and arylalkyl, wherein each of said heterocycloalkyl, heterocycloalkylalkyl, spiroheterocycloalkyl, heterocyclenyl, cycloalkyl, heteroaryl, aryl, alkyl, alkynyl, heterocyclenylalkyl, cycloalkylalkyl, heteroarylalkyl, heteroarylalkynyl, —N-heteroaryl and arylalkyl can be unsubstituted or substituted with one or moieties independently selected from the group X; X is alkoxyl, alkyl, —C(O)alkyl, —C(O)-hydroxyalkyl, —C(O)₂alkyl, —C(O)₂H, hydroxyalkyl, —S(O)₂alkyl, hydroxyl, heterocycloalkyl, —NH-heterocycloalkyl, -trihaloalkyl, -dihaloalkyl, -monohaloalkyl, —N—S(O)₂-alkyl, —C(O)-heteroaryl, -alkyl-C(O)₂H, -alkyl(CO)N(CH₃)—O—CH₃, -alkyl(CO)-heteroaryl, -alkyl-C(O)—NH₂, —NH₂, heteroaryl, -alkyl-CN, —C(O)₂-arylalkyl, halo, carboxyesteralkyl, —C(O)—NH₂, -alkyl-C(O)₂alkyl, heteroarylalkyl, —C(O)-heteroaryl, —C(O)-alkyl-O-alkyl, -alkyl(CO)NS(O)₂-cycloalkyl, -alkyl(CO)N—S(O)₂—CF₃, —N-alkyl, —SO₂-cycloalkyl, -alkyl(CO)NS(O)₂-alkyl, -alkyl-C(O)—N(alkyl)₂, -alkyl-NS(O)₂-alkyl, alkyl(CO)NS(O)₂-cycloalkyl, —CO—CO₂H, —C(O)₂-alkyl-aryl, —SO₂—CF₃ or —C(O)H, wherein each of said heterocycloalkyl, heteroaryl or —C(O)-heteroaryl can be unsubstituted or substituted with one or more alkyl; R² is heteroaryl or aryl, wherein each of said heteroaryl or aryl can be unsubstituted or independently substituted with one or more moieties independently selected from the group consisting of alkyl, alkoxyl, —CN, aryloxyl, aryl, halo, hydroxyl, —C(CH₃)₂CN, trifluoromethyl, difluoromethyl, monofluoromethyl, heterocycloalkyl, and arylalkyl; R³ is cycloalkyl or heteroaryl, wherein each of said cycloalkyl or heteroaryl can be unsubstituted or substituted with one or more moieties independently selected from the group consisting of X; and R⁴ is H, as described in U.S. Provisional Application Ser. No. 61/168,093, filed Apr. 9, 2009, the disclosure of which is incorporated herein by reference thereto.

Examples of mTOR inhibitors disclosed in U.S. Provisional Application Ser. No. 61/168,093 are:

Non-limiting examples of the compounds of the present invention include:

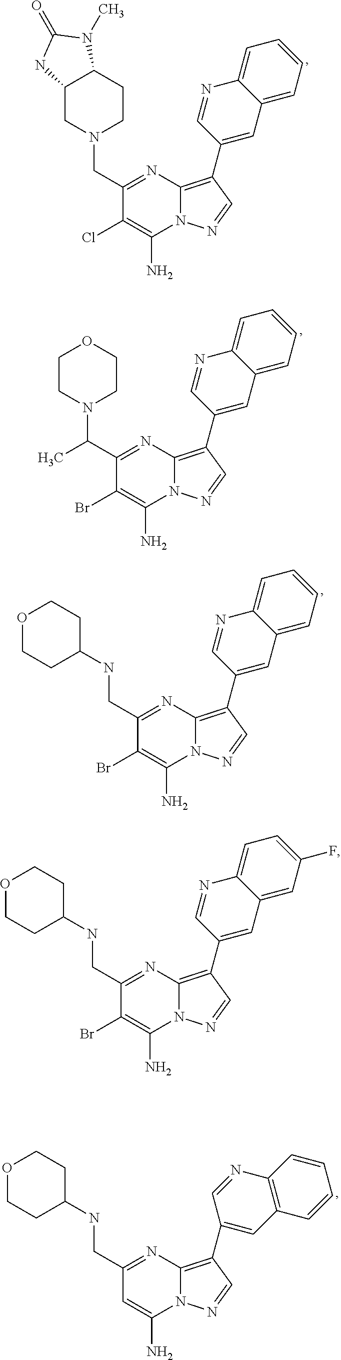

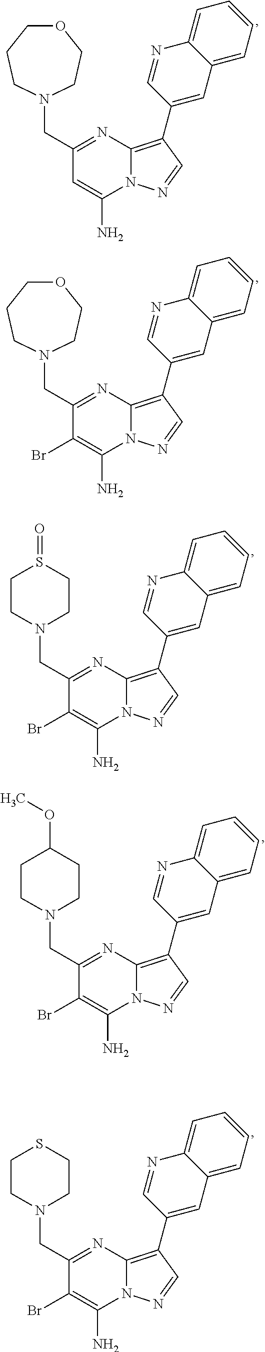

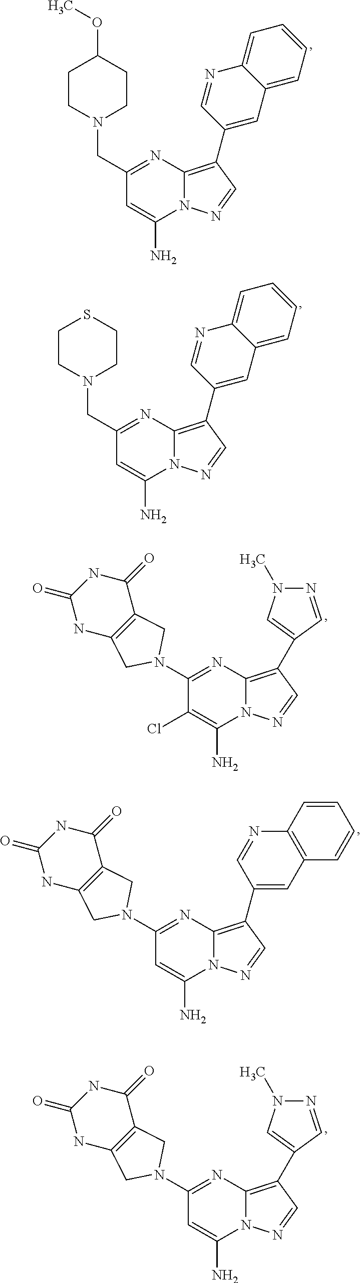

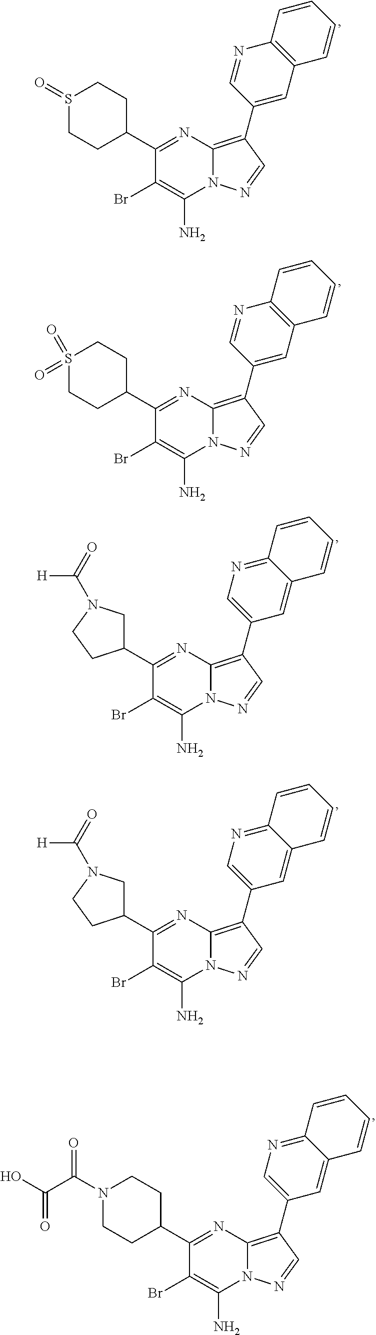

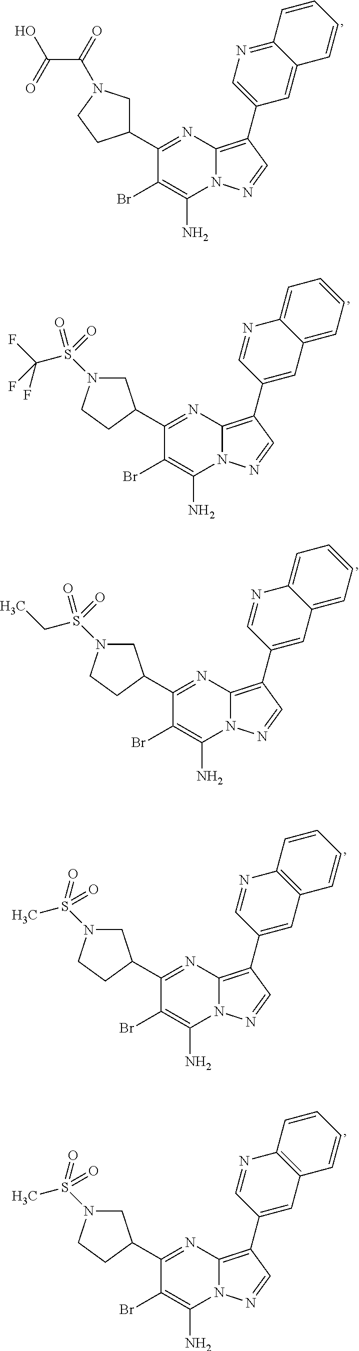

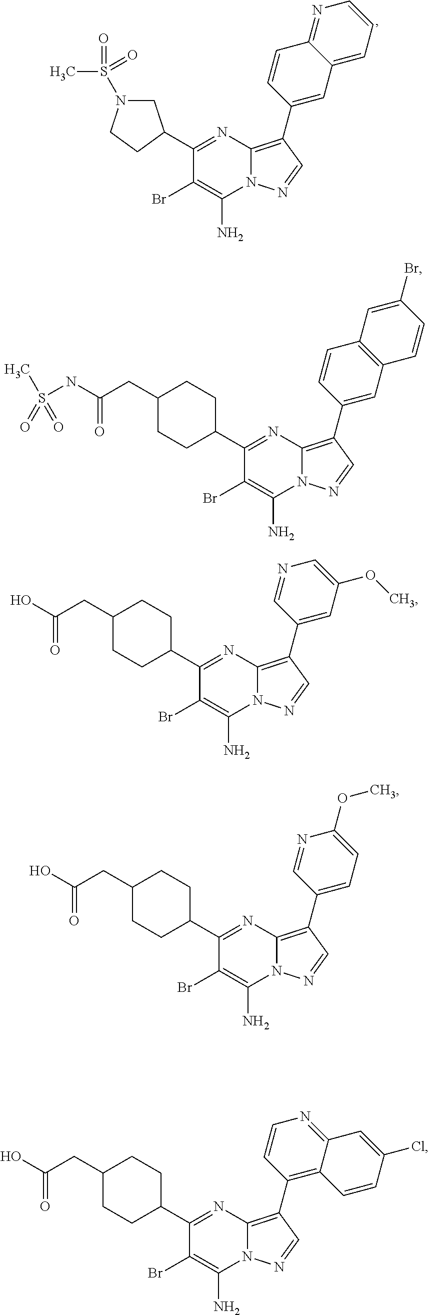

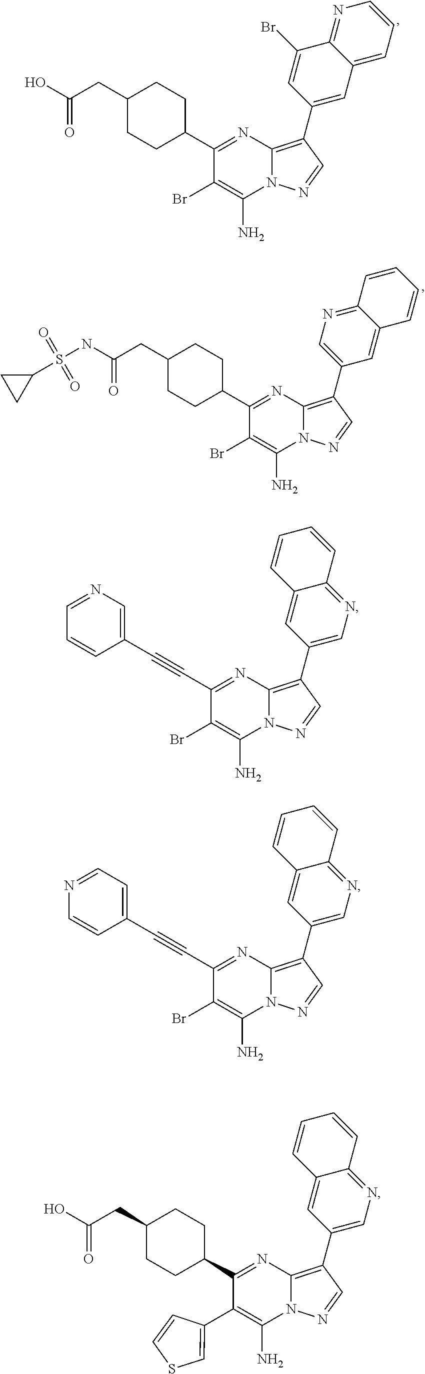

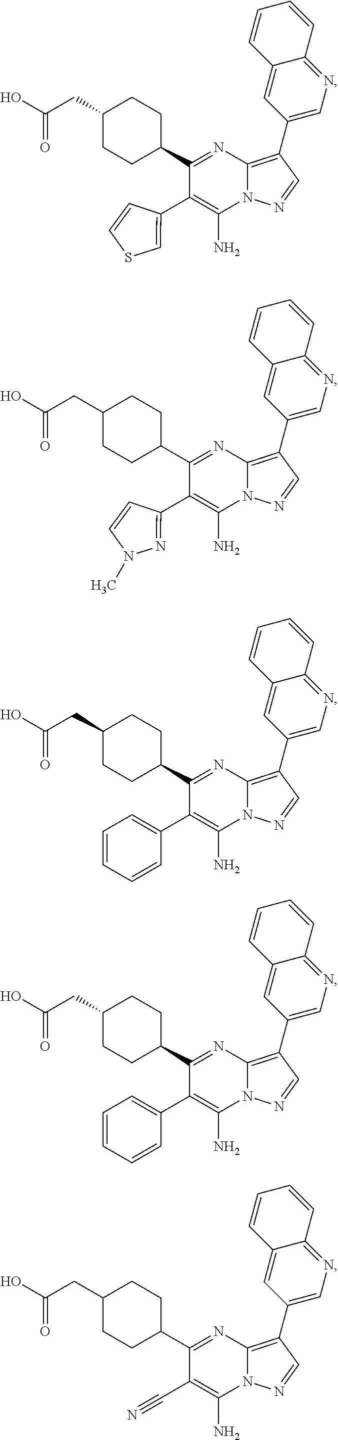

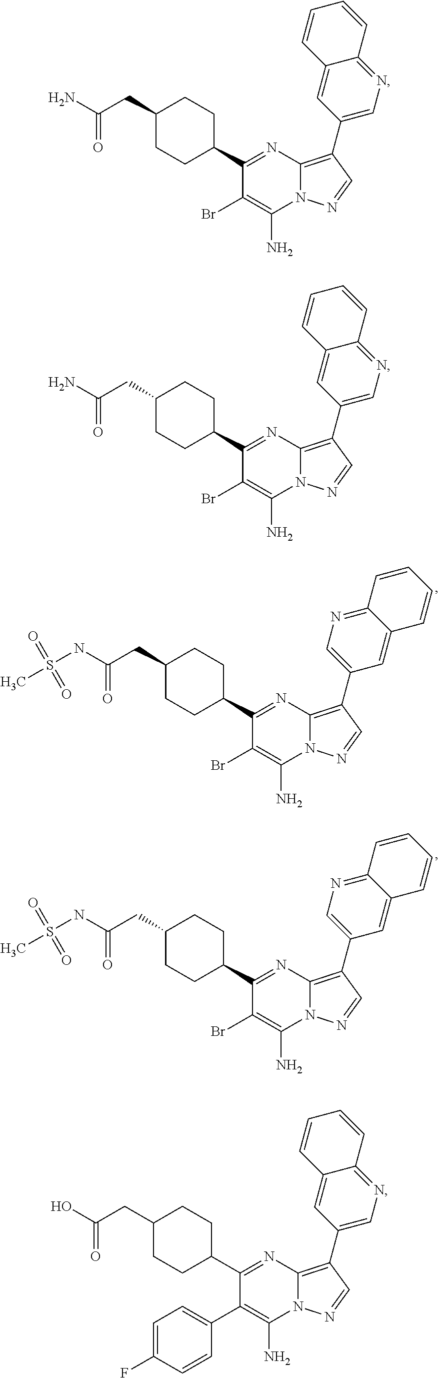

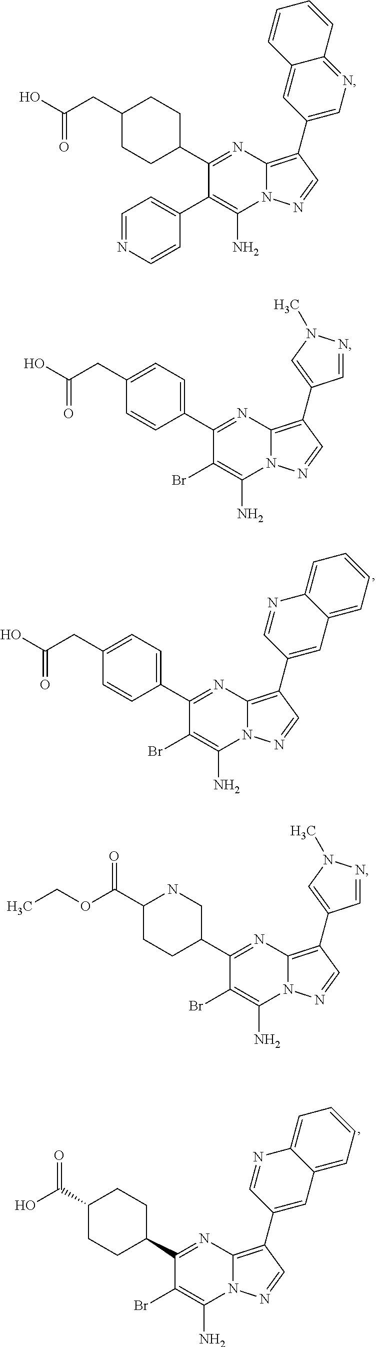

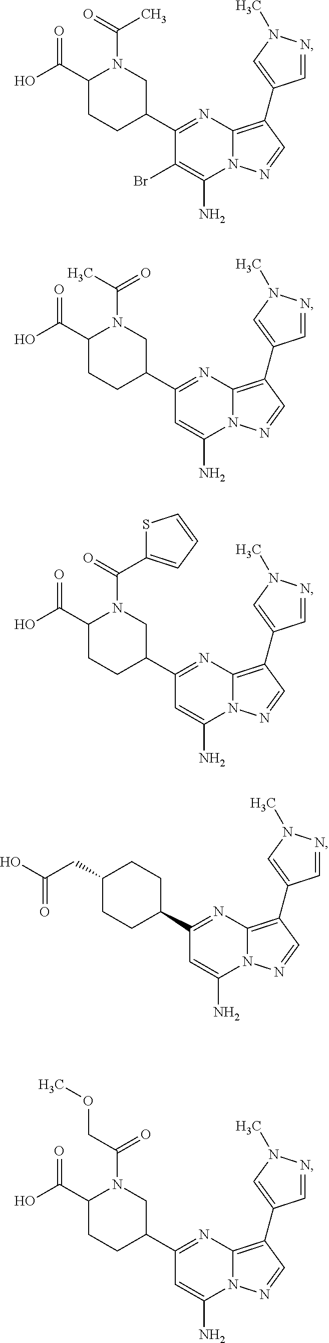

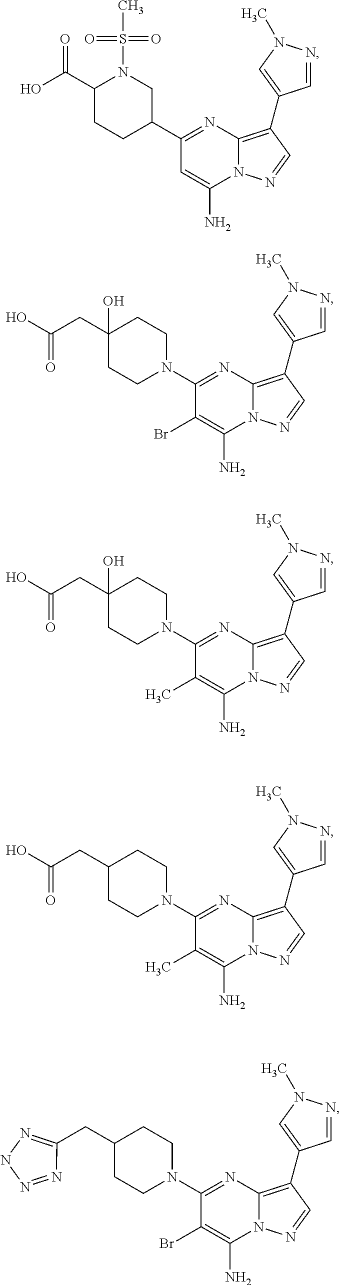

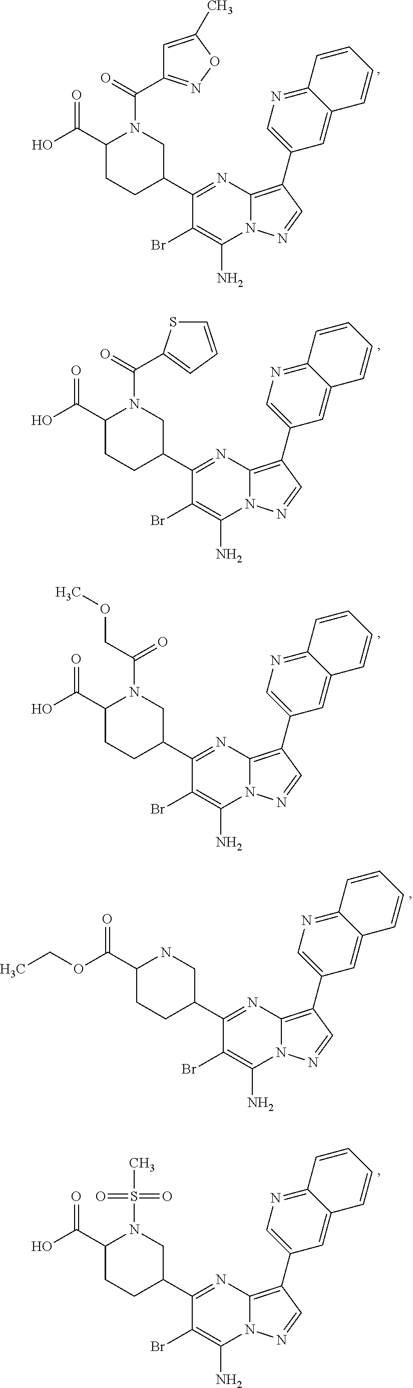

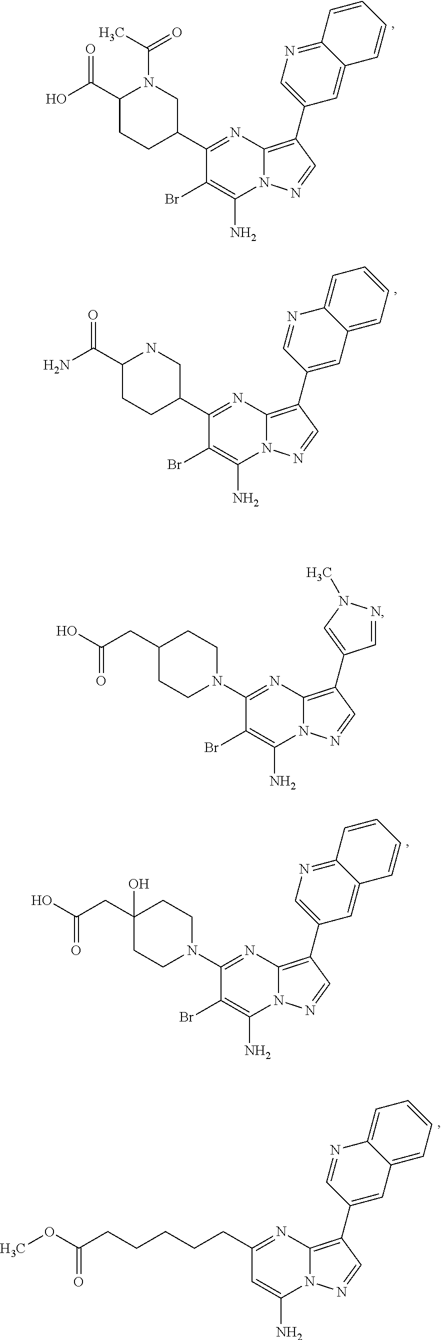

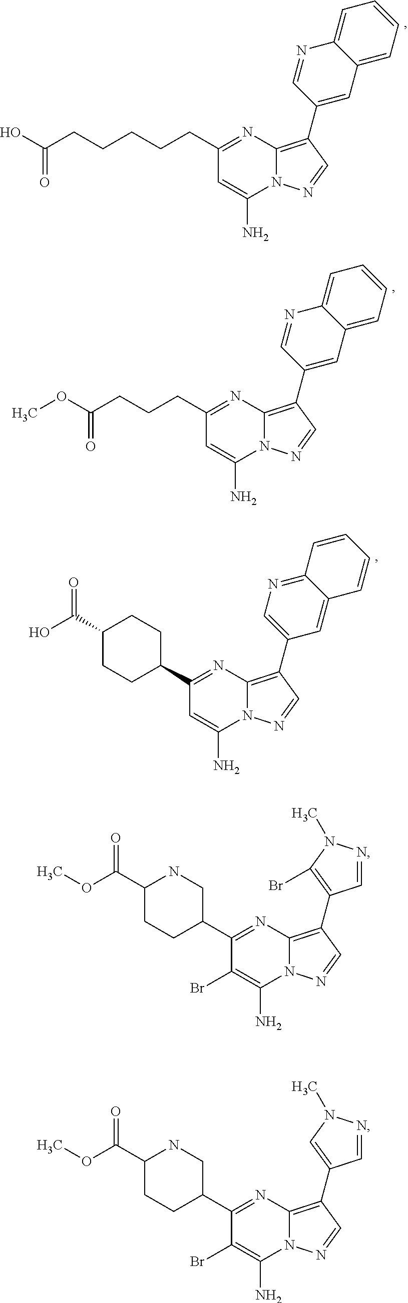

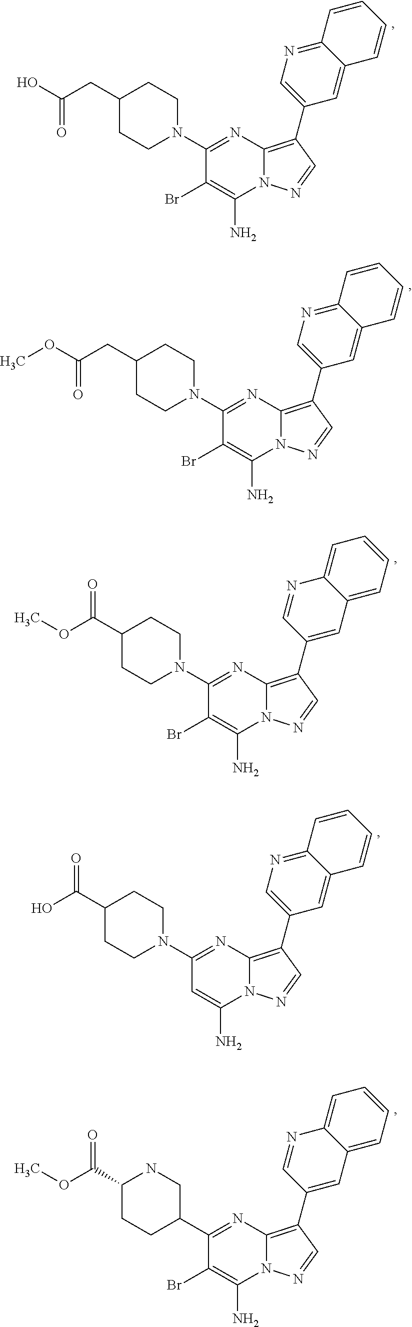

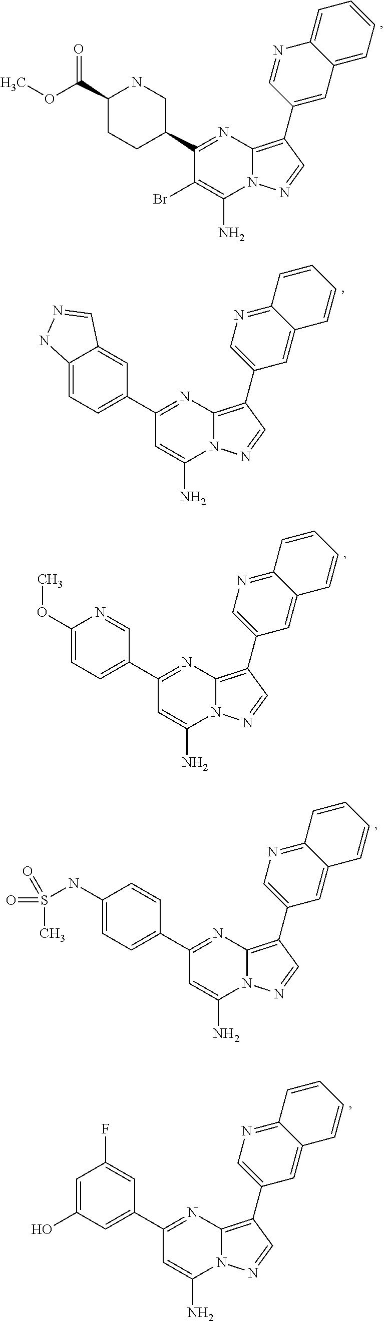

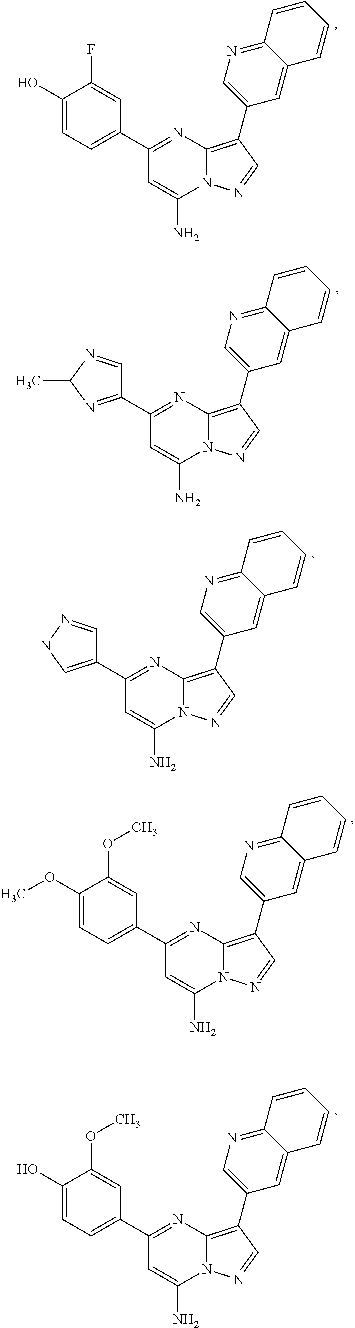

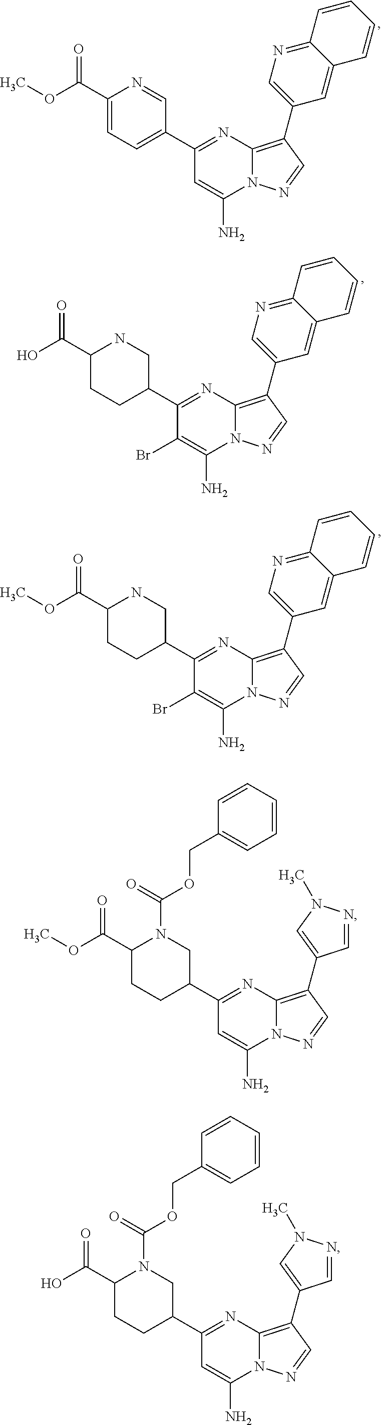

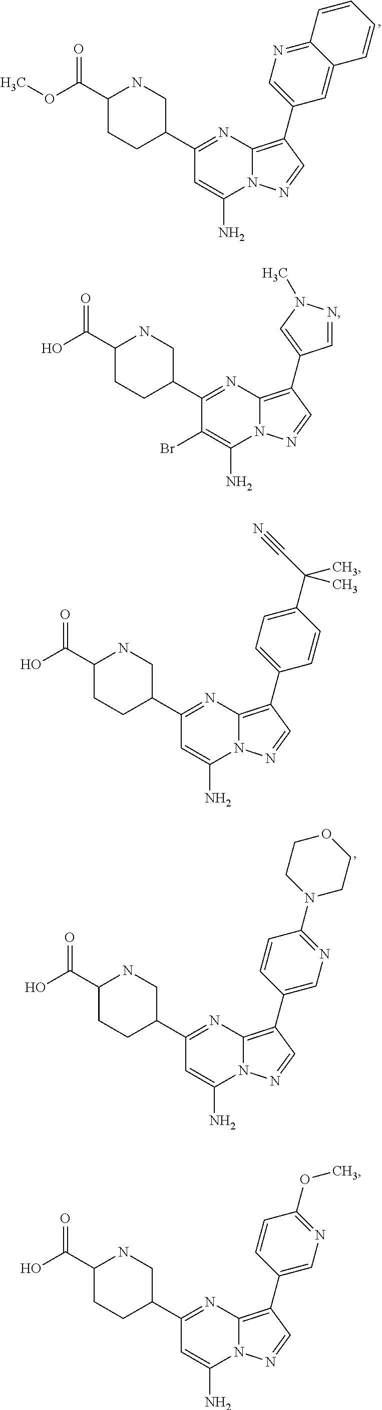

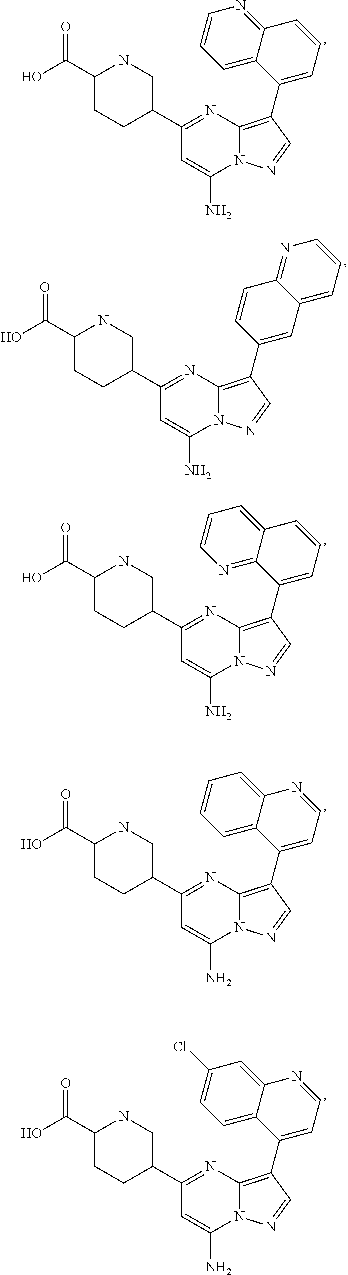

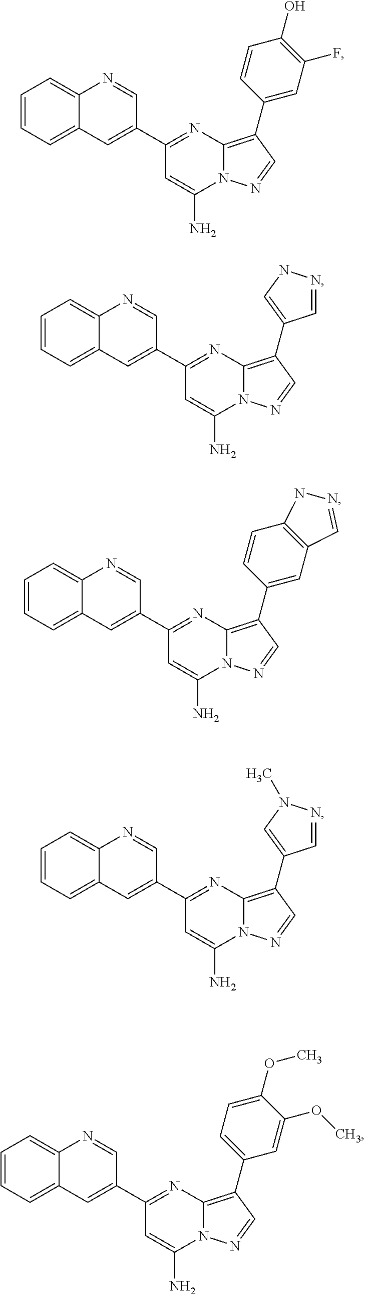

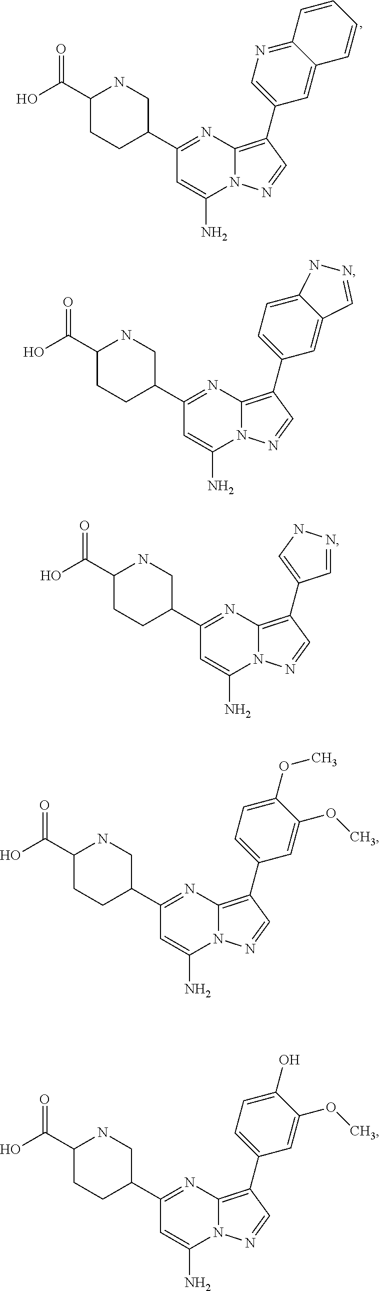

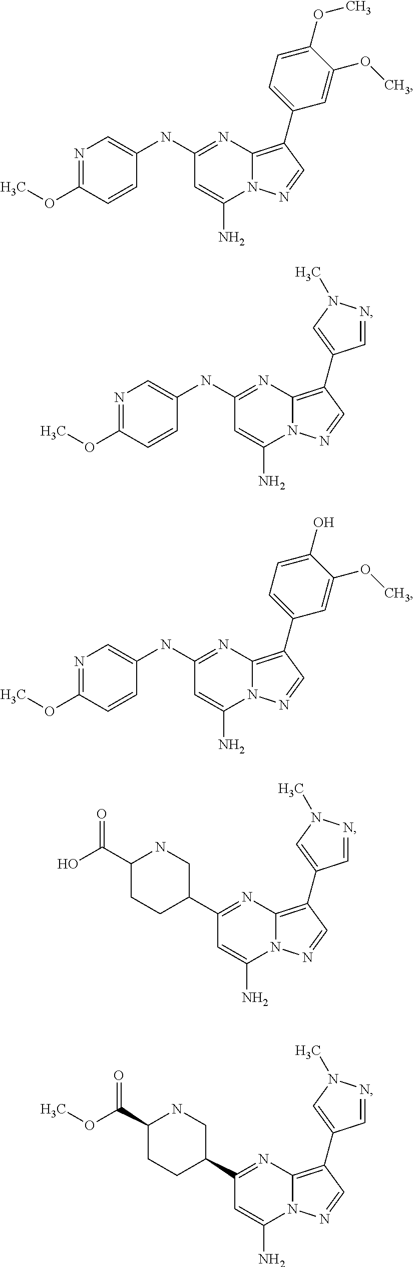

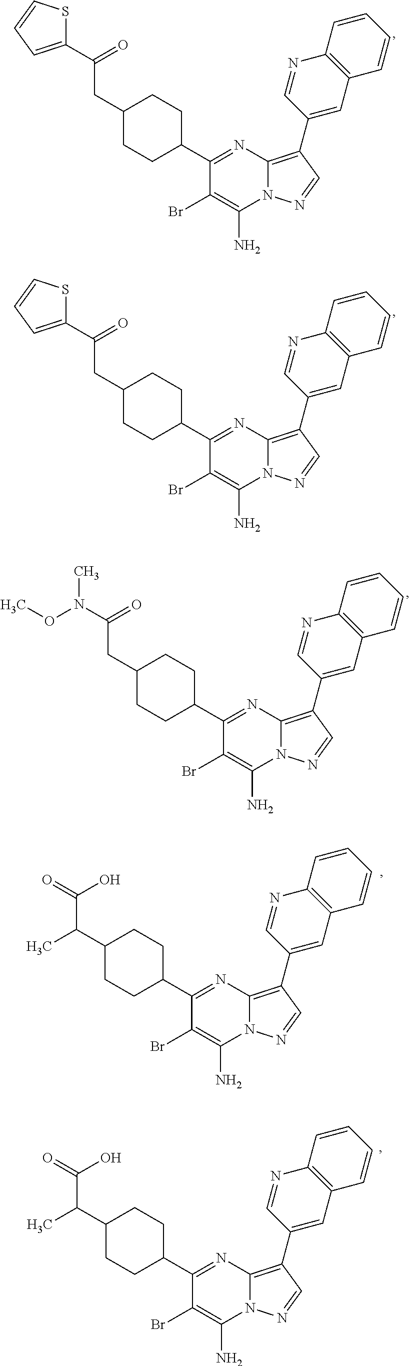

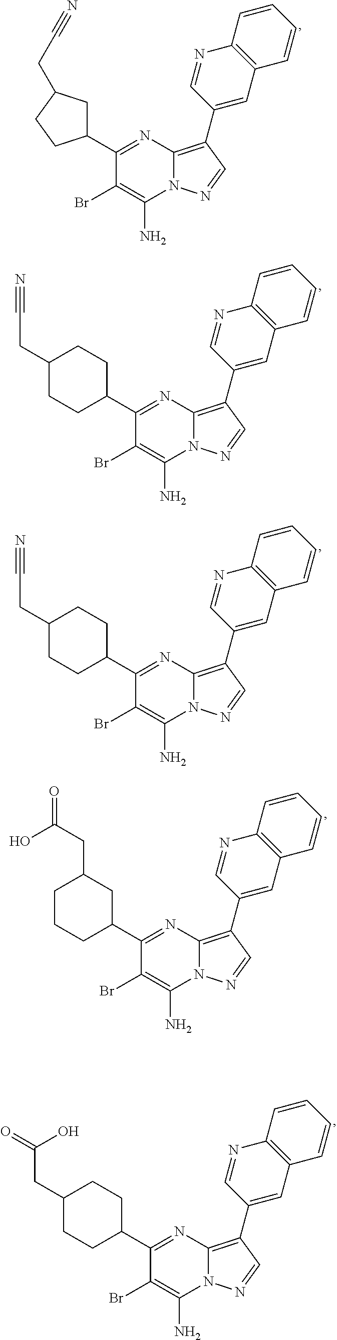

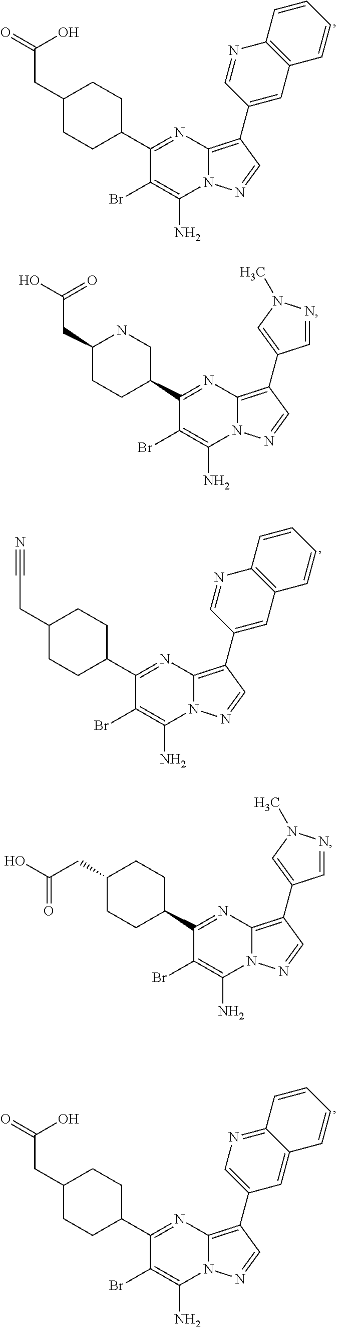

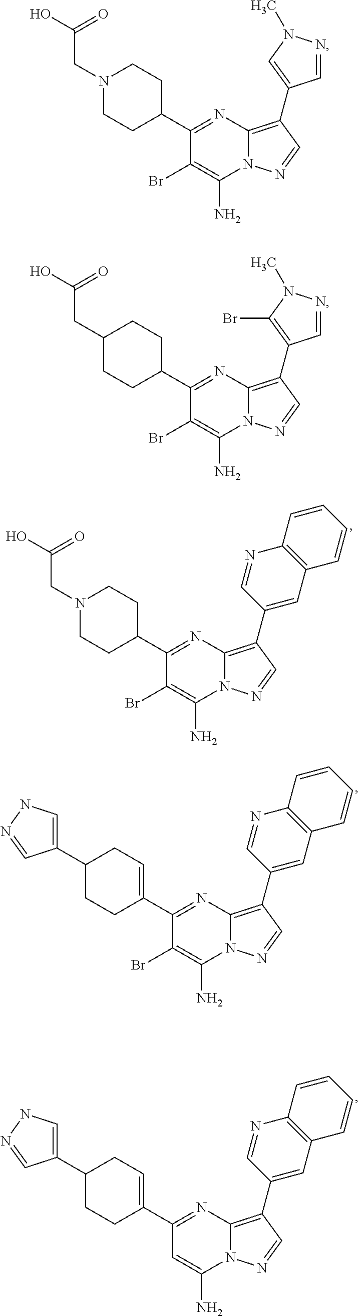

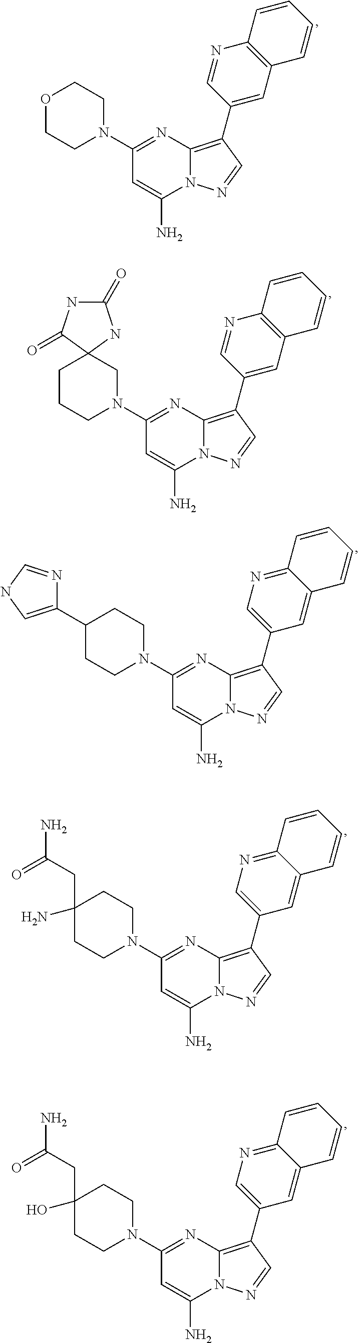

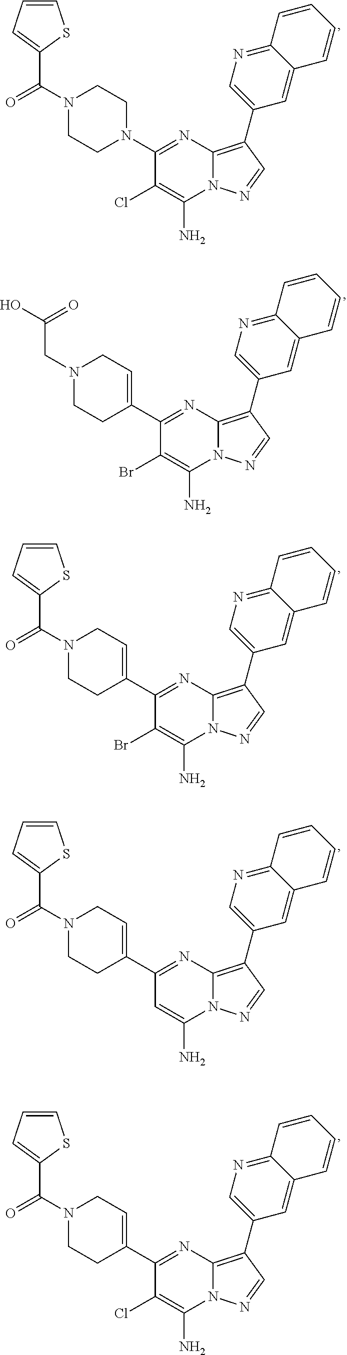

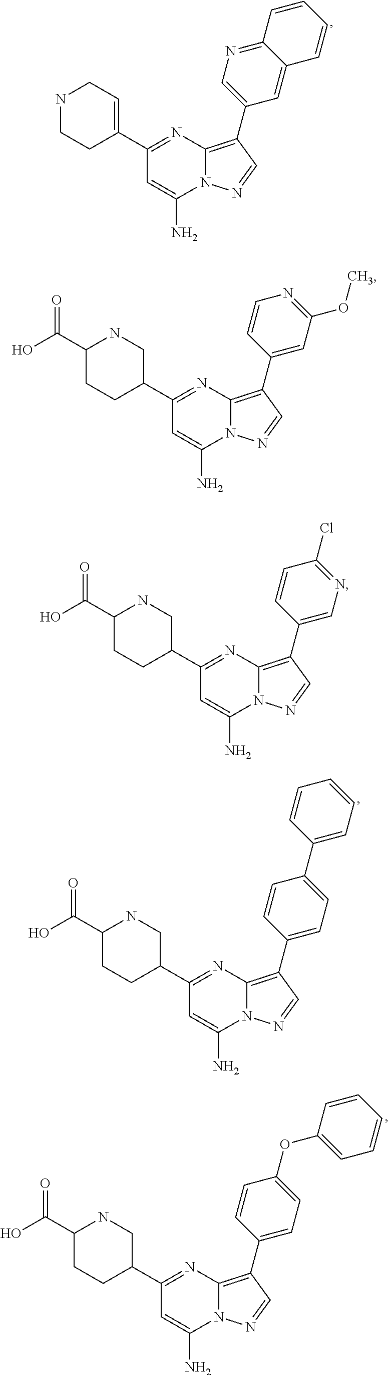

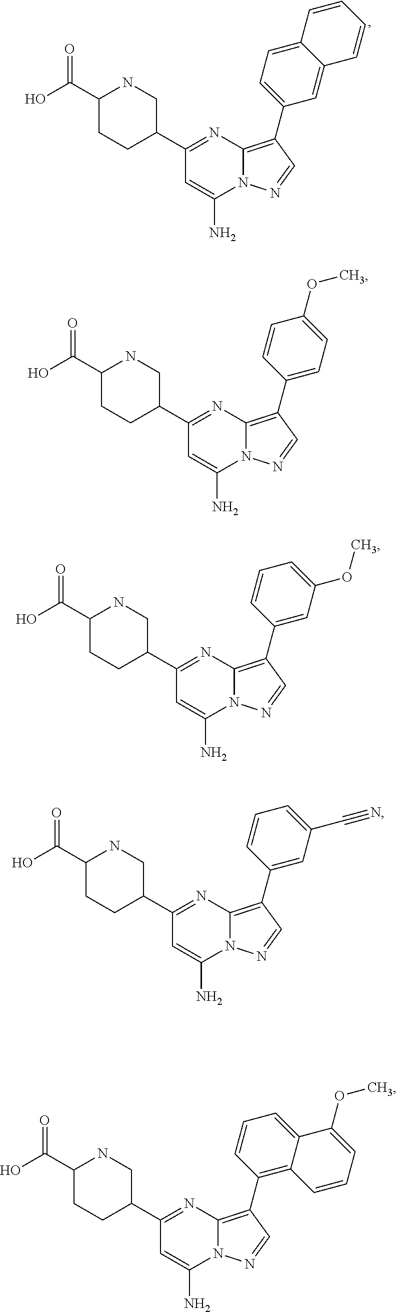

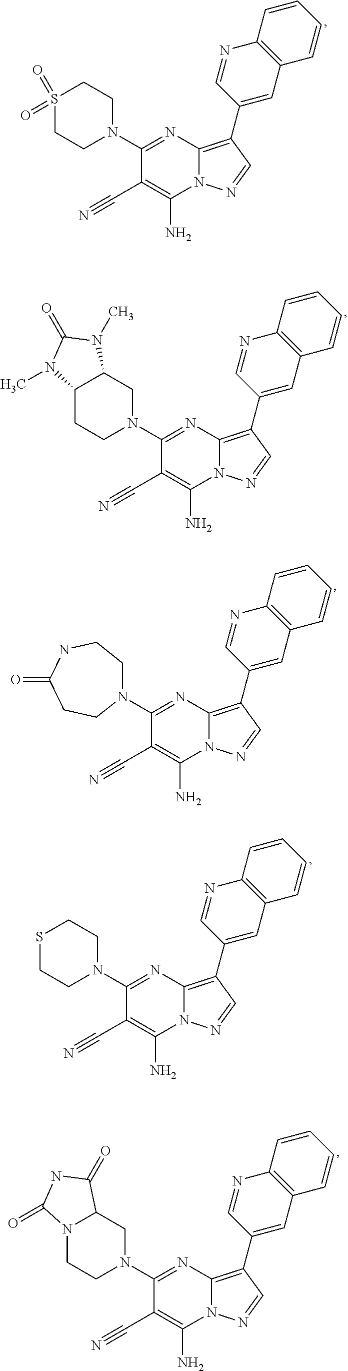

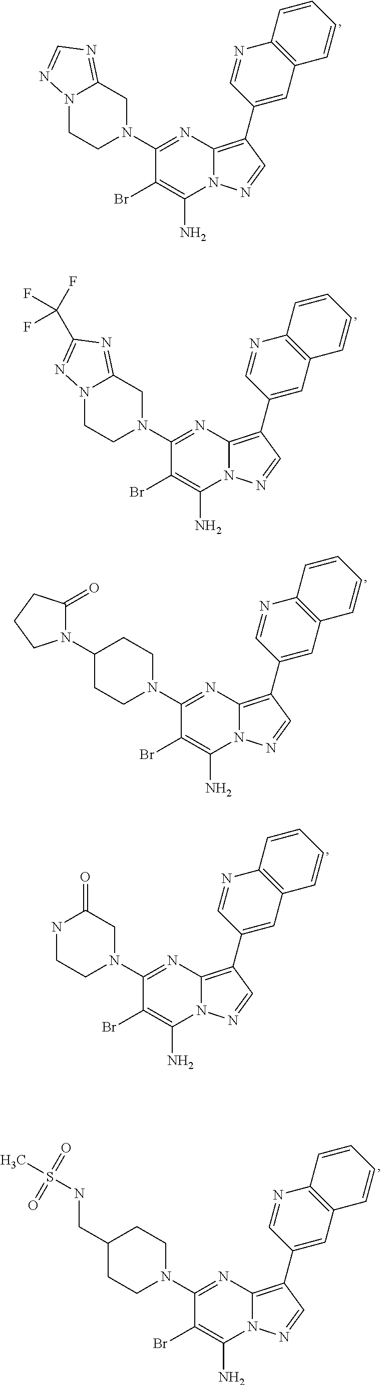

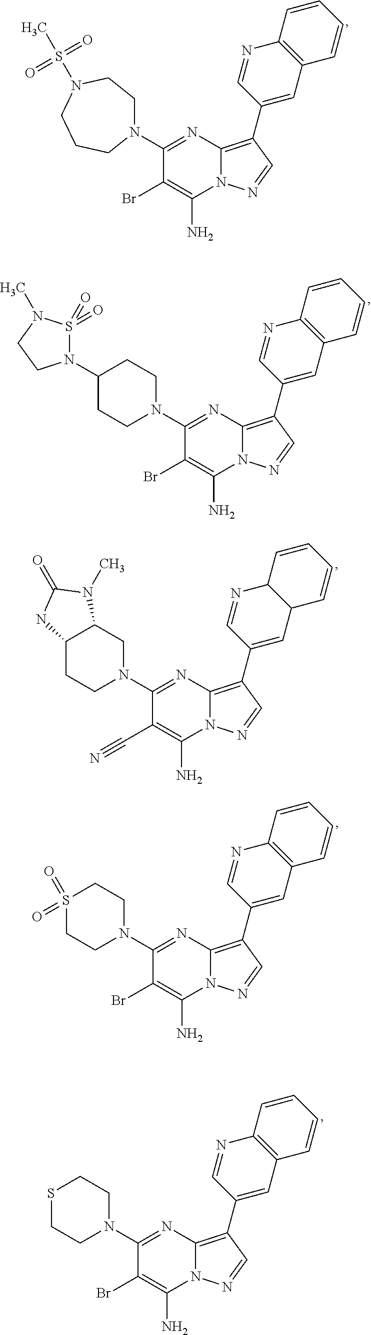

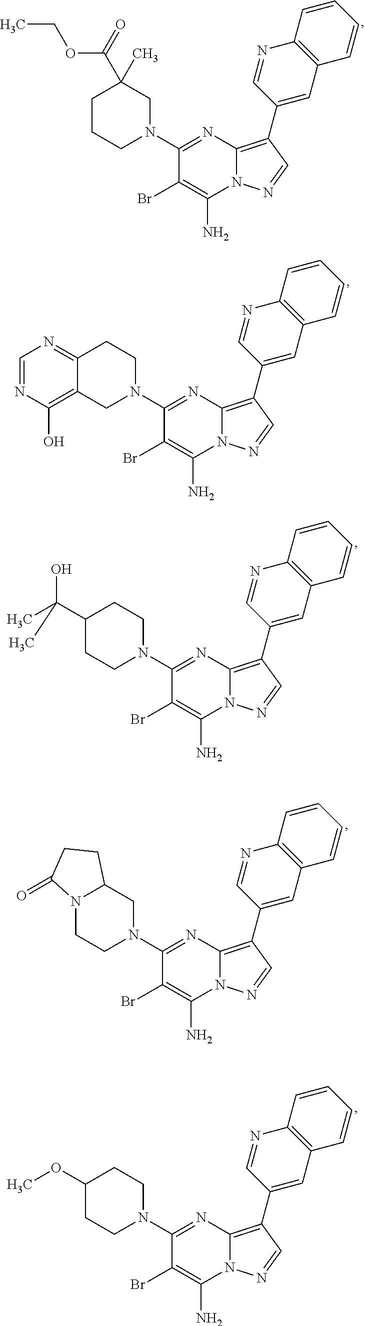

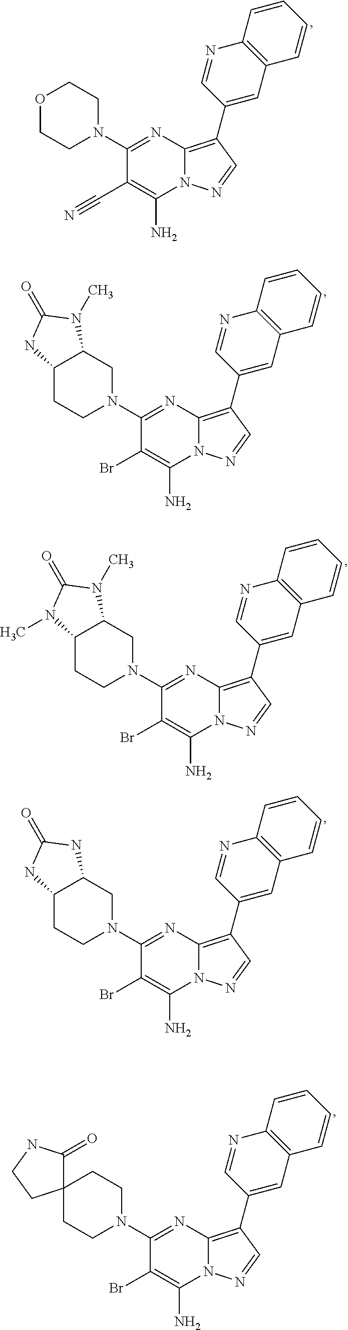

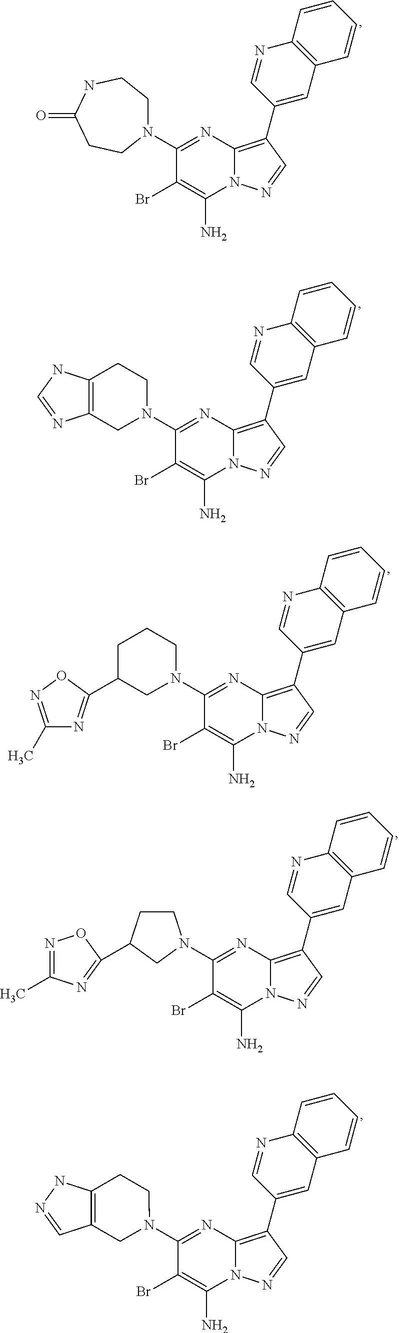

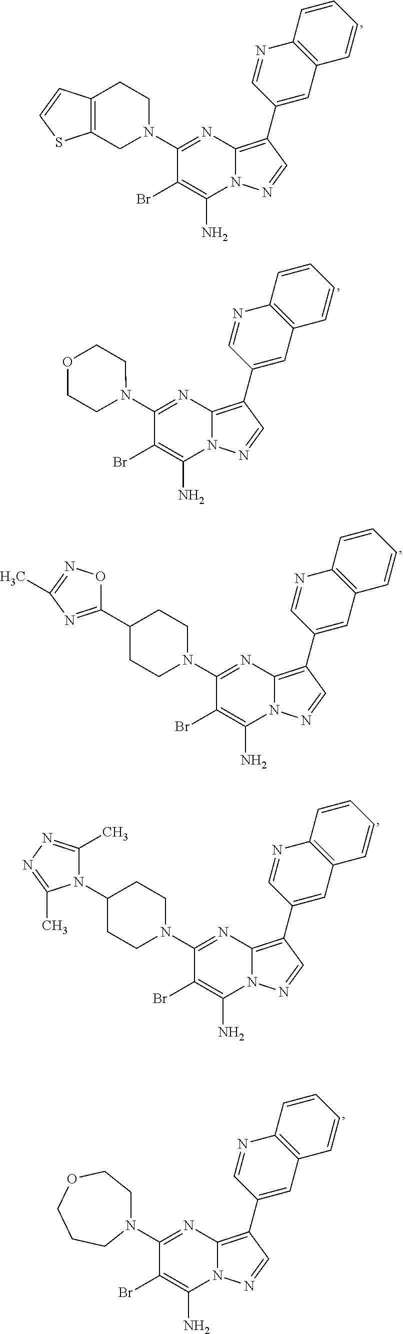

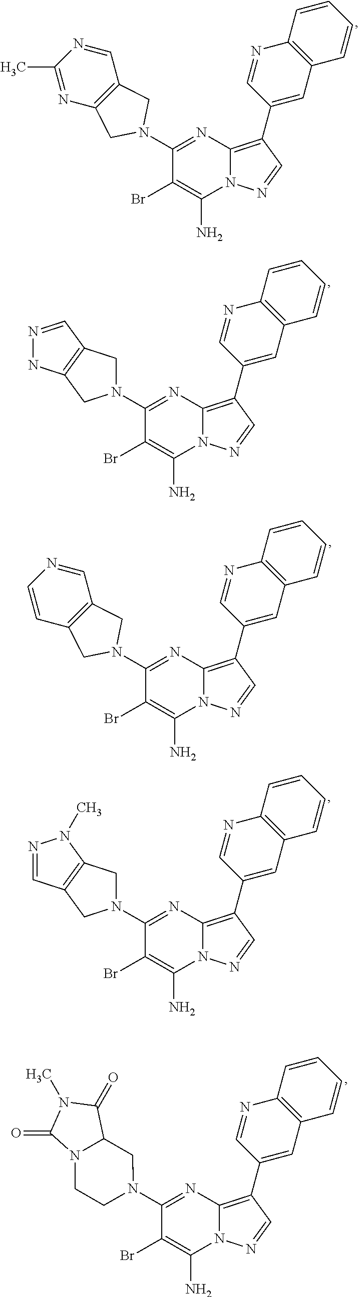

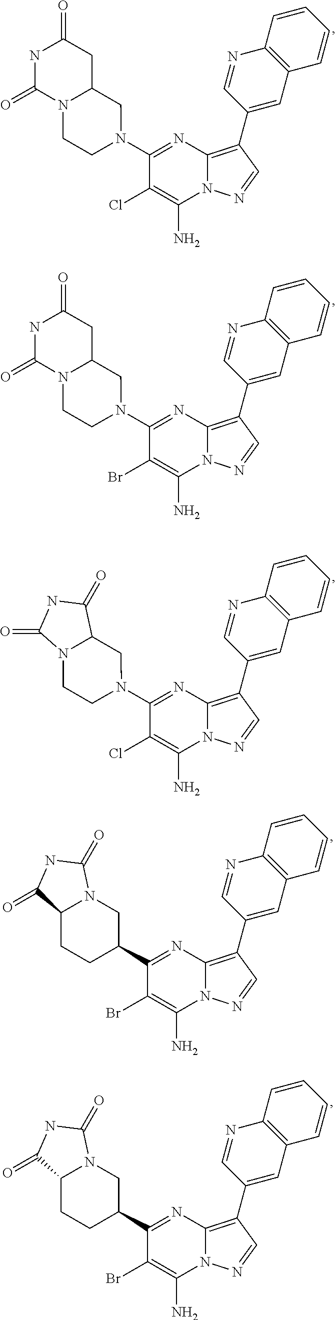

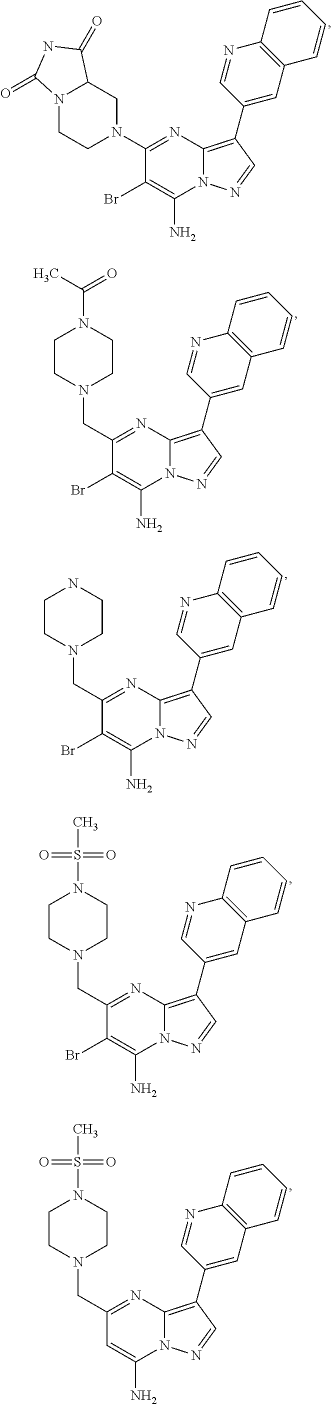

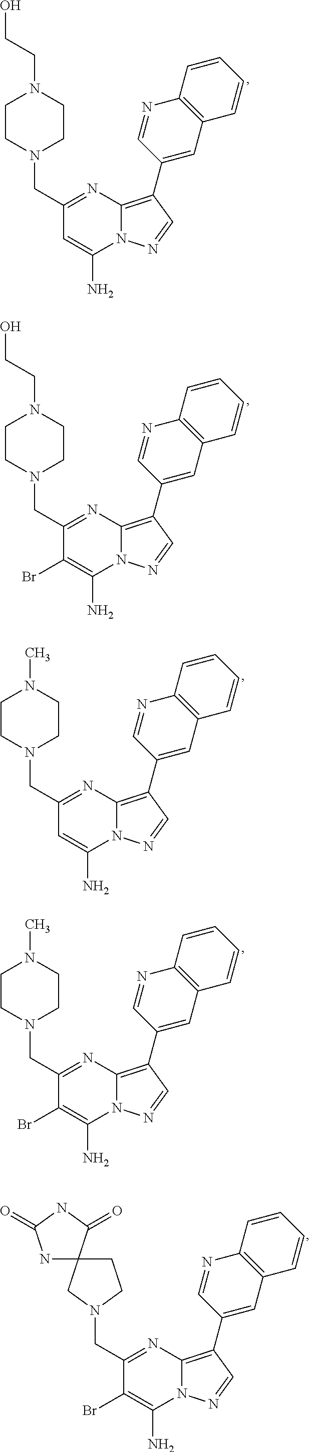

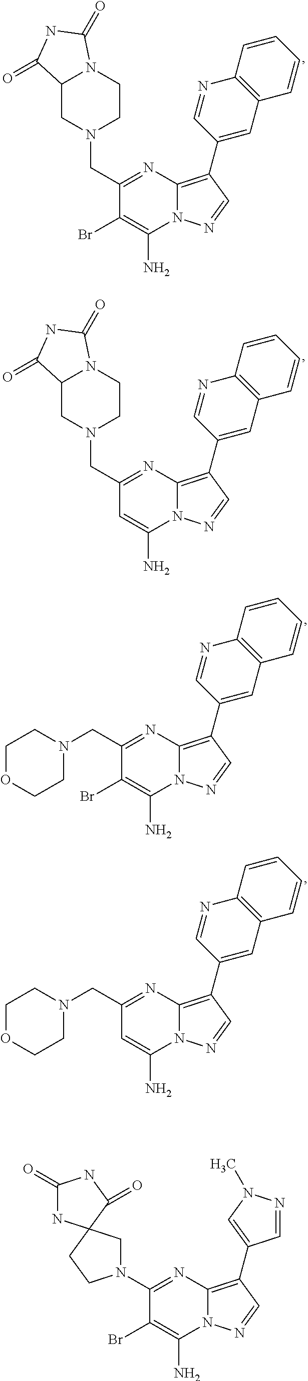

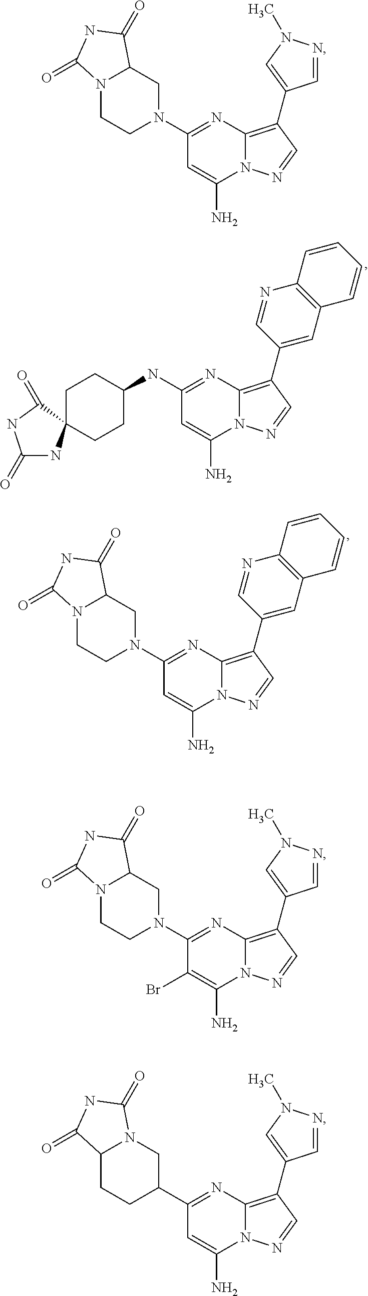

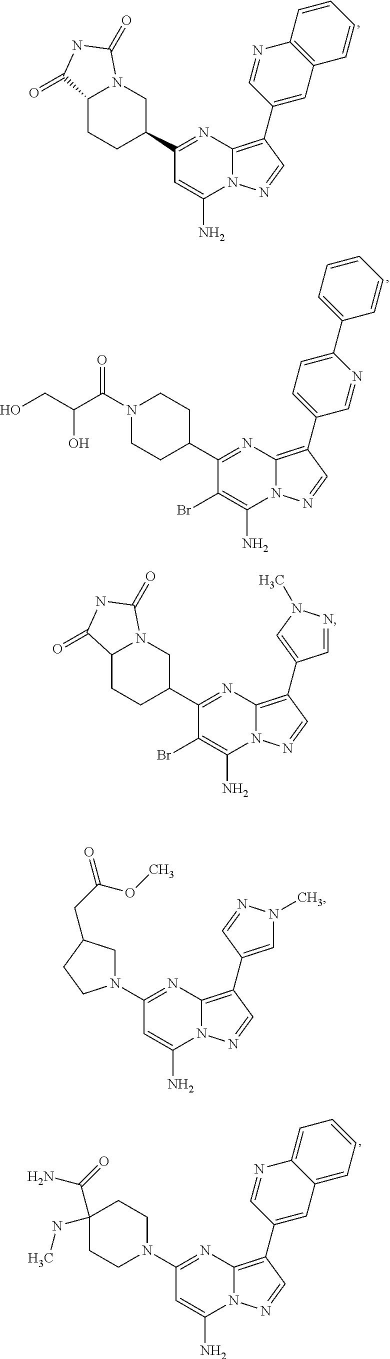

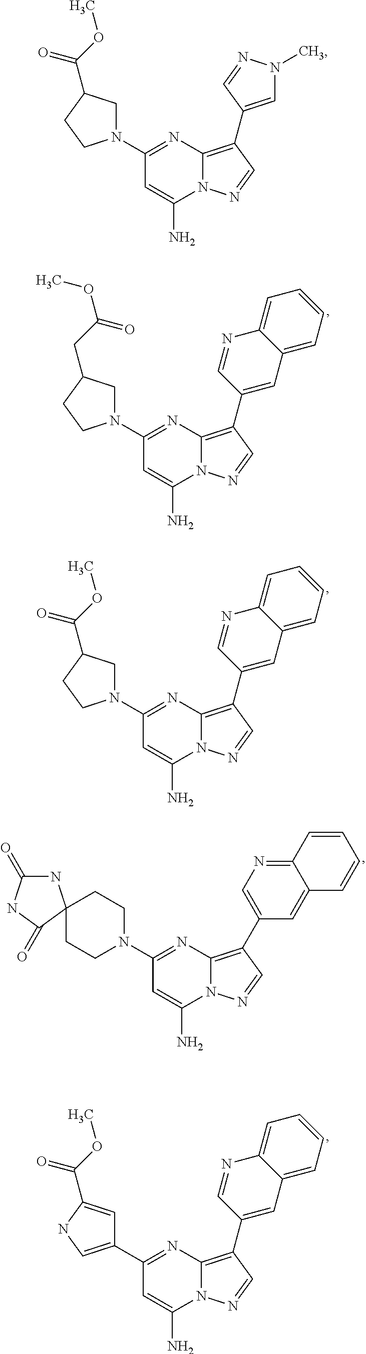

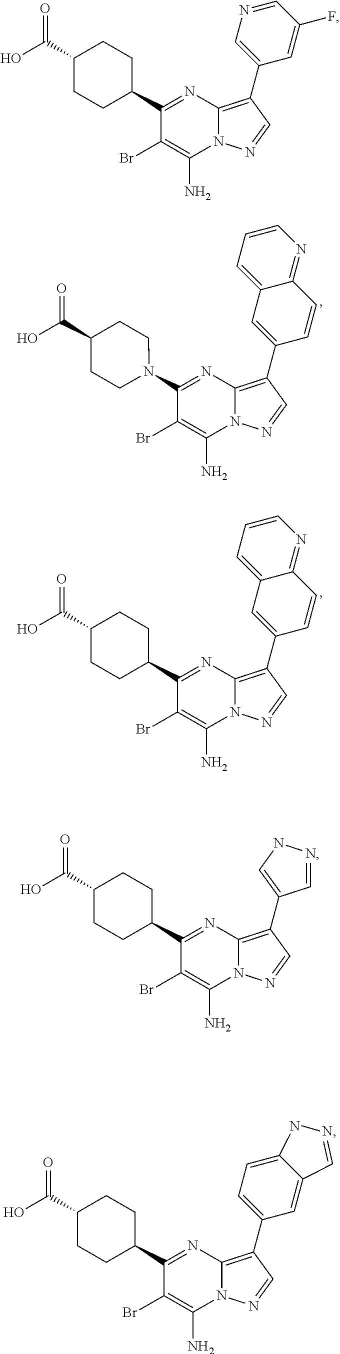

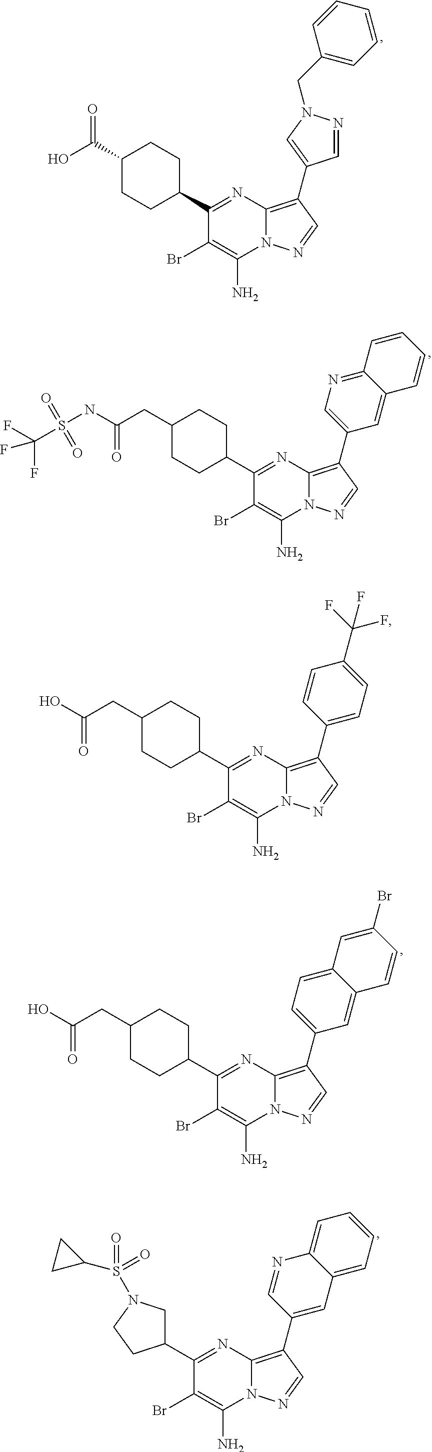

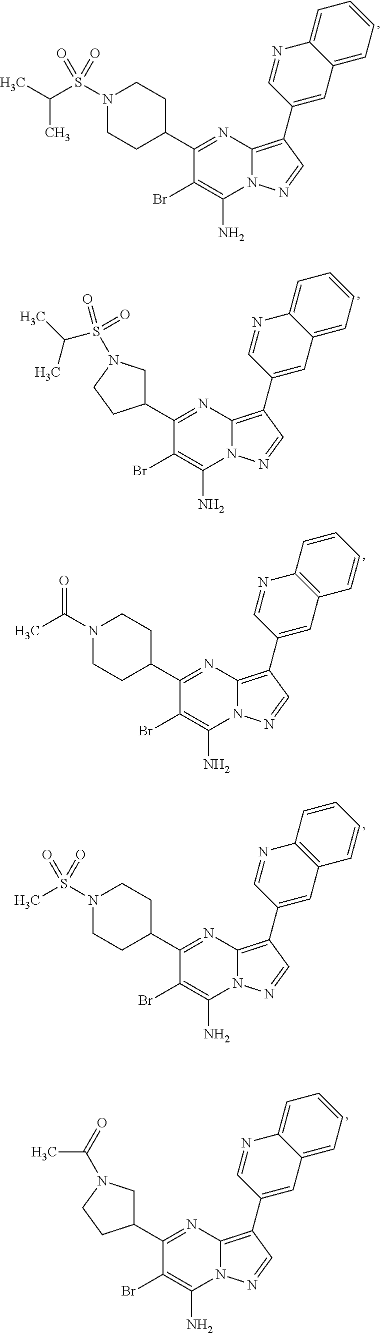

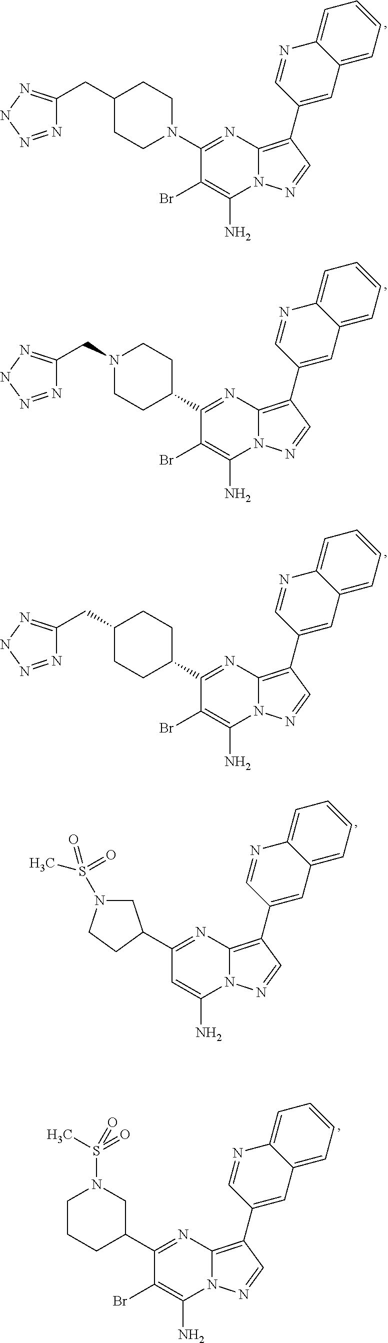

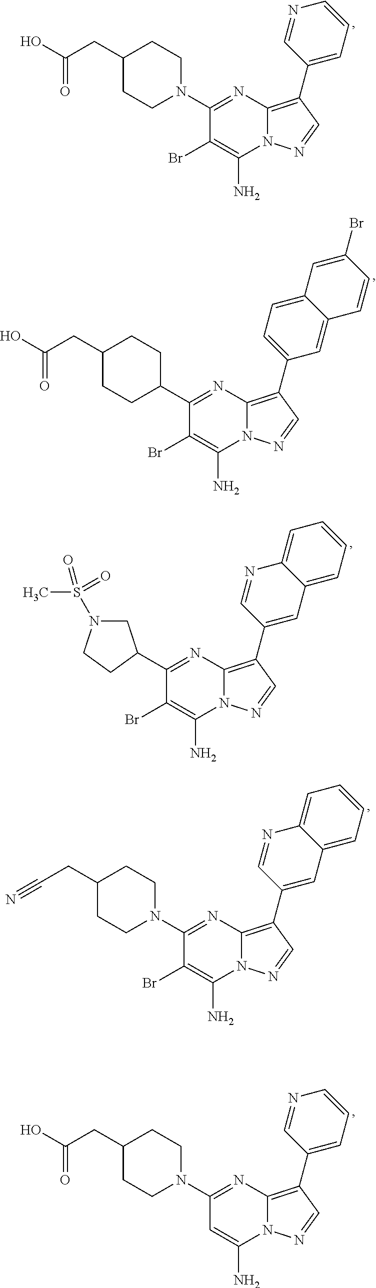

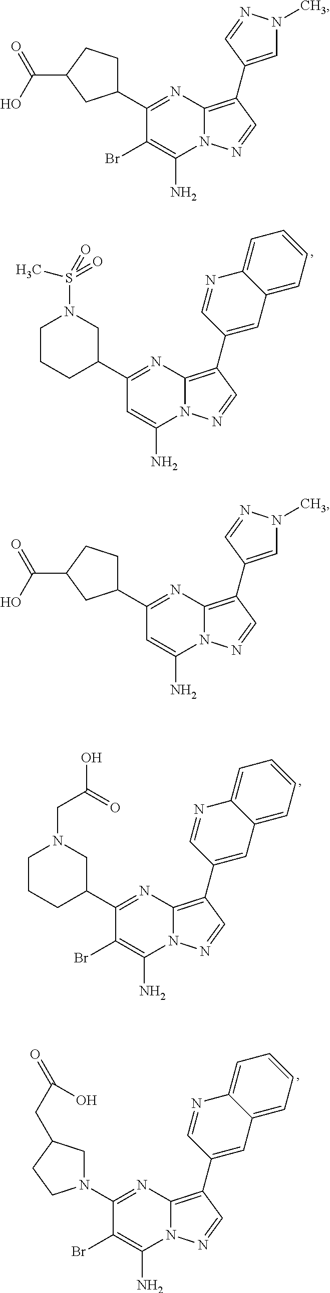

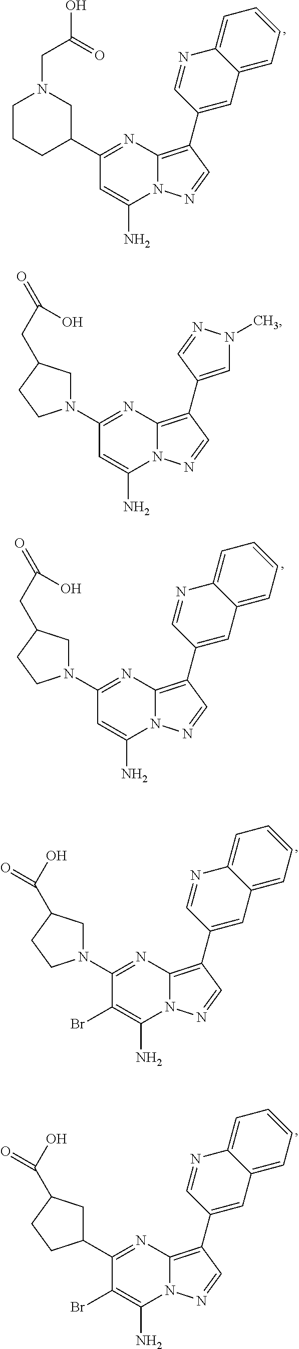

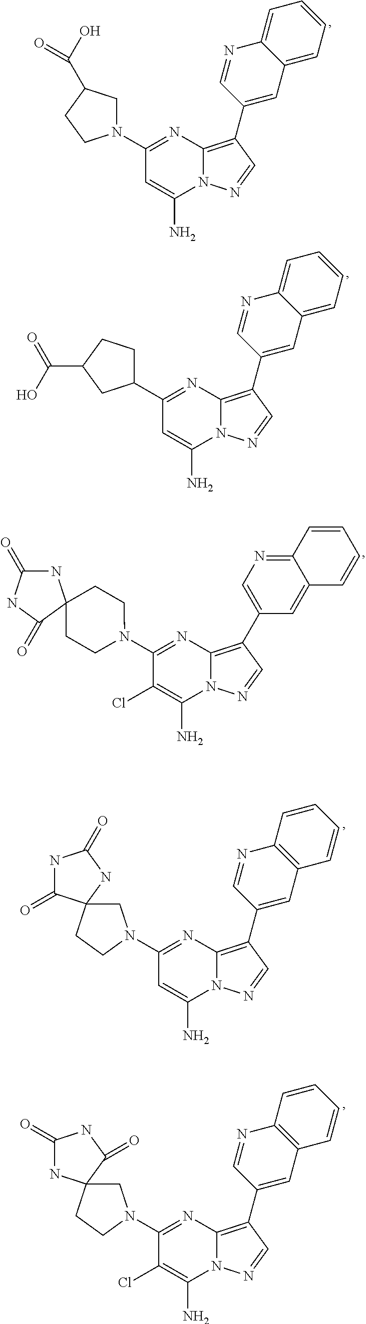

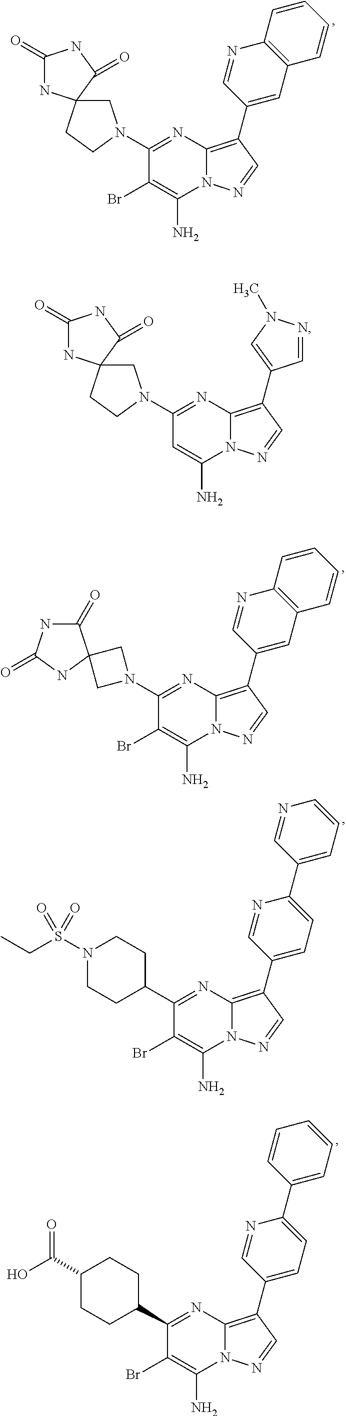

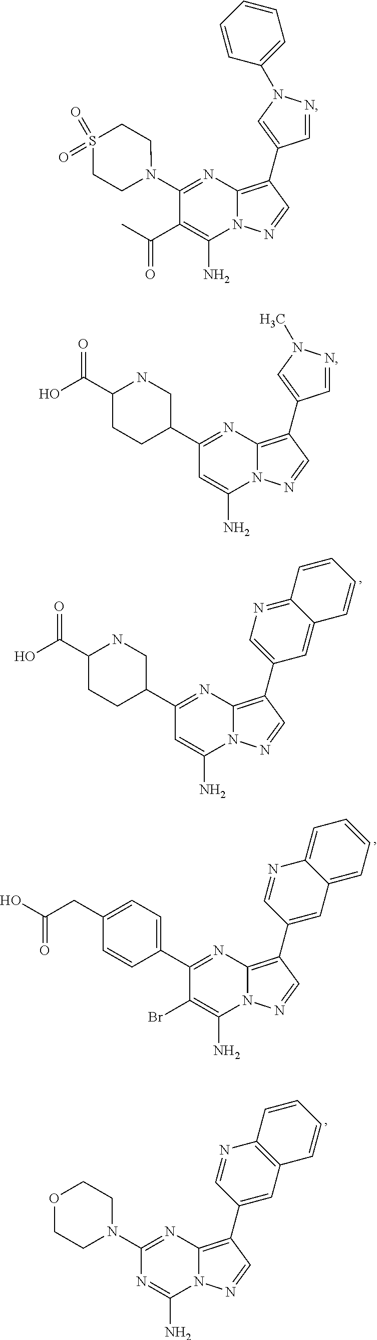

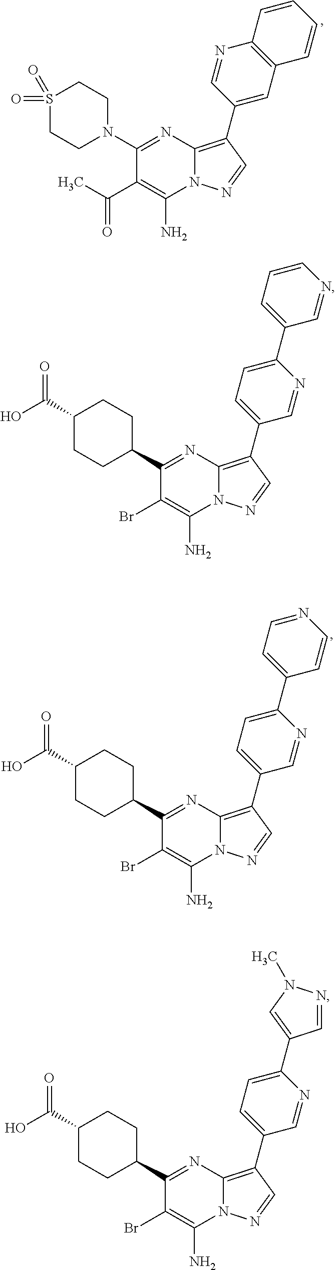

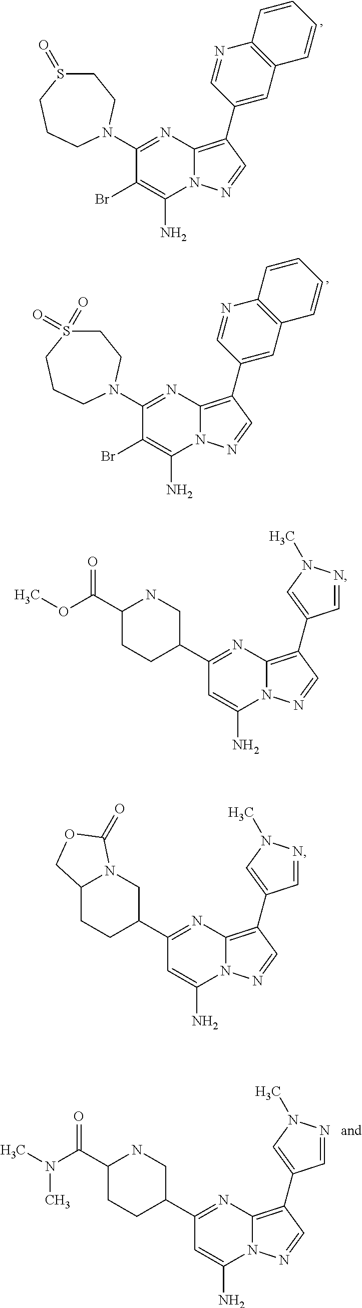

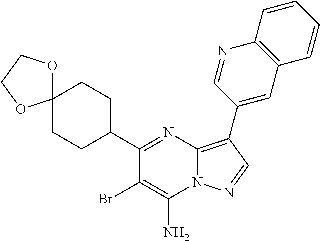

Examples of mTOR inhibitors also include compounds of the formula:

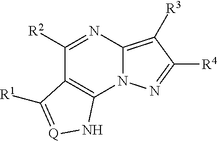

and pharmaceutically acceptable salts, solvates, prodrugs, esters, and stereoisomers thereof, wherein: Q is N or C(H); R¹ is H, halo, —NR⁵R⁶, —OR⁷, —SR⁸, —CN, alkyl, alkenyl, alkynyl, haloalkyl, cycloalkyl, aryl, heteroaryl, heterocyclyl, cycloalkylalkyl, arylalkyl, heteroarylalkyl, or heterocyclylalkyl, wherein said cycloalkyl, aryl, heteroaryl, heterocyclyl, cycloalkylalkyl, arylalkyl, heteroarylalkyl, or heterocyclylalkyl of R¹ is unsubstituted or substituted with one more moieties, which can be the same or different, each moiety being selected from the group consisting of consisting of halogen, alkyl, cycloalkyl, —CF₃, —CN, —C(O)OH, —(CH₂)ₓ—C(O)OH, —OCF₃, —OR⁷, —C(O)R¹³, —NR⁵R⁶, —C(O₂)-alkyl, —C(O)NR⁵R⁶, —SR⁸, and —S(O₂)R⁷; R² is selected from the group consisting of heterocyclyl, spiroheterocyclyl, heterocyclenyl, cycloalkyl, cycloalkenyl, heteroaryl, aryl, heterocyclylalkyl, spiroheterocyclylalkyl, heterocyclenylalkyl, cycloalkylalkyl, cycloalkenylalkyl, heteroarylalkyl, arylalkyl, —O-heterocyclyl, —S-heterocyclyl, —S(O)-heterocyclyl, S(O)₂-heterocyclyl, —N(R⁹)-heterocyclyl, and -alkyl-N(R⁹)-heterocyclyl, wherein said heterocyclyl, spiroheterocyclyl, heterocyclenyl, cycloalkyl, cycloalkenyl, heteroaryl, aryl, heterocyclylalkyl, spiroheterocyclylalkyl, heterocyclenylalkyl, cycloalkylalkyl, cycloalkenylalkyl, heteroarylalkyl, arylalkyl, —O-heterocyclyl, —S-heterocyclyl, —S(O)-heterocyclyl, —S(O)₂-heterocyclyl, —N(R⁹)-heterocyclyl, or -alkyl-N(R⁹)-heterocyclyl is unsubstituted or substituted with one to four moieties, which can be the same or different, each moiety being selected from group X; X is alkyl, halo, —CN, —NR⁵R⁶, SR⁸, —OR⁷, —C(O)alkyl, -trihaloalkyl, -dihaloalkyl, -monohaloalkyl, —C(O)₂alkyl, —C(O)₂H, hydroxyalkyl, —S(O)₂R⁸, hydroxyl, -alkyl-C(O)₂H, -alkyl(CO)N(CH₃)—O—CH₃, —C(O)₂-alkyl, -alkyl-C(O)—NH₂, -alkyl-CN, —C(O)—NR⁵R⁶, -alkyl-C(O)₂alkyl, —C(O)-hydroxyalkyl, —C(O)-alkyl-O-alkyl, -alkyl(CO)N(H)—S(O)₂-cycloalkyl, -alkyl(CO)N(H)—S(O)₂—CF₃, -alkyl(CO)N(H)—S(O)₂-alkyl, -alkyl-C(O)—N(alkyl)₂ᵢ-alkyl-N(H)—S(O)₂-alkyl, -alkyl(CO)N(H)—S(O)₂-cycloalkyl, —C(O)—CO₂H, —C(O)—CH(OH)—CH₃, —C(O)CH(OH)CH₂OH, —C(O)₂-alkyl-aryl, —SO₂—CF₃, or —C(O)H, or X is cycloalkyl, heterocyclyl, aryl, heteroaryl, heteroarylalkyl, —NH-heterocyclyl, —C(O)-heteroaryl, wherein each of said cycloalkyl, heterocyclyl, aryl, heteroaryl, heteroarylalkyl, —NH-heterocyclyl, —C(O)-heteroaryl of X is unsubstituted or substituted with one or more moieties, which can be the same or different, each moiety being selected from the group consisting of halogen, alkyl, cycloalkyl, —CF₃, —CN, —C(O)OH, —SO₃H, —P(O)(OH)₂, —(CH₂)ₓ—C(O)OH, —OCF₃, —OR⁷, —C(O)R¹⁰, —NR⁵R⁶, —C(O₂)-alkyl, —C(O)NR⁵R⁶, —SR⁸, and —S(O₂)R⁷, R³ is H, halogen, alkenyl, alkynyl, —CF₃, —C(O)R¹³, aryl, cycloalkyl, cycloalkenyl, heteroaryl, heterocyclyl, heterocycloalkenyl, arylalkyl, cycloalkylalkyl, cycloalkenylalkyl, heteroarylalkyl, heterocyclylalkyl, heterocycloalkenylalkyl, wherein each of said aryl, cycloalkyl, cycloalkenyl, heteroaryl, heterocyclyl, heterocycloalkenyl, arylalkyl, cycloalkylalkyl, cycloalkenylalkyl, heteroarylalkyl, heterocyclylalkyl, heterocycloalkenylalkyl of R³ is unsubstituted or substituted with one or more moieties which can be the same or different, each moiety being selected from the group consisting of Y, halogen, alkyl, cycloalkyl, —CF₃, —CN, —C(O)OH, —(CH₂)ₓ—C(O)OH, —OCF₃, —OR⁷, —C(O)R¹⁰, —NR⁵R⁶, —C(O₂)-alkyl, —C(O)NR⁵R⁶, —SR⁸, and —S(O₂)R⁷; Y is aryl, cycloalkyl, cycloalkenyl, heteroaryl, heterocyclyl, or heterocycloalkenyl, wherein each of said cycloalkyl, cycloalkenyl, heteroaryl, heterocyclyl, or heterocycloalkenyl of Y is unsubstituted or substituted with one more moieties, which can be the same or different, each moiety being selected from the group consisting of consisting of halogen, alkyl, cycloalkyl, —CF₃, —CN, —C(O)OH, —(CH₂)ₓ—C(O)OH, —OCF₃, —OR⁷, —C(O)R¹⁰, —NR⁵R⁶, —C(O₂)-alkyl, —C(O)NR⁵R⁶, —SR⁸, and —S(O₂)R⁷; R⁴ is H, halo, —NR⁵R⁶, —OR⁷, —OR⁸, —SR⁹, —CN, alkyl, alkenyl, alkynyl, haloalkyl, cycloalkyl, aryl, heteroaryl, heterocyclyl, cycloalkylalkyl, arylalkyl, heteroarylalkyl, or heterocyclylalkyl; each occurrence of R⁵ is independently H, alkyl, cycloalkyl, aryl, heteroaryl, or heterocyclyl; each occurrence of R⁶ is independently H, alkyl, cycloalkyl, aryl, heteroaryl, or heterocyclyl, or R⁵ and R⁶ together with the nitrogen atom to which they are attached form a heterocyclyl ring; each occurrence of R⁷ is independently H, alkyl, cycloalkyl, aryl, heteroaryl, or heterocyclyl; each occurrence of R⁸ is independently H, alkyl, cycloalkyl, aryl, heteroaryl, or heterocyclyl; each occurrence of R⁹ is independently H or alkyl; R¹⁰ is alkyl, cycloalkyl, or aryl; and x is an integer from 1 to 4; as described in U.S. Provisional Application No. 61/222,529 filed on Jul. 2, 2009, the disclosure of which is incorporated herein by reference thereto.

Examples of mTOR inhibitors disclosed in U.S. Provisional Application No. 61/222,529 are:

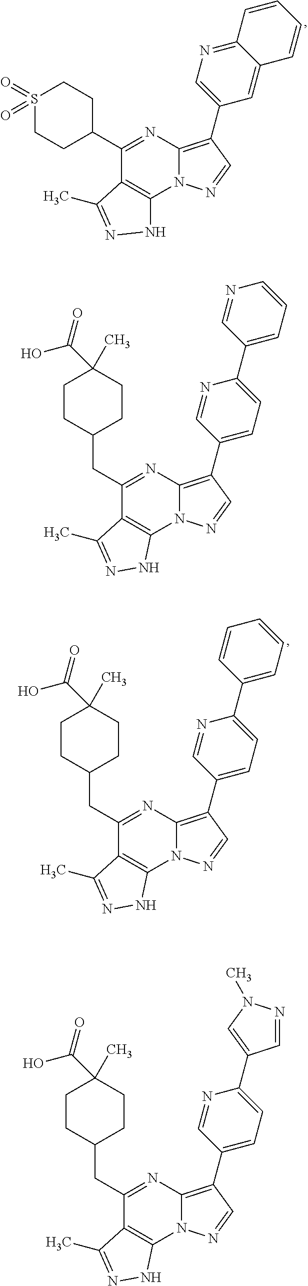

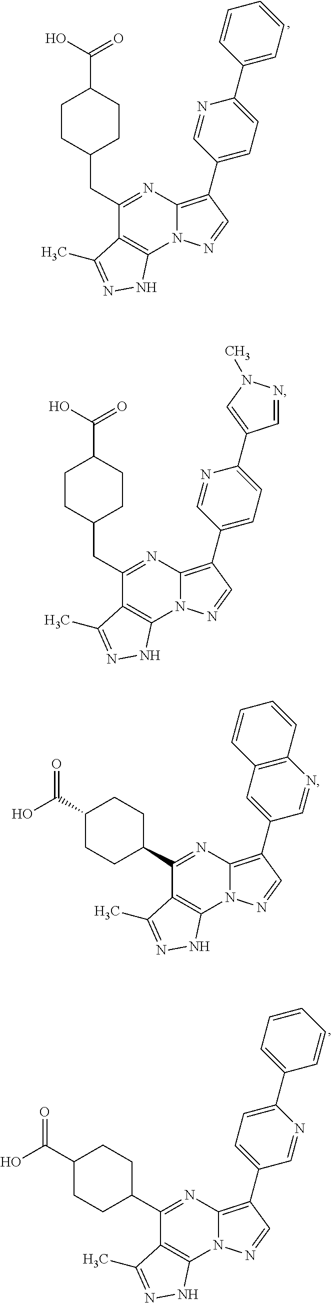

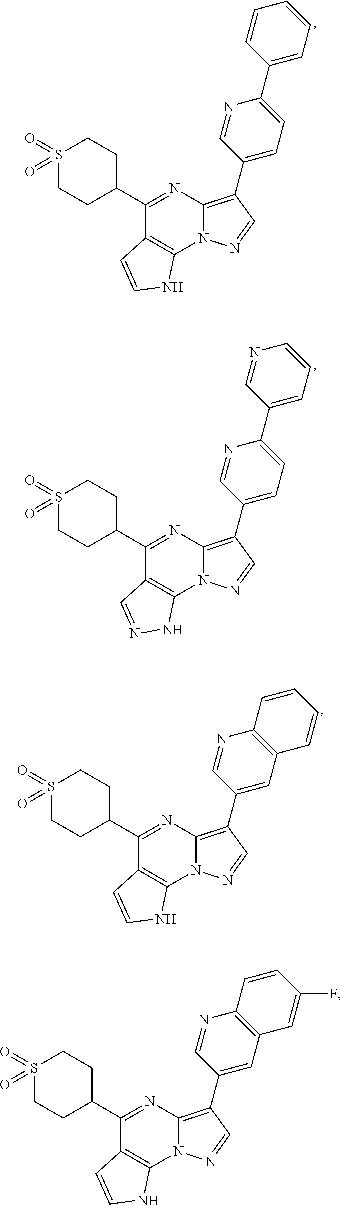

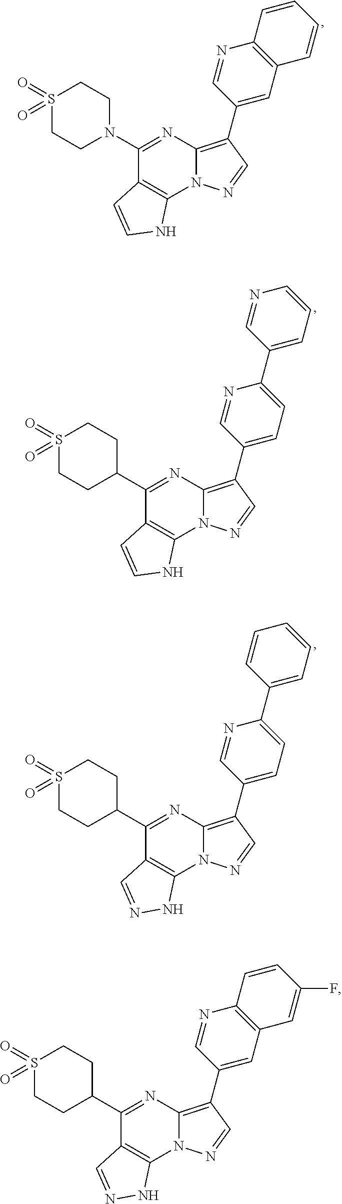

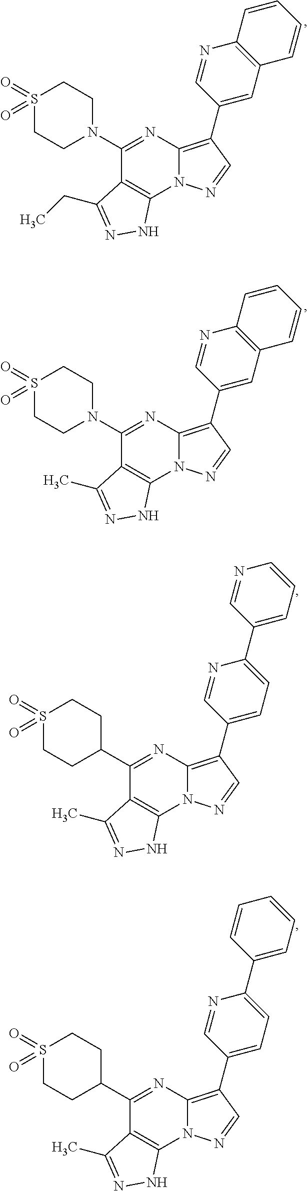

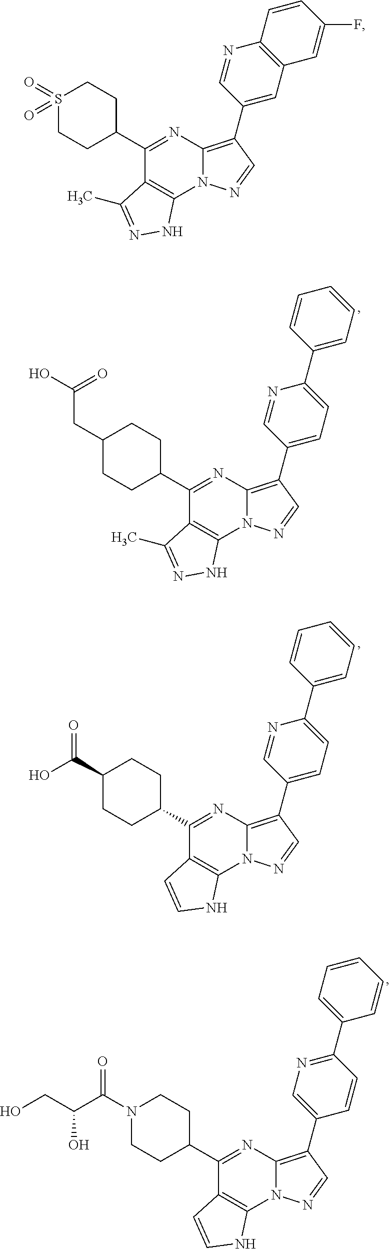

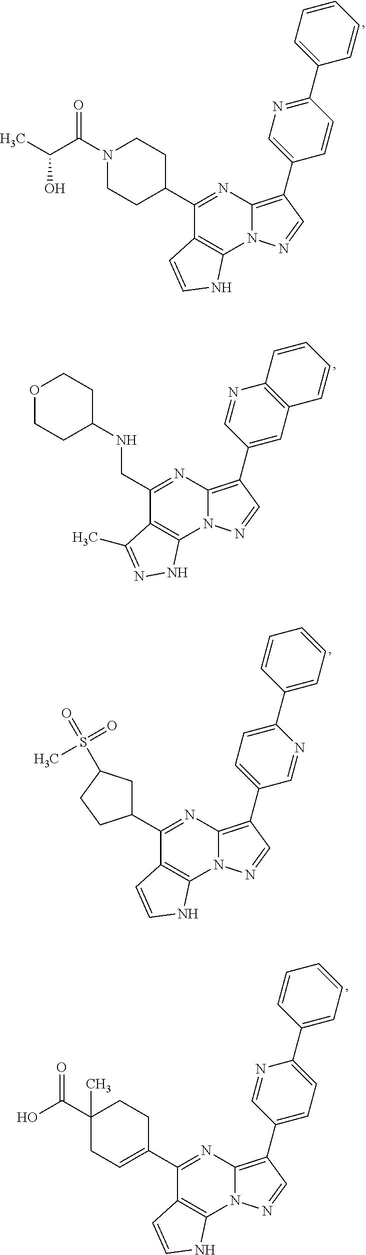

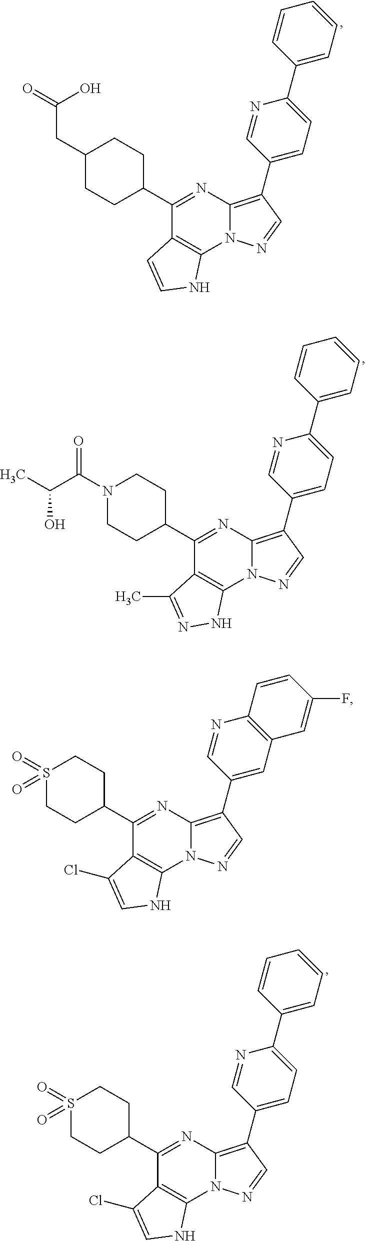

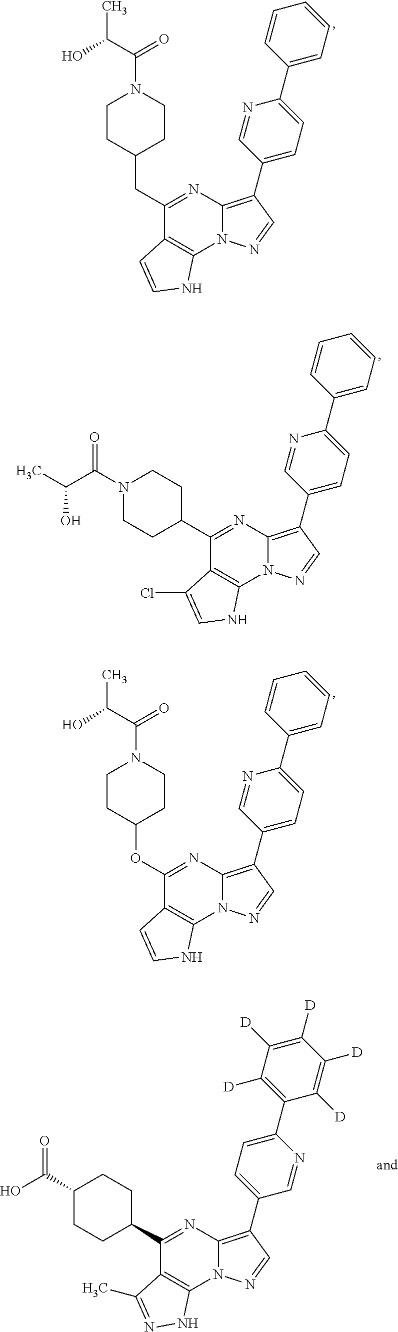

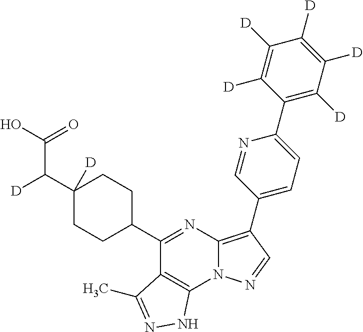

and pharmaceutically acceptable salts, solvates, esters, stereoisomers, or prodrugs thereof.

Other embodiments of this invention are described below. The embodiments have been numbered for ease of reference. When an Embodiment refers to “any one of” that Embodiment describes a separate embodiment to each group or embodiment listed. For example, Embodiment No. 1 is meant to describe: (1) an embodiment directed to compounds of formula (I) wherein Ar is unsubstituted aryl (e.g., unsubstituted phenyl), (2) an embodiment directed to any one of compounds (Ii) to (Ivii) wherein Ar is unsubstituted aryl (e.g., unsubstituted phenyl), (3) an embodiment directed to compounds of formula (I.A1) wherein Ar is unsubstituted aryl (e.g., unsubstituted phenyl), (4) an embodiment directed to any one of compounds (I.A1a) to (I.A1h) wherein Ar is unsubstituted aryl (e.g., unsubstituted phenyl), (5) an embodiment directed to compounds of formula (I.A2) wherein Ar is unsubstituted aryl (e.g., unsubstituted phenyl), and (6) an embodiment directed to any one of compounds (I.A2a) to (I.A2h) wherein Ar is unsubstituted aryl (e.g., unsubstituted phenyl). In Embodiment Numbers 1 to 105 at least one (e.g., 1-4, 1-3, 1-2, 2 or 1) H in the compound is replaced by deuterium (D).

Embodiment No. 1 is directed to any one of the compounds of formulas (I), (iI) to (Ivii), (I.A1), (I.A1a) to (I.A1h), (I.A2), and (I.A2a) to (I.A2h) wherein Ar is unsubstituted aryl (e.g., unsubstituted phenyl).

Embodiment No. 2 is directed to any one of the compounds of formulas (I), (iI) to (Ivii), (I.A1), (I.A1a) to (I.A1h), (I.A2), and (I.A2a) to (I.A2h) wherein Ar is substituted aryl (e.g., substituted phenyl). Examples of substituted phenyls include, for example, (1) halo substituted phenyl (such as, for example chloro substituted phenyl, such as, for example, p-Cl-phenyl-), (2) haloalkyl substituted phenyl (such as, for example, —CF₃ substituted phenyl, such as, for example, p-CF₃-phenyl-), and (3) cyano substituted phenyl (such as, for example, p-CN-phenyl-).

Embodiment No. 3 is directed to any one of the compounds of formulas (I), (iI) to (Ivii), (I.A1), (I.A1a) to (I.A1h), (I.A2), and (I.A2a) to (I.A2h) wherein Ar is unsubstituted heteroaryl (e.g., unsubstituted pyridyl).

Embodiment No. 4 is directed to any one of the compounds of formulas (I), (iI) to (Ivii), (I.A1), (I.A1a) to (I.A1h), (I.A2), and (I.A2a) to (I.A2h) wherein Ar is substituted heteroaryl (e.g., substituted pyridyl). Examples of substituted pyridyls include, for example, (1) halo substituted pyridyl (such as, for example chloro substituted pyridyl), (2) haloalkyl substituted pyridyl (such as, for example, —CF₃ substituted pyridyl), and (3) cyano substituted pyridyl.

Embodiment No. 5 is directed to any one of the compounds of formulas (I), (iI) to (Ivii), (I.A1), (I.A1a) to (I.A1h), (I.A2), and (I.A2a) to (I.A2h) wherein each L¹ is the same or different halo.

Embodiment No. 6 is directed to any one of the compounds of formulas (I), (iI) to (Ivii), (I.A1), (I.A1a) to (I.A1h), (I.A2), and (I.A2a) to (I.A2h) wherein n is 2 and each L¹ is the same or different halo.

Embodiment No. 7 is directed to any one of the compounds of formulas (I), (iI) to (Ivii), (I.A1), (I.A1a) to (I.A1h), (I.A2), and (I.A2a) to (I.A2h) wherein n is 2 and each L¹ is the same halo.

Embodiment No. 8 is directed to any one of the compounds of formulas (I), (iI) to (Ivii), (I.A1), (I.A1a) to (I.A1h), (I.A2), and (I.A2a) to (I.A2h) wherein n is 2 and each L¹ is F.

Embodiment No. 9 is directed to any one of the compounds of formulas (I), (iI) to (Ivii), (I.A1), (I.A1a) to (I.A1h), (I.A2), and (I.A2a) to (I.A2h), wherein each L¹ is the same or different halo, and Ar is unsubstituted aryl (e.g., unsubstituted phenyl).

Embodiment No. 10 is directed to any one of the compounds of formulas (I), (iI) to (Ivii), (I.A1), (I.A1a) to (I.A1h), (I.A2), and (I.A2a) to (I.A2h), wherein n is 2, each L¹ is the same or different halo, and Ar is unsubstituted aryl (e.g., unsubstituted phenyl).

Embodiment No. 11 is directed to any one of the compounds of formulas (I), (iI) to (Ivii), (I.A1), (I.A1a) to (I.A1h), (I.A2), and (I.A2a) to (I.A2h), wherein n is 2, each L¹ is the same halo, and Ar is unsubstituted aryl (e.g., unsubstituted phenyl).

Embodiment No. 12 is directed to any one of the compounds of formulas (I), (iI) to (Ivii), (I.A1), (I.A1a) to (I.A1h), (I.A2), and (I.A2a) to (I.A2h), wherein n is 2, each L¹ is F, and Ar is unsubstituted aryl (e.g., unsubstituted phenyl).

Embodiment No. 13 is directed to any one of the compounds of formulas (I), (iI) to (Ivii), (I.A1), (I.A1a) to (I.A1h), (I.A2), and (I.A2a) to (I.A2h), wherein each L¹ is the same or different halo, and Ar is substituted aryl (e.g., substituted phenyl). Examples of substituted phenyls include, for example, (1) halo substituted phenyl (such as, for example chloro substituted phenyl, such as, for example, p-Cl-phenyl-), (2) haloalkyl substituted phenyl (such as, for example, —CF₃ substituted phenyl, such as, for example, p-CF₃-phenyl-), and (3) cyano substituted phenyl (such as, for example, p-CN-phenyl-).

Embodiment No. 14 is directed to any one of the compounds of formulas (I), (iI) to (Ivii), (I.A1), (I.A1a) to (I.A1h), (I.A2), and (I.A2a) to (I.A2h), wherein n is 2, each L¹ is the same or different halo, and Ar is substituted aryl (e.g., substituted phenyl). Examples of substituted phenyls include, for example, (1) halo substituted phenyl (such as, for example chloro substituted phenyl, such as, for example, p-Cl-phenyl-), (2) haloalkyl substituted phenyl (such as, for example, —CF₃ substituted phenyl, such as, for example, p-CF₃-phenyl-), and (3) cyano substituted phenyl (such as, for example, p-CN-phenyl-).

Embodiment No. 15 is directed to any one of the compounds of formulas (I), (iI) to (Ivii), (I.A1), (I.A1a) to (I.A1h), (I.A2), and (I.A2a) to (I.A2h), wherein n is 2, each L¹ is the same halo, and Ar is substituted aryl (e.g., substituted phenyl). Examples of substituted phenyls include, for example, (1) halo substituted phenyl (such as, for example chloro substituted phenyl, such as, for example, p-Cl-phenyl-), (2) haloalkyl substituted phenyl (such as, for example, —CF₃ substituted phenyl, such as, for example, p-CF₃-phenyl-), and (3) cyano substituted phenyl (such as, for example, p-CN-phenyl-).

Embodiment No. 16 is directed to any one of the compounds of formulas (I), (iI) to (Ivii), (I.A1), (I.A1a) to (I.A1h), (I.A2), and (I.A2a) to (I.A2h), wherein n is 2, each L¹ is F, and Ar is substituted aryl (e.g., substituted phenyl). Examples of substituted phenyls include, for example, (1) halo substituted phenyl (such as, for example chloro substituted phenyl, such as, for example, p-Cl-phenyl-), (2) haloalkyl substituted phenyl (such as, for example, —CF3 substituted phenyl, such as, for example, p-CF₃-phenyl-), and (3) cyano substituted phenyl (such as, for example, p-CN-phenyl-).

Embodiment No. 17 is directed to any one of the compounds of formulas (I), (iI) to (Ivii), (I.A1), (I.A1a) to (I.A1h), (I.A2), and (I.A2a) to (I.A2h), wherein each L¹ is the same or different halo, and Ar is unsubstituted heteroaryl (e.g., unsubstituted pyridyl).

Embodiment No. 18 is directed to any one of the compounds of formulas (I), (iI) to (Ivii), (I.A1), (I.A1a) to (I.A1h), (I.A2), and (I.A2a) to (I.A2h), wherein n is 2, each L¹ is the same or different halo, and Ar is unsubstituted heteroaryl (e.g., unsubstituted pyridyl).

Embodiment No. 19 is directed to any one of the compounds of formulas (I), (iI) to (Ivii), (I.A1), (I.A1a) to (I.A1h), (I.A2), and (I.A2a) to (I.A2h), wherein n is 2, each 1 is the same halo, and Ar is unsubstituted heteroaryl (e.g., unsubstituted pyridyl).

Embodiment No. 20 is directed to any one of the compounds of formulas (I), (iI) to (Ivii), (I.A1), (I.A1a) to (I.A1h), (I.A2), and (I.A2a) to (I.A2h), wherein n is 2, each L¹ is F, and Ar is unsubstituted heteroaryl (e.g., unsubstituted pyridyl).

Embodiment No. 21 is directed to any one of the compounds of formulas (I), (iI) to (Ivii), (I.A1), (I.A1a) to (I.A1h), (I.A2), and (I.A2a) to (I.A2h), wherein each L¹ is the same or different halo, and Ar is substituted heteroaryl (e.g., substituted pyridyl). Examples of substituted pyridyls include, for example, (1) halo substituted pyridyl (such as, for example chloro substituted pyridyl), (2) haloalkyl substituted pyridyl (such as, for example, —CF₃ substituted pyridyl), and (3) cyano substituted pyridyl.

Embodiment No. 22 is directed to any one of the compounds of formulas (I), (iI) to (Ivii), (I.A1), (I.A1a) to (I.A1h), (I.A2), and (I.A2a) to (I.A2h), wherein n is 2, each L¹ is the same or different halo, and Ar is substituted heteroaryl (e.g., substituted pyridyl). Examples of substituted pyridyls include, for example, (1) halo substituted pyridyl (such as, for example chloro substituted pyridyl), (2) haloalkyl substituted pyridyl (such as, for example, —CF₃ substituted pyridyl), and (3) cyano substituted pyridyl.

Embodiment No. 23 is directed to any one of the compounds of formulas (I), (iI) to (Ivii), (I.A1), (I.A1a) to (I.A1h), (I.A2), and (I.A2a) to (I.A2h), wherein n is 2, each L¹ is the same halo, and Ar is substituted heteroaryl (e.g., substituted pyridyl). Examples of substituted pyridyls include, for example, (1) halo substituted pyridyl (such as, for example chloro substituted pyridyl), (2) haloalkyl substituted pyridyl (such as, for example, —CF₃ substituted pyridyl), and (3) cyano substituted pyridyl.

Embodiment No. 24 is directed to any one of the compounds of formulas (I), (iI) to (Ivii), (I.A1), (I.A1a) to (I.A1h), (I.A2), and (I.A2a) to (I.A2h), wherein n is 2, each L¹ is F, and Ar is substituted heteroaryl (e.g., substituted pyridyl). Examples of substituted pyridyls include, for example, (1) halo substituted pyridyl (such as, for example chloro substituted pyridyl), (2) haloalkyl substituted pyridyl (such as, for example, —CF₃ substituted pyridyl), and (3) cyano substituted pyridyl.

Embodiment No. 25 is directed to any one of the compounds of formulas (I), (iI) to (Ivii), (I.A1), (I.A1a) to (I.A1h), (I.A2), and (I.A2a) to (I.A2h), wherein n is 2, each L¹ is the same halo, and Ar is substituted aryl selected from the group consisting p-CH₃CH₂SO₂phenyl, p-Br-phenyl, m,p-di-F-phenyl, m,p-di-CN-phenyl, p-CH₃O-phenyl, and p-CF₃CH₂Ophenyl.

Embodiment No. 26 is directed to any one of the compounds of formulas (I), (iI) to (Ivii), (I.A1), (I.A1a) to (I.A1h), (I.A2), and (I.A2a) to (I.A2h), wherein n is 2, each L¹ is F, and Ar is substituted aryl selected from the group consisting p-CH₃CH₂SO₂phenyl, p-Br-phenyl, m,p-di-F-phenyl, m,p-di-CN-phenyl, p-CH₃O-phenyl, and p-CF₃CH₂Ophenyl.

Embodiment No. 27 is directed to any one of Embodiment Nos. 1 to 26 wherein R² is H.

Embodiment No. 28 is directed to any one of Embodiment Nos. 1 to 26 wherein R² is alkyl.

Embodiment No. 29 is directed to any one of Embodiment Nos. 1 to 26 wherein R² is methyl.

Embodiment No. 30 is directed to any one of Embodiment Nos. 1 to 26 wherein R¹ is -alkylene-(tetrahydrothiophene 1,1-dioxide).

Embodiment No. 31 is directed to any one of Embodiment Nos. 1 to 26 wherein R¹ is -alkenyl-S(O)₂—(C₁-C₆)alkyl.

Embodiment No. 32 is directed to any one of Embodiment Nos. 1 to 26 wherein R¹ is -cycloalkyl-S(O)₂—(C₁-C₆)alkyl.

Embodiment No. 33 is directed to any one of Embodiment Nos. 1 to 26 wherein R¹ is an -alkylene-S(O)₂—(C₁-C₆)alkyl group.

Embodiment No. 34 is directed to any one of Embodiment Nos. 1 to 26 wherein R¹ is a —(C₁ to C₂) alkylene-S(O)₂—(C₁-C₆)alkyl group.

Embodiment No. 35 is directed to any one of Embodiment Nos. 1 to 26 wherein R¹ is a —(C₁ to C₂) alkylene-S(O)₂—(C₁-C₃)alkyl group.

Embodiment No. 36 is directed to any one of Embodiment Nos. 1 to 26 wherein R¹ is a —(C₂) alkylene-S(O)₂—(C₁-C₆)alkyl group.

Embodiment No. 37 is directed to any one of Embodiment Nos. 1 to 26, wherein the compound is a compound of the formula (I), (I.A1), (I.A2), (I.A1a), (I.A1e), (I.A2a) or (I.A2e) wherein R¹ is selected from the group consisting of:

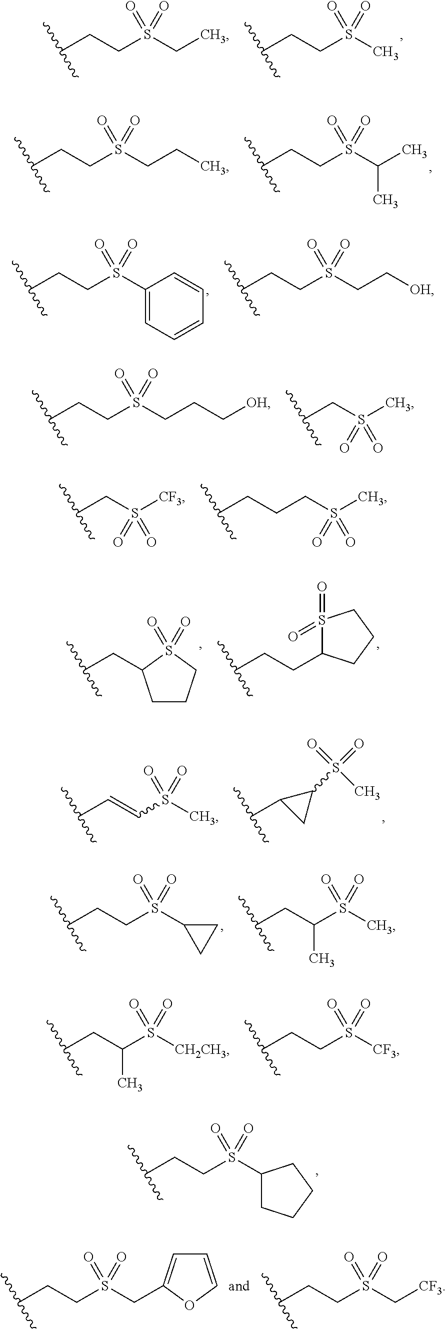

Embodiment No. 38 is directed to any one of Embodiment Nos. 1 to 26, wherein the compound is a compound of the formula (Ii), (Iii), (Iiii), (Iiv), (I.A1b), (I.A1c), (I.A1d), (I.A2b), (I.A2c), or (I.A1d), and R¹ is selected from the group consisting of:

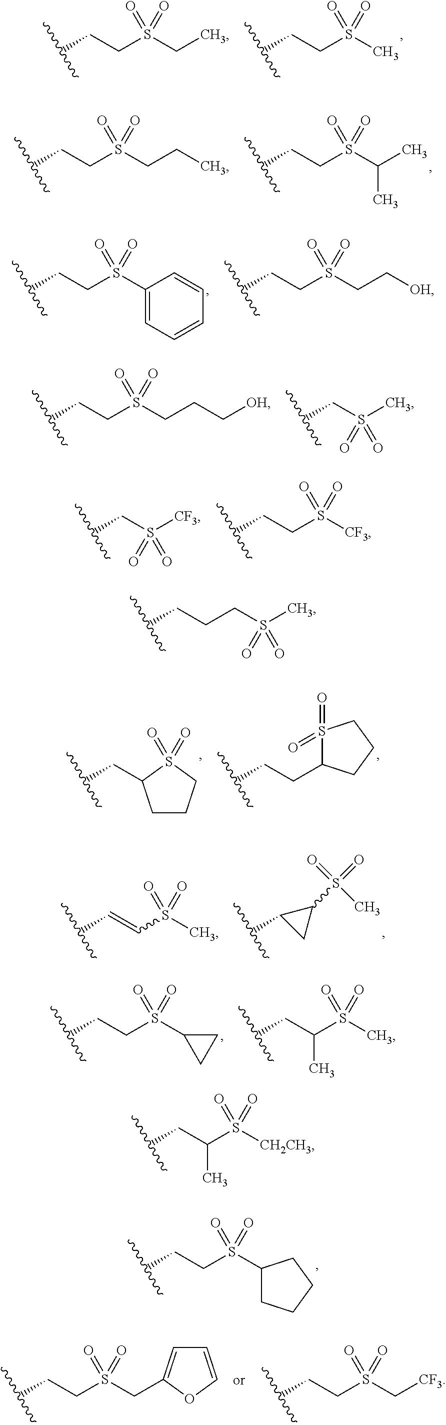

Embodiment No. 39 is directed to any one of Embodiment Nos. 1 to 26, wherein the compound is a compound of the formula (Iv), (Ivi), (Ivii), (I.A1f), (I.A1g), (I.A1h), (I.A2f), (I.A2g), or (I.A2h) wherein R¹ is selected from the group consisting of:

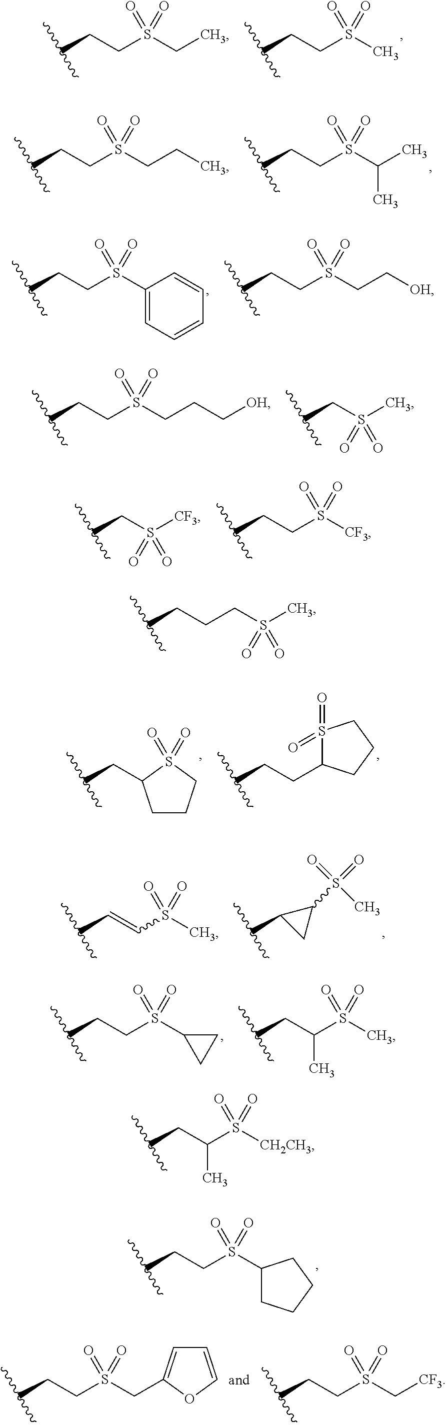

Embodiment No. 40 is directed to any one of Embodiment Nos. 1 to 26, wherein the compound is a compound of the formula (Ii), (Iii), (Iiii), (Iiv), (I.A1b), (I.A1c), (I.A1d), (I.A2b), (I.A2c), or (I.A1d), and R¹ is

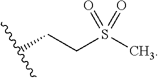

Embodiment No. 41 is directed to any one of Embodiment Nos. 1 to 26, wherein the compound is a compound of the formula (Ii), (Iii), (Iiii), (Iiv), (I.A1b), (I.A1c), (I.A1d), (I.A2b), (I.A2c), or (I.A1d), and R¹ is

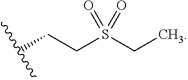

Embodiment No. 42 is directed to any one of Embodiment Nos. 1 to 26, wherein the compound is a compound of the formula (Ii), (Iii), (Iiii), (Iiv), (I.A1b), (I.A1c), (I.A1d), (I.A2b), (I.A2c), or (I.A1d), and R¹ is

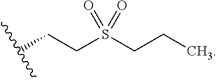

Embodiment No. 43 is directed to any one of Embodiment Nos. 1 to 26, wherein the compound is a compound of the formula (Ii), (Iii), (Iiii), (Iiv), (I.A1b), (I.A1c), (I.A1d), (I.A2b), (I.A2c), or (I.A1d), and R¹ is

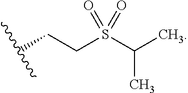

Embodiment No. 44 is directed to any one of Embodiment Nos. 1 to 26, wherein the compound is a compound of the formula (Ii), (Iii), (Iiii), (Iiv), (I.A1b), (I.A1c), (I.A1d), (I.A2b), (I.A2c), or (I.A1d), and R¹ is

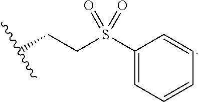

Embodiment No. 45 is directed to any one of Embodiment Nos. 1 to 26, wherein the compound is a compound of the formula (Ii), (Iii), (Iiii), (Iiv), (I.A1b), (I.A1c), (I.A1d), (I.A2b), (I.A2c), or (I.A1d), and R¹ is

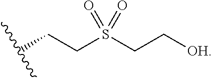

Embodiment No. 46 is directed to any one of Embodiment Nos. 1 to 26, wherein the compound is a compound of the formula (Ii), (Iii), (Iiii), (Iiv), (I.A1b), (I.A1c), (I.A1d), (I.A2b), (I.A2c), or (I.A1d), and R¹ is

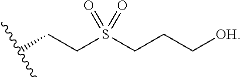

Embodiment No. 47 is directed to any one of Embodiment Nos. 1 to 26, wherein the compound is a compound of the formula (Ii), (Iii), (Iiii), (Iiv), (I.A1b), (I.A1c), (I.A1d), (I.A2b), (I.A2c), or (I.A1d), and R¹ is

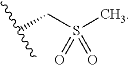

Embodiment No. 48 is directed to any one of Embodiment Nos. 1 to 26, wherein the compound is a compound of the formula (Ii), (Iii), (Iiii), (Iiv), (I.A1b), (I.A1c), (I.A1d), (I.A2b), (I.A2c), or (I.A1d), and R¹ is

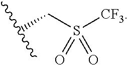

Embodiment No. 49 is directed to any one of Embodiment Nos. 1 to 26, wherein the compound is a compound of the formula (Ii), (Iii), (Iiii), (Iiv), (I.A1b), (I.A1c), (I.A1d), (I.A2b), (I.A2c), or (I.A1d), and R¹ is

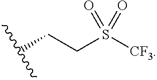

Embodiment No. 50 is directed to any one of Embodiment Nos. 1 to 26, wherein the compound is a compound of the formula (Ii), (Iii), (Iiii), (Iiv), (I.A1b), (I.A1c), (I.A1d), (I.A2b), (I.A2c), or (I.A1d), and R¹ is

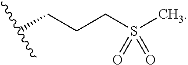

Embodiment No. 51 is directed to any one of Embodiment Nos. 1 to 26, wherein the compound is a compound of the formula (Ii), (Iii), (Iiii), (Iiv), (I.A1b), (I.A1c), (I.A1d), (I.A2b), (I.A2c), or (I.A1d), and R¹ is

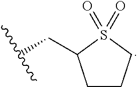

Embodiment No. 52 is directed to any one of Embodiment Nos. 1 to 26, wherein the compound is a compound of the formula (Ii), (Iii), (Iiii), (Iiv), (I.A1b), (I.A1c), (I.A1d), (I.A2b), (I.A2c), or (I.A1d), and R¹ is

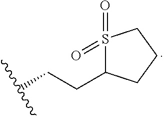

Embodiment No. 53 is directed to any one of Embodiment Nos. 1 to 26, wherein the compound is a compound of the formula (Ii), (Iii), (Iiii), (Iiv), (I.A1b), (I.A1c), (I.A1d), (I.A2b), (I.A2c), or (I.A1d), and R¹ is

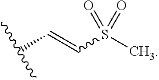

Embodiment No. 54 is directed to any one of Embodiment Nos. 1 to 26, wherein the compound is a compound of the formula (Ii), (Iii), (Iiii), (Iiv), (I.A1b), (I.A1c), (I.A1d), (I.A2b), (I.A2c), or (I.A1d), and R¹ is

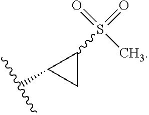

Embodiment No. 55 is directed to any one of Embodiment Nos. 1 to 26, wherein the compound is a compound of the formula (Ii), (Iii), (Iiii), (Iiv), (I.A1b), (I.A1c), (I.A1d), (I.A2b), (I.A2c), or (I.A1d), and R¹ is

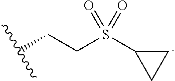

Embodiment No. 56 is directed to any one of Embodiment Nos. 1 to 26, wherein the compound is a compound of the formula (Ii), (Iii), (Iiii), (Iiv), (I.A1b), (I.A1c), (I.A1d), (I.A2b), (I.A2c), or (I.A1d), and R¹ is

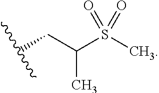

Embodiment No. 57 is directed to any one of Embodiment Nos. 1 to 26, wherein the compound is a compound of the formula (Ii), (Iii), (Iiii), (Iiv), (I.A1b), (I.A1c), (I.A1d), (I.A2b), (I.A2c), or (I.A1d), and R¹ is

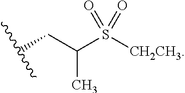

Embodiment No. 58 is directed to any one of Embodiment Nos. 1 to 26, wherein the compound is a compound of the formula (Ii), (Iii), (Iiii), (Iiv), (I.A1b), (I.A1c), (I.A1d), (I.A2b), (I.A2c), or (I.A1d), and R¹ is

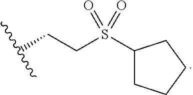

Embodiment No. 59 is directed to any one of Embodiment Nos. 1 to 26, wherein the compound is a compound of the formula (Ii), (Iii), (Iiii), (Iiv), (I.A1b), (I.A1c), (I.A1d), (I.A2b), (I.A2c), or (I.A1d), and R¹ is

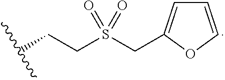

Embodiment No. 60 is directed to any one of Embodiment Nos. 1 to 26, wherein the compound is a compound of the formula (Ii), (Iii), (Iiii), (Iiv), (I.A1b), (I.A1c), (I.A1d), (I.A2b), (I.A2c), or (I.A1d), and R¹ is

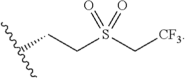

Embodiment No. 61 is directed to any one of Embodiment Nos. 1 to 26, wherein the compound is a compound of the formula (Iv), (Ivi), (Ivii), (I.A1f), (I.A1g), (I.A1h), (I.A2f), (I.A2g), or (I.A2h) wherein R¹ is

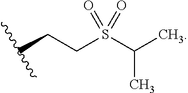

Embodiment No. 62 is directed to any one of Embodiment Nos. 1 to 26, wherein the compound is a compound of the formula (Iv), (Ivi), (Ivii), (I.A1f), (I.A1g), (I.A1h), (I.A2f), (I.A2g), or (I.A2h) wherein R¹ is

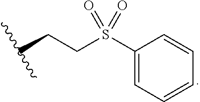

Embodiment No. 63 is directed to any one of Embodiment Nos. 1 to 26, wherein the compound is a compound of the formula (Iv), (Ivi), (Ivii), (I.A1f), (I.A1g), (I.A1h), (I.A2f), (I.A2g), or (I.A2h) wherein R¹ is

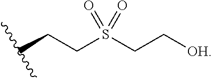

Embodiment No. 64 is directed to any one of Embodiment Nos. 1 to 26, wherein the compound is a compound of the formula (Iv), (Ivi), (Ivii), (I.A1f), (I.A1g), (I.A1h), (I.A2f), (I.A2g), or (I.A2h) wherein R¹ is

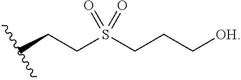

Embodiment No. 65 is directed to any one of Embodiment Nos. 1 to 26, wherein the compound is a compound of the formula (Iv), (Ivi), (Ivii), (I.A1f), (I.A1g), (I.A1h), (I.A2f), (I.A2g), or (I.A2h) wherein R¹ is

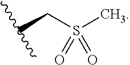

Embodiment No. 66 is directed to any one of Embodiment Nos. 1 to 26, wherein the compound is a compound of the formula (Iv), (Ivi), (Ivii), (I.A1f), (I.A1g), (I.A1h), (I.A2f), (I.A2g), or (I.A2h) wherein R¹ is

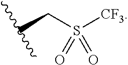

Embodiment No. 67 is directed to any one of Embodiment Nos. 1 to 26, wherein the compound is a compound of the formula (Iv), (Ivi), (Ivii), (I.A1f), (I.A1g), (I.A1h), (I.A2f), (I.A2g), or (I.A2h) wherein R¹ is

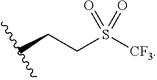

Embodiment No. 68 is directed to any one of Embodiment Nos. 1 to 26, wherein the compound is a compound of the formula (Iv), (Ivi), (Ivii), (I.A1f), (I.A1g), (I.A1h), (I.A2f), (I.A2g), or (I.A2h) wherein R¹ is

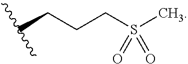

Embodiment No. 69 is directed to any one of Embodiment Nos. 1 to 26, wherein the compound is a compound of the formula (Iv), (Ivi), (Ivii), (I.A1f), (I.A1g), (I.A1h), (I.A2f), (I.A2g), or (I.A2h) wherein R¹ is

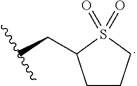

Embodiment No. 70 is directed to any one of Embodiment Nos. 1 to 26, wherein the compound is a compound of the formula (Iv), (Ivi), (Ivii), (I.A1f), (I.A1g), (I.A1h), (I.A2f), (I.A2g), or (I.A2h) wherein R¹ is

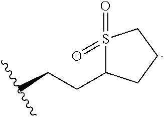

Embodiment No. 71 is directed to any one of Embodiment Nos. 1 to 26, wherein the compound is a compound of the formula (Iv), (Ivi), (Ivii), (I.A1f), (I.A1g), (I.A1h), (I.A2f), (I.A2g), or (I.A2h) wherein R¹ is

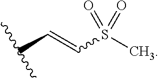

Embodiment No. 72 is directed to any one of Embodiment Nos. 1 to 26, wherein the compound is a compound of the formula (Iv), (Ivi), (Ivii), (I.A1f), (I.A1g), (I.A1h), (I.A2f), (I.A2g), or (I.A2h) wherein is

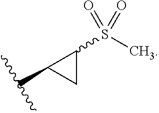

Embodiment No. 73 is directed to any one of Embodiment Nos. 1 to 26, wherein the compound is a compound of the formula (Iv), (Ivi), (Ivii), (I.A1f), (I.A1g), (I.A1h), (I.A2f), (I.A2g), or (I.A2h) wherein R¹ is

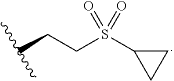

Embodiment No. 74 is directed to any one of Embodiment Nos. 1 to 26, wherein the compound is a compound of the formula (Iv), (Ivi), (Ivii), (I.A1f), (I.A1g), (I.A1h), (I.A2f), (I.A2g), or (I.A2h) wherein R¹ is

Embodiment No. 75 is directed to any one of Embodiment Nos. 1 to 26, wherein the compound is a compound of the formula (Iv), (Ivi), (Ivii), (I.A1f), (I.A1g), (I.A1h), (I.A2f), (I.A2g), or (I.A2h) wherein R¹ is

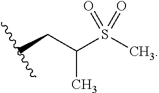

Embodiment No. 76 is directed to any one of Embodiment Nos. 1 to 26, wherein the compound is a compound of the formula (Iv), (Ivi), (Ivii), (I.A1f), (I.A1g), (I.A1h), (I.A2f), (I.A2g), or (I.A2h) wherein R¹ is

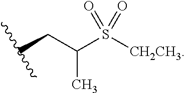

Embodiment No. 77 is directed to any one of Embodiment Nos. 1 to 26, wherein the compound is a compound of the formula (Iv), (Ivi), (Ivii), (I.A1f), (I.A1g), (I.A1h), (I.A2f), (I.A2g), or (I.A2h) wherein R¹ is

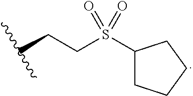

Embodiment No. 78 is directed to any one of Embodiment Nos. 1 to 26, wherein the compound is a compound of the formula (Iv), (Ivi), (Ivii), (I.A1f), (I.A1g), (I.A1h), (I.A2f), (I.A2g), or (I.A2h) wherein R¹ is

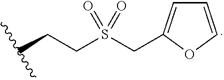

Embodiment No. 79 is directed to any one of Embodiment Nos. 1 to 26, wherein the compound is a compound of the formula (Iv), (Ivi), (Ivii), (I.A1f), (I.A1g), (I.A1h), (I.A2f), (I.A2g), or (I.A2h) wherein R¹ is

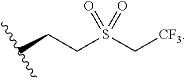

Embodiment No. 80 is directed to any one of Embodiment Nos. 37 to 79 wherein R² is H.

Embodiment No. 81 is directed to any one of Embodiment Nos. 37 to 79 wherein R² is alkyl.

Embodiment No. 82 is directed to any one of Embodiment Nos. 37 to 79 wherein R² is methyl.

Embodiment No. 83 is directed to any one of Embodiment Nos. 1 to 26 wherein R¹ is a -alkylene-S(O)₂—(C₁-C₆)haloalkyl group.

Embodiment No. 84 is directed to any one of Embodiment Nos. 1 to 26 wherein R¹ is a —(C₁ to C₂) alkylene-S(O)₂—(C₁-C₃)haloalkyl group.

Embodiment No. 85 is directed to any one of Embodiment Nos. 1 to 26 wherein R¹ is a —(C₁ to C₂) alkylene-S(O)₂—(C₁-C₂)haloalkyl group.

Embodiment No. 86 is directed to any one of Embodiment Nos. 1 to 26 wherein R¹ is a —(C₂) alkylene-S(O)₂—(C₁-C₂)haloalkyl group.

Embodiment No. 87 is directed to any one of Embodiment Nos. 1 to 26 wherein R¹ is a -alkylene-(tetrahydrothiophene 1,1-dioxide) group.

Embodiment No. 88 is directed to any one of Embodiment Nos. 1 to 26 wherein R¹ is a -alkenyl-S(O)₂—(C₁-C₆)alkyl group.

Embodiment No. 89 is directed to any one of Embodiment Nos. 1 to 26 wherein R¹ is a -cycloalkyl-S(O)₂—(C₁-C₆)alkyl group.

Embodiment No. 90 is directed to any one of Embodiment Nos. 1 to 26 wherein R¹ is a -alkylene-S(O)₂—(C₁-C₆)haloalkyl group, wherein the terminal carbon of said haloalkyl group is substituted with 1 to 3 of the same or different halo atoms. Examples of said haloalkyl groups include groups wherein the terminal carbon is substituted with 1 to 3 F atoms, or 2 to 3 F atoms, or 3 F atoms (with 3 F atoms being preferred).

Embodiment No. 91 is directed to any one of Embodiment Nos. 1 to 26 wherein R¹ is a —(C₁ to C₂) alkylene-S(O)₂—(C₁-C₃)haloalkyl group, wherein the terminal carbon of said haloalkyl group is substituted with 1 to 3 of the same or different halo atoms. Examples of said haloalkyl groups include groups wherein the terminal carbon is substituted with 1 to 3 F atoms, or 2 to 3 F atoms, or 3 F atoms (with 3 F atoms being preferred).

Embodiment No. 92 is directed to any one of Embodiment Nos. 1 to 26 wherein R¹ is a —(C₁ to C₂) alkylene-S(O)₂—(C₁-C₂)haloalkyl group, wherein the terminal carbon of said haloalkyl group is substituted with 1 to 3 of the same or different halo atoms. Examples of said haloalkyl groups include groups wherein the terminal carbon is substituted with 1 to 3 F atoms, or 2 to 3 F atoms, or 3 F atoms (with 3 F atoms being preferred).

Embodiment No. 93 is directed to any one of Embodiment Nos. 1 to 26 wherein R¹ is a —(C₂) alkylene-S(O)₂—(C₁-C₂)haloalkyl group, wherein the terminal carbon of said haloalkyl group is substituted with 1 to 3 of the same or different halo atoms. Examples of said haloalkyl groups include groups wherein the terminal carbon is substituted with 1 to 3 F atoms, or 2 to 3 F atoms, or 3 F atoms (with 3 F atoms being preferred).

Embodiment No. 94 is directed to any one of Embodiment Nos. 83 to 93 wherein the compound is a compound of formula (Ii), (Iii), (Iiii), (Iiv), (I.A1b), (I.A1c), (I.A1d), (I.A2b), (I.A2c), or (I.A1d), and R¹ is any one of the R¹ groups described in any one of Embodiment Nos. 40 to 60.

Embodiment No. 95 is directed to any one of Embodiment Nos. 1 to 26 wherein R¹ is an -alkylene-S(O)₂-substituted(C₁-C₆)alkyl group, such as, for example, an -alkylene-S(O)₂—(C₁-C₆)hydroxyalkyl group.

Embodiment No. 96 is directed to any one of Embodiment Nos. 1 to 26 wherein R¹ is a —(C₁ to C₂) alkylene-S(O)₂-substituted(C₁-C₃)alkyl group, such as, for example, a —(C₁ to C₂) alkylene-S(O)₂—(C₁-C₃)hydroxyalkyl group.

Embodiment No. 97 is directed to any one of Embodiment Nos. 1 to 26 wherein R¹ is a —(C₁ to C₂) alkylene-S(O)₂-substituted(C₁-C₂) alkyl group, such as, for example, a —(C₁ to C₂) alkylene-S(O)₂—(C₁-C₂)hydroxyalkyl group.

Embodiment No. 98 is directed to any one of Embodiment Nos. 1 to 26 wherein R¹ is a —(C₂) alkylene-S(O)₂-substituted(C₁-C₂)alkyl group, such as, for example, a —(C₂) alkylene-S(O)₂-substituted(C₁-C₂)hydroxyalkyl group.

Embodiment No. 99 is directed to any one of Embodiment Nos. 1 to 26 wherein R¹ is an -alkylene-S(O)₂-substituted(C₁-C₆)alkyl group, such as, for example, an -alkylene-S(O)₂—(C₁-C₆)hydroxyalkykl group, wherein the terminal carbon of said substituted alkyl group is substituted.

Embodiment No. 100 is directed to any one of Embodiment Nos. 1 to 26 wherein R¹ is a —(C₁ to C₂) alkylene-S(O)₂-substituted(C₁-C₃) alkyl group, such as, for example, a —(C₁ to C₂) alkylene-S(O)₂—(C₁-C₃)hydroxyalkyl group, wherein the terminal carbon of said alkyl group is substituted.

Embodiment No. 101 is directed to any one of Embodiment Nos. 1 to 26 wherein R¹ is a —(C₁ to C₂) alkylene-S(O)₂-substituted(C₁-C₂)alkyl group, such as, for example, a —(C₁ to C₂) alkylene-S(O)₂—(C₁-C₂)hydroxyalkyl group, wherein the terminal carbon of said alkyl group is substituted.

Embodiment No. 102 is directed to any one of Embodiment Nos. 1 to 26 wherein R¹ is a —(C₂) alkylene-S(O)₂-substituted(C₁-C₂)alkyl group, such as, for example, a —(C₁ to C₂) alkylene-S(O)₂—(C₁-C₂)hydroxyalkyl group, wherein the terminal carbon of said alkyl group is substituted.

Embodiment No. 103 is directed to any one of Embodiment Nos. 83 to 102 wherein R² is H.

Embodiment No. 104 is directed to any one of Embodiment Nos. 83 to 102 wherein R² is alkyl.

Embodiment No. 105 is directed to any one of Embodiment Nos. 83 to 102 wherein R² is methyl.

Embodiment No. 106 is directed to any one of the compounds of formulas (I.A1i), (I.A1l), (I.A1m), or (I.A1n) wherein Ar is unsubstituted aryl (e.g., unsubstituted phenyl).

Embodiment No. 108 is directed to any one of the compounds of formulas (I.A1i), (I.A1l), (I.A1m), or (I.A1n) wherein Ar is substituted aryl (e.g., substituted phenyl). Examples of substituted phenyls include, for example, (1) halo substituted phenyl (such as, for example chloro substituted phenyl, such as, for example, p-Cl-phenyl-), (2) haloalkyl substituted phenyl (such as, for example, —CF₃ substituted phenyl, such as, for example, p-CF₃-phenyl-), and (3) cyano substituted phenyl (such as, for example, p-CN-phenyl-).

Embodiment No. 109 is directed to any one of the compounds of formulas (I.A1i), (I.A1l), (I.A1m), or (I.A1n) wherein Ar is unsubstituted heteroaryl (e.g., unsubstituted pyridyl).

Embodiment No. 110 is directed to any one of the compounds of formulas (I.A1i), (I.A1l), (I.A1m), or (I.A1n) wherein Ar is substituted heteroaryl (e.g., substituted pyridyl). Examples of substituted pyridyls include, for example, (1) halo substituted pyridyl (such as, for example chloro substituted pyridyl), (2) haloalkyl substituted pyridyl (such as, for example, —CF₃ substituted pyridyl), and (3) cyano substituted pyridyl.

Embodiment No. 111 is directed to any one of the compounds of formulas (I.A1i), (I.A1l), (I.A1m), (I.A1n), (I.A1o) or (I.A1p) wherein each L¹ is the same or different halo.

Embodiment No. 112 is directed to any one of the compounds of formulas (I.A1i), (I.A1l), (I.A1m), (I.A1n), (I.A1o) or (I.A1p) wherein n is 2 and each L¹ is the same or different halo.

Embodiment No. 113 is directed to any one of the compounds of formulas (I.A1i), (I.A1l), (I.A1m), (I.A1n), (I.A1o) or (I.A1p) wherein n is 2 and each L¹ is the same halo.

Embodiment No. 114 is directed to any one of the compounds of formulas (I.A1i), (I.A1l), (I.A1m), (I.A1n), (I.A1o) or (I.A1p) wherein n is 2 and each L¹ is F.

Embodiment No. 115 is directed to any one of the compounds of formulas (I.A1i), (I.A1l), (I.A1m) or (I.A1n) wherein each L¹ is the same or different halo, and Ar is unsubstituted aryl (e.g., unsubstituted phenyl).

Embodiment No. 116 is directed to any one of the compounds of formulas (I.A1i), (I.A1l), (I.A1m) or (I.A1n), wherein n is 2, each L¹ is the same or different halo, and Ar is unsubstituted aryl (e.g., unsubstituted phenyl).

Embodiment No. 117 is directed to any one of the compounds of formulas (I.A1i), (I.A1l), (I.A1m) or (I.A1n), wherein n is 2, each L¹ is the same halo, and Ar is unsubstituted aryl (e.g., unsubstituted phenyl).

Embodiment No. 118 is directed to any one of the compounds of formulas (I.A1i), (I.A1l), (I.A1m) or (I.A1n), wherein n is 2, each L¹ is F, and Ar is unsubstituted aryl (e.g., unsubstituted phenyl).

Embodiment No. 119 is directed to any one of the compounds of formulas (I.A1i), (I.A1l), (I.A1m) or (I.A1n), wherein each L¹ is the same or different halo, and Ar is substituted aryl (e.g., substituted phenyl). Examples of substituted phenyls include, for example, (1) halo substituted phenyl (such as, for example chloro substituted phenyl, such as, for example, p-Cl-phenyl-), (2) haloalkyl substituted phenyl (such as, for example, —CF₃ substituted phenyl, such as, for example, p-CF₃-phenyl-), and (3) cyano substituted phenyl (such as, for example, p-CN-phenyl-).

Embodiment No. 120 is directed to any one of the compounds of formulas (I.A1i), (I.A1l), (I.A1m) or (I.A1n), wherein n is 2, each L¹ is the same or different halo, and Ar is substituted aryl (e.g., substituted phenyl). Examples of substituted phenyls include, for example, (1) halo substituted phenyl (such as, for example chloro substituted phenyl, such as, for example, p-Cl-phenyl-), (2) haloalkyl substituted phenyl (such as, for example, —CF₃ substituted phenyl, such as, for example, p-CF₃-phenyl-), and (3) cyano substituted phenyl (such as, for example, p-CN-phenyl-).

Embodiment No. 121 is directed to any one of the compounds of formulas (I.A1i), (I.A1l), (I.A1m) or (I.A1n), wherein n is 2, each L¹ is the same halo, and Ar is substituted aryl (e.g., substituted phenyl). Examples of substituted phenyls include, for example, (1) halo substituted phenyl (such as, for example chloro substituted phenyl, such as, for example, p-Cl-phenyl-), (2) haloalkyl substituted phenyl (such as, for example, —CF₃ substituted phenyl, such as, for example, p-CF₃-phenyl-), and (3) cyano substituted phenyl (such as, for example, p-CN-phenyl-).

Embodiment No. 122 is directed to any one of the compounds of formulas (I.A1i), (I.A1l), (I.A1m) or (I.A1n), wherein n is 2, each L is F, and Ar is substituted aryl (e.g., substituted phenyl). Examples of substituted phenyls include, for example, (1) halo substituted phenyl (such as, for example chloro substituted phenyl, such as, for example, p-Cl-phenyl-), (2) haloalkyl substituted phenyl (such as, for example, —CF₃ substituted phenyl, such as, for example, p-CF₃-phenyl-), and (3) cyano substituted phenyl (such as, for example, p-CN-phenyl-).

Embodiment No. 123 is directed to any one of the compounds of formulas (I.A1i), (I.A1l), (I.A1m) or (I.A1n), wherein each L¹ is the same or different halo, and Ar is unsubstituted heteroaryl (e.g., unsubstituted pyridyl).

Embodiment No. 124 is directed to any one of the compounds of formulas (I.A1i), (I.A1l), (I.A1m) or (I.A1n), wherein n is 2, each L¹ is the same or different halo, and Ar is unsubstituted heteroaryl (e.g., unsubstituted pyridyl).

Embodiment No. 125 is directed to any one of the compounds of formulas (I.A1i), (I.A1l), (I.A1m) or (I.A1n), wherein n is 2, each L¹ is the same halo, and Ar is unsubstituted heteroaryl (e.g., unsubstituted pyridyl).

Embodiment No. 126 is directed to any one of the compounds of formulas (I.A1i), (I.A1l), (I.A1m) or (I.A1n), wherein n is 2, each L¹ is F, and Ar is unsubstituted heteroaryl (e.g., unsubstituted pyridyl).

Embodiment No. 127 is directed to any one of the compounds of formulas (I.A1i), (I.A1l), (I.A1m) or (I.A1n), wherein each L¹ is the same or different halo, and Ar is substituted heteroaryl (e.g., substituted pyridyl). Examples of substituted pyridyls include, for example, (1) halo substituted pyridyl (such as, for example chloro substituted pyridyl), (2) haloalkyl substituted pyridyl (such as, for example, —CF₃ substituted pyridyl), and (3) cyano substituted pyridyl.

Embodiment No. 128 is directed to any one of the compounds of formulas (I.A1i), (I.A1l), (I.A1m) or (I.A1n), wherein n is 2, each L¹ is the same or different halo, and Ar is substituted heteroaryl (e.g., substituted pyridyl). Examples of substituted pyridyls include, for example, (1) halo substituted pyridyl (such as, for example chloro substituted pyridyl), (2) haloalkyl substituted pyridyl (such as, for example, —CF₃ substituted pyridyl), and (3) cyano substituted pyridyl.

Embodiment No. 129 is directed to any one of the compounds of formulas (I.A1i), (I.A1l), (I.A1m) or (I.A1n), wherein n is 2, each L¹ is the same halo, and Ar is substituted heteroaryl (e.g., substituted pyridyl). Examples of substituted pyridyls include, for example, (1) halo substituted pyridyl (such as, for example chloro substituted pyridyl), (2) haloalkyl substituted pyridyl (such as, for example, —CF₃ substituted pyridyl), and (3) cyano substituted pyridyl.

Embodiment No. 130 is directed to any one of the compounds of formulas (I.A1i), (I.A1l), (I.A1m) or (I.A1n), wherein n is 2, each L¹ is F, and Ar is substituted heteroaryl (e.g., substituted pyridyl). Examples of substituted pyridyls include, for example, (1) halo substituted pyridyl (such as, for example chloro substituted pyridyl), (2) haloalkyl substituted pyridyl (such as, for example, —CF₃ substituted pyridyl), and (3) cyano substituted pyridyl.

Embodiment No. 131 is directed to any one of the compounds of formulas (I.A1i), (I.A1l), (I.A1m) or (I.A1n), wherein n is 2, each L¹ is the same halo, and Ar is substituted aryl selected from the group consisting of p-CH₃CH₂SO₂phenyl, p-Br-phenyl, m,p-di-F-phenyl, m,p-di-CN-phenyl, p-CH₃O-phenyl, and p-CF₃CH₂Ophenyl.

Embodiment No. 132 is directed to any one of the compounds of formulas (I.A1i), (I.A1l), (I.A1m) or (I.A1n), wherein n is 2, each L¹ is F, and Ar is substituted aryl selected from the group consisting of p-CH₃CH₂SO₂phenyl, p-Br-phenyl, m,p-di-F-phenyl, m,p-di-CN-phenyl, p-CH₃O-phenyl, and p-CF₃CH₂Ophenyl.

Embodiment No. 133 is directed to any one of Embodiment Nos. 106 to 132 wherein R¹ is -alkylene-(tetrahydrothiophene 1,1-dioxide).

Embodiment No. 134 is directed to any one of Embodiment Nos. 106 to 132 wherein R¹ is -alkenyl-S(O)₂—(C₁-C₆)alkyl.

Embodiment No. 135 is directed to any one of Embodiment Nos. 106 to 132 wherein R¹ is -cycloalkyl-S(O)₂—(C₁-C₆)alkyl.

Embodiment No. 136 is directed to any one of Embodiment Nos. 106 to 132 wherein R¹ is an -alkylene-S(O)₂—(C₁-C₆)alkyl group.

Embodiment No. 137 is directed to any one of Embodiment Nos. 106 to 132 wherein R¹ is a —(C₁ to C₂) alkylene-S(O)₂—(C₁-C₆)alkyl group.

Embodiment No. 138 is directed to any one of Embodiment Nos. 106 to 132 wherein R¹ is a —(C₁ to C₂) alkylene-S(O)₂—(C₁-C₃)alkyl group.

Embodiment No. 139 is directed to any one of Embodiment Nos. 106 to 132 wherein R¹ is a —(C₂) alkylene-S(O)₂—(C₁-C₆)alkyl group.

Embodiment No. 140 is directed to any one of Embodiment Nos. 106 to 132 wherein R¹ is selected from the group consisting of:

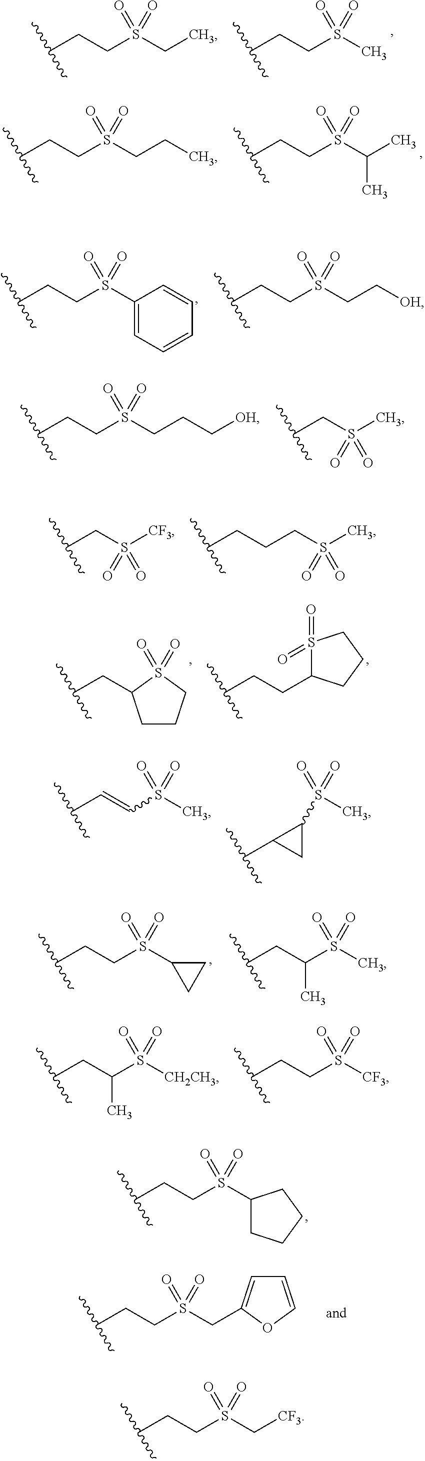

Embodiment No. 141 is directed to any one of Embodiment Nos. 106 to 132 wherein R¹ is selected from the group consisting of:

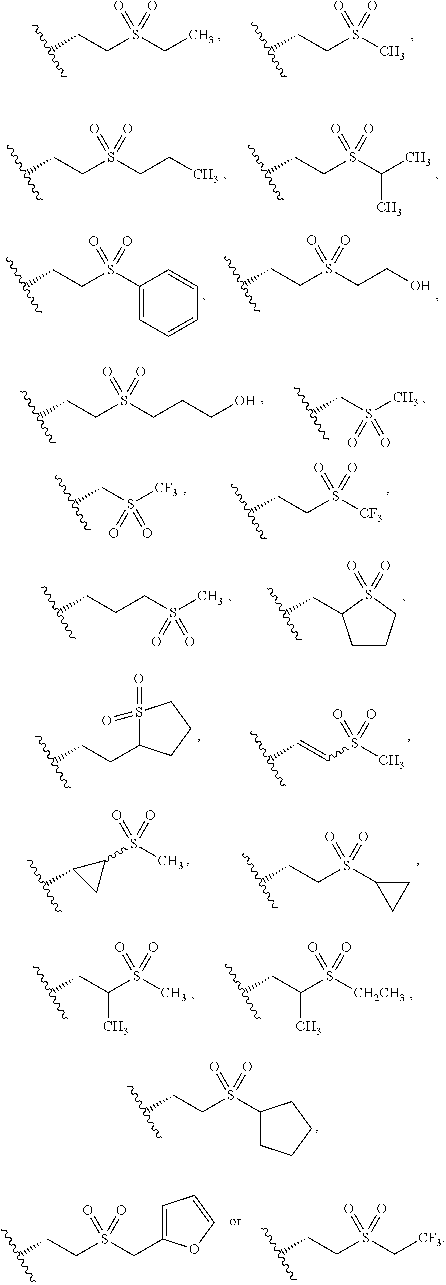

Embodiment No. 142 is directed to any one of Embodiment Nos. 106 to 132 wherein R¹ is selected from the group consisting of:

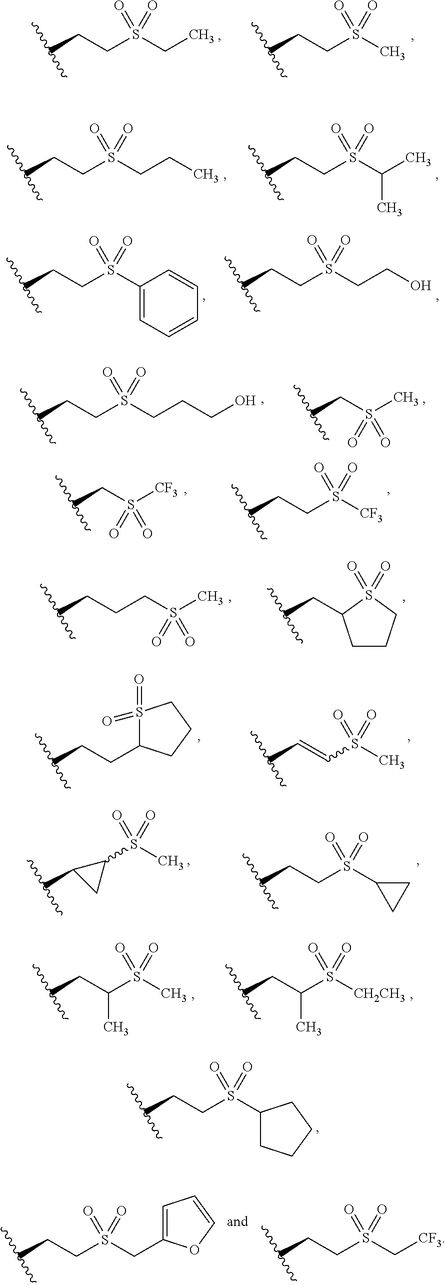

Embodiment No. 143 is directed to any one of Embodiment Nos. 106 to 132 wherein R¹ is

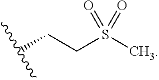

Embodiment No. 144 is directed to any one of Embodiment Nos. 106 to 132 wherein R¹ is

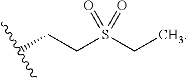

Embodiment No. 145 is directed to any one of Embodiment Nos. 106 to 132 wherein R¹ is

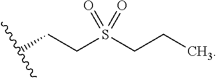

Embodiment No. 146 is directed to any one of Embodiment Nos. 106 to 132 wherein R¹ is

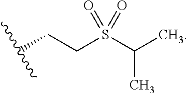

Embodiment No. 147 is directed to any one of Embodiment Nos. 106 to 132 wherein R¹ is

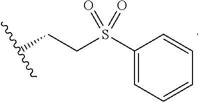

Embodiment No. 148 is directed to any one of Embodiment Nos. 106 to 132 wherein R¹ is

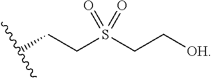

Embodiment No. 149 is directed to any one of Embodiment Nos. 106 to 132 wherein R¹ is

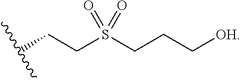

Embodiment No. 149 is directed to any one of Embodiment Nos. 106 to 132 wherein R¹ is

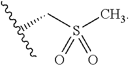

Embodiment No. 150 is directed to any one of Embodiment Nos. 106 to 132 wherein R¹ is

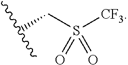

Embodiment No. 151 is directed to any one of Embodiment Nos. 106 to 132 wherein R¹ is

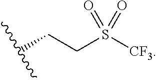

Embodiment No. 152 is directed to any one of Embodiment Nos. 106 to 132 wherein R¹ is

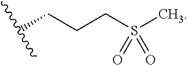

Embodiment No. 152 is directed to any one of Embodiment Nos. 106 to 132 wherein R¹ is

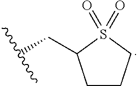

Embodiment No. 153 is directed to any one of Embodiment Nos. 106 to 132 wherein R¹ is

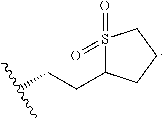

Embodiment No. 154 is directed to any one of Embodiment Nos. 106 to 132 wherein R¹ is

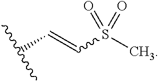

Embodiment No. 155 is directed to any one of Embodiment Nos. 106 to 132 wherein R¹ is

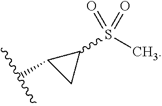

Embodiment No. 156 is directed to any one of Embodiment Nos. 106 to 132 wherein R¹ is

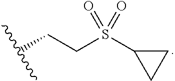

Embodiment No. 157 is directed to any one of Embodiment Nos. 106 to 132 wherein R¹ is

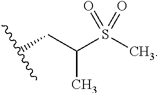

Embodiment No. 158 is directed to any one of Embodiment Nos. 106 to 132 wherein R¹ is

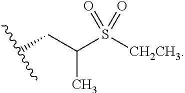

Embodiment No. 159 is directed to any one of Embodiment Nos. 106 to 132 wherein R¹ is

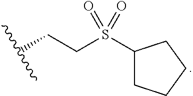

Embodiment No. 160 is directed to any one of Embodiment Nos. 106 to 132 wherein R¹ is

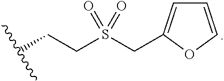

Embodiment No. 161 is directed to any one of Embodiment Nos. 106 to 132 wherein R¹ is

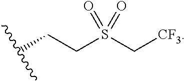

Embodiment No. 162 is directed to any one of Embodiment Nos. 106 to 132 wherein R¹ is

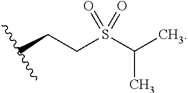

Embodiment No. 163 is directed to any one of Embodiment Nos. 106 to 132 wherein R¹ is

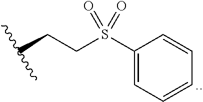

Embodiment No. 164 is directed to any one of Embodiment Nos. 106 to 132 wherein R¹ i

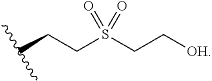

Embodiment No. 165 is directed to any one of Embodiment Nos. 106 to 132 wherein R¹ i

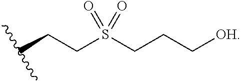

Embodiment No. 166 is directed to any one of Embodiment Nos. 106 to 132 wherein R¹ i

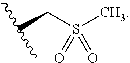

Embodiment No. 167 is directed to any one of Embodiment Nos. 106 to 132 wherein R¹ i

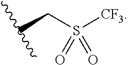

Embodiment No. 168 is directed to any one of Embodiment Nos. 106 to 132 wherein R¹ i

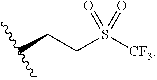

Embodiment No. 169 is directed to any one of Embodiment Nos. 106 to 132 wherein R¹ i

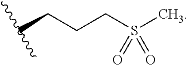

Embodiment No. 170 is directed to any one of Embodiment Nos. 106 to 132 wherein R¹ i

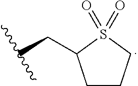

Embodiment No. 171 is directed to any one of Embodiment Nos. 106 to 132 wherein R¹ is

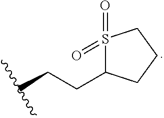

Embodiment No. 172 is directed to any one of Embodiment Nos. 106 to 132 wherein R¹ is

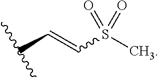

Embodiment No. 173 is directed to any one of Embodiment Nos. 106 to 132 wherein R¹ is

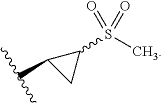

Embodiment No. 174 is directed to any one of Embodiment Nos. 106 to 132 wherein R¹ is

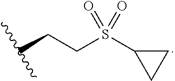

Embodiment No. 175 is directed to any one of Embodiment Nos. 106 to 132 wherein R¹ is

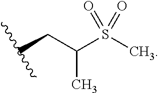

Embodiment No. 176 is directed to any one of Embodiment Nos. 106 to 132 wherein R¹ is

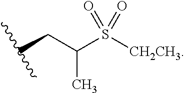

Embodiment No. 177 is directed to any one of Embodiment Nos. 106 to 132 wherein R¹ is

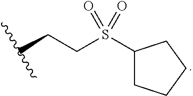

Embodiment No. 178 is directed to any one of Embodiment Nos. 106 to 132 wherein R¹ is

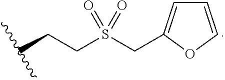

Embodiment No. 179 is directed to any one of Embodiment Nos. 106 to 132 wherein R¹ i

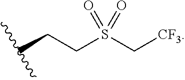

Embodiment No. 180 is directed to any one of Embodiment Nos. 106 to 132 wherein is a -alkylene-S(O)₂—(C₁-C₆)haloalkyl group.

Embodiment No. 181 is directed to any one of Embodiment Nos. 106 to 132 wherein R¹ is a —(C₁ to C₂) alkylene-S(O)₂—(C₁-C₃)haloalkyl group.

Embodiment No. 182 is directed to any one of Embodiment Nos. 106 to 132 wherein R¹ is a —(C₁ to C₂) alkylene-S(O)₂—(C₁-C₂)haloalkyl group.

Embodiment No. 183 is directed to any one of Embodiment Nos. 106 to 132 wherein R¹ is a —(C₂) alkylene-S(O)₂—(C₁-C₂)haloalkyl group.

Embodiment No. 184 is directed to any one of Embodiment Nos. 106 to 132 wherein R¹ is a -alkylene-(tetrahydrothiophene 1,1-dioxide) group.

Embodiment No. 185 is directed to any one of Embodiment Nos. 106 to 132 wherein R¹ is a -alkenyl-S(O)₂—(C₁-C₆)alkyl group.

Embodiment No. 186 is directed to any one of Embodiment Nos. 106 to 132 wherein R¹ is a -cycloalkyl-S(O)₂—(C₁-C₆)alkyl group.

Embodiment No. 187 is directed to any one of Embodiment Nos. 106 to 132 wherein R¹ is a -alkylene-S(O)₂—(C₁-C₆)haloalkyl group, wherein the terminal carbon of said haloalkyl group is substituted with 1 to 3 of the same or different halo atoms. Examples of said haloalkyl groups include groups wherein the terminal carbon is substituted with 1 to 3 F atoms, or 2 to 3 F atoms, or 3 F atoms (with 3 F atoms being preferred).

Embodiment No. 188 is directed to any one of Embodiment Nos. 106 to 132 wherein R¹ is a —(C₁ to C₂) alkylene-S(O)₂—(C₁-C₃)haloalkyl group, wherein the terminal carbon of said haloalkyl group is substituted with 1 to 3 of the same or different halo atoms. Examples of said haloalkyl groups include groups wherein the terminal carbon is substituted with 1 to 3 F atoms, or 2 to 3 F atoms, or 3 F atoms (with 3 F atoms being preferred).

Embodiment No. 189 is directed to any one of Embodiment Nos. 106 to 132 wherein R¹ is a —(C₁ to C₂) alkylene-S(O)₂—(C₁-C₂)haloalkyl group, wherein the terminal carbon of said haloalkyl group is substituted with 1 to 3 of the same or different halo atoms. Examples of said haloalkyl groups include groups wherein the terminal carbon is substituted with 1 to 3 F atoms, or 2 to 3 F atoms, or 3 F atoms (with 3 F atoms being preferred).

Embodiment No. 190 is directed to any one of Embodiment Nos. 106 to 132 wherein R¹ is a —(C₂) alkylene-S(O)₂—(C₁-C₂)haloalkyl group, wherein the terminal carbon of said haloalkyl group is substituted with 1 to 3 of the same or different halo atoms. Examples of said haloalkyl groups include groups wherein the terminal carbon is substituted with 1 to 3 F atoms, or 2 to 3 F atoms, or 3 F atoms (with 3 F atoms being preferred).

Embodiment No. 191 is directed to any one of Embodiment Nos. 106 to 132 wherein R¹ is an -alkylene-S(O)₂-substituted(C₁-C₆)alkyl group, such as, for example, an -alkylene-S(O)₂—(C₁-C₆)hydroxyalkyl group.

Embodiment No. 192 is directed to any one of Embodiment Nos. 106 to 132 wherein R¹ is a —(C₁ to C₂) alkylene-S(O)₂-substituted(C₁-C₃)alkyl group, such as, for example, a —(C₁ to C₂) alkylene-S(O)₂—(C₁-C₃)hydroxyalkyl group.

Embodiment No. 193 is directed to any one of Embodiment Nos. 106 to 132 wherein R¹ is a —(C₁ to C₂) alkylene-S(O)₂-substituted(C₁-C₂) alkyl group, such as, for example, a —(C₁ to C₂) alkylene-S(O)₂—(C₁-C₂)hydroxyalkyl group.

Embodiment No. 194 is directed to any one of Embodiment Nos. 106 to 132 wherein R¹ is a —(C₂) alkylene-S(O)₂-substituted(C₁-C₂)alkyl group, such as, for example, a —(C₂) alkylene-S(O)₂-substituted(C₁-C₂)hydroxyalkyl group.

Embodiment No. 195 is directed to any one of Embodiment Nos. 106 to 132 wherein R¹ is an -alkylene-S(O)₂-substituted(C₁-C₆)alkyl group, such as, for example, an -alkylene-S(O)₂—(C₁-C₆)hydroxyalkykl group, wherein the terminal carbon of said substituted alkyl group is substituted.

Embodiment No. 196 is directed to any one of Embodiment Nos. 106 to 132 wherein R¹ is a —(C₁ to C₂) alkylene-S(O)₂-substituted(C₁-C₃) alkyl group, such as, for example, a —(C₁ to C₂) alkylene-S(O)₂—(C₁-C₃)hydroxyalkyl group, wherein the terminal carbon of said alkyl group is substituted.

Embodiment No. 197 is directed to any one of Embodiment Nos. 106 to 132 wherein R¹ is a —(C₁ to C₂) alkylene-S(O)₂-substituted(C₁-C₂)alkyl group, such as, for example, a —(C₁ to C₂) alkylene-S(O)₂—(C₁-C₂)hydroxyalkyl group, wherein the terminal carbon of said alkyl group is substituted.

Embodiment No. 198 is directed to any one of Embodiment Nos. 106 to 132 wherein R¹ is a —(C₂) alkylene-S(O)₂-substituted(C₁-C₂)alkyl group, such as, for example, a —(C₁ to C₂) alkylene-S(O)₂—(C₁-C₂)hydroxyalkyl group, wherein the terminal carbon of said alkyl group is substituted.

As used above, and throughout this disclosure, the following terms, unless otherwise indicated, shall be understood to have the following meanings:

“RAC” (or “rac”) means racemate.

“Patient” includes both human and animals.

“Mammal” means humans and other mammalian animals.

“One or more” means at least one, for example, 1, 2 or 3, or 1 or 2, or 1, thus, for example, “one or more L³ groups” means at least one L³ group, and examples include 1-3 L³ groups, 1 or 2 L³ groups, and one L³ group.

“At least one” means there is at least one, and examples include 1, 2 or 3, or 1 or 2, or 1.

“Alkyl” means an aliphatic hydrocarbon group, which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain, which may be straight or branched. The term “substituted alkyl” means that the alkyl group may be substituted by one or more substituents which may be the same or different, each substituent being independently selected from the group consisting of halo, alkyl, aryl, cycloalkyl, cyano, hydroxy, alkoxy, alkylthio, amino, —NH(alkyl), —NH(cycloalkyl), —N(alkyl)₂, carboxy and —C(O)O-alkyl (unless expressly defined otherwise). Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain, which may be straight or branched. Non-limiting examples of suitable alkynyl groups include ethynyl, propynyl, 2-butynyl and 3-methylbutynyl. The term “substituted alkynyl” means that the alkynyl group may be substituted by one or more substituents which may be the same or different, each substituent being independently selected from the group consisting of alkyl, aryl and cycloalkyl.

“Alkylene” means a divalent aliphatic hydrocarbon radical derived from an alkyl group, as defined above. Both “open” valences may be on the same carbon atom, or on different carbon atoms. Examples of alkylene groups include C₁-C₆ alkylene groups, for example, C₁ to C₄ alkylene groups, and in another example, C₁-C₃ alkylene groups, and in another example C₁ to C₂ alkylene groups. Non-limiting examples of alkylene groups include —CH₂—, —CH₂—CH₂—, —CH(CH₃)—, etc.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain, which may be straight or branched. Non-limiting examples of suitable alkynyl groups include ethynyl, propynyl, 2-butynyl and 3-methylbutynyl. The term “substituted alkynyl” means that the alkynyl group may be substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined herein. Non-limiting examples of suitable saturated monocyclic cycloalkyls include cyclopropyl, cyclopentyl, cyclohexyl, cycloheptyl and the like, and non-limiting examples of non-aromatic, unsaturated monocyclic cycloalkyls include cyclopentenyl, cyclohexenyl, etc. Non-limiting examples of suitable multicyclic cycloalkyls include 1-decalinyl, norbornyl, adamantyl and the like, as well as partially saturated species such as, for example, indanyl, tetrahydronaphthyl and the like.

“Halogen” or “halo” means fluorine, chlorine, bromine, or iodine. Fluorine, chlorine and bromine are preferred.

“Heteroalkyl” means an alkyl group (as defined herein) wherein one or more carbon atoms have been replaced with heteroatoms, such as, for example, heteroatoms each independently selected from the group consisting of: N, —(NR^{13A})—, O, S(O), and S(O)₂.

“Ring system substituent” means a substituent attached to an aromatic or non-aromatic ring system, which, for example, replaces an available hydrogen on the ring system. Ring system substituents may be the same or different, each being independently selected from the group consisting of alkyl, alkenyl, alkynyl, aryl (substituted or unsubstituted, heteroaryl (substituted or unsubstituted, alkylene-aryl, heteroarylalkenyl, heteroarylalkynyl, hydroxy, hydroxyalkyl, alkoxy, aryloxy, aryl substituted alkoxy, acyl, aroyl, halo, nitro, cyano, carboxy, alkoxycarbonyl, aryloxycarbonyl, arylalkoxycarbonyl, alkylsulfonyl, arylsulfonyl, heteroarylsulfonyl, alkylthio, arylthio, heteroarylthio, arylalkylthio, heteroarylalkylthio, cycloalkyl, heterocycloalkyl, —C(═N—CN)—NH₂, —C(═NH)—NH₂, —C(═NH)—NH(alkyl), Y₁Y₂N—, Y₁Y₂N-alkyl-, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and -alkylene-aryl (unless expressly defined otherwise). The term “ring system substituent” may also mean a single moiety in which two available hydrogens on two adjacent carbon atoms are simultaneously replaced (e.g., one H on each carbon) on a ring system. Examples of such moiety are methylenedioxy, ethylenedioxy, —C(CH₃)₂— and the like which form moieties such as, for example:

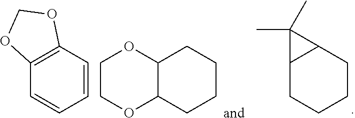

“Heterocycloalkyl” means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocycloalkyls contain about 5 to about 6 ring atoms. The prefix aza, oxa or thia before the heterocycloalkyl root name means that at least a nitrogen, oxygen or sulfur atom respectively is present as a ring atom. Any —NH in a heterocycloalkyl ring may exist in protected form, for example, as an —N(Boc), —N(CBz), —N(Tos) group and the like; such protected forms are also considered part of this invention. The heterocycloalkyl can be optionally substituted by one or more “ring system substituents” which may be the same or different, and are as defined herein. The nitrogen or sulfur atom of the heterocycloalkyl can be optionally oxidized to the corresponding N-oxide, S-oxide or S,S-dioxide. Non-limiting examples of suitable monocyclic heterocycloalkyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. Non-limiting examples of non-aromatic, unsaturated monocyclic heterocycloalkyl rings include thiazolinyl, 2,3-dihydrofuranyl, 2,3-dihydrothiophenyl, etc.

It should be noted that in the hetero-atom containing ring systems of this invention, there are no hydroxyl groups on carbon atoms adjacent to a N, O or S, as well as there are no N or S groups on carbon atoms adjacent to another heteroatom. Thus, for example, in the ring:

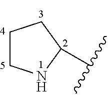

there is no —OH attached directly to carbons marked 2 and 5.

It should also be noted that tautomeric forms such as, for example, the moieties:

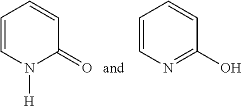

are considered equivalent in this invention.

“Hydroxyalkyl” means an alkyl group substituted with a hydroxyl (—OH) group in which alkyl is as previously defined. Preferred hydroxyalkyls contain lower alkyl. Non-limiting examples of suitable hydroxyalkyl groups include hydroxymethyl and 2-hydroxyethyl.

“Haloalkyl” means an alkyl group substituted with one or more independently selected halo atoms (e.g., F, Cl and Br, and in one example, one or more F atoms), and wherein one example is —CF₃.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an —O-alkyl; group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Alkoxyalkyl” means an -alkyl-O-alkyl group wherein alkyl is as previously described.

“Aryloxy” means an —O-aryl group in which the aryl group is as previously described. Non-limiting examples of suitable aryloxy groups include phenoxy and naphthoxy. The bond to the parent moiety is through the ether oxygen.

“Alkylthio” means an —S-alkyl group in which the alkyl group is as previously described. Non-limiting examples of suitable alkylthio groups include methylthio and ethylthio. The bond to the parent moiety is through the sulfur.

“Arylthio” means an —S-aryl group in which the aryl group is as previously described. Non-limiting examples of suitable arylthio groups include phenylthio and naphthylthio. The bond to the parent moiety is through the sulfur.

“Arylalkylthio” means an —S-alkylene-aryl group in which the alkylene and aryl groups are as previously described. A non-limiting example of a suitable arylalkylthio group is benzylthio. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Arylalkoxycarbonyl” means an —C(O)—O-alkylene-aryl group. A non-limiting example of a suitable arylalkoxycarbonyl group is benzyloxycarbonyl. The bond to the parent moiety is through the carbonyl.

“Alkylsulfonyl” means an alkyl-S(O₂)— group. Preferred groups are those in which the alkyl group is a lower alkyl. The bond to the parent moiety is through the sulfonyl.

“Arylsulfonyl” means an aryl-S(O₂)— group. The bond to the parent moiety is through the sulfonyl.

“Alkanoyl” means an alkyl-C(O)— group.

“Carbamoyl” means an NH₂—C(O)— group.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

When a group is substituted with “one or more” substituents, the indicated group may be substituted with one substituent, two substituents, etc., provided that the resulting substituted group forms a stable structure, as described above.

The term “optionally substituted” means optional substitution with the specified groups, radicals or moieties. For example, an aryl optionally substituted with an indicated group of substituents includes unsubstituted aryl as well as aryl substituted with any of the indicated substituents.

The term “isolated” or “in isolated form” for a compound refers to the physical state of said compound after being isolated from a synthetic process or natural source or combination thereof. The term “purified” or “in purified form” for a compound refers to the physical state of said compound after being obtained from a purification process or processes described herein or well known to the skilled artisan, in sufficient purity to be characterizable by standard analytical techniques described herein or well known to the skilled artisan.

It should also be noted that any carbon atom as well as any heteroatom with unsatisfied valences in the text, schemes, examples, Tables, etc. herein is assumed to have the sufficient number of hydrogen atom(s) to satisfy the valences.

When a functional group in a compound is termed “protected”, this means that the group is present in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York, herein incorporated by reference in its entirety.

When any variable (e.g., aryl, heterocycloalkyl, L¹, etc.) occurs more than one time in any constituent or in Formula (I), its definition on each occurrence is independent of its definition at every other occurrence (unless otherwise expressly indicated).

As used herein, the term “composition” is intended to encompass a product comprising the specified ingredients in the specified amounts, as well as any product which results, directly or indirectly, from combination of the specified ingredients in the specified amounts.

An “electron withdrawing group” (abbreviated as EWG), as used herein is a group that will draw electrons to itself more than a hydrogen atom would if it occupied the same position in the molecule (see, for example, Jerry March, “Advanced Organic Chemistry”, 4^{th} Edition, page 18, John Wiley & Sons, 1992)

Prodrugs and solvates of the compounds of the invention are also contemplated herein. The term “prodrug”, as employed herein, denotes a compound that is a drug precursor that, upon administration to a subject, undergoes chemical conversion by metabolic or chemical processes to yield a compound of Formula (I) or a salt and/or solvate thereof. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. A “hydrate” is a solvate wherein the solvent molecule(s) is/are H₂O.

“Effective amount” or “therapeutically effective amount” is meant to describe an amount of compound or a composition of the present invention effective in preventing the formation and/or deposition of amyloid protein, and thus producing the desired therapeutic, ameliorative, inhibitory or preventative effect.

The compounds of Formula (I) can form salts, which are also within the scope of this invention. Reference to a compound of Formula (I) herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula (I) contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula (I) may be formed, for example, by reacting a compound of Formula (I) with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Compounds of Formula (I), and salts, solvates and prodrugs thereof, may exist in their tautomeric form (for example, as an amide or imino ether). All such tautomeric forms are contemplated herein as part of the present invention.

All stereoisomers (for example, geometric isomers, optical isomers and the like) of the present compounds (including those of the salts, solvates and prodrugs of the compounds as well as the salts and solvates of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate” “prodrug” and the like, is intended to equally apply to the salt, solvate and prodrug of enantiomers, stereoisomers, rotamers, tautomers, positional isomers, racemates or prodrugs of the inventive compounds.

Polymorphic forms of the compounds of Formula (I), and of the salts, solvates and prodrugs of the compounds of Formula (I), are intended to be included in the present invention.

The compounds according to the invention have pharmacological properties; in particular, the compounds of Formula (I) can inhibit gamma-secretase, and are therefore useful in the treatment or prevention of neurodegenerative diseases, e.g., Alzheimer's Disease.

Representative compounds of the invention include but are not limited to the compounds and Examples described herein.

Pharmaceutical compositions can comprise one or more of the compounds of Formula (I). For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active compound. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa., herein incorporated by reference in its entirety.

Liquid form preparations include solutions, suspensions and emulsions. Water or water-propylene glycol solutions for parenteral injection or addition of sweeteners and opacifiers for oral solutions, suspensions and emulsions are examples. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

Preferably, the pharmaceutical preparation is in a unit dosage form. In such form, the preparation is subdivided into suitably sized unit doses containing appropriate quantities of the active compound, e.g., an effective amount to achieve the desired purpose.

The term “pharmaceutical composition” is also intended to encompass both the bulk composition and individual dosage units comprised of more than one (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a patient by administering a pharmaceutical composition of the present invention is also intended to encompass the administration of the afore-said bulk composition and individual dosage units.

The quantity of active compound in a unit dose of preparation may be varied or adjusted from about 0.01 mg to about 1000 mg, preferably from about 0.01 mg to about 750 mg, more preferably from about 0.01 mg to about 500 mg, and most preferably from about 0.01 mg to about 250 mg, according to the particular application. The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 0.04 mg/day to about 4000 mg/day, in one to four divided doses.

### EXAMPLES

The invention disclosed herein is exemplified by the following preparations and examples, which should not be construed to limit the scope of the disclosure. Alternative mechanistic pathways and analogous structures will be apparent to those skilled in the art.

The deuterated compounds of this invention can be made according to procedures well known in the art, and by the procedures described below. The deuterated compounds of this invention can be made by substituting the appropriate deuterated reagents in the procedures described in WO2009/008980 published Jan. 15, 2009, the disclosure of which is incorporated herein by reference thereto.

Deuterium can be incorporated into various positions in place of hydrogen using the methods described for making the hydrogen compounds. For instance, reducing agents such as lithium aluminum hydride, sodium borohydride, and hydrogen gas can be replaced by lithium aluminum deuteride, sodium borodeuteride, and deuterium gas to introduce deuterium in the same manner as hydrogen. In addition, other reagents used to prepare the hydrogen compounds can be prepared in deuterated form from appropriate deuterated precursors. For instance, a deuterated Grignard reagent can be prepared from the appropriate deuterated alkyl or aryl halide in the same manner as the undeuterated reagent. Similarly, deuterated alkyl and aryl thiols, sulfinic acids, sulfenic acids, sulfoxides, and sulfones can be prepared using well known reagents and used in the methods previously described for making hydrogen compounds. One skilled in the art will recognize that there are also additional methods for introducing deuterium. For instance, hydrogen can be replaced by halogen using well known halogenation conditions (eg halogen or N-halosuccinimide optionally in the presence of a lewis acid, light, or radical initiator) followed by reductive dehalogenation using deuterium gas or metal deuteride. One skilled in the art will also recognize that where any of these methods has the potential to introduce multiple deuteriums at different points on the molecule, there may be an unequal incorporation of deuterium depending on the specific reagents and substrates used and all potential sites of deuteration may not be deuterated.

The deuterated compounds of this invention would be prepared by following the methods in the table below. Methods 1 to 7 are described below the table. The Schemes referred to in Methods 1 to 7 are disclosed in WO2009/008980 published Jan. 15, 2009.

| Formula type | Method | Non-limiting example |
|---|---|---|
| 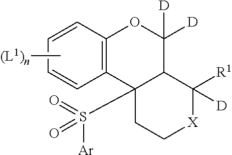 | Method 1 | 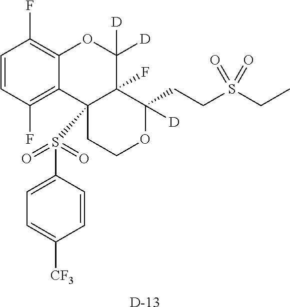 |
| 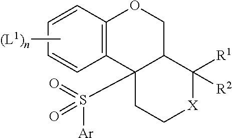 | Method 2 | 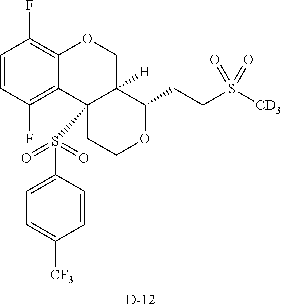 |
| 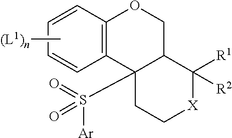 | Method 3 | 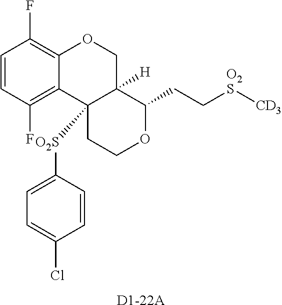 |
| 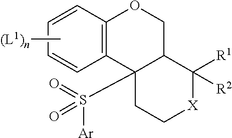 | Method 4 | 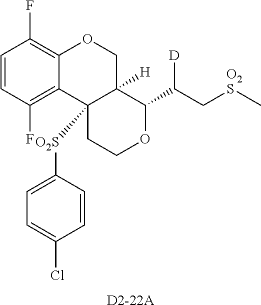 |
| 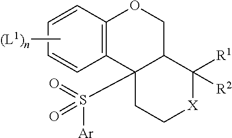 | Method 5 | 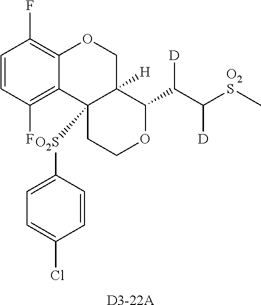 |
| 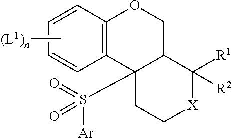 | Method 6. | 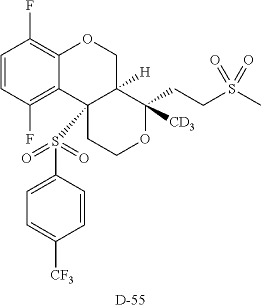 |
| 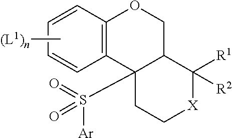 | Method 7 | 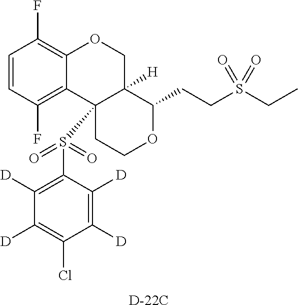 |

### Method 1

Prepare by following Scheme 1, Step 3 using LiAlD₄. Similarly, prepare by following Scheme 2, Step 3.

### Method 2

Prepare by following Scheme 1. In Step 13, use CD₃SO₂Na. Similarly prepare compounds b making corresponding changes to Scheme 2 (Step 13), Scheme 3 (Step 4), Scheme 5 (Step 3), Scheme 9 (Step 4), and Scheme 16 (Step 6).

### Method 3

Alternate to Method 2. Prepare by following Scheme 6, using (EtO)₂POCH₂SO₂CD₃ in Step 2.

### Method 4

Prepare compounds wherein R¹ is (-(deuterated alkylene))-SO₂—R^{A1} by following Scheme 6 Step 3, using NaBD₄.

R^{A1} is the substituent bound to the SO₂ group in the definition of R¹ for formula (I), i.e., R^{A1} is —(C₁-C₆)haloalkyl, R⁶, R⁸, or -substituted(C₁-C₆)alkyl.

### Method 5

Prepare compounds wherein R¹ is ((-deuterated alkylene))-SO₂—R^{A1} by following Scheme 6 Step 3, using deuterium gas and a catalyst such as palladium on carbon instead of NaBH₄. R^{A1} is as defined in Method 4 above.

### Method 6

R² is deuterated alkyl. Prepare compounds following Scheme 11 Step one, substituting the appropriate deuterated Grignard reagent, such as CD₃MgBr.

### Method 7

Ar is a deuterated aromatic. Prepare compounds following Scheme 2 Step 2, substituting the appropriate deuterated ArSH such as 4-chloro-2,3,5,6-tetradeuterobenzenethiol.

### Assay

The pharmacological properties of the compounds of this invention may be evaluated by a number of pharmacological assays. Gamma secretase activity can be determined by the assay described below.

Gamma-secretase activity is determined as described by Zhang et al. (Biochemistry, 40 (16), 5049-5055, 2001), which is herein incorporated by reference. Activity is expressed either as a percent inhibition or as the concentration of compound producing 50% inhibition of enzyme activity.

### Reagents.

Antibodies WO2, G2-10, and G2-11 are obtained from Dr. Konrad Beyreuther (University of Heidelberg, Heidelberg, Germany). WO2 recognizes residues 5-8 of Aβ peptide, while G2-10 and G2-11 recognize the specific C-terminal structure of Aβ 40 and Aβ 42, respectively. Biotin-4G8 is purchased from Senetec (St. Louis, Mo.). All tissue culture reagents to be used in this work are from Life Technologies, Inc., unless otherwise specified. Pepstatin A is purchased from Roche Molecular Biochemicals; DFK167 is from Enzyme Systems Products (Livermore, Calif.).

cDNA Constructs, Tissue Culture, and Cell Line Construction.

The construct SPC99-Ion, which contains the first 18 residues and the C-terminal 99 amino acids of APP carrying the London mutation, has been described (Zhang, L., Song, L., and Parker, E. (1999) J. Biol. Chem. 274, 8966-8972). Upon insertion into the membrane, the 17 amino acid signal peptide is processed, leaving an additional leucine at the N-terminus of Aβ. SPC99-lon is cloned into the pcDNA4/TO vector (Invitrogen) and is transfected into 293 cells which are stably transfected with pcDNA6/TR, which is provided in the T-REx system (Invitrogen). The transfected cells are selected in Dulbecco's modified Eagle's media (DMEM) supplemented with 10% fetal bovine serum, 100 units/mL penicillin, 100 g/mL streptomycin, 250 g/mL zeocin, and 5 g/mL blasticidin (Invitrogen). Colonies are screened for Aβ production by inducing C99 expression with 0.1 g/mL tetracycline for 16-20 h and analyzing conditioned media with a sandwich immunoassay (see below). One of the clones, designated as pTRE.15, is used in these studies.

### Membrane Preparation.

C99 expression in cells is induced with 0.1 g/mL tetracycline for 20 h. The cells are pretreated with 1 M phorbol 12-myristate 13-acetate (PMA) and 1 M brefeldin A (BFA) for 5-6 h at 37 C before harvesting. The cells are washed 3 times with cold phosphate-buffered saline (PBS) and are harvested in buffer A containing 20 mM Hepes (pH 7.5), 250 mM sucrose, 50 mM KCl, 2 mM EDTA, 2 mM EGTA, and Complete protease inhibitor tablets (Roche Molecular Biochemicals). The cell pellets are flash-frozen in liquid nitrogen and stored at −70° C. before use.

To make membranes, the cells are resuspended in buffer A and are lysed in a nitrogen bomb at 600 psi. The cell lysate is centrifuged at 1500 g for 10 min to remove nuclei and large cell debris. The supernatant is centrifuged at 100000 g for 1 h. The membrane pellet is resuspended in buffer A plus 0.5 M NaCl, and the membranes are collected by centrifugation at 200000 g for 1 h. The salt-washed membrane pellet is washed again in buffer A and centrifuged at 100000 g for 1 h. The final membrane pellet is resuspended in a small volume of buffer A using a Teflon-glass homogenizer. The protein concentration is determined, and membrane aliquots were flash-frozen in liquid nitrogen and stored at −70° C.

### γ-Secretase Reaction and Aβ Analysis.

To measure γ-secretase activity, membranes are incubated at 37° C. for 1 h in 50 μL of buffer containing 20 mM Hepes (pH 7.0) and 2 mM EDTA. At the end of the incubation, Aβ 40 and Aβ 42 are measured using an electrochemiluminescence (ECL)-based immunoassay. Aβ 40 is identified with antibody pairs TAG-G2-10 and biotin-WO2, while Aβ 42 is identified with TAG-G2-11 and biotin-408. The ECL signal is measured using an ECL-M8 instrument (IGEN International, Inc.) according to the manufacturer's instructions.

While the present invention has been described in conjunction with the specific embodiments set forth above, many alternatives, modifications and variations thereof will be apparent to those of ordinary skill in the art. All such alternatives, modifications and variations are intended to fall within the spirit and scope of the present invention.

What is claimed is:

1. A compound of the formula: 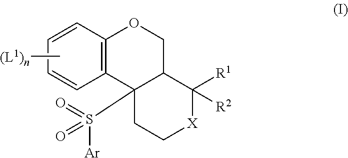
or a pharmaceutically acceptable salt, solvate, or ester thereof, wherein:
at least one H in formula (I) is replaced by deuterium;
X is selected from the group consisting of O and CH₂;
R¹ is selected from the group consisting of: (1) -alkylene-S(O)₂—(C₁-C₆)alkyl, (2) -alkylene-S(O)₂—(C₁-C₆)haloalkyl; (3) -alkylene-S(O)₂—R⁶, (4) -alkylene-S(O)₂—R⁸, (5) -alkylene-S(O)₂-substituted(C₁-C₆)alkyl, (6) -alkylene-(tetrahydrothiophene 1,1-dioxide), (7) -alkenyl-S(O)₂—(C₁-C₆)alkyl, and (8) -cycloalkyl-S(O)₂—(C₁-C₆)alkyl;
wherein said -alkylene-S(O)₂-substituted(C₁-C₆)alkyl R¹ group is substituted with one or more substituents independently selected from the group consisting of:
—OH, halo, —CN, —CF₃, —O—(C₁-C₆)alkyl, and —O-(halo(C₁-C₆)alkyl);
R² is selected from the group consisting of: H and alkyl;
R⁶ is selected from the group consisting of: (1) unsubstituted (C₆-C₁₄)aryl, (2) (C₆-C₁₄)aryl substituted with one or more L^{1A} groups, (3) unsubstituted (C₆-C₁₄)heteroaryl, (4) (C₅-C₁₄)heteroaryl substituted with one or more L^{1A} groups, (5) unsubstituted (C₅-C₁₄)heteroarylalkyl-, and (5) (C₅-C₁₄)heteroarylalkyl-substituted with one or more L^{1A} groups;
R⁸ is selected from the group consisting of unsubstituted cycloalkyl and cycloalkyl substituted with one or more L³ groups (wherein examples of said cycloalkyl groups (unsubstituted or substituted) include C₃-C₁₀ cycloalkyl rings);
each L³ is independently selected from the group consisting of: (1) —CN, (2) ═O, (3) —CH₂OH, (4) amino, (5) halo, (6) —CH₂NH₂, (7) —CH₂NHalkyl, (8) —C(O)OH, (9) -alkylene-C(O)NH(C₁ to C₆)alkyl, (10) -alkylene-C(O)N((C₁ to C₆)alkyl)₂ wherein each alkyl is independently selected, (11) -alkylene-C(O)NH(C₁ to C₆)haloalkyl, and (12) -alkylene-C(O)N((C₁ to C₆)haloalkyl)₂ wherein each alkyl is independently selected);
Ar is selected from the group consisting of: (1) unsubstituted aryl, (2) aryl substituted with one or more L^{1A} groups, (3) unsubstituted heteroaryl, and (4) substituted heteroaryl substituted with one or more L^{1A} groups;
each L¹ is independently selected from the group consisting of: halogen, alkyl (e.g., C₁-C₆ alkyl), —CN, —CF₃, —O—(C₁-C₆)alkyl, —O-(halo(C₁-C₆)alkyl), —C(O)—O—(C₁-C₆)alkyl, -alkylene-OH, halo(C₁-C₆)alkyl, hydroxyalkoxy-, alkoxyalkoxy-, and —S(O)₂(C₁-C₆)alkyl;
each L^{1A} is independently selected from the group consisting of: halogen, alkyl, —CN, —CF₃, —O—(C₁-C₆)alkyl, —O-(halo(C₁-C₆)alkyl), —C(O)—O—(C₁-C₆)alkyl, -alkylene-OH, halo(C₁-C₆)alkyl, hydroxyalkoxy-, alkoxyalkoxy-, and —S(O)₂(C₁-C₆)alkyl; and
n is 0, 1, 2 or 3.

2. The compound of claim 1 having a formula selected from the group consisting of: 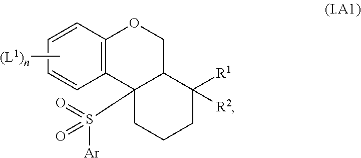
wherein at least one H in formula (I) is replaced by deuterium; 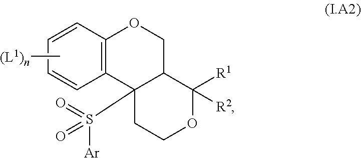
wherein at least one H in formula (I) is replaced by deuterium; 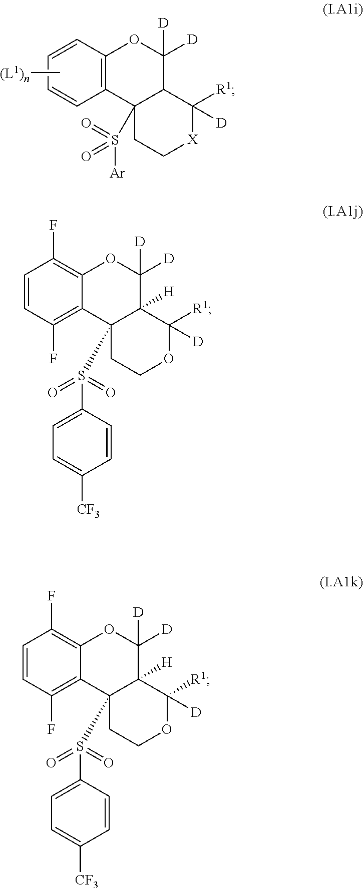
wherein 1 to 3H in the R¹ substituent are replaced with deuterium; 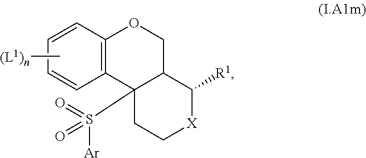
wherein 1 to 3H in the R¹ substituent are replaced with deuterium; 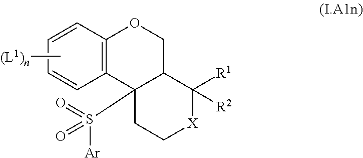
wherein R² is alkyl and 1 to 3H in said R² alkyl substituent are replaced with deuterium;

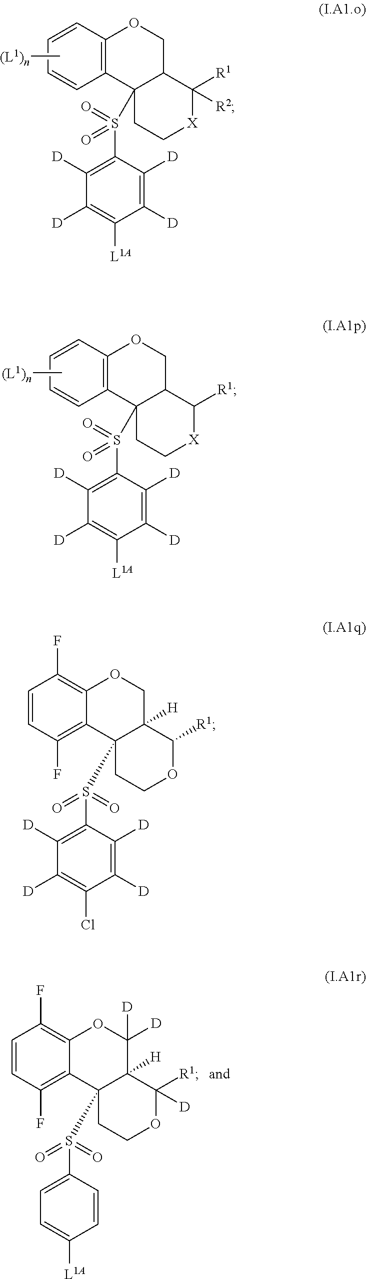

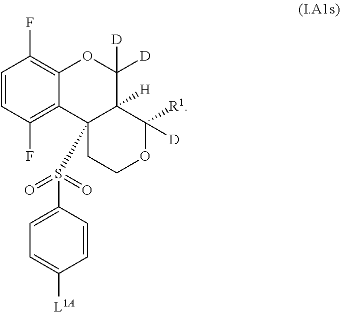

3. The compound of claim 1 selected from the group consisting of; 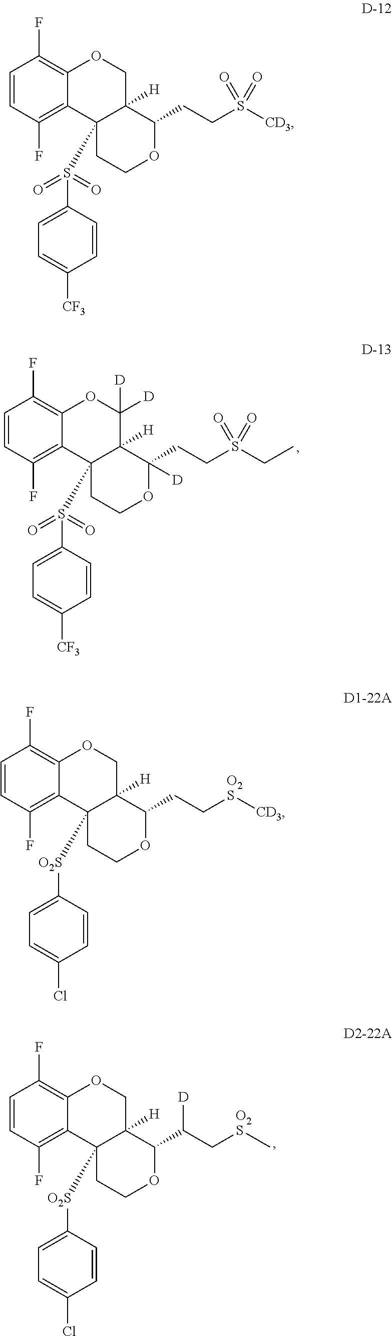 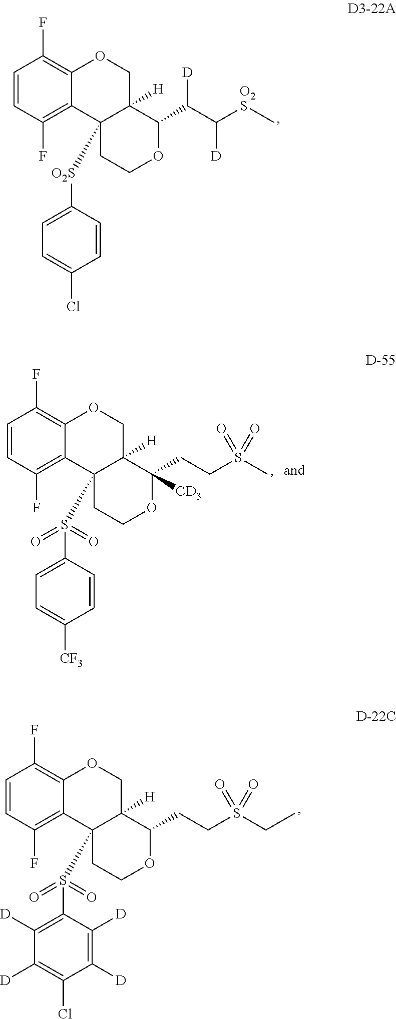
or a pharmaceutically acceptable salt or solvate thereof.

4. A pharmaceutical composition comprising an effective amount of at least one compound of claim 1 and at least one pharmaceutically acceptable carrier.

5. The pharmaceutical composition of claim 4, wherein said compound is selected from the group consisting of: D-12, D-13, D1-22A, D2-22A, D3-22A and D-55.

6. A method of inhibiting the deposition of beta amyloid protein in a patient in need of such treatment comprising administering to said patient a therapeutically effective amount of one or more compounds of claim 1.

7. The method of claim 6, wherein said one or more compounds is selected from the group consisting of compounds D-12, D-13, D1-22A, D2-22A, D3-22A and D-55.

8. A method of treating Alzheimer's disease in a patient in need of such treatment comprising administering to said patient a therapeutically effective amount of one or more compounds of claim 1.

9. The method of claim 8, wherein said one or more compounds is selected from the group consisting of compounds D-12, D-13, D1-22A, D2-22A, D3-22A and D-55.

10. A method of inhibiting the deposition of amyloid protein in a patient in need of such treatment comprising administering to said patient a therapeutically effective amount of one or more compounds of claim 1, and a therapeutically effective amount of one or more other drugs selected from the group consisting of: BACE inhibitors, muscarinic antagonists, cholinesterase inhibitors; gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors, cholesterol absorption inhibitors, and mTOR inhibitors.

11. The method of claim 10, wherein said compound is selected from the group consisting of compounds D-12, D-13, D1-22A, D2-22A, D3-22A and D-55.

12. A method of treating Alzheimer's disease in a patient in need of such treatment comprising administering to said patient a therapeutically effective amount of one or more compounds of claim 1, and a therapeutically effective amount of one or more other drugs selected from the group consisting of: BACE inhibitors, muscarinic antagonists, cholinesterase inhibitors; gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors, cholesterol absorption inhibitors, and mTOR inhibitors.

13. The method of claim 12, wherein said one or more compounds is selected from the group consisting of compounds D-12, D-13, D1-22A, D2-22A, D3-22A and D-55.
